(19) United States
(12) Patent Application Publication
(10) Pub. No.: US 2022/0033460 A1
(43) Pub. Date: Feb. 3, 2022

(54) IDENTIFICATION AND USE OF T CELL EPITOPES IN DESIGNING DIAGNOSTIC AND THERAPEUTIC APPROACHES FOR COVID-19

(71) Applicants: Repertoire Immune Medicines, Inc., Cambridge, MA (US); Repertoire Immune Medicines (Switzerland) Ltd., Schlieren (CH)

(72) Inventors: Daniel Pregibon, Cambridge, MA (US); Joshua Francis, Weymouth, MA (US); Del Leistritz-Edwards, Weston, MA (US); William Dunn, Boston, MA (US); Violeta Rayon Estrada, Brookline, MA (US); Gang Liu, Cary, NC (US); Jan Kisielow, Schlieren (CH); Franz-Josef Obermair, Oberengstringen (CH)

(21) Appl. No.: 17/397,841

(22) Filed: Aug. 9, 2021

Related U.S. Application Data
(63) Continuation of application No. PCT/US21/44063, filed on Jul. 30, 2021.
(60) Provisional application No. 63/059,144, filed on Jul. 30, 2020.
(60) Provisional application No. 63/089,487, filed on Oct. 8, 2020.
(60) Provisional application No. 63/148,475, filed on Feb. 11, 2021.
(60) Provisional application No. 63/154,878, filed on Mar. 1, 2021.
(60) Provisional application No. 63/155,107, filed on Mar. 1, 2021.
(60) Provisional application No. 63/178,377, filed on Apr. 22, 2021.
(60) Provisional application No. 63/178,383, filed on Apr. 22, 2021.

(51) Int. Cl. C07K 14/725; A61K 39/215; C12N 5/0783; C07K 14/74; A61K 35/17

(52) U.S. Cl. CPC C07K 14/7051; A61K 39/215; A61K 35/17; C07K 14/70539; C12N 5/0636

(57) ABSTRACT

Approaches for identifying T cell epitopes from SARS-CoV-2 are provided, along with the use of such T cell epitopes diagnostically and therapeutically. Compositions including T cell epitope vaccines and T cell epitope-display reagents are provided. Methods for identifying SARS-CoV-2 T cell epitopes, methods of identifying reactive T cells and methods of using epitopes and T cells for diagnostic purposes, such as identifying particular patient subpopulations are provided. Treatment methods, including administration of T cell epitope vaccines prophylactically and administration of activated T cells therapeutically are also provided.

### CROSS-REFERENCE TO RELATED APPLICATIONS

This application claims the benefit of and priority to U.S. Provisional Patent Application No. 63/059,144, filed on Jul. 30, 2020; U.S. Provisional Patent Application No. 63/089,487, filed on Oct. 8, 2020; U.S. Provisional Patent Application No. 63/178,377, filed on Apr. 22, 2021; U.S. Provisional Patent Application No. 63/148,475, filed on Feb. 11, 2021; U.S. Provisional Patent Application No. 63/154,878, filed on Mar. 1, 2021; U.S. Provisional Patent Application No. 63/155,107, filed on Mar. 1, 2021; and U.S. Provisional Patent Application No. 63/178,383, filed on Apr. 22, 2021, the disclosure of each of which is hereby incorporated by reference in its entirety for all purposes.

### SEQUENCE LISTING

The instant application contains a Sequence Listing which has been submitted electronically in ASCII format and is hereby incorporated by reference in its entirety. Said ASCII copy, created on Jul. 30, 2021, is named REPT-108WO_SL.txt and is 1,092,029 bytes in size.

### BACKGROUND OF THE INVENTION

Coronavirus Disease of 2019 (COVID-19) can lead to a severe acute respiratory syndrome (SARS) characterized by high fever, dry cough, fatigue, dyspnea, headache, frequent mild lymphopenia, hypoxemia, and characteristic pneumonia (Wu et al. (2020) Nature, 579:265-269; Lai et al. (2020) Int. J. Antimicrob. Agents, 55:105924; Zhou et al. (2020) Lancet, 395:P1054-1062). Meta-transcriptomic RNA sequencing of patient bronchoalveolar lavage fluid (BALF) or sputum has identified the likely causative agent as SARS-CoV-2, a novel betacoronavirus genus RNA virus related to SARS-CoV and SARS-MERS, which caused major SARS and Middle East Respiratory Syndrome (MERS) pandemics with 10%-30% mortality in the past 20 years (Wu et al. (2020) supra; Zhou et al. (2020) Nature, 579:270-273; Wu et al. (2020) Cell Host Microbe, 27:325-328; Chan et al. (2020) Lancet, 395:514-523; Grifoni et al. (2020) Cell Host Microbe, 27:1-10).

With over 190 million cases and over four million deaths across many countries in the short time from the diagnosis of the first patient in Wuhan, China in December 2019 to July 2021, COVID-19 could become the largest pandemic threat humankind has faced since the Spanish Flu (Johns Hopkins University Coronavirus Resource Center, 2021). High transmission rates, transmission by asymptomatic patients, high (˜15%) proportions of patients with severe disease, and mortality rates of up to 8% in some regions make this disease particularly dangerous. The elderly, individuals with co-morbidities, and an ill-understood subgroup of younger patients develop more severe disease with higher mortality rates (Lai et al. (2020) Int. J Antimicrob. Agents, 55:105924; Zhou et al. (2020) supra; Chan et al. (2020) Lancet, 395:514-523; Wang et al. (2020) Clin. Infect. Dis., 71:769-777).

The development of safe and effective therapies, in particular prophylactic vaccines, has become a critical tool in the management of the COVID-19 pandemic. Three different vaccines have been developed and approved for emergency use in the United States including two mRNA vaccines containing mRNA encoding viral spike glycoproteins of SARS-CoV-2 by Moderna and BioNTech/Pfizer, and an adenoviral vector vaccine modified to express the spike protein of SARS-CoV-2 by Johnson and Johnson. Nevertheless, multiple lines of evidence suggest important roles for T cells in productive immune responses to COVID-19. It has been observed that, in many SARS patients, B cell responses have been relatively short lived (1-2 years) and prone to antigen escape, raising the possibility of re-infection.

In contrast, T cell memory in survivors can be long-lived (>6-17 years) (Vabret et al. (2020) Immunity, 52:910-941; Zhao et al. (2016) Immunity, 44:1379-1391; Bert et al. (2020) bioRxiv 2020.2005.2026.115832). It is well known that T cells can engage antigen epitopes that are not targeted by B cells, including those derived from intracellular proteins, to provide broader protection which the virus can less easily circumvent through mutation (Zhao et al. (2016) Immunity, 44:1379-1391). T cells are especially necessary to clear severe virus infections. In addition to neutralizing antibody responses, a broad and long-lasting antiviral immunity requires the co-enrollment of CD4 and CD8 T cells and the generation of T cell memory (Zhao et al. (2016) Immunity, 44:1379-1391; Channappanavar and Perlman (2014) Immunol. Res. 59:118-128; Li et al. (2008) J. Immunol. 181:5490-5500; Vardhana and Wolchok (2020) J. Exp. Med., 217:e20200678; Channappanavar et al. (2014) J. Virol., 88:11034-11044).

Accordingly, there remains a need for identification of SARS-CoV-2 T cell epitopes, as well as approaches for using such epitopes in the diagnosis and treatment of COVID-19.

### SUMMARY OF THE INVENTION

The disclosure provides identified, isolated peptides comprising T cell epitopes from SARS-CoV-2 (see, e.g., TABLE 1 and TABLE 2, hereinbelow) together with an identification of the MHC class I molecules on antigen presenting cells that present the peptides to corresponding TCRs on CD8+ T cells. The disclosure also provides identified, isolated peptides comprising T cell epitopes from SARS-CoV-2 (see, e.g., TABLE 3 hereinbelow) together with an identification of the MHC class II molecules on antigen presenting cells that present the peptides to corresponding TCRs on CD4+ cells. The studies disclosed herein show the relationship of specific T cell epitopes to specific MHC molecules on antigen presenting cells and specific T cell receptors on specific T cells, which heretofore has not been possible on such a scale.

In one aspect, the disclosure provides an isolated peptide comprising a SARS-CoV-2 T cell epitope comprising an amino acid sequence set forth in TABLE 1, wherein the peptide is no more than 100 amino acids in length, and an optional pharmaceutically acceptable carrier. The T cell epitope can be a CD8+ epitope. In certain embodiments, the T cell epitope comprises an amino acid sequence set forth in TABLE 2. T cell epitope can be specific for a subject infected with SARS-CoV-2.

In another aspect, the disclosure provides a SARS-CoV-2 T cell epitope comprising an amino acid sequence set forth in TABLE 3, wherein the peptide is no more than 100 amino acids in length, and an optional pharmaceutically acceptable carrier. The T cell epitope can be a CD4+ epitope.

In another aspect, the disclosure provides an isolated peptide comprising a SARS-CoV-2 T cell epitope, wherein the T cell epitope comprises at least 13, at least 14, at leat 15, at least 16, at least 17, or at least 18 continuous amino acids of an epitope sequence set forth in TABLE 3 or at least 8 continuous amino acids of an epitope sequence set forth in TABLE 1 or TABLE 2, wherein the peptide is no more than 100 amino acids in length, or a pharmaceutically acceptable salt thereof.

In each of the foregoing aspects, the peptide is synthetic. Furthermore, the peptide can be no more than 50, 40, 30, or 20 amino acids in length. The amino acid sequence of each of the peptides consists essentially of or consists of an amino acid sequence set forth in (i) TABLE 1, (ii) TABLE 2, or (iii) TABLE 3. Under certain circumstances, the isolated peptide comprises an amino acid sequence set forth in TABLE 1 or TABLE 2, or at least 8 continuous amino acids thereof, and is presentable by a major histocompatibility complex (MHC) Class I molecule. Similarly, under certain circumstances, peptide comprises an amino acid sequence set forth in TABLE 3, or at least 13 continuous amino acids thereof, and is presentable by a MHC Class II molecule.

In another aspect, the disclosure provides a pharmaceutical composition comprising a peptide, e.g., a synthetic peptide, disclosed herein and a pharmaceutically acceptable carrier or excipient. The pharmaceutical composition optionally comprises a plurality of peptides (e.g., 2, 3, 4, 5, 6, 7, 8, 9, 10, or more) disclosed herein and a pharmaceutically acceptable carrier or excipient.

In another aspect, the disclosure provides a pharmaceutical composition comprising a nucleic acid, e.g., a synthetic nucleic acid, encoding the peptide disclosed herein and a pharmaceutically acceptable carrier or excipient. The pharmaceutical composition comprises one or more nucleic acids encoding a plurality of peptides (e.g., 2, 3, 4, 5, 6, 7, 8, 9, 10, or more) disclosed herein and a pharmaceutically acceptable carrier or excipient.

Each of the foregoing pharmaceutical compositions, can comprise liposome or lipid nanoparticle, wherein the peptide or nucleic acid encoding the peptide is disposed within the liposome or lipid nanoparticle. The pharmaceutical optionally further comprises an immunogenicity enhancing adjuvant.

In another aspect, the disclosure provides a vaccine that stimulates a T cell mediated immune response when administered to a subject, the vaccine comprising one of the foregoing peptides or pharmaceutical compositions. The vaccine can be a priming vaccine, a booster vaccine, or can function as both a priming vaccine and a booster vaccine. The vaccine can be a pan-coronavirus vaccine which is capable of eliciting an immune response against a plurality of coronaviruses, where one of the viruses can be SARS-CoV-2.

In each of the foregoing pharmaceutical compositions or vaccines, the compositions or vaccines can comprise one or more CD4 epitopes (i.e., T cell epitopes that is presentable by an MHC class II and capable of stimulating a CD4+ T cell response), one or more CD8 epitopes (i.e., T cell epitopes that is presentable by an MHC class I and capable of stimulating a CD8+ T cell response), or one or more CD4 epitopes and one or more CD8 epitopes.

In another aspect, the disclosure provides a method of stimulating a T cell immune response to SARS-CoV-2 in a subject in need thereof. The method comprises administering to the subject an effective amount of any one of the foregoing pharmaceutical compositions or vaccines. In certain embodiments, the subject expresses an MHC Class I and/or an MHC Class II that binds the epitope.

In another aspect, the disclosure provides a method of presenting a T cell epitope on the surface of an antigen-presenting cell (APC). The method comprises contacting the APC in vitro with any one or more of the peptides disclosed herein, wherein the APC expresses the MHC Class II. In another aspect, the disclosure provides a method of presenting a T cell epitope on the surface of an APC. The method comprises transfecting the APC in vitro with one or more of nucleic acids (e.g., mRNAs) encoding one or more of the peptides disclosed herein, wherein the APC expresses the MHC Class II. The disclosure also provides an antigen presenting cell (APC) produced by any one of the foregoing methods. The APC can be a dendritic cell, monocyte, macrophage or B cell. Alternatively, the APC can be an artificial APC.

The disclosure also provides a composition comprising one of the foregoing peptides and a cognate MHC Class II molecule (e.g. HLA type DPA1*02:02 DPB1*05:01, DRB1*07:01, DRB1*14:05, DRB1*11:01, and DRB1*08:03, e.g., as set forth in TABLE 3) or an extracellular portion thereof, wherein the peptide and the MHC Class II, or the extracellular portion thereof, are combined in a complex. The disclosure also provides a method of producing activated T cells, wherein the method comprises contacting a population of T cells in vitro with such an APC or with such a complex to permit activation of one or more T cells in the population for reactivity to a SARS-CoV-2 infected cell. A population of activated T cells produced by the method is also provided. The population of T cells can comprise CD4⁺ T cells. The T cells can be cultured to facilitate expansion of the T cells in the population reactive to a SARS-CoV-2 infected cell.

In another aspect, the disclosure provides a method of stimulating a T cell immune response to SARS-CoV-2 in a subject in need thereof. The method comprises administering to the subject a composition comprising the population of such activated T cells, wherein the subject expresses the MHC Class II.

In each of the foregoing methods, APCs, compositions, or activated T cells, it is contemplated that (a) the peptide comprises the amino acid sequence of SEQ ID NO: 688, and the MHC Class II is HLA-DPA1*02:02 or HLA-DPB1*05:01; (b) the peptide comprises the amino acid sequence of SEQ ID NO: 689, and the MHC Class II is FILA-DRB1*07:01; (c) the peptide comprises the amino acid sequence of SEQ ID NO: 690, and the MHC Class II is HLA-DRB1*07:01; (d) the peptide comprises the amino acid sequence of SEQ ID NO: 691, and the MHC Class II is HLA-DRB1*07:01; (e) the peptide comprises the amino acid sequence of SEQ ID NO: 692, and the MHC Class II is HLA-DRB1*07:01; (f) the peptide comprises the amino acid sequence of SEQ ID NO: 693, and the MHC Class II is HLA-DRB1*14:05; (g) the peptide comprises the amino acid sequence of SEQ ID NO: 694, and the MHC Class II is HLA-DRB1*11:01; and/or (h) the peptide comprises the amino acid sequence of SEQ ID NO: 695, and the MHC Class II is HLA-DRB1*08:03.

In each of the foregoing methods, the T cells are autologous and/or could be obtained from a healthy donor.

In another aspect, the disclosure provides a method of presenting a T cell epitope on the surface of an APC. The method comprising contacting the APC in vitro with a peptide disclosed herein or a nucleic acid (e.g., mRNA) encoding a peptide disclosed herein, wherein the APC expresses the MHC Class I. The disclosure provides an APC produced by any one of the foregoing methods. The APC can be a dendritic cell, monocyte, macrophage or B cell. Alternatively, the APC can be an artificial APC. Also provided is a composition comprising a peptide disclosed herein and an MHC Class I (e.g. HLA type A*01:01, A*02:01, A*24:02, A*32:01, B*07:02, or B*48:01, e.g., as set forth in TABLE 1 or 2), wherein the peptide and the MHC Class I are combined in a complex.

The disclosure provides a method of producing activated T cells. The method comprises contacting a population of T cells in vitro with such an APC or complex to permit activation of one or more T cells in the population for reactivity to a SARS-CoV-2 infected cell. A population of activated T cells produced by the method is also provided. The T cells can comprise CD8⁺ T cells. The T cells can be cultured to facilitate expansion of the T cells in the population reactive to a SARS-CoV-2 infected cell. The disclosure also provides a population of activated T cell produced by one of more of the foregoing methods.

The disclosure provides a method of stimulating a T cell immune response to SARS-CoV-2 in a subject in need thereof. The method comprises administering to the subject an effective amount of a composition comprising the population of such activated T cells wherein the subject expresses MHC Class I.

In each of the foregoing methods, APCs, compositions, or populations of activated T cells, (a) the peptide comprises the amino acid sequence of SEQ ID NO: 328, and the MHC Class I is HLA-A*01:01; (b) the peptide comprises the amino acid sequence of SEQ ID NO: 286, and the MHC Class I is HLA-A*02:01; (c) the peptide comprises the amino acid sequence of SEQ ID NO: 327, and the MHC Class I is HLA-A*01:01; (d) the peptide comprises the amino acid sequence of SEQ ID NO: 326, and the MHC Class I is HLA-B*07:02; (e) the peptide comprises the amino acid sequence of SEQ ID NO: 324, and the MHC Class I is HLA-B*07:02; and/or (0 the peptide comprises the amino acid sequence of SEQ ID NO: 288, and the MHC Class I is HLA-A*02:01.

In each of the foregoing methods, the T cells are autologous and/or could be obtained from a healthy donor.

In another aspect, the disclosure provides a composition comprising an isolated APC that presents on an outer cell surface of the APC a peptide disclosed herein. In certain embodiments, the composition comprises a plurality (e.g., 2, 3, 4, 5, 6, 7, 8, 9, 10, 11, 12, 13, 14, 15, 16, 17, 18, 19, 20, or more) of such APCs that present different peptides.

In certain embodiments, the isolated APC presents on an outer cell surface of the APC a peptide comprising a SARS-CoV-2 T cell epitope comprising an amino acid sequence set forth in TABLE 1, wherein the peptide is no more than 100 amino acids in length, and an optional pharmaceutically acceptable carrier. In certain embodiments, the T cell epitope is a CD8+ epitope with an amino acid sequence set forth in TABLE 2 and is presented by major histocompatibility complex (MHC) class I on the surface of the APC. In certain embodiments, the T cell epitope is specific for a subject infected with SARS-CoV-2.

In certain embodiments, the composition further comprises a second, different APC that presents on its outer cell surface of the APC a second, different peptide comprising a SARS-CoV-2 T cell epitope, wherein the second, different epitope optionally comprises an amino acid sequence set forth in any one of TABLE 1-3, and wherein the peptide is no more than 100 amino acids in length. In each of the foregoing APC compositions the T cell epitope comprises at least 8 continuous amino acids of an epitope sequence set forth in TABLE 1 or 2.

In certain embodiments, the isolated APC presents on an outer cell surface of the APC a peptide comprising a SARS-CoV-2 T cell epitope comprising an amino acid sequence set forth in TABLE 3, wherein the peptide is no more than 100 amino acids in length, and an optional pharmaceutically acceptable carrier. In the foregoing APC, the T cell epitope is a CD4+ T cell epitope and can be presented by a MHC class II molecule at the surface of the APC. In some embodiments, the T cell epitope comprises at least 13 continuous amino acids of an epitope sequence set forth in TABLE 3.

In certain embodiments, the composition further comprises a second different APC that presents on its outer cell surface of the APC a second, different peptide comprising a SARS-CoV-2 T cell epitope, wherein the second, different epitope optionally comprises an amino acid sequence set forth in any one of TABLES 1-3, and wherein the peptide is no more than 100 amino acids in length. In some embodiments, the peptide is no more than 30 amino acids in length or 20 amino acids in length. In any of the above composition the peptide is can be synthetic. The APC can be a dendritic cell, monocyte, macrophage or B cell. Alternatively, the APC can be an artificial APC.

In another aspect, the disclosure provides a pharmaceutical composition comprising any of the APC compositions disclosed herein and a pharmaceutically acceptable carrier.

In another aspect, the disclosure provides a composition comprising an isolated T cell that binds a peptide disclosed herein, optionally as presented by a cognate MHC disclosed herein. In certain embodiments, the composition comprises a plurality (e.g., 2, 3, 4, 5, 6, 7, 8, 9, 10, or more) of such T cells that are clonally different. In certain embodiments, the composition comprises such T cells that bind a plurality (e.g., 2, 3, 4, 5, 6, 7, 8, 9, 10, 11, 12, 13, 14, 15, 16, 17, 18, 19, 20, or more) of different peptides disclosed herein.

In certain embodiments, the T cell binds a peptide comprising a SARS-CoV-2 T cell epitope comprising an amino acid sequence set forth in TABLE 1, wherein the peptide is no more than 100 amino acids in length, and an optional pharmaceutically acceptable carrier. In certain embodiments, the T cell epitope is a CD8+ epitope with an amino acid sequence set forth in TABLE 2. In certain embodiments, the T cell epitope is specific for a subject infected with SARS-CoV-2. In each of the foregoing T cell compositions the T cell epitope can comprise at least 8 continuous amino acids of an epitope sequence set forth in TABLE 1 or 2 and the T cell can be a CD8+ T cell.

In certain embodiments, the composition further comprises a second different T cell that binds a second, different peptide comprising a SARS-CoV-2 T cell epitope, wherein the second, different epitope optionally comprises an amino acid sequence set forth in any one of TABLES 1-3, and wherein the peptide is no more than 100 amino acids in length.

In certain embodiments, the T cell binds a peptide comprising a SARS-CoV-2 T cell epitope comprising an amino acid sequence set forth in TABLE 3, wherein the peptide is no more than 100 amino acids in length, and an optional pharmaceutically acceptable carrier. In certain embodiments, the T cell epitope is a CD4+ epitope. In each of the foregoing T cell compositions the T cell epitope can comprise at least 13 continuous amino acids of an epitope sequence set forth in TABLE 3 and the T cell can be a CD4+ T cell.

In certain embodiments, the composition further comprises a second different T cell that binds a second, different peptide comprising a SARS-CoV-2 T cell epitope, wherein the second, different epitope optionally comprises an amino acid sequence set forth in any one of Tables 1-3, and wherein the peptide is no more than 100 amino acids in length.

In certain embodiments, the composition comprises a second different T cell that binds a second, different peptide comprising a SARS-CoV-2 T cell epitope, wherein the second, different epitope optionally comprises an amino acid sequence set forth in any one of TABLES 1-3, and wherein the peptide is no more than 100 amino acids in length. In some embodiments the peptide is no more than 30 amino acids in length or 20 amino acids in length. In any of the foregoing T cell compositions the peptide can be synthetic. In any of the foregoing T cell compositions the APC can be a dendritic cell, monocyte, macrophage, B cell or an artificial APC.

In another aspect, the disclosure provides a pharmaceutical composition comprising a T cell disclosed herein and a pharmaceutically acceptable carrier.

In another aspect, the disclosure provides the use of SARS-CoV-2 T cell epitopes identified by the methods described herein for designing T cell mediated therapies to treat COVID-19. For example, an identified SARS-CoV-2 T cell epitope can be used to determine the TCR sequence(s) that recognizes that epitope, and the TCR sequence(s) can then be used to design recombinant T cell therapies described hereinbelow.

In another aspect, the disclosure provides a T cell receptor (TCR), for example, an engineered TCR, having antigenic specificity for a SARS-CoV-2 antigen, the TCR have an alpha chain and a beta chain, wherein the TCR comprises corresponding CDR3 alpha and CDR3 beta sequences set forth in Table 5.

In certain embodiments, the TCR further comprises CDR1 alpha and CDR2 alpha sequences defined by the corresponding, respective alpha V gene, and CDR1 beta and CDR2 beta sequences defined by the corresponding, respective beta V gene as set forth in TABLE 5. In certain embodiments, the SARS-CoV-2 antigen is an T cell epitope.

In certain embodiments, the T cell epitope is a CD8+ T cell epitope. In certain embodiments, the TCR has antigenic specificity for the corresponding SAR-CoV-2 epitope set forth in TABLE 1. In certain embodiments, the TCR has antigenic specificity restricted by the corresponding HLA class set forth in TABLE 1.

In certain embodiments, the T cell epitope is a CD4+ T cell epitope. In certain embodiments, the TCR has antigenic specificity for the corresponding SAR-CoV-2 epitope set forth in TABLE 3. In certain embodiments, the TCR has antigenic specificity restricted by the corresponding HLA class set forth in TABLE 3.

In certain embodiments, the TCR is disposed on the surface of a T cell.

In another aspect, the disclosure provides a soluble TCR comprising the alpha chain variable region and the beta chain variable region of a TCR disclosed herein, wherein the soluble TCR does not comprise a functional transmembrane domain.

In another aspect, the disclosure provides a pharmaceutical composition comprising a TCR or soluble TCR disclosed herein and a pharmaceutically acceptable carrier.

In another aspect, the disclosure provides an engineered T cell, wherein the engineered T cell is transduced with one or more exogenous nucleic acid sequences that encode an engineered TCR disclosed herein.

In certain embodiments, the T cell is a CD8+ T cell. In certain embodiments, the T cell is a CD4+ T cell. In certain embodiments, the T cell is an autologous cell. In certain embodiments, the T cell is an allogeneic cell.

In another aspect, the disclosure provides a pharmaceutical composition comprising a T cell disclosed herein a pharmaceutically acceptable carrier.

In another aspect, the disclosure provides a method of ameliorating a symptom of SARS-CoV-2 infection in a subject in need thereof, the method comprising administering to the subject an effective amount of a pharmaceutical composition disclosed herein, thereby to ameliorate the symptom.

In another aspect, the disclosure provides a SARS-CoV-2 T cell epitope library comprising at least 500 peptide moieties, wherein said library comprises peptides moieties containing identified mutations in SARS-Co-V2 spike protein and optionally peptide moieties from at least one of the following categories:
(a) 8mer-12mer peptides (e.g., 9mer peptides) of SARS-CoV-2 full proteome (e.g., peptide having an IC50, measure or predicted, of less than 500 nM for one or multiple MHC alleles);
(b) peptides of SARS-CoV comprising a sequence at least 90% (or at least 95%, 96%, 97%, 98% or 99%) identical to homologous SARS-CoV-2 sequences;
(c) peptides from common cold coronaviruses;
(d) peptides comprising immunodominant epitopes of SARS-CoV (e.g., identified from the Immune Epitope Database (IEDB);
(e) SARS-Co-V2 peptides with predicted glycosylation sites;
(f) peptide highly conserved across multiple coronavirus species or strains;
(g) peptides of non-structural proteins with low observed mutation rates;
(h) peptides against which T cell reactivity has been detected in abundance in patients with mild disease but not severe disease (e.g., patient that perished or required ventilation);
(i) peptides against which T cell reactivity has been detected in abundance in asymptomatic patients but not symptomatic patients; and
(j) peptides that show T cell reactivity with broad clonal diversity in recovered patients.

In certain embodiments, the library comprises 9-mer peptides of SARS-CoV-2 full proteome. The 9-mer peptides optionally have an IC₅₀ of less than 500 nM.

In another aspect, the disclosure provides a MHC multimer library, where the library comprising MHC multimers loaded with the foregoing SARS-CoV-2 T cell epitope library. The MHC multimer library can comprise MHC Class I multimers and/or MHC Class II multimers. In certain embodiments, the disclosure provides a kit for identifying a T cell reactive to a SARS-CoV-2 T cell epitope. The kit comprises such an MHC multimer library packaged with instructions for use of the library so as to identify a T cell reactive to a SARS-CoV-2 T cell epitope.

The disclosure provides a method of identifying a T cell reactive to a SARS-CoV-2 T cell epitope. The method comprises contacting a sample of T cells with such a MHC multimer library and identifying a T cell within the sample that binds to at least one member of the MHC multimer library to thereby identify a T cell reactive with a SARS-CoV-2 T cell epitope. The disclosure also provides a method of identifying a SARS-CoV-2 T cell epitope. The method comprises contacting a T cell sample with such a MHC multimer library, identifying a T cell that binds to at least one member of the MHC multimer library, and determining the sequence of the peptide loaded onto the MHC multimer to which the T cell binds to thereby identify a SARS-CoV-2 T cell epitope. The disclosure provides a method of identifying a T cell immune response in a COVID-19 subject. The method comprises contacting a sample of T cells from the COVID-19 subject with such an MHC multimer library and identifying a T cell within the sample that binds to at least one member of the MHC multimer library to thereby identify a T cell immune response in the COVID-19 subject. The methods optionally further comprise determining the sequence of the peptide(s) loaded onto the MHC multimer(s) to which the T cell binds to thereby determine the antigenic specificity of the T cell response in the COVID-19 subject. Alternatively or in addition, the method further comprises selecting a treatment regimen for the subject with COVID-19 based on the antigenic specificity of the T cell response in the subject.

In another aspect, the disclosure provides a method of determining whether a subject has COVID-19. The method comprises detecting the presence and/or amount of (i) one or more peptides disclosed herein and/or (ii) T cells reactive with one or more peptides of any of the peptides disclosed herein, in a sample harvested from the subject thereby to determine whether the subject has COVID-19.

In another aspect, the disclosure provides a method of determining the potential severity of a COVID-19 infection in a subject. The method comprises detecting the presence and/or amount of (i) one or more peptides disclosed herein and/or (ii) T cells reactive with one or more peptides disclosed herein, in a sample harvested from the subject thereby to determine the potential severity of the COVID-19 infection. The method optionally further comprises selecting a treatment regimen based upon the potential severity of the COVID-19 infection.

In another aspect, the disclosure provides a method of determining therapeutic intervention of a subject with COVID-19. The method comprises detecting the presence and/or amount of one or more peptides disclosed herein in a sample harvested from the subject, wherein the presence and/or amount of the peptides is used to determine the therapeutic intervention for the subject.

In each of the foregoing methods of determining whether a subject has COVID-19, determining the potential severity of a COVID-19 infection, or determining therapeutic intervention of a subject with COVID-19, the presence or amount of the T cells can be determined by a PCR reaction, tetramer assay, Enzyme Linked Immuno Spot Assay (ELISpot), or an Activation Induced Marker (AIM) assay; the presence or amount of the peptide can be determined by an assay using binding moieties (e.g., antibody or soluble TCR that binds the peptide, optionally as presented by a cognate MHC, for example, on an outer surface of a cell) or by mass spectrometry. In certain embodiments, the sample is a tissue or body fluid sample harvested from the subject.

For a fuller understanding of the nature and advantages of the present disclosure, reference should be had to the ensuing detailed description taken in conjunction with the accompanying figures. The present disclosure is capable of modification in various respects without departing from the present disclosure. Accordingly, the figures and description of these embodiments are not restrictive.

### BRIEF DESCRIPTION OF THE FIGURES

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 exemplifies various click chemistry handles and reactions.

FIG. 2 illustrates various peptide exchange methods for HLA molecules.

FIG. 3A-3E show an exemplary SDS-PAGE or Western Blot analysis of conjugation reactions. Cartoon images depict SAv tetramer linked to one, two, three or four HLA molecules. Arrows indicate undesired side-products. FIG. 3A: Anti-His Western Blot analysis of SAv-conjugation reaction. A description of each lane is shown in the table. The extent of reaction is approximately 94-97% based on comparison with reference SA protein. FIG. 3B: SDS-PAGE image of HLA-A2-DBCO-SAv-Az. Lane 1: SeeBlue Plus Protein Standard, Lane 2: SA-Az (non-boiled), Lane 3: SA-Az (boiled) Lane 4: HLA-A2-DBCO-SAv-Az (non-boiled, non-reduced), Lane 5: HLA-A2-DBCO-SAv-Az (boiled, reduced). FIG. 3C: SDS-PAGE image of HLA-A2-Az-SAv-DBCO. Lane 1: SeeBlue Plus Protein Standard, Lane 2: HLA-A2-Az (non-boiled), Lane 3: HLA-A2-Az-SAv-DBCO, (non-boiled), Lane 4-7: HLA-A2-Az-SAv-DBCO reactions (non-boiled). FIG. 3D: SDS-PAGE image of HLA-A2-Alk-SAv-Az. Lane 1: SeeBlue Plus Protein Standard, Lane 3: HLA-A2-Alk-SAv-Az (non-boiled, non-reduced), Lane 5: HLA-A2-Alkyne-SAv-Az (boiled, reduced). FIG. 3E: SDS-PAGE images of HLA-A*01:01, HLA-A*03:01 and HLA-A*24:02 in the Conjugated Tetramer format. Samples were either non-boiled/non-reduced (NB/NR) or boiled/reduced (boiled/R).

FIG. 4. SDS-PAGE analysis of the intein splicing reaction between HLA-A2-N-intein/β2m/peptide complex and SAv-C-intein.

FIGS. 5A and 5B illustrates UV exchange monitored by differential scanning fluorimetry. FIG. 5A shows differential scanning fluorimetry (DSF) of HLA-A*02:01-Alk-SAv-Az Conjugated Tetramers produced as in Example 1 containing a placeholder GILGFVFJL peptide (SEQ ID NO:7), or after UV-exchange in the presence of excess NLVPMVATV peptide (SEQ ID NO:8), showing a 20° C. increase in stability indicative of exchange to a higher affinity peptide. FIG. 5B is a DSF of HLA-A*02 biotin-mediated tetramers produced by UV exchange on the monomer followed by tetramerization, or by UV exchange on the tetramer itself, and confirms that multimeric state has no impact on the efficiency of UV-exchange, and that multimers of the current invention have the same stability as the industry standard pMHC.

FIGS. 6A-6F depict flow cytometry after peptide exchange on biotinylated HLA-A*02 monomers and tetramers. Donor PBMCs expanded with NLVPMVGTV peptide (SEQ ID NO: 9) were stained with: Anti-CD8-BV785 and Anti-Flag-APC secondary only (FIG. 6A), 50 nM HLA-A*02 biotin-mediated tetramers loaded with placeholder peptide GILGFVFJL (SEQ ID NO:7) (FIG. 6B), 50 nM HLA-A*02 biotin-mediated tetramers refolded with NLVPMVATV peptide (SEQ ID NO:8) (FIG. 6C), 50 nM HLA-A*02 biotin-mediated tetramers loaded with NLVPMVATV peptide (SEQ ID NO: 8) via UV exchange on the monomeric form, followed by tetramerization with streptavidin (FIG. 6D), 50 nM HLA-A*02 biotin-mediated tetramers loaded with NLVPMVATV peptide (SEQ ID NO:8) via UV exchange on the tetrameric form itself (FIG. 6E) and 50 nM HLA-A*02 biotin-mediated tetramers loaded with NLVPMVATV peptide (SEQ ID NO: 8) via dipeptide exchange on the tetrameric form itself (FIG. 6F).

FIGS. 7A-7B depict flow cytometry after UV exchange on HLA-A*02:01-Alk-SAv-Az Conjugated Tetramers. Donor PBMCs expanded with NLVPMVATV peptide (SEQ ID NO: 8) were stained with: Anti-streptavidin-PE and Anti-Flag-APC secondaries only (FIG. 7A) or 1 nM HLA-A*02:01-Alk-SAv-Az Conjugated Tetramers loaded with NLVPMVATV peptide (SEQ ID NO: 8) via UV exchange directly on the tetrameric form (FIG. 7B).

FIGS. 8A-8C depict a comparison of ELISA and DSF as stability tests of UV-exchanged HLA-A*02 Tetramers. Specifically, FIG. 8A depicts an ELISA analysis of HLA-A*02:01-Alk-SAv-Az Conjugated Tetramers UV-exchanged to a 192-member peptide panel representing altered peptide ligands (APL) of the NLVPMVATV peptide (SEQ ID NO: 8). ELISA OD is plotted versus the netMHC predicted IC50 for each peptide. Different peptides span a range of ELISA signals. FIG. 8B shows DSF curves for a subset of NLVPMVATV (SEQ ID NO: 8) APL peptides UV-exchanged into biotin-mediated tetramers, demonstrating a span of stabilities. FIG. 8C shows a DSF/ELISA correlation for a subset of NLVPMVATV (SEQ ID NO: 8) APL peptides UV-exchanged into biotin-mediated tetramers.

FIGS. 9A-9D depict quality control analysis of HLA-A*01:01-Alk-SAv-Az Conjugated Tetramers. Specifically, FIG. 9A depicts an analytical SEC chromatogram of HLA-A*01:01 tetramers with low aggregate. FIG. 9B depicts an SDS-PAGE of HLA-A*01:01-Alk-SAv-Az Conjugated Tetramers non-boiled/non-reduced (NB/NR) or boiled/reduced (Boiled/R). FIG. 9C depicts DSF of HLA-A*01:01-Alk-SAv-Az Conjugated Tetramers loaded with placeholder peptide STAPGJLEY (SEQ ID NO: 16) (No UV), or after UV-exchange in the absence (UV no peptide) or presence (UV+VTEHDTLLY (SEQ ID NO: 10)) of rescue peptide. FIG. 9D depicts flow cytometry data for PBMC's expanded with VTEHDTLLY peptide (SEQ ID NO: 10), and stained with 20 nM HLA-A*01:01 biotin-mediated tetramers loaded with VTEHDTLLY peptide (SEQ ID NO: 10) by refolding (Refold VTE), HLA-A*01:01-Alk-SAv-Az Conjugated Tetramers loaded with STAPGJLEY (SEQ ID NO: 16) (No UV), or HLA-A*01:01-Alk-SAv-Az Conjugated Tetramers after UV-exchange in the presence of rescue peptide VTEHDTLLY (SEQ ID NO: 10) (UV+VTE). Both the fraction of tetramer positive cells (% Tetramer+) and mean fluorescence intensity (MFI) are depicted.

FIGS. 10A-10D depict quality control analysis of HLA-A*24:02-Alk-SAv-Az Conjugated Tetramers. Specifically, FIG. 10A depicts an analytical SEC chromatogram of HLA-A*24:02 tetramers with low aggregate. FIG. 10B depicts an SDS-PAGE of HLA-A*24:02-Alk-SAv-Az Conjugated Tetramers non-boiled/non-reduced (NB/NR) or boiled/reduced (Boiled/R). FIG. 10C depicts DSF of HLA-A*24:02-Alk-SAv-Az Conjugated Tetramers loaded with placeholder peptide VYGJVRACL (SEQ ID NO: 11) (No UV), or after UV-exchange in the absence (UV no peptide) or presence (UV+QYDPVAALF (SEQ ID NO: 12)) of rescue peptide. FIG. 10D depicts flow cytometry data for PBMC's expanded with QYDPVAALF peptide (SEQ ID NO: 12), and stained with secondary only, 20 nM HLA-A*24:02 biotin-mediated tetramers loaded with QYDPVAALF peptide (SEQ ID NO: 12) by refolding (Refold QYD), 20 nM HLA-A*24:02-Alk-SAv-Az Conjugated Tetramers loaded with VYGJVRACL (SEQ ID NO: 11) (No UV), or 20 nM HLA-A*24:02-Alk-SAv-Az Conjugated Tetramers after UV-exchange in the presence of rescue peptide QYDPVAALF (SEQ ID NO: 12) (UV+QYD). Both the fraction of tetramer positive cells (% Tetramer+) and mean fluorescence intensity (MFI) are depicted.

FIGS. 11A-11C depict quality control analysis of HLA-B*07:02-Alk-SAv-Az Conjugated Tetramers. Specifically, FIG. 11A depicts an analytical SEC chromatogram of HLA-B*07:02 tetramers with no aggregate. FIG. 11B depicts an SDS-PAGE of HLA-B*07:02-Alk-SAv-Az Conjugated Tetramers non-boiled/non-reduced (NB/NR). FIG. 11C depicts flow cytometry data for PBMC's expanded with RPHERNGFTVL peptide (SEQ ID NO: 13), and stained with secondary only, 20 nM HLA-B*07:02 biotin-mediated tetramers loaded with RPHERNGFTVL peptide (SEQ ID NO: 13) by refolding (Refold RPH), 20 nM HLA-B*07:02-Alk-SAv-Az Conjugated Tetramers loaded with AARGJTLAM (SEQ ID NO: 14), (No UV), or 20 nM HLA-B*07:02-Alk-SAv-Az Conjugated Tetramers after UV-exchange in the presence of rescue peptide RPHERNGFTVL (SEQ ID NO: 13), (UV+RPH). Both the fraction of tetramer positive cells (% Tetramer+) and mean fluorescence intensity (MFI) are depicted.

FIG. 12 depicts labeling HLA-A*02:01-Alk-SAv-Az Conjugated Tetramers with an identifying oligonucleotide tag. HLA-A*02:01-Alk-SAv-Az Conjugated Tetramers produced as described in Example 1 were incubated with 5′ biotinylated oligonucleotides and separated by Western probed with anti-Flag antibody. Shifted bands upon oligo addition indicated tetramer labeling.

FIG. 13 shows single cell sequencing of barcoded HLA-A*02:01-Alk-SAv-Az APL libraries. A heatmap of pMHC binding to individual T cells identified by single cell sequencing. Columns representing 2008 individual cells were clustered by TCR clonotype, and rows represent each of 192 APL variants of NLVPMATV (SEQ ID NO: 8). Warm colors indicate strong pMHC-TCR interactions read out by the identifying oligonucleotide tag.

FIG. 14 depicts PCR amplification of peptide-encoding template onto hydrogels under single template conditions. PCR was conducted on hydrogel beads either in bulk or after encapsulation in drops under single template conditions. Supernatant released upon breaking droplets after PCR was run next to product released from beads by Xbal or mock digest.

FIG. 15 shows the verification of single template amplification in drops. Hydrogels after PCR amplification of template in bulk or in drops under single template conditions were stained with streptavidin-PE. Fluorescent hydrogels were quantified relative to total hydrogels to confirm single template conditions.

FIGS. 16A-16B depict loading of HLA-A*02:01-Alk-SAv-Az Conjugated Tetramers onto PCR-amplified hydrogels. Signal to noise ratios for hydrogels stained with anti-Flag-APC or anti-β2M-Alexa488 after loading with Conjugated Tetramers or subsequent release with benzonase or SmaI (FIG. 16A). ELISA-determined concentrations of HLA-A*02:01-Alk-SAv-Az Conjugated Tetramers left in the supernatant after the hydrogel loading step, or released from loaded hydrogels by benzonase or SmaI (FIG. 16B).

FIGS. 17A-17B depict IVTT peptide production to generate functional UV-exchanged tetramers. Western probed with anti-SUMO domain antibody: Product of an IVTT reaction (+/−Ulp1 protease) driven by a PCR amplicon template encoding SUMO-NLVPMVATV (SEQ ID NO: 8) peptide fusion was run in lanes 10-11 (FIG. 17A). Lanes 2-9 contain a dilution series of a SUMO-domain-containing standard, which was used to quantify the yield of SUMO domain to ˜1 uM (FIG. 17A). Flow analysis of tetramers produced by UV-exchange from IVTT-produced peptide (FIG. 17B). Tetramers were UV-exchanged in the presence of equimolar synthetic NLVPMVATV (SEQ ID NO: 8) peptide (UV ex 1:1 NLV—synthetic) or an IVTT reaction (+Ulp1) driven by a SUMO-NLVPMVATV (SEQ ID NO: 8) peptide template (UV ex NLV-IVTT), and stained at 1 nM on NLVPMVATV (SEQ ID NO: 8)-expanded PBMCs (FIG. 17B). Positive and negative control tetramers refolded with NLVPMVATV (SEQ ID NO: 8) or GILGFVFJL (SEQ ID NO: 7) peptides were also stained at 1 nM as shown (FIG. 17B).

FIG. 18 is a schematic showing high throughput barcoded antigen library production using exchangeable barcodable tetramers.

FIG. 19 is a schematic showing use of sortags and click chemistry for conjugation of p*MHCII to SAy, cleavage of the peptide linker within the placeholder peptide, exchange of the placeholder peptide with a rescue peptide and binding to a TCR.

FIG. 20A-20E depicts the generation of p*MHCII multimer. FIG. 20A: Anti-Myc Western Blot analysis of GGG-Alkyne conjugation to the α-chain of monomeric p*MHCII. FIG. 20B: SDS-PAGE analysis following click reaction of p*MHCII-Alk and SAv-Az. FIG. 20C: HiLoad 26/600 Superdex 200 SEC elution chromatogram of the clicking reaction sample. FIG. 20D: Anti-FLAG Western Blot analysis of the main peaks obtained from SEC. Lane 1: Chameleon Duo Pre-Stained Protein Ladder (Licor), Lane 2: click reaction before loading the sample to the SEC column, lanes 3 and 4: SEC samples from peak I, lanes 5 and 6: SEC samples from peak II, lane 7: free SAy. Lane numbers correspond to non-boiled samples while lane numbers that are labeled with an asterisk correspond to boiled samples. FIG. 20E: Anti-His Western Blot analysis of the main peaks obtained following SEC. Lane numbers are the same as described in FIG. 20D.

FIG. 21A-21C illustrates the digestion, exchange and TCR binding of pMHCII. FIG. 21A: SDS-PAGE analysis of boiled and non-boiled samples of pre- and post-factor Xa cleavage. FIG. 21B: An ELISA assay that detects the ability of biotinylated exchanged peptide to bind to p↓MHCII multimer. FIG. 21C: BLI assay that measures the interaction between an HA-specific TCR and p↓MHCII multimer that was exchanged to display a cognate HA peptide. The black, light gray and dark gray curves correspond to the signal obtained from moving the TCR-loaded biosensors into wells containing either exchanged NMHCII, non-exchanged p*MHCII and BLI buffer, respectively. The dashed line defines the transfer of the biosensors to wells that are devoid of analytes (dissociation).

FIG. 22A-22B show results of MCR analysis of SARS-CoV-2 Spike Protein epitopes using HLA Class II DRB 1*07:01 (black), 1*04:04 (dark grey), 1*15:01 (grey) and 1*10:01 (green), with five T cell epitopes indicated in FIG. 22A (SEQ ID NOs: 271-275) and three T cell epitopes indicated in FIG. 22B (SEQ ID NOs: 276-278).

FIG. 23 show results of MCR analysis of SARS-CoV-2 Nucleocapsid Protein epitopes using HLA Class II DRB 1*07:01 (black), 1*04:04 (dark grey), 1*15:01 (grey) and 1*10:01 (green), with seven T cell epitopes indicated (SEQ ID NOs: 279-285).

FIG. 24A-24C shows analyses of SARS-CoV-2 antigen peptide library binding to six different MHC Class I alleles. FIG. 24A shows the percentage binding and total number of peptide bound by each allele. FIG. 24B shows the overlap in peptide binding between the A1101, A0101 and A0301 alleles. FIG. 24C shows the overlap in peptide binding between the A0201, A0101 and A0301 alleles.

FIG. 25 shows representative results of SARS-CoV-2 peptide-MHC tetramer library screening for A*02:01 patient samples, showing number of samples, clones or cells bound to each peptide from the indicated antigens. The sequences of the peptide epitopes are shown in SEQ ID NOs: 286-305.

FIG. 26 shows the results of mapping T cell reactive epitopes identified by peptide-MHC tetramer library screening across related viruses. The four top epitopes identified by library screening (SEQ ID NOs: 286-289) are highlighted (arrows).

FIG. 27 is a schematic diagram of the chimeric MHC/TcR receptor used in the MCR™ system.

FIG. 28 is a schematic diagram of the MCR™ system for identifying T cell epitopes.

FIG. 29 shows additional results of SARS-CoV-2 peptide-MHC tetramer library screening for A*02:01 patient samples, showing number of samples, clones or cells bound to each peptide from the indicated antigens.

FIG. 30 illustrates the abundant CD8 and CD4 T cell clonotypes from the lungs of COVID 19-infected patients and the HLA-I and HLA-II alleles tested using the MCR™ system to identify T cell epitopes.

FIG. 31A-31D illustrates results from the MCR™ system screening of patient T cells. FIG. 31A illustrates selection of a representative CD4+ T cell clonotype expressing TCR115 for analysis. FIG. 31B illustrates screening results from the MCR™ system. FIG. 31C illustrates identification of a 20mer epitope (SEQ ID NO: 306) common to multiple 23mers in the library that bound to multiple clones (SEQ ID NOs: 307-310). FIG. 31D shows results confirming that T cells expressing TCR115 strongly recognized the 20mer epitope, whereas negative control T cells expressing a different receptor (TCR117) did not.

FIG. 32 shows results of the analysis of the peptide presentation capacity of five different HLA-II molecules for four different M protein epitopes (SEQ ID NOs: 307-310) recognized by TCR115, as well as highly immunogenic control peptide (SEQ ID NO: 312).

FIG. 33 shows results of analysis of the top 20 hits from screening 9mer epitopes using peptide-MHC tetramer libraries and T cells from COVID-19 convalescent patients.

FIG. 34 shows results of analysis of the top 20 hits from screening 9mer epitopes using peptide-MHC tetramer libraries and T cells from COVID-19 unexposed subjects.

FIG. 35A-35D shows the results of MEDi analysis of Spike peptide presentation by different HLAs. FIG. 35A shows results of an exemplary flow cytometric analysis and sorting of MCR2⁺ reporter cells, transduced with an MCR2 library and stained for CD3e. Based on the surface expression of the MCR2, four fractions (neg, low, mid and hi) were sorted and re-analyzed. Positive and negative controls are indicated. FIG. 35B shows MEDi MA⁸⁵ score traces for all Spike-derived peptides presented by 5 different HLAs (thick grey line). The thin grey line oscillating around the x-axis indicates error (uncertainty factor) related to data quality of the MEDi scores (more oscillation indicates less reliable data, see Materials and Methods for detailed explanation). FIG. 35C and FIG. 35D show schematics and interpretation of the MEDi traces, with MEDi analysis for the membrane (FIG. 35C) and nucleocapsid (FIG. 35D) proteins with indicated 15aa peptides falling into an example MEDi MA⁸⁵ peak. The extended peptides are recognized by COVID-19 specific TCRs analyzed in this study.

FIG. 36A-36D show the results of experiments for MEDi analysis of Spike peptide presentation by DRB1*07:01 compared to netMHCIIpan and MHC binding IC₅₀. FIG. 36A shows sequence comparison of Spike peptides representative for the individual MEDi MA85 peaks containing at least 3 peptides. Residues matching the HLA binding consensus are highlighted in grey. FIG. 36B shows MEDi MA score traces (grey) and the error (thin light grey) for all Spike-derived peptides presented by DRB1*07:01. Arrows indicate peptides chosen for HLA-binding IC₅₀ calculation by the fluorescence polarization assay. FIG. 36C shows results of the competitive peptide binding fluorescence polarization assay for individual peptides. IC₅₀ and R² values are shown. FIG. 36D shows ROC curves of the MEDi MA and netMHCIIpan scores qualifying peptides as HLA-binders. Calculations were done for peptides analyzed in FIG. 36C, positive binding thresholds at IC₅₀ of 500 nM, 1 μM or 5 μM.

FIG. 37A-37F show results of experiments on de-orphaning TCRs from the BAL of COVID-19 patients by MCR2 screening. FIG. 37A shows a schematic diagram of the MCR workflow. FIG. 37B shows results of experiments in which MCR2-SARS-CoV-2⁺ or SCT-SARS-CoV-2⁺ 16.2× reporter cells (GFP+), carrying all possible SARS-CoV-2-derived peptides in the context of all 12 patient-specific HLA alleles (complexity up to 120.000 individual pMHC combinations) were co-cultured with 16.2A2 cells transduced with individual TCRs from patients. Responding (NFAT⁺) reporter cells were sorted, expanded and co-cultured 4 times. FIG. 37C shows results of experiments in which individual responding reporter clones were isolated and re-analyzed by an additional co-culture. FIG. 37D shows sequences of the de-orphaned TCR chains, specific peptides and HLA restriction. FIG. 37E shows results of experiments in which 16.2× reporter cells carrying the MCR2-S₇₁₄₋₇₂₈ or MCR2-N₂₂₁₋₂₄₂ were analyzed on FACS for MCR2 expression (by anti-CD3 staining). FIG. 37F shows result of experiments in which 16.2× reporter cells carrying the MCR2-S₇₁₄₋₇₂₈ or MCR2-S_{714-728(F716I)}(top) and MCR2-N₂₂₁₋₂₄₂ or MCR2-N_{221-242 (S235F)}(bottom) were co-cultured with 16.2A2 cells transduced with TCR007 or TCR132 respectively and NFAT activation was measured on FACS.

FIG. 38A-38C shows results of experiments on presentation of immunogenic peptides by MEDi. FIG. 38A and FIG. 38B show results for MEDi MA score profiles (black) compared to netMHCIIpan prediction scores (scaled to fit on the same plot, thin black) for the HLAs presenting CD4 T cell specific peptides found in this study. MEDi MA⁸⁵ is indicated as a black line, T cell specific peptides are indicated as grey shades. FIG. 38C show results for MEDi MA traces for the membrane protein presented by the indicated alleles. Results of the competitive peptide binding assay for the indicated peptides are shown below. M₁₄₆₋₁₆₅ peptide (recognized by the TCR091 in the context of DRB1*11:01) is indicated next to the shaded areas.

FIG. 39A-39H show results of experiments in which MEDi reveals candidate immune-escape mutants. FIG. 39A shows results for experiments in which micro MCR2 libraries, containing all 15 (15aa long) peptides spanning the indicated mutations were cloned for each indicated HLA and transduced into the 16.2× reporter cells. Shown are individual MEDi MA scores for the WT (dark grey) and mutated (light grey) peptides. For context MEDi traces for full ORF8 and Spike are shown. Grey shaded squares indicate differences seen in all repeat experiments (n=2 or 3). FIG. 39B shows example peptide sequences from ORF8 with indicated starting residues and the MHC binding motif for DBR1*04:04. FIG. 39C shows a detailed view of the MEDi MA scores for the WT and D1118D Spike mutated peptides in the context of DRB1*07:01. FIG. 39D shows a detailed view of the MEDi-MA scores for the WT and T716I Spike mutated peptides in the context of DRB1*07:01. FIG. 39E shows 15 peptides spanning the T716I mutation with indicated starting residues and the different DBR1*07:01binding motifs. FIG. 39F shows S₇₁₄₋₇₂₈ peptide sequences with indicated different binding registers forced by several DBR1*07:01 binding motifs present in the WT and/or mutated peptide. TCR facing residues are shown in grey. FIG. 39G shows FACS analysis and sorting of reporter cells transduced with DRB1*07:01-MCR2 carrying the 12mer peptides: S714-725, S714-725(T716I) and S717-728. FIG. 39H Reporter cells from F, were co-cultured with 16.2A2 cells transduced with TCR007 and NFAT activation was measured on FACS.

FIG. 40 shows a list of potentially presentable peptides derived from the Spike protein for four different HLA molecules.

FIG. 41 shows a list of all MHC Class I alleles carrying 10 amino acid peptides across the whole SARS-CoV-2 genome (1 aa shifts) and all MHC Class II alleles carrying 15 or 23 amino acid peptides across the whole SARS-CoV-2 genome (1 aa shifts) for different CD4 TCRs or CD8 TCRs.

FIG. 42A-42B show additional results of MEDi experiments using MHC Class II molecules DRB1*07:01 and DRB1*11:01 (FIG. 42A) or DRB1*07:01, DRB1*14:05 and DRB1*08:03 (FIG. 42B).

FIG. 43A-43D show the results of experiments for MEDi analysis of Spike peptide presentation by DRB1*15:01 compared to netMHCIIpan and MHC binding IC₅₀. FIG. 43A shows sequence comparison of Spike peptides representative for the individual MEDi MA peaks containing at least 3 peptides. Residues matching the HLA binding consensus are highlighted in grey. FIG. 43B shows MEDi MA score traces (grey) and the error (thin grey) for all Spike-derived peptides presented by DRB1*15:01. Arrows indicate peptides chosen for HLA-binding IC₅₀ calculation by the fluorescence polarization assay. FIG. 43C shows results of the competitive peptide binding fluorescence polarization assay for individual peptides. IC₅₀ and R² values are shown. FIG. 43D shows ROC curves of the MEDi MA and netMHCIIpan scores qualifying peptides as HLA-binders.

FIG. 44 shows results of the competitive peptide binding fluorescence polarization assay for the indicated peptides and MHC Class II molecules. IC₅₀ and R² values are shown.

FIG. 45A-45C show an overview of the experimental approach used to decode CD8+ response to SARS-CoV-2. FIG. 45A is a schematic of method where encoded tetramer libraries, designed independently by HLA allele to span the entire SARS-2-proteome, are used to stain enriched CD8+ cells from subject PBMCs, which are then sorted and subjected to single-cell sequencing (left). Using this approach, TCR sequence, specificity and transcriptomic features are simultaneously acquired for each cell (right). FIG. 45B shows clonotype specificity detected by HLA allele and epitope across the SARS-CoV-2 proteome. FIG. 45C shows single-cell transcriptomic analysis showing global UMAP clustering, scoring by functional gene set, and projections onto the transcriptomic UMAP for T cells with specificity toward select epitopes in convalescent individuals. QYI-A24, PTD-A01, and LLY-A02 correspond to QYIKWPWYI (SEQ ID NO: 318) in A*24:02, PTDNYITTY (SEQ ID NO: 327) in A*01:01, and LLYDANYFL (SEQ ID NO: 286) in A*02:01, respectively.

FIG. 46A-46C shows the specificity to SARS-CoV-2 epitopes across HLA, cohort, and subject. FIG. 46A shows the frequency of T cell response detected (cells per million CD8+ interrogated) by subject and cohort. FIG. 46B shows T cell specificity observed in unexposed versus convalescent cohorts represented as percentage of cohort with any detectable frequency of T cell specificity against each epitope. The size of each dot represents the mean frequency detected across convalescent and unexposed subjects. FIG. 46C shows sequence alignment between SARS-CoV-2 proteome and common cold coronaviruses, shown for select epitopes. Mismatches are represented in dark grey and HLA anchor residues with a grey background. Arrows indicate sequences where anchor and all internal residues are conserved between SARS-CoV and HCoV species.

FIG. 47A-47D show functional assays used to characterize recombinant TCR (rTCR) activation upon stimulation with SARS-CoV-2 and homologous epitopes. FIG. 47A is a schematic showing lentiviral transduction of TCRs into a J76 cell line, stimulation of APCs with synthetic peptide, and quantification of activated J76 cells expressing CD69. FIG. 47B shows dose-response curves for TCR-pMHC interactions observed across several canonical epitopes in A*02:01 and B*07:02. Shown are fractions of CD69(+) cells after a 16 hour stimulation. FIG. 47C shows functional activation of TCRs by canonical and homologous epitopes, represented by fraction of CD69(+) cells after 16 hour stimulation with 10 uM peptide. FIG. 47D shows dose-response curves for several rTCRs from COVID patients (left) or unexposed subjects (right) stimulated with peptide from SARS-CoV-2 or HCoV HKU1/OC43.

FIG. 48A-48D show analysis of TCR sequences from cells specific to the most immunodominant epitopes for each allele tested. FIG. 48A shows network plots showing TCR biochemical similarity of alpha or beta CDR3s in unexposed subjects (left) or COVID patients (right). Unique subjects are identified by node color. Each node is a unique clonotype within a subject, and the size of the node the relative frequency of response detected. Edges drawn between nodes represent CDR3 homology, and the size of each node represents relative cell frequency. FIG. 48B shows V gene usage for alpha and beta chains across all sequences represented in FIG. 48A with the most frequently used gene labeled. FIG. 48C shows distributions of CDR3 lengths. FIG. 48D shows alpha beta paired CDR3 motifs for the most interconnected nodes identified in the network analysis.

FIG. 49A-49C show transcriptomic clustering of T cells based on function-specific gene sets. FIG. 49A shows single cell gene expression of single cells specific to SARS-CoV-2, CMV, EBV, Influenza, or with no observed (N.O.) specificity. Units are ln(TP10K). Kmeans clustering was used to identify seven distinct clusters showing gene expression consistent with a range of functional states. FIG. 49B shows specificity tick strips indicating the location and cohort assignment of individual cells with specificity to CEF or SARS-CoV-2 epitopes. FIG. 49C shows gene expression of single cells with individual specificities. In cases where specificity was detected in the unexposed cohort, pie charts are shown to indicate the fraction of cells corresponding to each cluster identified in FIG. 49A.

FIG. 50A-50B shows the results of a receiver-operator analysis for TCR-pMHC hit identification.

FIG. 51 shows the results of the overall reactivity to T cells to CMV, EBV, influenza, and SARS-CoV-2 by cohort.

FIG. 52 shows the transcriptomic clustering of T cells from SARS-CoV-2 acute patients, convalescent patients, and unexposed donors. Exemplary T cell populations are shown. T cell types are indicated: naïve T cells, central memory T cell (Tcm), 127+ memory T cell, effector memory (Tem) chronically active T cells, chronically stimulated 1 T cells, chronically stimulated 2 T cells, and cytotoxic effector T cells.

FIG. 53 shows effects of SARS-CoV-2 mutations on presentability of peptides. HLA allele and SARS-CoV-2 mutations are indicated.

FIG. 54 shows an exemplary supplementary MEDi analysis of mutated peptides present in arising SARS-CoV-2 variants.

### DETAILED DESCRIPTION

### I. Definitions

All technical and scientific terms used herein, unless otherwise defined below, are intended to have the same meaning as commonly understood by one of ordinary skill in the art. Mention of techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

As used herein, “about” will be understood by persons of ordinary skill and will vary to some extent depending on the context in which it is used. If there are uses of the term which are not clear to persons of ordinary skill given the context in which it is used, “about” will mean up to plus or minus 10% of the particular value.

As used herein, an “altered peptide ligand” or “APL” refers to an altered or mutated version of a peptide ligand, such as an MHC binding peptide. The altered or mutated version of the peptide ligand contains at least one structural modification (e.g., amino acid substitution) as compared to the peptide ligand from which it is derived. For example, a panel of APLs can be prepared by systematic or random mutation of a known MHC binding peptide, to thereby create a pool of APLs that can be used as a library of MHC binding peptides for loading onto MHC Conjugated Multimers as described herein.

As used herein, the term “and/or” when used in the context of a list of entities, refers to the entities being present singly or in any possible combination or subcombination.

The term “antigenic determinant” or “epitope” refers to a site on an antigen to which the variable domain of a T cell receptor, an MHC molecule or antibody specifically binds. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods for determining what epitopes are bound by a given TCR or antibody (i.e., epitope mapping) are well known in the art and include, for example, immunoblotting and immunoprecipitation assays, wherein overlapping or contiguous peptides from the antigen are tested for reactivity with the given TCR or immunoglobulin. Methods of determining spatial conformation of epitopes include techniques in the art and those described herein, for example, x-ray crystallography nuclear magnetic resonance, cryogenic electron microscopy (cryo-EM), hydrogen deuterium exchange mass spectrometry (HDX-MS), and site-directed mutagenesis (see, e.g., Epitope Mapping Protocols in Methods in Molecular Biology, Vol. 66, G. E. Morris, Ed. (1996)). A T cell epitope refers to a portion of an antigen (e.g., antigenic protein) that binds to (interacts with or is recognized by) a T cell receptor.

The term “avidity” as used herein, refers to the binding strength of as a function of the cooperative interactivity of multiple binding sites of a multivalent molecule (e.g., a soluble multimeric pMHC-immunoglobulin protein) with a target molecule. A number of technologies exist to characterize the avidity of molecular interactions including switchSENSE and surface plasmon resonance (Gjelstrup et al., J. Immunol. 188:1292-1306, 2012); Vorup-Jensen, Adv. Drug. Deliv. Rev. 64:1759-1781, 2012).

As used herein a “barcode”, also referred to as an oligonucleotide barcode, is a short nucleotide sequence (e.g., about 1, 2, 3, 4, 5, 6, 7, 8, 9, 10, 11, or 12 nucleotides long) that identifies a molecule to which it is conjugated. Barcodes can be used, for example, to identify molecules in a reaction mixture. Barcodes uniquely identify the molecule to which it is conjugated, for example, by performing reverse transcription using primers that each contain a “unique molecular identifier” barcode. In other embodiment, primers can be utilized that contain “molecular barcodes” unique to each molecule. The process of labeling a molecule with a barcode is referred to herein as “barcoding.” A “DNA barcode” is a DNA sequence used to identify a target molecule during DNA sequencing. In some embodiments, a library of DNA barcodes is generated randomly, for example, by assembling oligos in pools. In other embodiments, the library of DNA barcodes is rationally designed in silico and then manufactured.

“Binding affinity” generally refers to the strength of the sum total of noncovalent interactions between a single binding site of a molecule (e.g., a TCR, pMHC) and its binding partner. Unless indicated otherwise, as used herein, “binding affinity” refers to intrinsic binding affinity which reflects a 1:1 interaction between members of a binding pair (e.g., TCR and antigen). The affinity of a molecule X for its partner Y can generally be represented by the dissociation constant (Kd). For example, the Kd can be about 200 nM, 150 nM, 100 nM, 60 nM, 50 nM, 40 nM, 30 nM, 20 nM, 10 nM, 8 nM, 6 nM, 4 nM, 2 nM, 1 nM, or stronger, including up to 1 μM Affinity can be measured by common methods known in the art, including those described herein. Low-affinity TCRs generally bind antigen slowly and tend to dissociate readily, whereas high-affinity TCRs generally bind antigen faster and tend to remain bound longer. A variety of methods of measuring binding affinity are known in the art, any of which can be used for purposes of the present disclosure.

The term “bioorthogonal chemistry” refers to any chemical reaction that can occur inside of living systems without interfering with native biochemical processes. The term includes chemical reactions that are chemical reactions that occur in vitro at physiological pH in, or in the presence of water. To be considered bioorthogonal, the reactions are selective and avoid side-reactions with other functional groups found in the starting compounds. In addition, the resulting covalent bond between the reaction partners should be strong and chemically inert to biological reactions and should not affect the biological activity of the desired molecule.

As used herein, the terms “carrier” and “pharmaceutically acceptable carrier” includes any and all solvents, dispersion media, coatings, antibacterial and antifungal agents, isotonic and absorption delaying agents, and the like that are physiologically compatible.

The term “chelator ligand” as used herein refers to a bifunctional conjugating moiety that covalently links a radiolabeled prosthetic group to a biologically active targeting molecule (e.g., peptide or protein). Bifunctional conjugating moiety utilize functional groups such as carboxylic acids or activated esters for amide couplings, isothiocyanates for thiourea couplings and maleimides for thiol couplings.

As used herein, the term “cleavable moiety” refers to a motif or sequence that is cleavable. In some embodiments, the cleavage moiety comprises a protein, e.g., enzymatic, cleavage site. In some embodiments, the cleavage moiety comprises a chemical cleavage site, e.g., through exposure to oxidation/reduction conditions, light/sound, temperature, pH, pressure, etc.

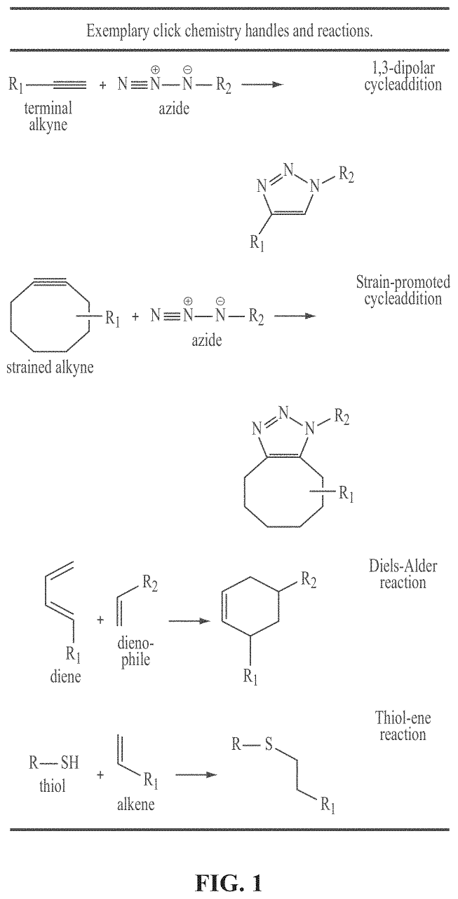

The term “click chemistry” refers to a set of reliable and selective bioorthogonal reactions for the rapid synthesis of new compounds and combinatorial libraries. Properties of click reactions include modularity, wideness in scope, high yielding, stereospecificity and simple product isolation (separation from inert by-products by non-chromatographic methods) to produce compounds that are stable under physiological conditions. In radiochemistry and radiopharmacy, click chemistry is a generic term for a set of labeling reactions which make use of selective and modular building blocks and enable chemoselective ligations to radiolabel biologically relevant compounds in the absence of catalysts. A “click reaction” can be with copper, or it can be a copper-free click reaction. Non-limiting examples of click chemistry handles and reactions are shown in FIG. 1.

As used herein, the term “conditions sufficient for covalent conjugation” refers to reaction conditions, including but not limited to temperature, pH and concentrations of the reaction components, that are suitable such that the desired covalent conjugation chemical reaction occurs.

As used herein, the term “Conjugated Multimer”, also referred to as a pMHC Conjugated Multimer, refers to the reaction product that results from the reaction of pMHC monomers comprising a conjugation moiety with a multimerization domain comprising a conjugation moiety, wherein the two conjugation moieties react with each other to form a covalent linkage between the pMHC monomers and the multimerization domain, thereby forming Conjugated Multimers. In one embodiment, the Conjugated Multimer is a Conjugated Tetramer, in which four pMHC monomers are reacted with the multimerization domain, through their conjugation moieties, to thereby form a tetramer. In one embodiment, the Conjugated Multimer is a pMHCI Conjugated Multimer (e.g., Tetramer), in which pMHC Class I monomers are multimerized. In one embodiment, the Conjugated Multimer is a pMHCII Conjugated Multimer (e.g., Tetramer) in which pMHC Class II monomers are multimerized.

As used herein, the term “cross-linking unit” can refer to a molecule that links to another (same or different) molecule. In some embodiments, the cross-linking unit is a monomer. In some embodiments, the cross-link is a chemical bond. In some embodiments, the cross-link is a covalent bond. In some embodiments, the cross-link is an ionic bond. In some embodiments, the cross-link alters at least one physical property of the linked molecules, e.g., a polymer's physical property.

As used herein, the term “endoprotease” refers to a protease that cleaves a peptide bond of a non-terminal amino acid.

The terms “exchangeable pMHC polypeptide”, “exchangeable pMHC multimers”, and “placeholder-peptide loaded MHC polypeptide”, which are used interchangeably herein, refer to MHC monomers and MHC multimers, comprising a placeholder peptide in the binding groove of the MHC polypeptide, and are also referred to as “p*MHC” monomers or multimers. “Exchangeable” refers to the property of a p*MHC monomer or p*MHC multimer allowing for the exchange of the placeholder peptide with an antigenic peptide. In one embodiment, the exchangeable pMHC or p*MHC polypeptide comprises an MHC Class I molecule with an MHC Class I-binding peptide in the binding groove of the MHC Class I molecule. In another embodiment, the exchangeable pMHC or p*MHC polypeptide comprises an MHC Class II molecule with an MHC Class II-binding peptide in the binding groove of the MHC Class II molecule.

A “fusion protein” or “fusion polypeptide” as used interchangeably herein refers to a recombinant protein prepared by linking or fusing two polypeptides into a single protein molecule.

The term “isolated” as applied, for example to MHC monomers herein refers to an MHC glycoprotein, which is in other than its native state, for example, not associated with the cell membrane of a cell that normally expresses MHC. This term embraces a full length subunit chain, as well as a functional fragment of the MHC monomer. A functional fragment is one comprising an antigen binding site and sequences necessary for recognition by the appropriate T cell receptor. It typically comprises at least about 60-80%, typically 90-95% of the sequence of the full-length chain. An “isolated” MHC subunit component may be recombinantly produced or solubilized from the appropriate cell source. In one embodiment, the “isolated” MHC monomer is an MHC Class I monomer, such as a soluble form of the MHC Class I heavy chain (a chain) associated with β2-microglobulin. In another embodiment, the “isolated” MHC monomer is an MHC Class II monomer, such as a soluble form of the MHC Class II α/β chains.

As used herein, the term “identifier” refers to a readable representation of data that provides information, such as an identity, that corresponds with the identifier.

As used herein, the terms “linked,” “conjugated,” “fused,” or “fusion,” are used interchangeably when referring to the joining together of two more elements or components or domains, by whatever means including recombinant or chemical means.

The term “Major Histocompatibility Complex” or “MHC” refers to genomic locus containing a group of genes that encode the polymorphic cell-membrane-bound glycoproteins known as MHC class I and class II molecules that regulate the immune response by presenting peptides of fragmented proteins to circulating cytotoxic and helper T lymphocytes, respectively. In humans, this group of genes is also called the “human leukocyte antigen” or “HLA” system. Human MHC class I genes encode, for example, HLA-A, HL-B and HLA-C molecules. HLA-A is one of three major types of human MHC class I cell surface receptors. The others are HLA-B and HLA-C. The HLA-A protein is a heterodimer, and is composed of a heavy α chain and smaller β chain. The α chain is encoded by a variant HLA-A gene, and the β chain is an invariant β2 microglobulin (β2m) polypeptide. The β2 microglobulin polypeptide is coded for by a separate region of the human genome. For example, HLA-A*02 (A*02) is a human leukocyte antigen serotype within the HLA-A serotype group. The serotype is determined by the antibody recognition of the α2 domain of the HLA-A α-chain. For A*02, the α chain is encoded by the HLA-A*02 gene and the β chain is encoded by the B2M locus. Other exemplary HLA serotypes include HLA-A*01:01, HLA-A*02:01, HLA-A*24:02, HLA-B*07:02, A*32:01, B*48:01, and the other HLAs identified in TABLEs 1 and 2. Human MHC class II genes encode, for example, HLA-DPA1, HLA-DPB1, HLA-DQA1, HLA-DQB1, HLA-DRA and HLA-DRB1. Exemplary MHC class II serotypes include DPA1*02:02, DPB1*05:01, DRB1*07:01, DRB1*14:05, DRB1*11:01, DRB1*08:03, and the other HLAs identified in TABLEs 3 and 4. The complete nucleotide sequence and gene map of the human major histocompatibility complex is publicly available (e.g., The MHC sequencing consortium, Nature 401:921-923, 1999).

As used herein, the terms “MHC molecule” and “MHC protein” are used herein to refer to the polymorphic glycoproteins encoded by the MHC class I and MHC class II genes, which are involved in the presentation of peptide epitopes to T cells. The terms “MHC class I” or “MHC I” are used interchangeably to refer to protein molecules comprising an a chain composed of three domains (α1, α2 and α3), and a second, invariant (β2-microglobulin. The α3 domain is transmembrane, anchoring the MHC class I molecule to the cell membrane. Antigen-derived peptide epitopes, which are located in the peptide-binding groove, in the central region of the α1/α2 heterodimer. MHC Class I molecules such as HLA-A are part of a process that presents short polypeptide antigens to the immune system. These polypeptides are typically 8-11 amino acids in length and originate from proteins being expressed by the cell. MHC class I molecules present antigen to CD8+ cytotoxic T cells. The terms “MHC class II” and “MHC II” are used interchangeably to refer to protein molecules containing an a chain with two domains (α1 and α2) and a β chain with two domains (β1 and β2). The peptide-binding groove is formed by the α1/β1 heterodimer. MHC class II molecules present polypeptide antigens to specific CD4+ T cells. These antigens can be 13-25 amino acids long, but typically are 15-24 amino acids long. Antigens delivered endogenously to APCs are processed primarily for association with MHC class I. Antigens delivered exogenously to APCs are processed primarily for association with MHC class II.

As used herein, MHC proteins (MHC Class I or Class II proteins) also includes MHC variants which contain amino acid substitutions, deletions or insertions and yet which still bind MHC peptide epitopes (MHC Class I or MHC Class II peptide epitopes). The term also includes fragments of all these proteins, for example, the extracellular domain, which retain peptide binding.

The term “MHC protein” also includes MHC proteins of non-human species of vertebrates. MHC proteins of non-human species of vertebrates play a role in the examination and healing of diseases of these species of vertebrates, for example, in veterinary medicine and in animal tests in which human diseases are examined on an animal model, for example, EAE (experimental autoimmune encephalomyelitis) in mice (Mus musculus), which is an animal model of the human disease multiple sclerosis. Non-human species of vertebrates are, for example, and more specifically mice (Mus musculus), rats (Rattus norvegicus), cows (Bos taurus), horses (Equus equus) and green monkeys (Macaca mulatta). MHC proteins of mice are, for example, referred to as H-2-proteins, wherein the MHC class I proteins are encoded by the gene loci H2K, H2L and H2D and the MHC class II proteins are encoded by the gene loci H21.

A “peptide free MHC polypeptide” or “peptide free MHC multimer” as used herein refers to an MHC monomer or MHC multimer which does not contain a peptide in binding groove of the MHC polypeptide. Peptide free MHC monomers and multimers are also referred to as “empty”. In one embodiment, the peptide free MHC polypeptide or multimer is an MHC Class I polypeptide or multimer. In another embodiment, the peptide free MHC polypeptide or multimer is an MHC Class II polypeptide or multimer.

As used herein, the term “multimer” refers to a plurality of units. In some embodiments, the multimer comprises one or more different units. In some embodiments, the units in the multimer are the same. In some embodiments, the units in the multimer are different. In some embodiments, the multimer comprises a mixture of units that are the same and different.

The terms “peptide epitope”, “MHC peptide epitope”, “MHC peptide antigen” and “MHC ligand” are used interchangeably herein and refer to an MHC ligand that can bind in the peptide binding groove of an MHC molecule. The peptide epitope can typically be presented by the MHC molecule. A peptide epitope typically has between 8 and 24 amino acids that are linked via peptide bonds. The peptide can contain one or more modifications such as, but not limited to, the side chains of the amino acid residues, the presence of a label or tag, the presence of a synthetic amino acid, a functional equivalent of an amino acid, or the like. Typical modifications include those as produced by the cellular machinery, such as glycan addition and phosphorylation. However, other types of modification are also within the scope of the disclosure.

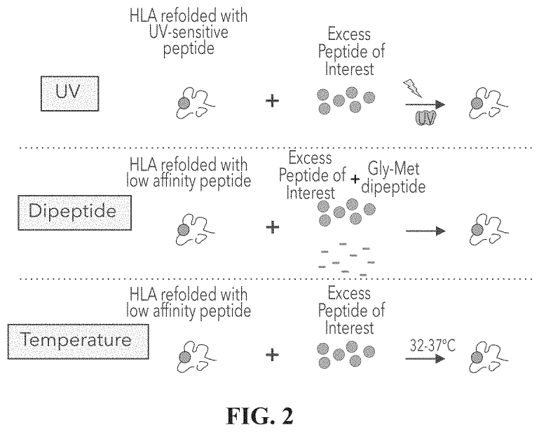

As used herein, the terms “peptide exchange” refers to a competition assay wherein a placeholder peptide is removed and replaced by a “exchanged peptide” (or “exchange peptide epitope”) also referred to herein as a “rescue peptide” (or “rescue peptide epitope”) or “competitor peptide” (or “competitor peptide epitope). Typically, peptide exchange occurs under conditions in which the placeholder peptide is released by cleavage of the peptide or under suitable conditions allowing rescue peptides to compete for binding to the binding pocket of an MHC monomer or multimer. For example, peptide exchange can be accomplished by UV-induced exchange, dipeptide-induced exchange, temperature-induced exchange, or other exchange methods known in the art, and disclosed herein. Exemplary methods of peptide exchange are set forth in FIG. 2.

As used herein, the term “peptide library” refers to a plurality of peptides. In some embodiments, the library comprises one or more peptides with unique sequences. In some embodiments, each peptide in the library has a different sequence. In some embodiments, the library comprises a mixture of peptides with the same and different sequences.

As used herein, the term “high diversity peptide library” refers to a peptide library with a high degree of peptide variety. For example, a high diversity peptide library comprises about 10³, about 10⁴, about 10⁵, about 10⁶, about 10⁷, about 10⁸, about 10⁹, about 10¹⁰, about 10¹¹, about 10¹², about 10¹³, about 10¹⁴, about 10¹⁵, about 10¹⁶, about 10¹⁷, about 10¹⁸, about 10¹⁹, about 10²⁰, or more different peptides.

As used herein, the term “library peptide” refers to a single peptide in the library.

As used herein, the terms “placeholder peptide” or “exchangeable peptide” are used interchangeably to refer to a peptide or peptide-like compound that binds with sufficient affinity to an MHC protein (e.g., MHCI or MHCII protein) and which causes or promotes proper folding of the MHC protein from the unfolded state or stabilization of the folded MHC protein. The placeholder peptide can subsequently be exchanged with a different peptide of interest (referred to as an exchange peptide or rescue peptide). This exchange can be accomplished by UV-induced exchange, dipeptide-induced exchange, temperature-induced exchange, or other exchange methods known in the art.

The terms “peptide,” polypeptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Peptides, polypeptides and proteins contain a series of amino acid residues, connected one to the other typically by peptide bonds between the alpha-amino and carbonyl groups of the adjacent amino acids, or a salt thereof. The terms “isolated peptide,” “isolated protein” and “isolated polypeptide” are used interchangeably to refer to a protein (e.g., a soluble, multimeric protein) which has been separated or purified from other components (e.g., proteins, cellular material) and/or chemicals. Typically, a polypeptide is purified when it constitutes at least 60 (e.g., at least 65, 70, 75, 80, 85, 90, 92, 95, 97, or 99) % by weight of the total protein in the sample.

As used herein, the term “protein folding” refers to spatial organization of a peptide. In some embodiments, the amino acid sequence influences the spatial organization or folding of the peptide. In some embodiments, a peptide may be folded in a functional conformation. In some embodiments, a folded peptide has one or more biological functions. In some embodiments, a folded peptide acquires a three-dimensional structure.

As used herein, the term “N-terminus amino acid residue” refers to one or more amino acids at the N-terminus of a polypeptide.

As used herein, the terms “small ubiquitin-like modifier moiety” or “SUMO domain” or “SUMO moiety” are used interchangeably and refer to a specific protease recognition moiety.

As used herein, the term “tag” refers to an oligonucleotide component, generally DNA, that provides a means of addressing a target molecule (e.g., a Conjugated Multimer) to which it is joined. For example, in some embodiments, a tag comprises a nucleotide sequence that permits identification, recognition, and/or molecular or biochemical manipulation of the molecule to which the tag is attached (e.g., by providing a unique sequence, and/or a site for annealing an oligonucleotide, such as a primer for extension by a DNA polymerase, or an oligonucleotide for capture or for a ligation reaction). The process of joining the tag to the target molecule is sometimes referred to herein as “tagging” and a target molecule that undergoes tagging or that contains a tag is referred to as “tagged” (e.g., a “tagged Conjugated Multimer”).” A tag can be a barcode, an adapter sequence, a primer hybridization site, or a combination thereof.

The term “T cell” refers to a type of white blood cell that can be distinguised from other white blood cells by the presence of a T cell receptor on the cell surface. There are several subsets of T cells, including, but not limited to, T helper cells (a.k.a. TH cells or CD4+ T cells) and subtypes, including T_{H}1, T_{H}2, T_{H}3, T_{H}17, T_{H}9, and T_{FH} cells, cytotoxic T cells (a.k.a Tc cells, CD8+ T cells, cytotoxic T lymphocytes, T-killer cells, killer T cells), memory T cells and subtypes, including central memory T cells (T_{CM} cells), effector memory T cells (T_{EM} and T_{EMRA} cells), and resident memory T cells (T_{RM} cells), regulatory T cells (a.k.a. T_{reg} cells or suppressor T cells) and subtypes, including CD4⁺ FOXP3⁺ T_{reg} cells, CD4⁺FOXP3⁻ T_{reg} cells, Tr1 cells, Th3 cells, and T_{reg}17 cells, natural killer T cells (a.k.a. NK T cells), mucosal associated invariant T cells (MAITs), and gamma delta T cells (γδ T cells), including Vγ9/Vδ2 T cells. The term “T cell cytotoxicity” includes any immune response that is mediated by CD8+ T cell activation.

As used herein, the phrase “T cell receptor” and the term “TCR” refer to a surface protein of a T cell that allows the T cell to recognize an antigen and/or an epitope thereof, typically bound to one or more major histocompatibility complex (MHC) molecules. A TCR functions to recognize an antigenic determinant and to initiate an immune response. Typically, TCRs are heterodimers comprising two different protein chains. In the vast majority of T cells, the TCR comprises an alpha (a) chain and a beta (β) chain. Each chain comprises two extracellular domains: a variable (V) region and a constant (C) region, the latter of which is membrane-proximal. The variable domains of α-chains and of β-chains consist of three hypervariable regions that are also referred to as the complementarity determining regions (CDRs). The CDRs, in particular CDR3, are primarily responsible for contacting antigens and thus define the specificity of the TCR, although CDR1 of the α-chain can interact with the N-terminal part of the antigen, and CDR1 of the β-chain interacts with the C-terminal part of the antigen. Approximately 5% of T cells have TCRs made up of gamma and delta (γ/δ) chains. All numbering of the amino acid sequences and designation of protein loops and sheets of the TCRs is according to the IMGT numbering scheme (IMGT, the international ImMunoGeneTics information system@imgt.cines.fr; http://imgt.cines.fr; Lefranc et al., (2003) Dev Comp Immunol 27:55 77; Lefranc et al. (2005) Dev Comp Immunol 29:185-203).

As used herein, the term “engineered TCR” is understood to mean a modified TCR, e.g., a recombinantly modified TCR. For example, the TCR may contain a modified binding cassette (e.g., where one or more CDR sequences or other elements is modified, for example, by introducing corresponding sequences from a different TCR). For example, the alpha and/or beta chain CDR3 sequence of a first TCR identified herein may be introduced into a second, different TCR present in or derived from a given T cell. The TCR may also contain modification, truncation, or deletion of its constant region, transmembrane region, and/or intracellular region. For example, at least the transmembrane region and the intracellular region can be deleted to generate a soluble form of a TCR.

As used herein, the terms “soluble T cell receptor” refers to heterodimeric truncated variants of TCRs, which comprise extracellular portions of the TCR α-chain and β-chain (e.g., linked by a disulfide bond), but which lack the transmembrane and cytosolic domains of the full-length protein. The sequence (amino acid or nucleic acid) of the soluble TCR α-chain and β-chains may be identical to the corresponding sequences in a native TCR or may comprise variant soluble TCR α-chain and β-chain sequences, as compared to the corresponding native TCR sequences. The term “soluble T cell receptor” as used herein encompasses soluble TCRs with variant or non-variant soluble TCR α-chain and β-chain sequences. The variations may be in the variable or constant regions of the soluble TCR α-chain and β-chain sequences and can include, but are not limited to, amino acid deletion, insertion, substitution mutations as well as changes to the nucleic acid sequence, which do not alter the amino acid sequence. Variants retain the binding functionality of their parent molecules.

As used herein, the terms “subject” and “patient” are used interchangeably and include human and non-human animals. Non-human animals include all vertebrates, e.g., mammals and non-mammals, such as non-human primates, sheep, dog, cow, chickens, amphibians, and reptiles.

As used herein, a “TCR/pMHC complex” refers to a protein complex formed by binding between T cell receptor (TCR), or soluble portion thereof, and a peptide-loaded MHC molecule. Accordingly, a “component of a TCR/pMHC complex” refers to one or more subunits of a TCR (e.g., Vα, Vβ, Cα, Cβ), or to one or more subunits of an MHC or pMHC class I or II molecule.

As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “unbiased” refers to lacking one or more selective criteria.

### II. Overview

Several companies are currently providing vaccines designed to induce humoral responses against the spike protein of SARS-CoV-2. However, for long lasting protection, it is contemplated that the generation of T cell memory will be required (Peng et al. (2020) Nature Immunol. doi: 10.1038/s41590-020-782-6), even if pre-existing T cell immunity to common cold coronavirus might play a role (Nelde et al. (2020) Nature Immunol. doi: 10.1038/s41590-020-00808-x; Grifoni et al. (2020) Cell 181, 1489-1501.e15). Protection by T cells, unlike the humoral response, relies entirely on T cell receptor recognition of pathogen-derived peptides presented by MHC and is mostly independent of physiological function or localization of the target protein. Consequently, while only particular epitopes of surface proteins allow targeting by neutralizing antibodies, many peptides can serve as T cell targets (T cell epitopes), providing a much broader coverage of the SARS-CoV-2 proteome space for therapeutic development.

Notwithstanding the foregoing, the prediction of antigen presentation by MHC molecules to T cells has been challenging given the diversity in both MHC and peptide space across which sophisticated analytical methods are traditionally required to generate sufficient data to train computational models. Furthermore, the breadth and nature of the cellular immune response to SARS-CoV-2 infection is driven by diversity in both T cell receptor repertoire and human leukocyte antigen (HLA) genetics. For example, mammalian cells express up to six different HLA class I alleles that shape antigen presentation in disease, and allelic diversity has been associated with both disease susceptibility and outcome of viral infections (MacDonald et al. 2000) J. Infect Dis. 181, 1581-1589; Ochoa et al. (2020) Vivol. J. 17, 128).

The work described herein leverages two different approaches that interrogate the interactions between specific peptide antigens associated with SARS-CoV-2 that are presented by specific MHC molecules encoded by certain HLA genes to specific T cell receptors expressed on certain T cells. In a first approach described in detail in Example 20, the capacity of certain HLA alleles to present SARS-CoV-2 virus peptides was interrogated using a mammalian epitope display known as MEDi. The findings were validated by studying T cell recognition of the SARS-CoV-2 virus in acute COVID-19 patients and by analyzing the impact of mutations carried by novel SARS-CoV-2 strains. Among other things, the studies suggest that immune evasion is based on shifting peptide presentation away from well recognized CD4 epitopes. Given the importance of CD4 T cells in controlling B cell and CD8 T cell responses in COVID-19 patients, the results described herein guide the generation of vaccines or therapeutics designed to elicit efficient and long lasting cellular immunity. In a second approach described in detail in Example 21, the connections between T cell specificity, HLA variation, conserved features of paired a/0 TCR repertoires, and cellular phenotype observed in CD8+ T cell responses to SARS-CoV-2 infection were elucidated at single-cell resolution using a single-cell, multi-omic technology. In this study, over a 100 million CD8+ T cells were profiled ex vivo across 76 acute, convalescent, or unexposed individuals, which identified T cell specificity for over 600 epitopes presented by four HLA alleles across the SARSCoV-2 proteome. The data suggest a strong association between HLA genotype and the CD8+ T cell response to SARS-CoV-2, which can also guide the generation of vaccines designed to confer long-term immunity to protect against SARS-CoV-2 variants and related viral pathogens.

As a result of the foregoing, provided herein are specific, identified SARS-CoV-2 T cell epitopes that are presented or are presentable to the immune system. In particular, the specific SARS-CoV-2 T cell epitopes disclosed herein represent T cell epitopes of SARS-CoV-2 proteins that can be presented via certain MHC class I and MHC class II molecules on antigen presenting cells to certain T cells, e.g., CD8+ and CD4+ T cells, via the T cell receptors expressed on such T cells.

### III. T Cell Epitopes and Antigen Presentation

Provided herein are specific, identified SARS-CoV-2 T cell epitopes that are presented or are presentable to the immune system. In particular, the specific SARS-CoV-2 T cell epitopes disclosed herein represent T cell epitopes of SARS-CoV-2 proteins that can be presented via certain MHC class I and MHC class II molecules on antigen presenting cells to certain T cells, e.g., CD8+ and CD4+ T cells, via the T cell receptors expressed on such T cells. The following sections discuss the T cell epitopes, the MHC molecules that present the T cell epitopes and the T cell receptors that bind the T cell epitopes, and their use in various applications.

In certain embodiments, a SARS-CoV-2 T cell epitope comprises an amino acid sequence selected from the amino acid sequences set forth in TABLES 1-4.

In certain embodiments, the T cell epitope is 8-10, 8-11, 8-12, 8-13, 8-14, 8-15, 8-16, 8-17, 8-18, 8-19, 8-20, 8-21, 8-22, 8-23, 8-24, 8-25, 8-30, or 8-35 amino acids in length. In certain embodiments, the T cell epitope is an MHC Class I-restricted epitope and is 8-10 amino acids in length. In certain embodiments, the T cell epitope is an MHC Class II-restricted epitope and is 13-14, 13-15, 13-16, 13-17, 13-18, 13-19, 13-20, 13-21, 13-22, 13-23, 13-24, 13-25, 13-30, or 13-35 amino acids in length.

Exemplary CD8+ T cell epitopes including their corresponding MHC class I alleles on antigen presenting cells, and the corresponding T cell receptors on CD8+ T cells or are set forth in TABLE 1.

TABLE 1

| | | | Epitope SEQ ID | |
|---|---|---|---|---|
| TCR ID¹ | HLA class I | SARS-CoV-2 epitope | NO: | No. of Cells |
| TCR_1 | A*02:01 | TLMNVITLV | 366 | 114 |
| TCR_2 | A*02:01 | LLLDRLNQL | 340 | 89 |
| TCR_3 | A*24:02 | TSQWLTNIF | 331 | 55 |
| TCR_4 | B*07:02 | SPRWYFYYL | 323 | 47 |
| TCR_5 | A*02:01 | YLQPRTFLL | 288 | 36 |
| TCR_6 | A*02:01 | MLDMYSVML | 314 | 22 |
| TCR_7 | A*02:01 | YLQPRTFLL | 288 | 14 |
| TCR_8 | A*24:02 | YYTSNPTTF | 319 | 13 |
| TCR_9 | A*24:02 | NYMPYFFTL | 316 | 12 |
| TCR_10 | B*07:02 | SPRWYFYYL | 323 | 11 |
| TCR_11 | B*07:02 | QPGQTFSVL | 326 | 10 |
| TCR_12 | A*02:01 | LLLDRLNQL | 340 | 10 |
| TCR_13 | A*24:02 | VYIGDPAQL | 317 | 9 |
| TCR_14 | A*02:01 | YLQPRTFLL | 288 | 9 |
| TCR_15 | A*02:01 | KLNEEIAII | 315 | 9 |
| TCR_16 | A*02:01 | YLQPRTFLL | 288 | 8 |
| TCR_17 | B*07:02 | SPRWYFYYL | 323 | 7 |
| TCR_18 | B*07:02 | SPRWYFYYL | 323 | 7 |
| TCR_19 | A*24:02 | VYAWNRKRI | 322 | 7 |
| TCR_20 | A*02:01 | YLQPRTFLL | 288 | 7 |
| TCR_21 | B*07:02 | SPRWYFYYL | 323 | 6 |
| TCR_22 | A*02:01 | LLYDANYFL | 286 | 6 |
| TCR_23 | A*24:02 | VYIGDPAQL | 317 | 6 |
| TCR_24 | A*24:02 | VMHANYIFW | 349 | 5 |
| TCR_25 | A*01:01 | FTSDYYQLY | 328 | 5 |
| TCR_26 | A*01:01 | PTDNYITTY | 327 | 5 |
| TCR_27 | A*02:01 | LLYDANYFL | 286 | 5 |
| TCR_28 | A*02:01 | YLQPRTFLL | 288 | 5 |
| TCR_29 | B*07:02 | SPRWYFYYL | 323 | 4 |
| TCR_30 | B*07:02 | SPRWYFYYL | 323 | 4 |
| TCR_31 | A*24:02 | QYIKWPWYI | 318 | 4 |
| TCR_32 | A*02:01 | MLDMYSVML | 314 | 4 |
| TCR_33 | A*02:01 | YLQPRTFLL | 288 | 4 |
| TCR_34 | A*02:01 | KLWAQCVQL | 287 | 4 |
| TCR_35 | A*02:01 | KLWAQCVQL | 287 | 4 |
| TCR_36 | B*07:02 | SPRWYFYYL | 323 | 4 |
| TCR_37 | A*02:01 | TQWSLFFFL | 408 | 3 |
| TCR_38 | B*07:02 | SPRWYFYYL | 323 | 3 |
| TCR_39 | A*02:01 | FLLNKEMYL | 289 | 3 |
| TCR_40 | A*24:02 | VYIGDPAQL | 317 | 3 |
| TCR_41 | A*01:01 | FTSDYYQLY | 328 | 3 |
| TCR_42 | B*07:02 | SPRWYFYYL | 323 | 3 |
| TCR_43 | A*02:01 | YLQPRTFLL | 288 | 3 |
| TCR_44 | A*24:02 | NYMPYFFTL | 316 | 3 |
| TCR_45 | B*07:02 | SPRWYFYYL | 323 | 3 |
| TCR_46 | A*02:01 | ALWEIQQVV | 297 | 3 |
| TCR_47 | A*02:01 | LLYDANYFL | 286 | 3 |
| TCR_48 | A*24:02 | TSQWLTNIF | 331 | 3 |
| TCR_49 | A*24:02 | NYMPYFFTL | 316 | 3 |
| TCR_50 | A*02:01 | KLWAQCVQL | 287 | 3 |
| TCR_51 | A*02:01 | KLWAQCVQL | 287 | 3 |
| TCR_52 | A*02:01 | ALWEIQQVV | 297 | 3 |
| TCR_53 | A*01:01 | PTDNYITTY | 327 | 3 |
| TCR_54 | A*01:01 | PTDNYITTY | 327 | 3 |
| TCR_55 | A*02:01 | ALWEIQQVV | 297 | 2 |
| TCR_56 | A*24:02 | STQWSLFFF | 342 | 2 |
| TCR_57 | A*02:01 | ALWEIQQVV | 297 | 2 |
| TCR_58 | A*02:01 | ILFTRFFYV | 393 | 2 |
| TCR_59 | A*02:01 | KLWAQCVQL | 287 | 2 |
| TCR_60 | A*02:01 | KSVNITFEL | 338 | 2 |
| TCR_61 | A*24:02 | TSAMQTMLF | 355 | 2 |
| TCR_62 | A*02:01 | ALWEIQQVV | 297 | 2 |
| TCR_63 | A*02:01 | YLQPRTFLL | 288 | 2 |
| TCR_64 | A*02:01 | ALWEIQQVV | 297 | 2 |
| TCR_65 | A*02:01 | LLYDANYFL | 286 | 2 |
| TCR_66 | A*24:02 | LFTRFFYVL | 357 | 2 |
| TCR_67 | A*02:01 | GLMWLSYFV | 293 | 2 |
| TCR_68 | A*24:02 | FYWFFSNYL | 352 | 2 |
| TCR_69 | A*02:01 | LLYDANYFL | 286 | 2 |
| TCR_70 | A*02:01 | YLQPRTFLL | 288 | 2 |
| TCR_71 | A*02:01 | LLLDRLNQL | 340 | 2 |
| TCR_72 | A*02:01 | LLLDRLNQL | 340 | 2 |
| TCR_73 | A*24:02 | CLAYYFMRF | 334 | 2 |
| TCR_74 | A*02:01 | GLMWLSYFI | 294 | 2 |
| TCR_75 | A*02:01 | KLWAQCVQL | 287 | 2 |
| TCR_76 | A*24:02 | LWLLWPVTL | 337 | 2 |
| TCR_77 | A*02:01 | KLWAQCVQL | 287 | 2 |
| TCR_78 | A*02:01 | ALWEIQQVV | 297 | 2 |
| TCR_79 | A*02:01 | VMVELVAEL | 350 | 2 |
| TCR_80 | A*24:02 | NYMPYFFTL | 316 | 2 |
| TCR_81 | A*02:01 | YLQPRTFLL | 288 | 2 |
| TCR_82 | B*07:02 | SPRWYFYYL | 323 | 2 |
| TCR_83 | A*24:02 | NYMPYFFTL | 316 | 2 |
| TCR_84 | A*02:01 | LLYDANYFL | 286 | 2 |
| TCR_85 | A*02:01 | KSVNITFEL | 338 | 2 |
| TCR_86 | A*02:01 | YLQPRTFLL | 288 | 2 |
| TCR_87 | A*02:01 | YLGGMSYYC | 381 | 2 |
| TCR_88 | A*02:01 | YLQPRTFLL | 288 | 2 |
| TCR_89 | B*07:02 | SPRWYFYYL | 323 | 2 |
| TCR_90 | A*02:01 | YLQPRTFLL | 288 | 2 |
| TCR_91 | B*07:02 | SPRWYFYYL | 323 | 2 |
| TCR_92 | B*07:02 | SPRWYFYYL | 323 | 2 |
| TCR_93 | A*02:01 | RQLLFVVEV | 360 | 2 |
| TCR_94 | A*02:01 | YLQPRTFLL | 288 | 2 |
| TCR_95 | A*02:01 | VFLVLLPLV | 362 | 2 |
| TCR_96 | A*02:01 | VMVELVAEL | 350 | 2 |
| TCR_97 | B*07:02 | RIRGGDGKM | 324 | 2 |
| TCR_98 | A*02:01 | YLQPRTFLL | 288 | 2 |
| TCR_99 | A*02:01 | YLQPRTFLL | 288 | 2 |
| TCR_100 | A*24:02 | QYIKWPWYI | 318 | 2 |
| TCR_101 | A*24:02 | LWLLWPVTL | 337 | 2 |
| TCR_102 | A*02:01 | YLQPRTFLL | 288 | 2 |
| TCR_103 | A*02:01 | YLQPRTFLL | 288 | 2 |
| TCR_104 | A*02:01 | LACFVLAAV | 363 | 2 |
| TCR_105 | A*24:02 | QYIKWPWYI | 318 | 2 |
| TCR_106 | A*02:01 | YLQPRTFLL | 288 | 2 |
| TCR_107 | B*07:02 | RIRGGDGKM | 324 | 2 |
| TCR_108 | A*02:01 | YLQPRTFLL | 288 | 2 |
| TCR_109 | A*24:02 | LWLLWPVTL | 337 | 2 |
| TCR_110 | A*02:01 | ALWEIQQVV | 297 | 5 |
| TCR_111 | A*24:02 | QYIKWPWYI | 318 | 4 |
| TCR_112 | B*07:02 | QPGQTFSVL | 326 | 4 |
| TCR_113 | A*02:01 | YLQPRTFLL | 288 | 4 |
| TCR_114 | A*24:02 | YYQLYSTQL | 321 | 3 |
| TCR_115 | B*07:02 | SPRWYFYYL | 323 | 3 |
| TCR_116 | A*02:01 | KLWAQCVQL | 287 | 2 |
| TCR_117 | A*02:01 | LLLDRLNQL | 340 | 2 |
| TCR_118 | B*07:02 | SPRWYFYYL | 323 | 2 |
| TCR_119 | A*02:01 | YLYALVYFL | 354 | 2 |
| TCR_120 | A*02:01 | ALWEIQQVV | 297 | 5 |
| TCR_121 | A*24:02 | QYIKWPWYI | 318 | 4 |
| TCR_122 | B*07:02 | QPGQTFSVL | 326 | 4 |
| TCR_123 | A*02:01 | YLQPRTFLL | 288 | 4 |
| TCR_124 | A*24:02 | YYQLYSTQL | 321 | 3 |
| TCR_125 | B*07:02 | SPRWYFYYL | 323 | 3 |
| TCR_126 | A*02:01 | KLWAQCVQL | 287 | 2 |
| TCR_127 | A*02:01 | LLLDRLNQL | 340 | 2 |
| TCR_128 | B*07:02 | SPRWYFYYL | 323 | 2 |
| TCR_129 | A*02:01 | YLYALVYFL | 354 | 2 |
| TCR_130 | A*32:01 | RLFARTRSMW | 679 | N/A² |
| TCR_131 | B*07:02 | QSAPHGVVFL | 680 | N/A² |
| TCR_132 | B*48:01 | RKHFSMMILS | 681 | N/A² |

¹Denotes exemplary T cell receptors set forth in TABLES 5 and 6.
²Denotes epitopes that were identified in Example 21 (MEDi)

Exemplary immunodominant CD8+ T cell epitopes including their corresponding MHC class I alleles on antigen presenting cells, and the corresponding T cell receptors on CD8+ T cells are set forth in TABLE 2.

TABLE 2

| SARS- | | | | No. of | | | | |
|---|---|---|---|---|---|---|---|---|
| CoV-2 | Epitope | | Total | Clono | No. of | | COVID | Immuno |
| Epitope | SEQ ID | HLA | Cells | Types | subjects | TCR IDs | ONLY^{$} | dominant |
| CDR3alpha | | | | | | | | |
| FTSDYY | 328 | A*01:01 | 5 | 1 | 2 | TCR_25 | | YES |
| QLY | | | | | | | | |
| LLYDAN | 286 | A*02:01 | 30 | 20 | 9 | TCR_22, | YES | YES |
| YFL | | | | | | TCR_27, | | |
| | | | | | | TCR_65, | | |
| | | | | | | TCR_107 | | |
| PTDNYIT | 327 | A*01:01 | 13 | 9 | 2 | TCR_53, | YES | YES |
| TY | | | | | | TCR_54 | | |
| QPGQTF | 326 | B*07:02 | 10 | 1 | 2 | TCR_11, | | YES |
| SVL | | | | | | TCR_122 | | |
| RIRGGD | 324 No: | B*07:02 | 3 | 2 | 2 | TCR_22, | YES | YES |
| GKM | | | | | | TCR_27, | | |
| | | | | | | TCR_65, | | |
| | | | | | | TCR_107 | | |
| YLQPRT | 288 | A*02:01 | 24 | 18 | 8 | TCR_43, | YES | YES |
| FLL | | | | | | TCR_88, | | |
| | | | | | | TCR_94, | | |
| | | | | | | TCR_103, | | |
| | | | | | | TCR_108 | | |
| YLQPRT | 288 | A*02:01 | 38 | 3 | 4 | TCR_5, | YES | YES |
| FLL | | | | | | TCR_63, | | |
| | | | | | | TCR_106 | | |
| YLQPRT | 288 | A*02:01 | 6 | 4 | 4 | TCR_45, | YES | YES |
| FLL | | | | | | TCR_63, | | |
| | | | | | | TCR_106 | | |
| YLQPRT | 288 | A*02:01 | 5 | 5 | 4 | TCR_14, | YES | YES |
| FLL | | | | | | TCR_33 | | |
| YLQPRT | 288 | A*02:01 | 3 | 3 | 3 | TCR_43, | YES | YES |
| FLL | | | | | | TCR_88, | | |
| | | | | | | TCR_94, | | |
| | | | | | | TCR_103 | | |
| | | | | | | TCR_108 | | |
| YLQPRT | 288 | A*02:01 | 13 | 2 | 2 | TCR_16, | YES | YES |
| FLL | | | | | | TCR_28 | | |
| CDR3beta | | | | | | | | |
| FTSDYY | 328 | A*01:01 | 10 | 2 | 2 | TCR_25 | YES | |
| QLY | | | | | | | | |
| LLYDAN | 286 | A*02:01 | 9 | 8 | 5 | TCR_22, | YES | YES |
| YFL | | | | | | TCR_27, | | |
| | | | | | | TCR_65, | | |
| | | | | | | TCR_77 | | |
| LLYDAN | 286 | A*02:01 | 7 | 3 | 3 | TCR_27 | YES | YES |
| YFL | | | | | | | | |
| LLYDAN | 286 | A*02:01 | 10 | 5 | 3 | TCR_22, | YES | YES |
| YFL | | | | | | TCR_27, | | |
| | | | | | | TCR_77 | | |
| PTDNYIT | 327 | A*01:01 | 10 | 7 | 2 | TCR_54 | YES | YES |
| TY | | | | | | | | |
| QPGQTF | 326 | B*07:02 | 26 | 5 | 3 | TCR_11, | | YES |
| SVL | | | | | | TCR_112 | | |
| | | | | | | TCR_122 | | |
| RIRGGD | 324 | B*07:02 | 3 | 2 | 2 | TCR_16, | YES | YES |
| GKM | | | | | | TCR_107 | | |
| YLQPRT | 288 | A*02:01 | 15 | 13 | 8 | TCR_63, | YES | YES |
| FLL | | | | | | TCR_106 | | |
| YLQPRT | 288 | A*02:01 | 14 | 10 | 6 | TCR_20, | YES | YES |
| FLL | | | | | | TCR_43, | | |
| | | | | | | TCR_98, | | |
| | | | | | | TCR_103 | | |
| YLQPRT | 288 | A*02:01 | 9 | 7 | 5 | TCR_81, | YES | YES |
| FLL | | | | | | TCR_86 | | |
| YLQPRT | 288 | A*02:01 | 17 | 5 | 4 | TCR_14, | YES | YES |
| FLL | | | | | | TCR_33, | | |
| | | | | | | TCR_70 | | |
| YLQPRT | 288 | A*02:01 | 42 | 3 | 3 | TCR_5, | YES | YES |
| FLL | | | | | | TCR_63, | | |
| | | | | | | TCR_106 | | |
| YLQPRT | 288 | A*02:01 | 6 | 4 | 3 | TCR_90, | YES | YES |
| FLL | | | | | | TCR_98 | | |
| YLQPRT | 288 | A*02:01 | 5 | 4 | 3 | TCR_43, | YES | YES |
| FLL | | | | | | TCR_88, | | |
| | | | | | | TCR_103, | | |
| | | | | | | TCR_108 | | |
| YLQPRT | 288 | A*02:01 | 8 | 4 | 3 | TCR_28 | YES | YES |
| FLL | | | | | | | | |

^{$}Covid Only-specific epitopes not present in patients infected with SARS-Cov-2 as described in Example 22 below.

Exemplary CD4+ T cell epitopes including their corresponding MHC class II alleles on antigen presenting cells, and the corresponding T cell receptors on CD4+ T cells are set forth in TABLE 3.

TABLE 3

| | | | Epitope |
|---|---|---|---|
| | HLA Class | | SEQ ID |
| TCR ID | II | SARS-CoV-2 Epitope | NO: |
| TCR_133 | DPA1*02:02 | NAQALNTLVKQLSSNFG | 688 |
| | DPB1*05:01 | | |
| TCR_134 | DRB1*07:01 | IPTNFTISVTTEILP | 689 |
| TCR_135 | DRB1*07:01 | SGTWLTYTGAIKLDDKDPNFK | 690 |
| TCR_136 | DRB1*07:01 | ASFSTFKCYGVSPTK | 691 |
| | | LNDLCFT | |
| TCR_137 | DRB1*07:01 | LSYYKLGASQRVAGD | 692 |
| TCR_138 | DRB1*14:05 | LLLLDRLNQLESKMSG | 693 |
| | | KGQQQQ | |
| TCR_139 | DRB1*11:01 | RGHLRIAGEHLGRCDIKDLP | 694 |
| TCR_140 | DRB1*08:03 | DQVILLNKHIDAY | 695 |

Exemplary CD4+ T cell epitopes including their corresponding MHC class II alleles on antigen presenting cells are set forth in TABLE 4.

TABLE 4

| HLA Class II | SARS-CoV-2 Protein | 15aa Peptide | SEQ ID NO: |
|---|---|---|---|
| DRB10701 | spike glycoprotein - | YTNSFTRGVYYPDKV | 696 |
| DRB10701 | spike glycoprotein - | VFRSSVLHSTQDLFL | 697 |
| DRB10701 | spike glycoprotein - | DSKTQSLLIVNNATN | 698 |
| DRB10701 | spike glycoprotein - | KIYSKHTPINLVRDL | 699 |
| DRB10701 | spike glycoprotein - | NFRVQPTESIVRFPN | 700 |
| DRB10701 | spike glycoprotein - | NVYADSFVIRGDEVR | 701 |
| DRB10701 | spike glycoprotein - | SPRRARSVASQSIIA | 702 |
| DRB10701 | spike glycoprotein - | VASQSIIAYTMSLGA | 703 |
| DRB10701 | spike glycoprotein - | SIIAYTMSLGAENSV | 704 |
| DRB10701 | spike glycoprotein - | YTMSLGAENSVAYSN | 705 |
| DRB10701 | spike glycoprotein - | SVAYSNNSIAIPTNF | 706 |
| DRB10701 | spike glycoprotein - | QYTSALLAGTITSGW | 707 |
| DRB10701 | spike glycoprotein - | IRASANLAATKMSEC | 708 |
| DRB10701 | ORF8 - | YIRVGARKSAPLIEL | 709 |
| DRB10701 | ORF7a - | KHVYQLRARSVSPKL | 710 |
| DRB10701 | ORF3a - | PSDFVRATATIPIQA | 711 |
| DRB10701 | ORF3a - | KKRWQLALSKGVHFV | 712 |
| DRB10701 | ORF3a - | GVVNPVMEPIYDEPT | 713 |
| DRB10701 | ORF1a polyprotein - | SYELQTPFEIKLAKK | 714 |
| DRB10701 | ORF1a polyprotein - | NFVFPLNSIIKTIQP | 715 |
| DRB10701 | ORF1a polyprotein - | GRIRSVYPVASPNEC | 716 |
| DRB10701 | ORF1a polyprotein - | ASFSASTSAFVETVK | 717 |
| DRB10701 | ORF1a polyprotein - | KKGAWNIGEQKSILS | 718 |
| DRB10701 | ORF1a polyprotein - | RVVRSIFSRTLETAQ | 719 |
| DRB10701 | ORF1a polyprotein - | RVLQKAAITILDGIS | 720 |
| DRB10701 | ORF1a polyprotein - | LRLIDAMMFTSDLAT | 721 |
| DRB10701 | ORF1a polyprotein - | KYCALAPNMMVTNNT | 722 |
| DRB10701 | ORF1a polyprotein - | LEFGATSAALQPEEE | 723 |
| DRB10701 | ORF1a polyprotein - | TVVVNAANVYLKHGG | 724 |
| DRB10701 | ORF1a polyprotein - | ALNKATNNAMQVESD | 725 |
| DRB10701 | ORF1a polyprotein - | YENFNQHEVLLAPLL | 726 |
| DRB10701 | ORF1a polyprotein - | NVYLAVFDKNLYDKL | 727 |
| DRB10701 | ORF1a polyprotein - | KPFITESKPSVEQRK | 728 |
| DRB10701 | ORF1a polyprotein - | TFLKKDAPYIVGDVV | 729 |
| DRB10701 | ORF1a polyprotein - | LRKVPTDNYITTYPG | 730 |
| DRB10701 | ORF1a polyprotein - | FYILPSIISNEKQEI | 731 |
| DRB10701 | ORF1a polyprotein - | IQRKYKGIKIQEGVV | 732 |
| DRB10701 | ORF1a polyprotein - | YTSKTTVASLINTLN | 733 |
| DRB10701 | ORF1a polyprotein - | EEAARYMRSLKVPAT | 734 |
| DRB10701 | ORF1a polyprotein - | KSVYYTSNPTTFHLD | 735 |
| DRB10701 | ORF1a polyprotein - | DVRETMSYLFQHANL | 736 |
| DRB10701 | ORF1a polyprotein - | KLLHKPIVWHVNNAT | 737 |
| DRB10701 | ORF1a polyprotein - | ILKPANNSLKITEEV | 738 |
| DRB10701 | ORF1a polyprotein - | NELSRVLGLKTLATH | 739 |
| DRB10701 | ORF1a polyprotein - | RIKASMPTTIAKNTV | 740 |
| DRB10701 | ORF1a polyprotein - | IINLVQMAPISAMVR | 741 |
| DRB10701 | ORF1a polyprotein - | VNTFSSTFNVPMEKL | 742 |
| DRB10701 | ORF1a polyprotein - | KLSHQSDIEVTGDSC | 743 |
| DRB10701 | ORF1a polyprotein - | HINAQVAKSHNIALI | 744 |
| DRB10701 | ORF1a polyprotein - | RQVVNVVTTKIALKG | 745 |
| DRB10701 | ORF1a polyprotein - | IAAVITREVGFVVPG | 746 |
| DRB10701 | ORF1a polyprotein - | VFSAVGNICYTPSKL | 747 |
| DRB10701 | ORF1a polyprotein - | ICYTPSKLIEYTDFA | 748 |
| DRB10701 | ORF1a polyprotein - | PLIQPIGALDISASI | 749 |
| DRB10701 | ORF1a polyprotein - | ALDISASIVAGGIVA | 750 |
| DRB10701 | ORF1a polyprotein - | FSNSGSDVLYQPPQT | 751 |
| DRB10701 | ORF1a polyprotein - | QTSITSAVLQSGFRK | 752 |
| DRB10701 | ORF1a polyprotein - | NFLVQAGNVQLRVIG | 753 |
| DRB10701 | ORF1a polyprotein - | KKLKKSLNVAKSEFD | 754 |
| DRB10701 | ORF1a polyprotein - | IIPLTTAAKLMVVIP | 755 |
| DRB10701 | ORF1a polyprotein - | LIVTALRANSAVKLQ | 756 |
| DRB10701 | ORF1b polyprotein - | MVPHISRQRLTKYTM | 757 |
| DRB10701 | ORF1b polyprotein - | DFIQTTPGSGVPVVD | 758 |
| DRB10701 | ORF1b polyprotein - | ILTLTRALTAESHVD | 759 |
| DRB10701 | ORF1b polyprotein - | FVVSTGYHFRELGVV | 760 |
| DRB10701 | ORF1b polyprotein - | TNNVAFQTVKPGNFN | 761 |
| DRB10701 | ORF1b polyprotein - | DFAVSKGFFKEGSSV | 762 |
| DRB10701 | ORF1b polyprotein - | QDALFAYTKRNVIPT | 763 |
| DRB10701 | ORF1b polyprotein - | NALLSTDGNKIADKY | 764 |
| DRB10701 | ORF1b polyprotein - | AYLRKHFSMMILSDD | 765 |
| DRB10701 | ORF1b polyprotein - | CFNSTYASQGLVASI | 766 |
| DRB10701 | ORF1b polyprotein - | ASIKNFKSVLYYQNN | 767 |
| DRB10701 | ORF1b polyprotein - | ERFVSLAIDAYPLTK | 768 |
| DRB10701 | ORF1b polyprotein - | DAYPLTKHPNQEYAD | 769 |
| DRB10701 | ORF1b polyprotein - | DHVISTSHKLVLSVN | 770 |
| DRB10701 | ORF1b polyprotein - | TFKLSYGIATVREVL | 771 |
| DRB10701 | ORF1b polyprotein - | TGYRVTKNSKVQIGE | 772 |
| DRB10701 | ORF1b polyprotein - | YFVLTSHTVMPLSAP | 773 |
| DRB10701 | ORF1b polyprotein - | YVRITGLYPTLNISD | 774 |
| DRB10701 | ORF1b polyprotein - | IVDTVSALVYDNKLK | 775 |
| DRB10701 | ORF1b polyprotein - | MFYKGVITHDVSSAI | 776 |
| DRB10701 | ORF1b polyprotein - | QHMVVKAALLADKFP | 777 |
| DRB10701 | ORF1b polyprotein - | KRNIKPVPEVKILNN | 778 |
| DRB10701 | ORF1b polyprotein - | VKILNNLGVDIAANT | 779 |
| DRB10701 | ORF1b polyprotein - | LFRNARNGVLITEGS | 780 |
| DRB10701 | ORF1b polyprotein - | LPETYFTQSRNLQEF | 781 |
| DRB10701 | ORF1b polyprotein - | YGDFSHSQLGGLHLL | 782 |
| DRB10701 | ORF1b polyprotein - | QYLNTLTLAVPYNMR | 783 |
| DRB10701 | ORF1b polyprotein - | RQWLPTGTLLVDSDL | 784 |
| DRB10701 | ORF1b polyprotein - | QKLALGGSVAIKITE | 785 |
| DRB10701 | ORF1b polyprotein - | HANYIFWRNTNPIQL | 786 |
| DRB10701 | ORF1b polyprotein - | KFPLKLRGTAVMSLK | 787 |
| DRB10701 | nucleocapsid | DLKFPRGQGVPINTN | 788 |
| | phosphoprotein - | | |
| DRB10701 | nucleocapsid | TATKAYNVTQAFGRR | 789 |
| | phosphoprotein - | | |
| DRB10701 | nucleocapsid | AQFAPSASAFFGMSR | 790 |
| | phosphoprotein - | | |
| DRB10701 | nucleocapsid | EVTPSGTWLTYTGAI | 791 |
| | phosphoprotein - | | |
| DRB10701 | nucleocapsid | WLTYTGAIKLDDKDP | 792 |
| | phosphoprotein - | | |
| DRB10701 | nucleocapsid | ETQALPQRQKKQQTV | 793 |
| | phosphoprotein - | | |
| DRB10701 | membrane glycoprotein | PKEITVATSRTLSYY | 794 |
| DRB10701 | envelope protein - | SRVKNLNSSRVPDLL | 795 |
| DRB10404 | spike glycoprotein - | VYFASTEKSNIIRGW | 796 |
| DRB10404 | spike glycoprotein - | NLVKNKCVNFNFNGL | 797 |
| DRB10404 | spike glycoprotein - | DLLFNKVTLADAGFI | 798 |
| DRB10404 | spike glycoprotein - | LGKLQDVVNQNAQAL | 799 |
| DRB10404 | spike glycoprotein - | QNAQALNTLVKQLSS | 800 |
| DRB10404 | spike glycoprotein - | IDRLITGRLQSLQTY | 801 |
| DRB10404 | spike glycoprotein - | RLQSLQTYVTQQLIR | 802 |
| DRB10404 | spike glycoprotein - | FPQSAPHGVVFLHVT | 803 |
| DRB10404 | ORF8 - | CLPFTINCQEPKLGS | 804 |
| DRB10404 | ORF7a - | LFIRQEEVQELYSPI | 805 |
| DRB10404 | ORF3a - | ALLAVFQSASKIITL | 806 |
| DRB10404 | ORF3a - | YYQLYSTQLSTDTGV | 807 |
| DRB10404 | ORF3a - | PVMEPIYDEPTTTTS | 808 |
| DRB10404 | ORF1a polyprotein - | TLGVLVPHVGEIPVA | 809 |
| DRB10404 | ORF1a polyprotein - | PLECIKDLLARAGKA | 810 |
| DRB10404 | ORF1a polyprotein - | VESCGNFKVTKGKAK | 811 |
| DRB10404 | ORF1a polyprotein - | KALNLGETFVTHSKG | 812 |
| DRB10404 | ORF1a polyprotein - | IVEEAKKVKPTVVVN | 813 |
| DRB10404 | ORF1a polyprotein - | APLLSAGIFGADPIH | 814 |
| DRB10404 | ORF1a polyprotein - | TAVVIPTKKAGGTTE | 815 |
| DRB10404 | ORF1a polyprotein - | CKSAFYILPSIISNE | 816 |
| DRB10404 | ORF1a polyprotein - | ETKAIVSTIQRKYKG | 817 |
| DRB10404 | ORF1a polyprotein - | TSKTTVASLINTLND | 818 |
| DRB10404 | ORF1a polyprotein - | HFIETISLAGSYKDW | 819 |
| DRB10404 | ORF1a polyprotein - | LGRYMSALNHTKKWK | 820 |
| DRB10404 | ORF1a polyprotein - | VQQESPFVMMSAPPA | 821 |
| DRB10404 | ORF1a polyprotein - | IKPVTYKLDGVVCTE | 822 |
| DRB10404 | ORF1a polyprotein - | VETSNSFDVLKSEDA | 823 |
| DRB10404 | ORF1a polyprotein - | PVSEEVVENPTIQKD | 824 |
| DRB10404 | ORF1a polyprotein - | DIILKPANNSLKITE | 825 |
| DRB10404 | ORF1a polyprotein - | NELSRVLGLKTLATH | 826 |
| DRB10404 | ORF1a polyprotein - | TFTRSTNSRIKASMP | 827 |
| DRB10404 | ORF1a polyprotein - | SLIYSTAALGVLMSN | 828 |
| DRB10404 | ORF1a polyprotein - | DLSLQFKRPINPTDQ | 829 |
| DRB10404 | ORF1a polyprotein - | HSLSHFVNLDNLRAN | 830 |
| DRB10404 | ORF1a polyprotein - | VKMFDAYVNTFSSTF | 831 |
| DRB10404 | ORF1a polyprotein - | KTLVATAEAELAKNV | 832 |
| DRB10404 | ORF1a polyprotein - | LIWNVKDFMSLSEQL | 833 |
| DRB10404 | ORF1a polyprotein - | QVVNVVTTKIALKGG | 834 |
| DRB10404 | ORF1a polyprotein - | LIQPIGALDISASIV | 835 |
| DRB10404 | ORF1a polyprotein - | YLKRRVVFNGVSFST | 836 |
| DRB10404 | ORF1a polyprotein - | AKALNDFSNSGSDVL | 837 |
| DRB10404 | ORF1a polyprotein - | CVLKLKVDTANPKTP | 838 |
| DRB10404 | ORF1a polyprotein - | KYKFVRIQPGQTFSV | 839 |
| DRB10404 | ORF1a polyprotein - | LEDEFTPFDVVRQCS | 840 |
| DRB10404 | ORF1a polyprotein - | ATVAYFNMVYMPASW | 841 |
| DRB10404 | ORF1a polyprotein - | GGKPCIKVATVQSKM | 842 |
| DRB10404 | ORF1a polyprotein - | FSSLPSYAAFATAQE | 843 |
| DRB10404 | ORF1a polyprotein - | PLNIIPLTTAAKLMV | 844 |
| DRB10404 | ORF1a polyprotein - | PDYNTYKNTCDGTTF | 845 |
| DRB10404 | ORF1a polyprotein - | AVKLQNNELSPVALR | 846 |
| DRB10404 | ORF1a polyprotein - | ALRQMSCAAGTTQTA | 847 |
| DRB10404 | ORF1a polyprotein - | ALAYYNTTKGGRFVL | 848 |
| DRB10404 | ORF1b polyprotein - | ETIYNLLKDCPAVAK | 849 |
| DRB10404 | ORF1b polyprotein - | ILRVYANLGERVRQA | 850 |
| DRB10404 | ORF1b polyprotein - | VDSYYSLLMPILTLT | 851 |
| DRB10404 | ORF1b polyprotein - | PILTLTRALTAESHV | 852 |
| DRB10404 | ORF1b polyprotein - | LGVVHNQDVNLHSSR | 853 |
| DRB10404 | ORF1b polyprotein - | VIPTITQMNLKYAIS | 854 |
| DRB10404 | ORF1b polyprotein - | FHQKLLKSIAATRGA | 855 |
| DRB10404 | ORF1b polyprotein - | VFNICQAVTANVNAL | 856 |
| DRB10404 | ORF1b polyprotein - | VASIKNFKSVLYYQN | 857 |
| DRB10404 | ORF1b polyprotein - | STSHKLVLSVNPYVC | 858 |
| DRB10404 | ORF1b polyprotein - | LANTCTERLKLFAAE | 859 |
| DRB10404 | ORF1b polyprotein - | HTVMPLSAPTLVPQE | 860 |
| DRB10404 | ORF1b polyprotein - | AVFISPYNSQNAVAS | 861 |
| DRB10404 | ORF1b polyprotein - | LHPTQAPTHLSVDTK | 862 |
| DRB10404 | ORFlb polyprotein - | LISMMGFKMNYQVNG | 863 |
| DRB10404 | ORF1b polyprotein - | EAIRHVRAWIGFDVE | 864 |
| DRB10404 | ORF1b polyprotein - | STGVNLVAVPTGYVD | 865 |
| DRB10404 | ORF1b polyprotein - | FPVLHDIGNPKAIKC | 866 |
| DRB10404 | ORF1b polyprotein - | KAIKCVPQADVEWKF | 867 |
| DRB10404 | ORF1b polyprotein - | FDKSAFVNLKQLPFF | 868 |
| DRB10404 | ORF1b polyprotein - | YNLWNTFTRLQSLEN | 869 |
| DRB10404 | ORF1b polyprotein - | TRLQSLENVAFNVVN | 870 |
| DRB10404 | ORF1b polyprotein - | PVPEVKILNNLGVDI | 871 |
| DRB10404 | ORF1b polyprotein - | PAHISTIGVCSMTDI | 872 |
| DRB10404 | ORF1b polyprotein - | TVKNYFITDAQTGSS | 873 |
| DRB10404 | ORF1b polyprotein - | VEIIKSQDLSVVSKV | 874 |
| DRB10404 | ORF1b polyprotein - | LNTLTLAVPYNMRVI | 875 |
| DRB10404 | ORF1b polyprotein - | AFLIGCNYLGKPREQ | 876 |
| DRB10404 | ORF1b polyprotein - | ANYIFWRNTNPIQLS | 877 |
| DRB10404 | ORF1b polyprotein - | KFPLKLRGTAVMSLK | 878 |
| DRB10404 | ORF1b polyprotein - | KGRLIIRENNRVVIS | 879 |
| DRB10404 | nucleocapsid | NKDGIIWVATEGALN | 880 |
| | phosphoprotein - | | |
| DRB10404 | nucleocapsid | ATKAYNVTQAFGRRG | 881 |
| | phosphoprotein - | | |
| DRB10404 | nucleocapsid | PQIAQFAPSASAFFG | 882 |
| | phosphoprotein - | | |
| DRB10404 | nucleocapsid | FGMSRIGMEVTPSGT | 883 |
| | phosphoprotein - | | |
| DRB10404 | membrane glycoprotein | FRLFARTRSMWSFNP | 884 |
| DRB10404 | membrane glycoprotein | LNVPLHGTILTRPLL | 885 |
| DRB10404 | membrane glycoprotein | GDSGFAAYSRYRIGN | 886 |
| DRB10404 | envelope protein - | VYSRVKNLNSSRVPD | 887 |
| DRB11501 | spike glycoprotein - | NIDGYFKIYSKHTPI | 888 |
| DRB11501 | spike glycoprotein - | GINITRFQTLLALHR | 889 |
| DRB11501 | spike glycoprotein - | QTLLALHRSYLTPGD | 890 |
| DRB11501 | spike glycoprotein - | EVFNATRFASVYAWN | 891 |
| DRB11501 | spike glycoprotein - | FASVYAWNRKRISNC | 892 |
| DRB11501 | spike glycoprotein - | TESNKKFLPFQQFGR | 893 |
| DRB11501 | spike glycoprotein - | ASQSIIAYTMSLGAE | 894 |
| DRB11501 | spike glycoprotein - | VFAQVKQIYKTPPIK | 895 |
| DRB11501 | spike glycoprotein - | QILPDPSKPSKRSFI | 896 |
| DRB11501 | spike glycoprotein - | IPFAMQMAYRFNGIG | 897 |
| DRB11501 | spike glycoprotein - | LITGRLQSLQTYVTQ | 898 |
| DRB11501 | spike glycoprotein - | QLIRAAEIRASANLA | 899 |
| DRB11501 | ORF8 - | PIHFYSKWYIRVGAR | 900 |
| DRB11501 | ORF8 - | WYIRVGARKSAPLIE | 901 |
| DRB11501 | ORF7a - | KHVYQLRARSVSPKL | 902 |
| DRB11501 | ORF3a - | DFVRATATIPIQASL | 903 |
| DRB11501 | ORF3a - | AVFQSASKIITLKKR | 904 |
| DRB11501 | ORF3a - | KIITLKKRWQLALSK | 905 |
| DRB11501 | ORF1a polyprotein - | VFIKRSDARTAPHGH | 906 |
| DRB11501 | ORF1a polyprotein - | VGEIPVAYRKVLLRK | 907 |
| DRB11501 | ORF1a polyprotein - | YRKVLLRKNGNKGAG | 908 |
| DRB11501 | ORF1a polyprotein - | LLRKNGNKGAGGHSY | 909 |
| DRB11501 | ORF1a polyprotein - | QTPFEIKLAKKFDTF | 910 |
| DRB11501 | ORF1a polyprotein - | NSIIKTIQPRVEKKK | 911 |
| DRB11501 | ORF1a polyprotein - | KKKLDGFMGRIRSVY | 912 |
| DRB11501 | ORF1a polyprotein - | SGLKTILRKGGRTIA | 913 |
| DRB11501 | ORF1a polyprotein - | VETVKGLDYKAFKQI | 914 |
| DRB11501 | ORF1a polyprotein - | FKVTKGKAKKGAWNI | 915 |
| DRB11501 | ORF1a polyprotein - | EAARVVRSIFSRTLE | 916 |
| DRB11501 | ORF1a polyprotein - | AQNSVRVLQKAAM | 917 |
| DRB11501 | ORF1a polyprotein - | IIGGAKLKALNLGET | 918 |
| DRB11501 | ORF1a polyprotein - | VVNAANVYLKHGGGV | 919 |
| DRB11501 | ORF1a polyprotein - | GALNKATNNAMQVES | 920 |
| DRB11501 | ORF1a polyprotein - | ETKAIVSTIQRKYKG | 921 |
| DRB11501 | ORF1a polyprotein - | EAARYMRSLKVPATV | 922 |
| DRB11501 | ORF1a polyprotein - | LKTLLSLREVRTIKV | 923 |
| DRB11501 | ORF1a polyprotein - | REVRTIKVFTTVDNI | 924 |
| DRB11501 | ORF1a polyprotein - | QESPFVMMSAPPAQY | 925 |
| DRB11501 | ORF1a polyprotein - | LNQLTGYKKPASREL | 926 |
| DRB11501 | ORF1a polyprotein - | VVAIDYKHYTPSFKK | 927 |
| DRB11501 | ORF1a polyprotein - | RVLGLKTLATHGLAA | 928 |
| DRB11501 | ORF1a polyprotein - | TIANYAKPFLNKVVS | 929 |
| DRB11501 | ORF1a polyprotein - | LINIIIWFLLLSVCL | 930 |
| DRB11501 | ORF1a polyprotein - | NLVQMAPISAMVRMY | 931 |
| DRB11501 | ORF1a polyprotein - | STCMMCYKRNRATRV | 932 |
| DRB11501 | ORF1a polyprotein - | RDLSLQFKRPINPTD | 933 |
| DRB11501 | ORF1a polyprotein - | LSTFISAARQGFVDS | 934 |
| DRB11501 | ORF1a polyprotein - | SHQSDIEVTGDSCNN | 935 |
| DRB11501 | ORF1a polyprotein - | MLTYNKVENMTPRDL | 936 |
| DRB11501 | ORF1a polyprotein - | ARHINAQVAKSHNIA | 937 |
| DRB11501 | ORF1a polyprotein - | LSEQLRKQIRSAAKK | 938 |
| DRB11501 | ORF1a polyprotein - | VTTKIALKGGKIVNN | 939 |
| DRB11501 | ORF1a polyprotein - | IIGYKAIDGGVTRDI | 940 |
| DRB11501 | ORF1a polyprotein - | DVLLPLTQYNRYLAL | 941 |
| DRB11501 | ORF1a polyprotein - | QYNRYLALYNKYKYF | 942 |
| DRB11501 | ORF1a polyprotein - | YLALYNKYKYFSGAM | 943 |
| DRB11501 | ORF1a polyprotein - | SAVLQSGFRKMAFPS | 944 |
| DRB11501 | ORF1a polyprotein - | LLIRKSNHNFLVQAG | 945 |
| DRB11501 | ORF1a polyprotein - | QAGNVQLRVIGHSMQ | 946 |
| DRB11501 | ORF1a polyprotein - | GVTFQSAVKRTIKGT | 947 |
| DRB11501 | ORF1a polyprotein - | MSAFAMMFVKHKHAF | 948 |
| DRB11501 | ORF1a polyprotein - | EFRYMNSQGLLPPKN | 949 |
| DRB11501 | ORF1a polyprotein - | LNIKLLGVGGKPCIK | 950 |
| DRB11501 | ORF1a polyprotein - | EVVLKKLKKSLNVAK | 951 |
| DRB11501 | ORF1a polyprotein - | ADQAMTQMYKQARSE | 952 |
| DRB11501 | ORF1a polyprotein - | IVTALRANSAVKLQN | 953 |
| DRB11501 | ORF1a polyprotein - | ALAYYNTTKGGRFVL | 954 |
| DRB11501 | ORF1b polyprotein - | YFVVKRHTFSNYQHE | 955 |
| DRB11501 | ORF1b polyprotein - | GDMVPHISRQRLTKY | 956 |
| DRB11501 | ORF1b polyprotein - | DILRVYANLGERVRQ | 957 |
| DRB11501 | ORF1b polyprotein - | ILTLTRALTAESHVD | 958 |
| DRB11501 | ORF1b polyprotein - | FPFNKWGKARLYYDS | 959 |
| DRB11501 | ORF1b polyprotein - | DALFAYTKRNVIPTI | 960 |
| DRB11501 | ORF1b polyprotein - | PTITQMNLKYAISAK | 961 |
| DRB11501 | ORF1b polyprotein - | LKYAISAKNRARTVA | 962 |
| DRB11501 | ORF1b polyprotein - | KLLKSIAATRGATVV | 963 |
| DRB11501 | ORF1b polyprotein - | PNMLRIMASLVLARK | 964 |
| DRB11501 | ORF1b polyprotein - | SQGLVASIKNFKSVL | 965 |
| DRB11501 | ORF1b polyprotein - | HTVMPLSAPTLVPQE | 966 |
| DRB11501 | ORF1b polyprotein - | NVANYQKVGMQKYST | 967 |
| DRB11501 | ORF1b polyprotein - | FAIGLALYYPSARIV | 968 |
| DRB11501 | ORF1b polyprotein - | NYDLSVVNARLRAKH | 969 |
| DRB11501 | ORF1b polyprotein - | ALVYDNKLKAHKDKS | 970 |
| DRB11501 | ORF1b polyprotein - | CFKMFYKGVITHDVS | 971 |
| DRB11501 | ORF1b polyprotein - | VVREFLTRNPAWRKA | 972 |
| DRB11501 | ORF1b polyprotein - | VFISPYNSQNAVASK | 973 |
| DRB11501 | ORF1b polyprotein - | GIPKDMTYRRLISMM | 974 |
| DRB11501 | ORF1b polyprotein - | LISMMGFKMNYQVNG | 975 |
| DRB11501 | ORF1b polyprotein - | DQFKHLIPLMYKGLP | 976 |
| DRB11501 | ORF1b polyprotein - | FELTSMKYFVKIGPE | 977 |
| DRB11501 | ORF1b polyprotein - | QHMVVKAALLADKFP | 978 |
| DRB11501 | ORF1b polyprotein - | VAFELWAKRNIKPVP | 979 |
| DRB11501 | ORF1b polyprotein - | KRNIKPVPEVKILNN | 980 |
| DRB11501 | ORF1b polyprotein - | KPVPEVKILNNLGVD | 981 |
| DRB11501 | ORF1b polyprotein - | NTVIWDYKRDAPAHI | 982 |
| DRB11501 | ORF1b polyprotein - | TQFNYYKKVDGVVQQ | 983 |
| DRB11501 | ORF1b polyprotein - | LLIGLAKRFKESPFE | 984 |
| DRB11501 | ORF1b polyprotein - | PGVAMPNLYKMQRML | 985 |
| DRB11501 | ORF1b polyprotein - | LNTLTLAVPYNMRVI | 986 |
| DRB11501 | ORF1b polyprotein - | RVIHFGAGSDKGVAP | 987 |
| DRB11501 | ORF1b polyprotein - | KFPLKLRGTAVMSLK | 988 |
| DRB11501 | ORF1b polyprotein - | LSLLSKGRLIIRENN | 989 |
| DRB11501 | ORF1b polyprotein - | RLIIRENNRVVISSD | 990 |
| DRB11501 | ORF1b polyprotein - | RENNRVVISSDVLVN | 991 |
| DRB11501 | nucleocapsid | SSPDDQIGYYRRATR | 992 |
| | phosphoprotein - | | |
| DRB11501 | nucleocapsid | GYYRRATRRIRGGDG | 993 |
| | phosphoprotein - | | |
| DRB11501 | nucleocapsid | RATRRIRGGDGKMKD | 994 |
| | phosphoprotein - | | |
| DRB11501 | nucleocapsid | LNKHIDAYKTFPPTE | 995 |
| | phosphoprotein - | | |
| DRB11501 | membrane glycoprotein | SFRLFARTRSMWSFN | 996 |
| DRB11501 | membrane glycoprotein | AVILRGHLRIAGHHL | 997 |
| DRB11501 | membrane glycoprotein | GDSGFAAYSRYRIGN | 998 |
| DRB11501 | envelope protein - | VKPSFYVYSRVKNLN | 999 |
| DPA10202B10501 | spike glycoprotein - | PDKVFRSSVLHSTQD | 1000 |
| DPA10202B10501 | spike glycoprotein - | INITRFQTLLALHRS | 1001 |
| DPA10202B10501 | spike glycoprotein - | YRLFRKSNLKPFERD | 1002 |
| DPA10202B10501 | spike glycoprotein - | VLTESNKKFLPFQQF | 1003 |
| DPA10202B10501 | spike glycoprotein - | RRARSVASQSIIAYT | 1004 |
| DPA10202B10501 | spike glycoprotein - | IAYTMSLGAENSVAY | 1005 |
| DPA10202B10501 | spike glycoprotein - | TNFTISVTTEILPVS | 1006 |
| DPA10202B10501 | spike glycoprotein - | VFAQVKQIYKTPPIK | 1007 |
| DPA10202B10501 | spike glycoprotein - | FSQILPDPSKPSKRS | 1008 |
| DPA10202B10501 | spike glycoprotein - | IPFAMQMAYRFNGIG | 1009 |
| DPA10202B10501 | spike glycoprotein - | GKIQDSLSSTASALG | 1010 |
| DPA10202B10501 | spike glycoprotein - | ALNTLVKQLSSNFGA | 1011 |
| DPA10202B10501 | spike glycoprotein - | LITGRLQSLQTYVTQ | 1012 |
| DPA10202B10501 | spike glycoprotein - | QLIRAAEIRASANLA | 1013 |
| DPA10202B10501 | spike glycoprotein - | IRASANLAATKMSEC | 1014 |
| DPA10202B10501 | ORF8 - | KWYIRVGARKSAPLI | 1015 |
| DPA10202B10501 | ORF7a - | KHVYQLRARSVSPKL | 1016 |
| DPA10202B10501 | ORF6 - | NLIIKNLSKSLTENK | 1017 |
| DPA10202B10501 | ORF3a - | SDFVRATATIPIQAS | 1018 |
| DPA10202B10501 | ORF3a - | AVFQSASKIITLKKR | 1019 |
| DPA10202B10501 | ORF3a - | KIITLKKRWQLALSK | 1020 |
| DPA10202B10501 | ORF1a polyprotein - | SLPVLQVRDVLVRGF | 1021 |
| DPA10202B10501 | ORF1a polyprotein - | LEQPYVFIKRSDART | 1022 |
| DPA10202B10501 | ORF1a polyprotein - | PVAYRKVLLRKNGNK | 1023 |
| DPA10202B10501 | ORF1a polyprotein - | PFEIKLAKKFDTFNG | 1024 |
| DPA10202B10501 | ORF1a polyprotein - | FPLNSIIKTIQPRVE | 1025 |
| DPA10202B10501 | ORF1a polyprotein - | FMGRIRSVYPVASPN | 1026 |
| DPA10202B10501 | ORF1a polyprotein - | NESGLKTILRKGGRT | 1027 |
| DPA10202B10501 | ORF1a polyprotein - | LEILQKEKVNINIVG | 1028 |
| DPA10202B10501 | ORF1a polyprotein - | LSPLYAFASEAARVV | 1029 |
| DPA10202B10501 | ORF1a polyprotein - | ARVVRSIFSRTLETA | 1030 |
| DPA10202B10501 | ORF1a polyprotein - | TAQNSVRVLQKAAIT | 1031 |
| DPA10202B10501 | ORF1a polyprotein - | RLIDAMMFTSDLATN | 1032 |
| DPA10202B10501 | ORF1a polyprotein - | ALAPNMMVTNNTFTL | 1033 |
| DPA10202B10501 | ORF1a polyprotein - | VIKTLQPVSELLTPL | 1034 |
| DPA10202B10501 | ORF1a polyprotein - | SGYLKLTDNVYIKNA | 1035 |
| DPA10202B10501 | ORF1a polyprotein - | KPTVVVNAANVYLKH | 1036 |
| DPA10202B10501 | ORF1a polyprotein - | VAGALNKATNNAMQV | 1037 |
| DPA10202B10501 | ORF1a polyprotein - | ITESKPSVEQRKQDD | 1038 |
| DPA10202B10501 | ORF1a polyprotein - | TFLKKDAPYIVGDVV | 1039 |
| DPA10202B10501 | ORF1a polyprotein - | TAVVIPTKKAGGTTE | 1040 |
| DPA10202B10501 | ORF1a polyprotein - | ARYMRSLKVPATVSV | 1041 |
| DPA10202B10501 | ORF1a polyprotein - | KVPATVSVSSPDAVT | 1042 |
| DPA10202B10501 | ORF1a polyprotein - | LQQIELKFNPPALQD | 1043 |
| DPA10202B10501 | ORF1a polyprotein - | PSFKKGAKLLHKPIV | 1044 |
| DPA10202B10501 | ORF1a polyprotein - | KLLHKPIVWFWNNAT | 1045 |
| DPA10202B10501 | ORF1a polyprotein - | SLTIKKPNELSRVLG | 1046 |
| DPA10202B10501 | ORF1a polyprotein - | VLGLKTLATHGLAAV | 1047 |
| DPA10202B10501 | ORF1a polyprotein - | LNKVVSTTTNIVTRC | 1048 |
| DPA10202B10501 | ORF1a polyprotein - | CTFTRSTNSRIKASM | 1049 |
| DPA10202B10501 | ORF1a polyprotein - | RIKASMPTTIAKNTV | 1050 |
| DPA10202B10501 | ORF1a polyprotein - | RDLSLQFKRPINPTD | 1051 |
| DPA10202B10501 | ORF1a polyprotein - | NAQVAKSHNIALIWN | 1052 |
| DPA10202B10501 | ORF1a polyprotein - | ATTRQVVNVVTTKIA | 1053 |
| DPA10202B10501 | ORF1a polyprotein - | KIALKGGKIVNNWLK | 1054 |
| DPA10202B10501 | ORF1a polyprotein - | CPLIAAVITREVGFV | 1055 |
| DPA10202B10501 | ORF1a polyprotein - | GTILRTTNGDFLHFL | 1056 |
| DPA10202B10501 | ORF1a polyprotein - | NYLKRRVVFNGVSFS | 1057 |
| DPA10202B10501 | ORF1a polyprotein - | DLLIRKSNHNFLVQA | 1058 |
| DPA10202B10501 | ORF1a polyprotein - | LVQAGNVQLRVIGHS | 1059 |
| DPA10202B10501 | ORF1a polyprotein - | PKYKFVRIQPGQTFS | 1060 |
| DPA10202B10501 | ORF1a polyprotein - | IISVTSNYSGVVTTV | 1061 |
| DPA10202B10501 | ORF1a polyprotein - | SIDAFKLNIKLLGVG | 1062 |
| DPA10202B10501 | ORF1a polyprotein - | CIKVATVQSKMSDVK | 1063 |
| DPA10202B10501 | ORF1a polyprotein - | VLKKLKKSLNVAKSE | 1064 |
| DPA10202B10501 | ORF1a polyprotein - | AMTQMYKQARSEDKR | 1065 |
| DPA10202B10501 | ORF1a polyprotein - | DKRAKVTSAMQTMLF | 1066 |
| DPA10202B10501 | ORF1a polyprotein - | FTMLRKLDNDALNNI | 1067 |
| DPA10202B10501 | ORF1a polyprotein - | IPLTTAAKLMVVIPD | 1068 |
| DPA10202B10501 | ORF1a polyprotein - | IVTALRANSAVKLQN | 1069 |
| DPA10202B10501 | ORF1a polyprotein - | YNTTKGGRFVLALLS | 1070 |
| DPA10202B10501 | ORF1a polyprotein - | FIKGLNNLNRGMVLG | 1071 |
| DPA10202B10501 | ORF1a polyprotein - | LGSLAATVRLQAGNA | 1072 |
| DPA10202B10501 | ORF1b polyprotein - | SYFVVKRHTFSNYQH | 1073 |
| DPA10202B10501 | ORF1b polyprotein - | PILTLTRALTAESHV | 1074 |
| DPA10202B10501 | ORF1b polyprotein - | PAMHAASGNLLLDKR | 1075 |
| DPA10202B10501 | ORF1b polyprotein - | ALFAYTKRNVIPTIT | 1076 |
| DPA10202B10501 | ORF1b polyprotein - | IPTITQMNLKYAISA | 1077 |
| DPA10202B10501 | ORF1b polyprotein - | NLKYAISAKNRARTV | 1078 |
| DPA10202B10501 | ORF1b polyprotein - | QKLLKSIAATRGATV | 1079 |
| DPA10202B10501 | ORF1b polyprotein - | VIGTSKFYGGWHNML | 1080 |
| DPA10202B10501 | ORF1b polyprotein - | MASLVLARKHTTCCS | 1081 |
| DPA10202B10501 | ORF1b polyprotein - | LLSTDGNKIADKYVR | 1082 |
| DPA10202B10501 | ORF1b polyprotein - | ASQGLVASIKNFKSV | 1083 |
| DPA10202B10501 | ORF1b polyprotein - | VISTSHKLVLSVNPY | 1084 |
| DPA10202B10501 | ORF1b polyprotein - | LSYGIATVREVLSDR | 1085 |
| DPA10202B10501 | ORF1b polyprotein - | TSHTVMPLSAPTLVP | 1086 |
| DPA10202B10501 | ORF1b polyprotein - | VANYQKVGMQKYSTL | 1087 |
| DPA10202B10501 | ORF1b polyprotein - | IVDTVSALVYDNKLK | 1088 |
| DPA10202B10501 | ORF1b polyprotein - | HDVSSAINRPQIGVV | 1089 |
| DPA10202B10501 | ORF1b polyprotein - | ASKILGLPTQTVDSS | 1090 |
| DPA10202B10501 | ORF1b polyprotein - | RRLISMMGFKMNYQV | 1091 |
| DPA10202B10501 | ORF1b polyprotein - | PPGDQFKHLIPLMYK | 1092 |
| DPA10202B10501 | ORF1b polyprotein - | HMVVKAALLADKFPV | 1093 |
| DPA10202B10501 | ORF1b polyprotein - | VDLFRNARNGVLITE | 1094 |
| DPA10202B10501 | ORF1b polyprotein - | IGLAKRFKESPFELE | 1095 |
| DPA10202B10501 | ORF1b polyprotein - | ATLPKGIMMNVAKYT | 1096 |
| DPA10202B10501 | ORF1b polyprotein - | QWLPTGTLLVDSDLN | 1097 |
| DPA10202B10501 | ORF1b polyprotein - | LGGSVAIKITEHSWN | 1098 |
| DPA10202B10501 | ORF1b polyprotein - | PLKLRGTAVMSLKEG | 1099 |
| DPA10202B10501 | ORF1b polyprotein - | ILSLLSKGRLIIREN | 1100 |
| DPA10202B10501 | ORF1b polyprotein - | GRLIIRENNRVVISS | 1101 |
| DPA10202B10501 | nucleocapsid | DQIGYYRRATRRIRG | 1102 |
| | phosphoprotein - | | |
| DPA10202B10501 | nucleocapsid | IGTRNPANNAAIVLQ | 1103 |
| | phosphoprotein - | | |
| DPA10202B10501 | nucleocapsid | QLESKMSGKGQQQQG | 1104 |
| | phosphoprotein - | | |
| DPA10202B10501 | nucleocapsid | ASAFFGMSRIGMEVT | 1105 |
| | phosphoprotein - | | |
| DPA10202B10501 | nucleocapsid | VILLNKHIDAYKTFP | 1106 |
| | phosphoprotein - | | |
| DPA10202B10501 | nucleocapsid | PQRQKKQQTVTLLPA | 1107 |
| | phosphoprotein - | | |
| DPA10202B10501 | membrane glycoprotein | IASFRLFARTRSMWS | 1108 |
| DPA10202B10501 | membrane glycoprotein | IGAVILRGHLRIAGH | 1109 |
| DPA10202B10501 | envelope protein - | SFVSEETGTLIVNSV | 1110 |

The TCRs set forth in TABLE 1, including their clonotypes, are set forth in TABLE 5, and nucleotide sequences encoding exemplary corresponding TCRs are set forth in TABLE 6.

TABLE 5

| | CDR3 alpha | | | | CDR3 beta | | | |
|---|---|---|---|---|---|---|---|---|
| | | SEQ | Alpha | Alpha | | SEQ | Beta | Beta |
| TCR ID | Sequence | ID NO | V gene | J gene | Sequence | ID NO | V gene | J gene |
| TCR_1 | CAMYEGNEKL | 417 | TRAV14/ | TRAJ48 | CASGGLMRGL | 538 | TRBV9 | TRBJ2-3 |
| | TF | | DV4 | | DTQYF | | | |
| TCR_2 | CLVGNNARLM | 418 | TRAV4 | TRAJ31 | CASSLDRESM | 539 | TRBV5- | TRBJ1-1 |
| | F | | | | NIEAFF | | 4 | |
| TCR_3 | CAVRSHGSGG | 419 | TRAV41 | TRAJ53 | CASRELGQETQ | 540 | TRBV19 | TRBJ2-5 |
| | SNYKLTF | | | | YF | | | |
| TCR_4 | CAVVTPPARL | 420 | TRAV39 | TRAJ31 | CASSPLTGQGL | 541 | TRBV28 | TRBJ2-7 |
| | MF | | | | GGAYEQYF | | | |
| TCR_5 | CVVNEGDKIIF | 421 | TRAV12- | TRAJ30 | CASSADIEQYF | 542 | TRBV7- | TRBJ2-7 |
| | | | 1 | | | | 9 | |
| TCR_6 | CAMREASASG | 422 | TRAV14/ | TRAJ6 | CASSTYRGQPQ | 543 | TRBV28 | TRBJ1-5 |
| | GSYIPTF | | DV4 | | HF | | | |
| TCR_7 | CAAVNNNARL | 423 | TRAV38- | TRAJ31 | CASSEGESNTG | 544 | TRBV9 | TRBJ2-2 |
| | MF | | 2/DV8 | | ELFF | | | |
| TCR_8 | CVVNSGNKLV | 424 | TRAV12- | TRAJ47 | CASSADAGTSS | 545 | TRBV9 | TRBJ2-1 |
| | F | | 1 | | EQFF | | | |
| TCR_9 | CAGRTFDKIIF | 425 | TRAV25 | TRAJ30 | CASSIVPGSPSR | 546 | TRBV19 | TRBJ1-2 |
| | | | | | GYTF | | | |
| TCR_10 | CVVTPRGSTLG | 426 | TRAV12- | TRAJ18 | CASSTEDRVSY | 547 | TRBV5- | TRBJ2-1 |
| | RLYF | | 1 | | NEQFF | | 1 | |
| TCR_11 | CAVQAPMSAR | 427 | TRAV20 | TRAJ42 | CASSSKGGGQ | 548 | TRBV7- | TRBJ2-5 |
| | GSQGNLIF | | | | QTQYF | | 2 | |
| TCR_12 | CAVKTDMRF | 428 | TRAV12- | TRAJ43 | CASSDRDSVN | 549 | TRBV7- | TRBJ1-2 |
| | | | 2 | | YGYTF | | 8 | |
| TCR_13 | CVVTRSQGNLI | 429 | TRAV12- | TRAJ42 | CASRASGGFNE | 550 | TRBV12- | TRBJ2-1 |
| | F | | 1 | | QFF | | 5 | |
| TCR_14 | CVVNAPSGNT | 430 | TRAV12- | TRAJ29 | CSARDRQGQN | 551 | TRBV20- | TRBJ2-2 |
| | PLVF | | 1 | | TGELFF | | 1 | |
| TCR_15 | CAGLGTGRRA | 431 | TRAV3 | TRAJ5 | CAISERDFQET | 552 | TRBV10- | TRBJ2-5 |
| | LTF | | | | QYF | | 3 | |
| TCR_16 | CVVNKEDRLM | 432 | TRAV12- | TRAJ31 | CASHSDRNTG | 553 | TRBV7- | TRBJ2-2 |
| | F | | 1 | | ELFF | | 8 | |
| TCR_17 | CAMREGPAEG | 433 | TRAV14/ | TRAJ56 | CASNLLTGGD | 554 | TRBV27 | TRBJ2-3 |
| | GANSKLTF | | DV4 | | ADTQYF | | | |
| TCR_18 | CAVRQNDKIIF | 434 | TRAV21 | TRAJ30 | CASSLAGAQG | 555 | TRBVS- | TRBJ1-2 |
| | | | | | YTF | | 1 | |
| TCR_19 | CVVTGAGSYQ | 435 | TRAV8-2 | TRAJ28 | CATSDDSEKLF | 556 | TRBV24- | TRBJ1-4 |
| | LTF | | | | F | | 1 | |
| TCR_20 | CVVNMATDKL | 436 | TRAV12- | TRAJ34 | CASGGTNTGE | 557 | TRBV2 | TRBJ2-2 |
| | IF | | 1 | | LFF | | | |
| TCR_21 | CAGGPSRNND | 437 | TRAV27 | TRAJ43 | CASSPALGREQ | 558 | TRBV27 | TRBJ2-7 |
| | MRF | | | | YF | | | |
| TCR_22 | CAVIVAGNTPL | 438 | TRAV8-1 | TRAJ29 | CASSFGGSTEA | 559 | TRBV11- | TRBJ1-1 |
| | VF | | | | FF | | 1 | |
| TCR_23 | CVVNPTGTAS | 439 | TRAV12- | TRAJ44 | CASSVAGGLY | 560 | TRBV9 | TRBJ2-1 |
| | KLTF | | 1 | | EQFF | | | |
| TCR_24 | CVVITGGYNK | 440 | TRAV12- | TRAJ4 | CASSLIGAGST | 561 | TRBVS- | TRBJ2-3 |
| | LIF | | 1 | | DTQYF | | 1 | |
| TCR_25 | CAAIGGSTLGR | 441 | TRAV29/ | TRAJ18 | CASSPIKDTRQ | 562 | TRBV28 | TRBJ2-2 |
| | LYF | | DVS | | EYTGELFF | | | |
| TCR_26 | CAVGDGNNRL | 442 | TRAV8-3 | TRAJ7 | CASSLGTASTD | 563 | TRBV27 | TRBJ2-3 |
| | AF | | | | TQYF | | | |
| TCR_27 | CAWILSGNTPL | 443 | TRAV8-1 | TRAJ29 | CASSLGTGGTE | 564 | TRBV11- | TRBJ1-1 |
| | VF | | | | AFF | | 2 | |
| TCR_28 | CVVNKEDRLM | 432 | TRAV12- | TRAJ31 | CSVDRDRNTG | 565 | TRBV29- | TRBJ2-2 |
| | F | | 1 | | ELFF | | 1 | |
| TCR_29 | CALGNTGGFK | 444 | TRAV24 | TRAJ9 | CASSVEVQARS | 566 | TRBV9 | TRBJ2-3 |
| | TIF | | | | DTQYF | | | |
| TCR_30 | CALGALTGQN | 445 | TRAV16 | TRAJ26 | CASSRLESLGN | 567 | TRBV25- | TRBJ1-3 |
| | FVF | | | | TIYF | | 1 | |
| TCR_31 | CAFPSGGSNY | 446 | TRAV24 | TRAJ53 | CSARDIGTGAH | 568 | TRBV20- | TRBJ2-7 |
| | KLTF | | | | YEQYF | | 1 | |
| TCR_32 | CGTAFFNAGG | 447 | TRAV30 | TRAJ52 | CASSPGASYNE | 569 | TRBV6- | TRBJ2-1 |
| | TSYGKLTF | | | | QFF | | 6 | |
| TCR_33 | CVVNDPNSGN | 448 | TRAV12- | TRAJ29 | CSARDQASQN | 570 | TRBV20- | TRBJ2-2 |
| | TPLVF | | 1 | | TGELFF | | 1 | |
| TCR_34 | CAYMDNNDM | 449 | TRAV38- | TRAJ43 | CASSDGQGGY | 571 | TRBV9 | TRBJ1-2 |
| | RF | | 2/DV8 | | GYTF | | | |
| TCR_35 | CAYHERALTF | 450 | TRAV38- | TRAJ5 | CASSHGTTTYN | 572 | TRBV5- | TRBJ2-1 |
| | | | 2/DV8 | | EQFF | | 1 | |
| TCR_36 | CLAVNTDKLIF | 451 | TRAV4 | TRAJ34 | CASSRTGGSYN | 573 | TRBV5- | TRBJ2-1 |
| | | | | | EQFF | | 1 | |
| TCR_37 | CAMQSDSWG | 452 | TRAV12- | TRAJ24 | CSASPLLEQYF | 574 | TRBV20- | TRBJ2-7 |
| | KLQF | | 3 | | | | 1 | |
| TCR_38 | CAGAGGQKLL | 453 | TRAV27 | TRAJ16 | CASSQDSGTGS | 575 | TRBV4- | TRBJ1-3 |
| | F | | | | KNTIYF | | 1 | |
| TCR_39 | CGTESPGNND | 454 | TRAV30 | TRAJ43 | CASSSSALEDP | 576 | TRBV5- | TRBJ2-7 |
| | MRF | | | | EQYF | | 4 | |
| TCR_40 | CLVGGWVSGG | 455 | TRAV4 | TRAJ6 | CASKTGGAAK | 577 | TRBV2 | TRBJ2-4 |
| | SYIPTF | | | | NIQYF | | | |
| TCR_41 | CALMEANQGG | 456 | TRAV19 | TRAJ23 | CASSVGGSWT | 578 | TRBV9 | TRBJ2-3 |
| | KLIF | | | | DTQYF | | | |
| TCR_42 | CAASTGGQKL | 457 | TRAV13- | TRAJ16 | CASSDLAQYL | 579 | TRBV5- | TRBJ2-2 |
| | LF | | 2 | | NTGELFF | | 4 | |
| TCR_43 | CVVNKDNDM | 458 | TRAV12- | TRAJ43 | CASQDINTGEL | 580 | TRBV6- | TRBJ2-2 |
| | RF | | 1 | | FF | | 2 | |
| TCR_44 | CAGARSGNTG | 459 | TRAV27 | TRAJ37 | CASSLGPGQG | 581 | TRBV7- | TRBJ2-1 |
| | KLIF | | | | YNEQFF | | 6 | |
| TCR_45 | CAAGRDDKIIF | 460 | TRAV12- | TRAJ30 | CASSSMTSGIR | 582 | TRBV6- | TRBJ2-7 |
| | | | 2 | | YEQYF | | 2 | |
| TCR_46 | CAMRGAINTG | 461 | TRAV14/ | TRAJ44 | CAISESWTSGI | 583 | TRBV10- | TRBJ2-5 |
| | TASKLTF | | DV4 | | GREETQYF | | 3 | |
| TCR_47 | CALSVRIQGAQ | 462 | TRAV19 | TRAJ54 | CASSLYEDRA | 584 | TRBV13 | TRBJ2-7 |
| | KINF | | | | NWEQYF | | | |
| TCR_48 | CAVWRFQKLV | 463 | TRAV21 | TRAJ8 | CSAQSQLRVLE | 585 | TRBV20- | TRBJ2-5 |
| | F | | | | ETQYF | | 1 | |
| TCR_49 | CVVNSGNTPL | 464 | TRAV12- | TRAJ29 | CASSYLYRVA | 586 | TRBV6- | TRBJ2-2 |
| | VF | | 1 | | GELFF | | 1 | |
| TCR_50 | CAYIETGNQFY | 465 | TRAV38- | TRAJ49 | CASSTGTVGYE | 587 | TRBV27 | TRBJ2-7 |
| | F | | 2/DV8 | | QYF | | | |
| TCR_51 | CAYIENNARL | 466 | TRAV38- | TRAJ31 | CASSLQGTGY | 588 | TRBV27 | TRBJ1-2 |
| | MF | | 2/DV8 | | GYTF | | | |
| TCR_52 | CAESALGGSQ | 467 | TRAV5 | TRAJ42 | CASRSWVRAP | 589 | TRBV6- | TRBJ1-5 |
| | GNLIF | | | | NQPQHF | | 1 | |
| TCR_53 | CVVRYDSWGK | 468 | TRAV12- | TRAJ24 | CASSLASNNYE | 590 | TRBV28 | TRBJ2-7 |
| | LQF | | 1 | | QYF | | | |
| TCR_54 | CVVRADSWGK | 469 | TRAV12- | TRAJ24 | CASSFSSNSYE | 591 | TRBV28 | TRBJ2-7 |
| | LQF | | 1 | | QYF | | | |
| TCR_55 | CALIGGSNDY | 470 | TRAV9-2 | TRAJ20 | CAKLVTGAVS | 592 | TRBV2 | TRBJ2-1 |
| | KLSF | | | | GEQFF | | | |
| TCR_56 | CATGNNNDMR | 471 | TRAV17 | TRAJ43 | CASSSSTGGNY | 593 | TRBV12- | TRBJ1-2 |
| | F | | | | GYTF | | 3 | |
| TCR_57 | CAILQGAQKL | 472 | TRAV12- | TRAJ54 | CASSLVSGELF | 594 | TRBV19 | TRBJ2-2 |
| | VF | | 3 | | F | | | |
| TCR_58 | CALSESGSTGD | 473 | TRAV19 | TRAJ20 | CATNPAGGPY | 595 | TRBV19 | TRBJ2-1 |
| | YKLSF | | | | NEQFF | | | |
| TCR_59 | CAGQRDDMRF | 474 | TRAV35 | TRAJ43 | CATSGDRGWQ | 596 | TRBV12- | TRBJ2-7 |
| | | | | | YF | | 3 | |
| TCR_60 | CALPTSRNSGN | 475 | TRAV6 | TRAJ29 | CASSSIWTSVN | 597 | TRBV5- | TRBJ2-1 |
| | TPLVF | | | | EQFF | | 6 | |
| TCR_61 | CAYSGTASKL | 476 | TRAV38- | TRAJ44 | CSVVDRGRFY | 598 | TRBV29- | TRBJ2-1 |
| | TF | | 2/DV8 | | NEQFF | | 1 | |
| TCR_62 | CAMRRPGANN | 477 | TRAV14/ | TRAJ36 | CASSAQEGRIE | 599 | TRBV4- | TRBJ1-1 |
| | LFF | | DV4 | | MNTEAFF | | 3 | |
| TCR_63 | CAVNRDDKIIF | 478 | TRAV12- | TRAJ30 | CASSPDIEQYF | 600 | TRBV7- | TRBJ2-7 |
| | | | 2 | | | | 9 | |
| TCR_64 | CAVNQAGTAL | 479 | TRAV22 | TRAJ15 | CASSIGLGGGY | 601 | TRBV19 | TRBJ1-2 |
| | IF | | | | TF | | | |
| TCR_65 | CAVIYSGNTPL | 480 | TRAV8-1 | TRAJ29 | CASSLGGAEAF | 602 | TRBV11- | TRBJ1-1 |
| | VF | | | | F | | 3 | |
| TCR_66 | CAALSGGGAD | 481 | TRAV21 | TRAJ45 | CASSEALSGGA | 603 | TRBV2 | TRBJ2-5 |
| | GLTF | | | | PAETQYF | | | |
| TCR_67 | CAVRDLGGQK | 482 | TRAV1-2 | TRAJ16 | CASSLGQGSPA | 604 | TRBV5- | TRBJ2-6 |
| | LLF | | | | GANVLTF | | 6 | |
| TCR_68 | CAVSTGTASK | 483 | TRAV13- | TRAJ44 | CATRGVGETQ | 605 | TRBV15 | TRBJ2-5 |
| | LTF | | 1 | | YF | | | |
| TCR_69 | CSKTSYDKVIF | 484 | TRAV13- | TRAJ50 | CASSVVDRNN | 606 | TRBV9 | TRBJ2-1 |
| | | | 1 | | EQFF | | | |
| TCR_70 | CVVNRGDGLT | 485 | TRAV12- | TRAJ45 | CSARDQQGQN | 607 | TRBV20- | TRBJ2-2 |
| | F | | 1 | | TGELFF | | 1 | |
| TCR_71 | CLVANNARLM | 486 | TRAV4 | TRAJ31 | CASSQDRDNL | 608 | TRBV5- | TRBJ1-1 |
| | F | | | | NTEAFF | | 4 | |
| TCR_72 | CAVYGGSQGN | 487 | TRAV1-2 | TRAJ42 | CASSESNYGYT | 609 | TRBV7- | TRBJ1-2 |
| | LIF | | | | F | | 2 | |
| TCR_73 | CAVNQAGTAL | 479 | TRAV12- | TRAJ15 | CASSLLGSNQE | 610 | TRBV7- | TRBJ2-5 |
| | IF | | 2 | | TQYF | | 2 | |
| TCR_74 | CAVRDLGGQK | 482 | TRAV1-2 | TRAJ16 | CASSLGQGSPA | 604 | TRBV5- | TRBJ2-6 |
| | LLF | | | | GANVLTF | | 6 | |
| TCR_75 | CAVNEGGGSY | 488 | TRAV12- | TRAJ6 | CASSLAMGTS | 611 | TRBV5- | TRBJ2-1 |
| | IPTF | | 2 | | GGPYNEQFF | | 1 | |
| TCR_76 | CAASNRGGSE | 489 | TRAV23/ | TRAJ57 | CASSYHAGDR | 612 | TRBV6- | TRBJ1-2 |
| | KLVF | | DV6 | | GFGYTF | | 5 | |
| TCR_77 | CAGNTGTASK | 490 | TRAV38- | TRAJ44 | CASSLWGGYT | 613 | TRBV5- | TRBJ1-1 |
| | LTF | | 2/DV8 | | EAFF | | 5 | |
| TCR_78 | CAAPTNNNDM | 491 | TRAV13- | TRAJ43 | CASSYVTGAS | 614 | TRBV6- | TRBJ2-7 |
| | RF | | 1 | | YEQYF | | 2 | |
| TCR_79 | CAERTALGYS | 492 | TRAV5 | TRAJ11 | CASSTTGGEEQ | 615 | TRBV18 | TRBJ2-1 |
| | TLTF | | | | FF | | | |
| TCR_80 | CADDYKLSF | 493 | TRAV13- | TRAJ20 | CASKKTPVGIE | 616 | TRBV19 | TRBJ1-1 |
| | | | 1 | | AFF | | | |
| TCR_81 | CVVNDPAGSY | 494 | TRAV12- | TRAJ28 | CASSPPSGGNT | 617 | TRBV3- | TRBJ2-2 |
| | QLTF | | 1 | | GELFF | | 1 | |
| TCR_82 | CAERGGGFKTI | 495 | TRAV5 | TRAJ9 | CASSSRTAPSD | 618 | TRBV5- | TRBJ2-3 |
| | F | | | | TQYF | | 1 | |
| TCR_83 | CAVEGNYGGS | 496 | TRAV2 | TRAJ42 | CASSINPSSYN | 619 | TRBV19 | TRBJ2-1 |
| | QGNLIF | | | | EQFF | | | |
| TCR_84 | CALTGNTGGF | 497 | TRAV19 | TRAJ9 | CASSLDSSLGY | 620 | TRBV11- | TRBJ1-2 |
| | KTIF | | | | GYTF | | 2 | |
| TCR_85 | CAESKEGKLIF | 498 | TRAV5 | TRAJ37 | CAYQPPGGGNI | 621 | TRBV30 | TRBJ2-4 |
| | | | | | QYF | | | |
| TCR_86 | CVVNMGRGYS | 499 | TRAV12- | TRAJ11 | CASSPVAGGN | 622 | TRBV5- | TRBJ2-2 |
| | TLTF | | 1 | | TGELFF | | 6 | |
| TCR_87 | CAMTWGGTSY | 500 | TRAV12- | TRAJ52 | CSAIEEGTEVF | 623 | TRBV20- | TRBJ1-2 |
| | GKLTF | | 3 | | GYTF | | 1 | |
| TCR_88 | CVVNQYNDM | 501 | TRAV12- | TRAJ43 | CASFQDQNTG | 624 | TRBV12- | TRBJ2-2 |
| | RF | | 1 | | ELFF | | 3 | |
| TCR_89 | CAVIGGSSNTG | 502 | TRAV21 | TRAJ37 | CASSLAGAQQ | 625 | TRBV5- | TRBJ1-1 |
| | KLIF | | | | AFF | | 1 | |
| TCR_90 | CVVNNAGNM | 503 | TRAV12- | TRAJ39 | CATQNLNTGE | 626 | TRBV15 | TRBJ2-2 |
| | LTF | | 1 | | LFF | | | |
| TCR_91 | CALSYSDGQK | 504 | TRAV16 | TRAJ16 | CASSLAGGWG | 627 | TRBV5- | TRBJ1-1 |
| | LLF | | | | TEAFF | | 1 | |
| TCR_92 | CAMSANAGN | 505 | TRAV12- | TRAJ39 | CASRPLTGGPL | 628 | TRBV27 | TRBJ2-4 |
| | MLTF | | 3 | | AKNIQYF | | | |
| TCR_93 | CALDYGQNFV | 506 | TRAV24 | TRAJ26 | CASSIGSREAFF | 629 | TRBV19 | TRBJ1-1 |
| | F | | | | | | | |
| TCR_94 | CVVNNNNDM | 507 | TRAV12- | TRAJ43 | CATTDLDSGEL | 630 | TRBV15 | TRBJ2-2 |
| | RF | | 1 | | FF | | | |
| TCR_95 | CAAPMTTDSW | 508 | TRAV20 | TRAJ24 | CASSSAARTGN | 631 | TRBV5- | TRBJ2-1 |
| | GKLQF | | | | EQFF | | 1 | |
| TCR_96 | CAERTALGYS | 492 | TRAV4 | TRAJ11 | CASSTTGGEEQ | 615 | TRBV18 | TRBJ2-1 |
| | TLTF | | | | FF | | | |
| TCR_97 | CALSLIIYNQG | 509 | TRAV19 | TRAJ23 | CASSPTSGSRE | 632 | TRBV6- | TRBJ2-5 |
| | GKLIF | | | | TQYF | | 2 | |
| TCR_98 | CVVNYDTDKL | 510 | TRAV12- | TRAJ34 | CATGGLNTGE | 633 | TRBV2 | TRBJ2-2 |
| | IF | | 1 | | LFF | | | |
| TCR_99 | CVVNEEDKLIF | 511 | TRAV12- | TRAJ34 | CAGSTSLTGEL | 634 | TRBV7- | TRBJ2-2 |
| | | | 1 | | FF | | 9 | |
| TCR_100 | CAFTNYNQGG | 512 | TRAV24 | TRAJ23 | CASSPAGGAV | 635 | TRBV5- | TRBJ1-1 |
| | KLIF | | | | LQAFF | | 6 | |
| TCR_101 | CAVHLNDYKL | 513 | TRAV20 | TRAJ20 | CASSRPSGQGN | 636 | TRBV18 | TRBJ1-2 |
| | SF | | | | NGYTF | | | |
| TCR_102 | CVVNRDTGNQ | 514 | TRAV12- | TRAJ49 | CAWLRDMNT | 637 | TRBV30 | TRBJ2-2 |
| | FYF | | 1 | | GELFF | | | |
| TCR_103 | CVVNRDNDM | 515 | TRAV12- | TRAJ43 | CASMDLNTGE | 638 | TRBV19 | TRBJ2-2 |
| | RF | | 1 | | LFF | | | |
| TCR_104 | CLVGAESGGY | 516 | TRAV4 | TRAJ4 | CASSQGDYRSS | 639 | TRBV14 | TRBJ2-7 |
| | NKLIF | | | | TYEQYF | | | |
| TCR_105 | CAFPRGGSNY | 517 | TRAV24 | TRAJ53 | CSARDVVSGG | 640 | TRBV20- | TRBJ2-7 |
| | KLTF | | | | HYEQYF | | 1 | |
| TCR_106 | CAVNRDDKIIF | 478 | TRAV12- | TRAJ30 | CASSPDIEQFF | 641 | TRBV7- | TRBJ2-1 |
| | | | 2 | | | | 9 | |
| TCR_107 | CAARYSGNTP | 518 | TRAV29/ | TRAJ29 | CASSTGSNTGE | 642 | TRBV12- | TRBJ2-2 |
| | LVF | | DV5 | | LFF | | 4 | |
| TCR_108 | CVVNGNNDM | 519 | TRAV12- | TRAJ43 | CARQDSNTGE | 643 | TRBV12- | TRBJ2-2 |
| | RF | | 1 | | LFF | | 3 | |
| TCR_109 | CAMRDSNSNS | 520 | TRAV14/ | TRAJ41 | CASSDGHQGL | 644 | TRBV10- | TRBJ2-5 |
| | GYALNF | | DV4 | | QETQYF | | 1 | |
| TCR_110 | CAASQISDGQK | 521 | TRAV23/ | TRAJ16 | CASSYQPGVA | 645 | TRBV6- | TRBJ1-4 |
| | LLF | | DV6 | | TNEKLFF | | 5 | |
| TCR_111 | CAFPSGGSNY | 446 | TRAV24 | TRAJ53 | CASSETGSSSY | 646 | TRBV6- | TRBJ2-7 |
| | KLTF | | | | EQYF | | 1 | |
| TCR_112 | CAENARLMF | 522 | TRAV13- | TRAJ31 | CASSSKGGGQ | 548 | TRBV7- | TRBJ2-5 |
| | | | 2 | | QTQYF | | 2 | |
| TCR_113 | CAVRGADNAR | 523 | TRAV1-2 | TRAJ31 | CASSSVSLGNE | 647 | TRBV6- | TRBJ2-1 |
| | LMF | | | | QFF | | 5 | |
| TCR_114 | CALMDSSYKLI | 524 | TRAV16 | TRAJ12 | CASSLEMQGA | 648 | TRBV5- | TRBJ1-2 |
| | F | | | | LYGYTF | | 1 | |
| TCR_115 | CAAMNNTNA | 525 | TRAV23/ | TRAJ27 | CASSLFSSGQG | 649 | TRBV7- | TRBJ1-2 |
| | GKSTF | | DV6 | | NGYTF | | 2 | |
| TCR_116 | CAMREGVIYN | 526 | TRAV14/ | TRAJ23 | CAWSRGSAYN | 650 | TRBV30 | TRBJ2-1 |
| | QGGKLIF | | DV4 | | EQFF | | | |
| TCR_117 | CAVLGGDKIIF | 527 | TRAV12- | TRAJ30 | CASSESNYGYT | 609 | TRBV7- | TRBJ1-2 |
| | | | 2 | | F | | 2 | |
| TCR_118 | CAEVGSQGNLI | 528 | TRAV5 | TRAJ42 | CASSYYPSASG | 651 | TRBV6- | TRBJ2-5 |
| | F | | | | RADETQYF | | 6 | |
| TCR_119 | CAMSQMDSSY | 529 | TRAV14/ | TRAJ12 | CSAPGTGYNE | 652 | TRBV29- | TRBJ2-1 |
| | KLIF | | DV4 | | QFF | | 1 | |
| TCR_120 | CAMRGLQGGK | 530 | TRAV14/ | TRAJ23 | CASSYQPGVA | 645 | TRBV6- | TRBJ1-4 |
| | LIF | | DV4 | | TNEKLFF | | 5 | |
| TCR_121 | CASGNTPLVF | 531 | TRAV12- | TRAJ29 | CASSETGSSSY | 646 | TRBV6- | TRBJ2-7 |
| | | | 2 | | EQYF | | 1 | |
| TCR_122 | CAVQAPMSAR | 427 | TRAV20 | TRAJ42 | CASSSKGGGQ | 548 | TRBV7- | TRBJ2-5 |
| | GSQGNLIF | | | | QTQYF | | 2 | |
| TCR_123 | CVVNGYNTDK | 532 | TRAV12- | TRAJ34 | CASSSVSLGNE | 647 | TRBV6- | TRBJ2-1 |
| | LIF | | 1 | | QFF | | 5 | |
| TCR_124 | CAMSPNNAGN | 533 | TRAV14/ | TRAJ39 | CASSLEMQGA | 648 | TRBV5- | TRBJ1-2 |
| | MLTV | | DV4 | | LYGYTF | | 1 | |
| TCR_125 | CALNQDRGST | 534 | TRAV9-2 | TRAJ18 | CASSLFSSGQG | 649 | TRBV7- | TRBJ1-2 |
| | LGRLYF | | | | NGYTF | | 2 | |
| TCR_126 | CAYKENYKYI | 535 | TRAV38- | TRAJ40 | CAWSRGSAYN | 650 | TRV30 | TRBJ2-1 |
| | F | | 2/D V8 | | EQFF | | | |
| TCR_127 | CAVYGGSQGN | 487 | TRAV1-2 | TRAJ42 | CASSESNYGYT | 609 | TRBV7- | TRBJ1-2 |
| | LIF | | | | F | | 2 | |
| TCR_128 | CAMSASPNDM | 536 | TRAV12- | TRAJ43 | CASSYYPSASG | 651 | TRBV6- | TRBJ2-5 |
| | RF | | 3 | | RADETQYF | | 6 | |
| TCR_ 129 | CAVNAPSSAS | 537 | TRAV8-1 | TRAJ3 | CSAPGTGYNE | 652 | TRBV29- | TRBJ2-1 |
| | KIIF | | | | QFF | | 1 | |
| TCR_130 | CAVSLNNAGN | 682 | | | CASSQETAGV | 685 | | |
| | MLTF* | | | | NEQFF* | | | |
| TCR_131 | CATDTGRRAL | 683 | | | CASSLGQGDTE | 686 | | |
| | TF* | | | | AFF* | | | |
| TCR_132 | CGTWEDQGAQ | 684 | | | CASSLAQGPY | 687 | | |
| | KLVF* | | | | NEQFF* | | | |

*TCRs identified in Example 21

TABLE 6

| SEQ ID NO: | TCR_ID chain |
|---|---|
| 1111 | TCR_1Alpha chain |
| 1112 | TCR_2Alpha chain |
| 1113 | TCR_3Alpha chain |
| 1114 | TCR_4Alpha chain |
| 1115 | TCR_5Alpha chain |
| 1116 | TCR_6Alpha chain |
| 1117 | TCR_7Alpha chain |
| 1118 | TCR_8Alpha chain |
| 1119 | TCR_9Alpha chain |
| 1120 | TCR_10Alpha chain |
| 1121 | TCR_11Alpha chain |
| 1122 | TCR_12Alpha chain |
| 1123 | TCR_13Alpha chain |
| 1124 | TCR_14Alpha chain |
| 1125 | TCR_15Alpha chain |
| 1126 | TCR_16Alpha chain |
| 1127 | TCR_17Alpha chain |
| 1128 | TCR_18Alpha chain |
| 1129 | TCR_19Alpha chain |
| 1130 | TCR_20Alpha chain |
| 1131 | TCR_21Alpha chain |
| 1132 | TCR_22Alpha chain |
| 1133 | TCR_23Alpha chain |
| 1134 | TCR_24Alpha chain |
| 1135 | TCR_25Alpha chain |
| 1136 | TCR_26Alpha chain |
| 1137 | TCR_27Alpha chain |
| 1138 | TCR_28Alpha chain |
| 1139 | TCR_29Alpha chain |
| 1140 | TCR_30Alpha chain |
| 1141 | TCR_31Alpha chain |
| 1142 | TCR_32Alpha chain |
| 1143 | TCR_33Alpha chain |
| 1144 | TCR_34Alpha chain |
| 1145 | TCR_35Alpha chain |
| 1146 | TCR_36Alpha chain |
| 1147 | TCR_37Alpha chain |
| 1148 | TCR_38Alpha chain |
| 1149 | TCR_39Alpha chain |
| 1150 | TCR_40Alpha chain |
| 1151 | TCR_41Alpha chain |
| 1152 | TCR_42Alpha chain |
| 1153 | TCR_43Alpha chain |
| 1154 | TCR_44Alpha chain |
| 1155 | TCR_45Alpha chain |
| 1156 | TCR_46Alpha chain |
| 1157 | TCR_47Alpha chain |
| 1158 | TCR_48Alpha chain |
| 1159 | TCR_49Alpha chain |
| 1160 | TCR_50Alpha chain |
| 1161 | TCR_51Alpha chain |
| 1162 | TCR_52Alpha chain |
| 1163 | TCR_53Alpha chain |
| 1164 | TCR_54Alpha chain |
| 1165 | TCR_55Alpha chain |
| 1166 | TCR_56Alpha chain |
| 1167 | TCR_57Alpha chain |
| 1168 | TCR_58Alpha chain |
| 1169 | TCR_59Alpha chain |
| 1170 | TCR_60Alpha chain |
| 1171 | TCR_61Alpha chain |
| 1172 | TCR_62Alpha chain |
| 1173 | TCR_63Alpha chain |
| 1174 | TCR_64Alpha chain |
| 1175 | TCR_65Alpha chain |
| 1176 | TCR_66Alpha chain |
| 1177 | TCR_67Alpha chain |
| 1178 | TCR_68Alpha chain |
| 1179 | TCR_69Alpha chain |
| 1180 | TCR_70Alpha chain |
| 1181 | TCR_71Alpha chain |
| 1182 | TCR_72Alpha chain |
| 1183 | TCR_73Alpha chain |
| 1184 | TCR_74Alpha chain |
| 1185 | TCR_75Alpha chain |
| 1186 | TCR_76Alpha chain |
| 1187 | TCR_77Alpha chain |
| 1188 | TCR_78Alpha chain |
| 1189 | TCR_79Alpha chain |
| 1190 | TCR_80Alpha chain |
| 1191 | TCR_81Alpha chain |
| 1192 | TCR_82Alpha chain |
| 1193 | TCR_83Alpha chain |
| 1194 | TCR_84Alpha chain |
| 1195 | TCR_85Alpha chain |
| 1196 | TCR_86Alpha chain |
| 1197 | TCR_87Alpha chain |
| 1198 | TCR_88Alpha chain |
| 1199 | TCR_89Alpha chain |
| 1200 | TCR_90Alpha chain |
| 1201 | TCR_91Alpha chain |
| 1202 | TCR_92Alpha chain |
| 1203 | TCR_93Alpha chain |
| 1204 | TCR_94Alpha chain |
| 1205 | TCR_95Alpha chain |
| 1206 | TCR_96Alpha chain |
| 1207 | TCR_97Alpha chain |
| 1208 | TCR_98Alpha chain |
| 1209 | TCR_99Alpha chain |
| 1210 | TCR_100Alpha chain |
| 1211 | TCR_101Alpha chain |
| 1212 | TCR_102Alpha chain |
| 1213 | TCR_103Alpha chain |
| 1214 | TCR_104Alpha chain |
| 1215 | TCR_105Alpha chain |
| 1216 | TCR_106Alpha chain |
| 1217 | TCR_107Alpha chain |
| 1218 | TCR_108Alpha chain |
| 1219 | TCR_1Beta chain |
| 1220 | TCR_2Beta chain |
| 1221 | TCR_3Beta chain |
| 1222 | TCR_4Beta chain |
| 1223 | TCR_5Beta chain |
| 1224 | TCR_6Beta chain |
| 1225 | TCR_7Beta chain |
| 1226 | TCR_8Beta chain |
| 1227 | TCR_9Beta chain |
| 1228 | TCR_10Beta chain |
| 1229 | TCR_11Beta chain |
| 1230 | TCR_12Beta chain |
| 1231 | TCR_13Beta chain |
| 1232 | TCR_14Beta chain |
| 1233 | TCR_15Beta chain |
| 1234 | TCR_16Beta chain |
| 1235 | TCR_17Beta chain |
| 1236 | TCR_18Beta chain |
| 1237 | TCR_19Beta chain |
| 1238 | TCR_20Beta chain |
| 1239 | TCR_21Beta chain |
| 1240 | TCR_22Beta chain |
| 1241 | TCR_23Beta chain |
| 1242 | TCR_24Beta chain |
| 1243 | TCR_25Beta chain |
| 1244 | TCR_26Beta chain |
| 1245 | TCR_27Beta chain |
| 1246 | TCR_28Beta chain |
| 1247 | TCR_29Beta chain |
| 1248 | TCR_30Beta chain |
| 1249 | TCR_31Beta chain |
| 1250 | TCR_32Beta chain |
| 1251 | TCR_33Beta chain |
| 1252 | TCR_34Beta chain |
| 1253 | TCR_35Beta chain |
| 1254 | TCR_36Beta chain |
| 1255 | TCR_37Beta chain |
| 1256 | TCR_38Beta chain |
| 1257 | TCR_39Beta chain |
| 1258 | TCR_40Beta chain |
| 1259 | TCR_41Beta chain |
| 1260 | TCR_42Beta chain |
| 1261 | TCR_43Beta chain |
| 1262 | TCR_44Beta chain |
| 1263 | TCR_45Beta chain |
| 1264 | TCR_46Beta chain |
| 1265 | TCR_47Beta chain |
| 1266 | TCR_48Beta chain |
| 1267 | TCR_49Beta chain |
| 1268 | TCR_50Beta chain |
| 1269 | TCR_51Beta chain |
| 1270 | TCR_52Beta chain |
| 1271 | TCR_53Beta chain |
| 1272 | TCR_54Beta chain |
| 1273 | TCR_55Beta chain |
| 1274 | TCR_56Beta chain |
| 1275 | TCR_57Beta chain |
| 1276 | TCR_58Beta chain |
| 1277 | TCR_59Beta chain |
| 1278 | TCR_60Beta chain |
| 1279 | TCR_61Beta chain |
| 1280 | TCR_62Beta chain |
| 1281 | TCR_63Beta chain |
| 1282 | TCR_64Beta chain |
| 1283 | TCR_65Beta chain |
| 1284 | TCR_66Beta chain |
| 1285 | TCR_67Beta chain |
| 1286 | TCR_68Beta chain |
| 1287 | TCR_69Beta chain |
| 1288 | TCR_70Beta chain |
| 1289 | TCR_71Beta chain |
| 1290 | TCR_72Beta chain |
| 1291 | TCR_73Beta chain |
| 1292 | TCR_74Beta chain |
| 1293 | TCR_75Beta chain |
| 1294 | TCR_76Beta chain |
| 1295 | TCR_77Beta chain |
| 1296 | TCR_78Beta chain |
| 1297 | TCR_79Beta chain |
| 1298 | TCR_80Beta chain |
| 1299 | TCR_81Beta chain |
| 1300 | TCR_82Beta chain |
| 1301 | TCR_83Beta chain |
| 1302 | TCR_84Beta chain |
| 1303 | TCR_85Beta chain |
| 1304 | TCR_86Beta chain |
| 1305 | TCR_87Beta chain |
| 1306 | TCR_88Beta chain |
| 1307 | TCR_89Beta chain |
| 1308 | TCR_90Beta chain |
| 1309 | TCR_91Beta chain |
| 1310 | TCR_92Beta chain |
| 1311 | TCR_93Beta chain |
| 1312 | TCR_94Beta chain |
| 1313 | TCR_95Beta chain |
| 1314 | TCR_96Beta chain |
| 1315 | TCR_97Beta chain |
| 1316 | TCR_98Beta chain |
| 1317 | TCR_99Beta chain |
| 1318 | TCR_100Beta chain |
| 1319 | TCR_101Beta chain |
| 1320 | TCR_102Beta chain |
| 1321 | TCR_103Beta chain |
| 1322 | TCR_104Beta chain |
| 1323 | TCR_105Beta chain |
| 1324 | TCR_106Beta chain |
| 1325 | TCR_107Beta chain |
| 1326 | TCR_108Beta chain |

In TABLES 1-6, when a given column refers to a particular TCR, e.g., TCR_1, the terminology is used consistently throughout the various tables. As a result, the TCR designated as TCR_1 in TABLE 1, is the same TCR that appears in TABLES 5 and 6. Furthermore, in the context of an engineered T cell receptor having an alpha chain and a beta chain, wherein the TCR comprises corresponding CDR3 alpha and CDR3 beta sequences set forth, e.g., in TABLE 5, it is understood that the two CDR3 sequences belong to the same TCR, e.g., TCR_1. Furthermore, in the context of an engineered TCR further comprising CDR1 alpha and CDR2 alpha sequences defined by the corresponding alpha V gene, it is understood that the CDR1 and CDR2 sequences are encoded by the same V gene, e.g., for TCR_1, and belong to the same TCR as the CDR3 sequence, e.g., TCR_1. The CDR1 and CDR2 sequences can be determined by methods known in the art based on the sequence of the V gene (see, e.g., Gowthaman and Pierce, Nucleic Acids Res. (2018) 46: W396-W401). As a result, in a given row in, e.g., TABLES 1, 5 and 6, a particular TCR can have, as the relevant context dictates, the features, e.g., CDR 3 amino acid sequence, or is encoded by specified V and J gene sequences in that row of the table. For example, a soluble TCR does not necessarily have all the domains and functionality as an entire, membrane bound TCR.

Also provided herein are SARS-CoV-2 T cell epitopes comprising amino acid sequences SEQ ID NOs: 271-310 and 313-326, and combinations thereof. In certain embodiments, the T cell epitope comprises an amino acid sequence selected from SEQ ID NOs: 286-310 and 313-326. In certain embodiments, a plurality of T cell epitopes comprise amino acid sequences selected from SEQ ID NOs: 286-310 and 313-326, and combinations thereof.

Also contemplated are variants of the T cell epitopes, for example, a peptide comprising an amino acid sequence that differs by 1, 2, or 3 amino acids relative to a T cell epitope disclosed herein. Such variants can be derived from, for example, mutant SARS-CoV-2 strains that arise in the human population over time. It is understood, according to scientific literature and databases (Rammensee et al., 1999; Godkin et al., 1997), that certain positions of T cell epitopes are typically anchor residues forming a core sequence fitting to the binding groove of the MHC. Thus, a skilled person in the art would be able to modify the amino acid sequences of the T cell epitopes disclosed herein, by maintaining the known anchor residues, and determine whether such variants maintain the ability to bind the MHC. The T cell epitopes disclosed herein including the variants, as well as peptides (e.g., isolated peptides) comprising such epitopes, are useful for stimulating T cell immune responses in vitro, ex vivo, or in vivo.

In certain embodiments, the epitope is an MHC Class I-restricted T cell epitope. In certain embodiment, the epitope, when complexed with a cognate MHC Class I, is capable of activating CD8⁺ T cells. In certain embodiments, the epitope is an MHC Class II-restricted T cell epitope. In certain embodiments, the epitope, when complexed with a cognate MHC Class II, is capable of activating CD4⁺ T cells. In certain embodiments, the epitope can bind an MHC Class I and an MHC Class II and, when complexed with the cognate MHCs, is capable of activating CD8⁺ and CD4⁺ T cells, respectively. In one embodiment, the epitope is derived from a SARS-CoV-2 antigen, e.g., selected from the group consisting of ORF1AB, Spike protein, N protein, M protein, 3A protein and E protein. In one embodiment, the epitope is derived from a SARS-CoV-2 antigen selected from the group consisting of a protein encoded by a non-canonical ORF described by Finkel et al. (2020) Nature 589:125-130, including, for example, 1a.uORF1.ext, 1a.uORF1, 1a.uORF2.ext, 1a.uORF2, 1a.iORF, S.iORF1, S.iORF2, 3a.iORF1 (ORF3c), 3a.iORF2, E.iORF, M.ext, M.iORF, 6.iORF, 7a.iORF1, 7a.iORF2, 7a.iORF3, 7b.iORF1, 7b.iORF2, 8.iORF, N.iORF1 (ORF9b), N.iORF2, 10.uORF, and 10.iORF.

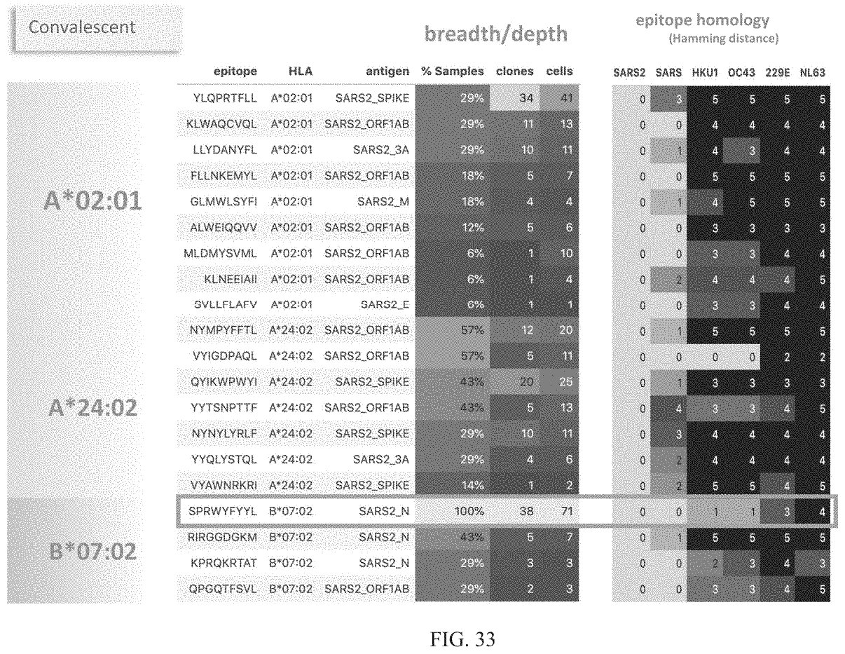

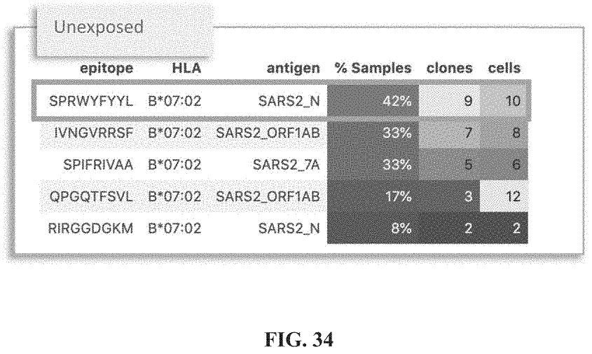

In certain embodiments, the epitope is a crossreacting epitope that is homologous across two or more coronavirus members, e.g., SARS-CoV-2 and at least one additional coronavirus, such as SARS-CoV-1, HCoV-OC43, HCoV-HKU1, HCoV-229E and/or HCoV-NL63. Certain subjects not exposed to COVID-19 have been found to have T cells reactive to SARS-CoV-2 antigens, implying crossreactivity from exposure to an endemic coronavirus (see e.g., Example 20 and Example 22). Moreover, crossreactive memory T cells are implicated in playing a role in herd immunity to SARS-CoV-2 (Lipsitch et al. (2020) Nature Reviews, published Oct. 6, 2020). Accordingly, in one embodiment, the disclosure provides a SARS-CoV-2 T cell epitope that is recognized by T cells from COVID-19 T patients as well as T cells from nonexposed subjects, i.e., a cross-reactive epitope that is homologous across at least two or more coronavirus members. Epitope homology for T cell epitopes across various coronavirus sequences can be determined using the Hamming Distance between the sequences being compared (see e.g. FIG. 33). For example, the Hamming Distance value of the epitope for SARS-CoV-2 is set as 0 and then a “homologous” epitope across another coronavirus is a sequence with a Hamming Distance value of 2 or less, more preferably 1 or less, most preferably 0. As described herein in Example 20 and Example 22, a T cell epitope derived from the SARS-CoV-2 N protein (SPRWYFYYL; SEQ ID NO: 323) has been identified whose sequence is homologous (Hamming Distance of 2 or less) in SARS-CoV-1, HCoV-HKU1 and HCoV-OC43. This T cell epitope was recognized by T cells from 100% of the COVID-19 convalescent patients tested, as well as by T cells from almost half of the non-exposed subjects.

It is understood that a peptide comprising a T cell epitope is useful in stimulating a T cell immune response. Where the peptide consists of the T cell epitope sequence, the peptide can be loaded directly on the surface of an APC to form a complex with an MHC (e.g., MHC Class I). Where the peptide includes additional amino acids on the N-terminus and/or C-terminus of the T cell epitope, the peptide can be expressed or delivered in an antigen-presenting cell (APC) and be processed by the APC to present the epitope on the cell surface. Accordingly, the peptides useful in the present invention comprise the epitope sequences disclosed herein and may be greater in length. In certain embodiments, the peptide is no more than 100 amino acids in length, for example, no more than 90 amino acids, no more than 80 amino acids, no more than 70 amino acids, no more than 60 amino acids, no more than 50 amino acids, no more than 40 amino acids, no more than 35 amino acids, no more than 30 amino acids, no more than 25 amino acids, no more than 20 amino acids, no more than 19 amino acids, no more than 18 amino acids, no more than 17 amino acids, no more than 16 amino acids, no more than 15 amino acids, no more than 14 amino acids, no more than 13 amino acids, no more than 12 amino acids, no more than 11 amino acids, or no more than 10 amino acids in length. In certain embodiments, where the epitope in the peptide is expected to bind MHC Class I, the peptide is no more than 10 amino acids in length. In certain embodiments, where the epitope in the peptide is expected to bind MHC Class II, the peptide is no more than 25 amino acids in length. In certain embodiments, the amino acid sequence of the peptide consists of the amino acid sequence of the corresponding T cell epitope.

In certain embodiments, a peptide of the present invention comprises two or more T cell epitopes, e.g., two or more of the T cell epitopes disclosed herein. In certain embodiments, the two or more T cell epitopes are partially overlapping, and the peptide comprises the entire amino acid sequence of the two or more T cell epitopes aligned. In certain embodiments, the two or more T cell epitopes are incorporated in a hotspot region.

In certain embodiments, the peptide further comprises a moiety (e.g., an amino acid sequence) that improves one or more characteristics of the T cell epitope or its manufacture or function. For example, in certain embodiments, the peptide further comprises an amino acid sequence that facilitates delivery of the T cell epitope into APCs. In certain embodiments, the peptide further comprises a moiety (e.g., an antibody or an antigen-binding fragment thereof) that specifically targets an APC. In certain embodiments, the peptide further comprises a moiety that improves stability and/or binding to an MHC to elicit a stronger immune response. In certain embodiments, the peptide comprises a cell penetrating peptide, which facilitates cell uptake in a manner that does not require a cell membrane protein. In certain embodiments, the peptide is modified, for example, to mimic the post-translational modification of the corresponding SARS-CoV-2 protein when expressed in the APC.

In certain embodiments, the peptide binds an MHC to form a complex. The ability of a peptide to bind an MHC can be assessed by various assays known in the art or described herein, such as by analysis of MHC-eluted peptides by liquid chromatography with tandem mass spectrometry (LC-MS/MS) and in silico prediction algorithms (see, e.g., Sofron et al. (2016) Eur. J. Immunol. 46:319-328), fluorescence polarization assays (see, e.g., Yin et al. (2014) Curr. Protoc. Immunol. 106:5.10.1-5.10.12), ELISA (see, e.g., Sylvester-Hvid et al., (2002) Tissue Antigens, 59:251-58), UV-mediated peptide exchange (see, e.g., Rodenko et al., Nat. Protoc. (2006) 1(3):1120-32), LC/MS (see, e.g., Obermair et al. (2021) bioRxiv posted Mar. 4, 2021 under doi.org/10.1101/2021.03.02.43352), or using a chimeric MHC/TcR system described in Section III below.

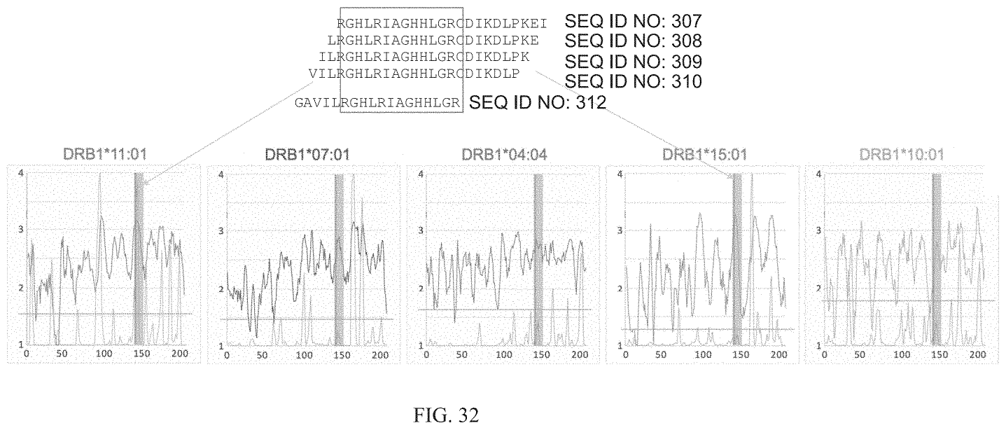

As described in Example 19, starting from a published TCR sequence from a T cell obtained from an acute COVID-19 patient, a 20-mer epitope having the sequence RGHLRIAGHEILGRCDIKDLP (SEQ ID NO: 306) has been identified that is an MHC Class II-restricted CD4 T cell epitope derived from the SARS-CoV-2 membrane glycoprotein (M protein). Analysis of this epitope revealed it was displayed across multiple human MHC Class II alleles, including DRB1*11:01, DRB1*07:01, DRB1*04:04, DRB1*15:01 and DRB1*10:01 (shown in FIG. 32). Patient T cells also showed reactivity to longer peptides that contained the 20-mer sequence, including the 23-mers having the amino acid sequences shown in in SEQ ID NOs: 307-310. Accordingly, in certain embodiments, the SARS-CoV-2 T cell epitope is a peptide having the amino acid sequence set forth in in SEQ ID NOs: 307-310. In certain embodiments, the peptide of the present invention is 20-30 amino acids in length, or 20-25 amino acids in length, or 20-23 amino acids in length, comprising the amino acid sequence shown in SEQ ID NO: 306. In various embodiments, the peptide is a 20mer, a 21mer, a 22mer, a 23mer, a 24mer, a 25mer, a 26mer, a 27mer, a 28mer, a 29mer or a 30mer comprising the amino acid sequence of SEQ ID NO: 306.

As described in Example 20, screening of peptide-MHC tetramers loaded with 9-mer epitope libraries led to the identification of 20 high confidence MHC Class I epitopes with reactivity against T cells from convalescent COVID-19 patients, and for certain epitopes reactivity against T cells from unexposed patients as well. These 20 MHC Class I epitopes (shown in TABLE 8) have the amino acid sequences shown in SEQ ID NOs: 286-289, 294, 297 and 313-326. Epitopes having the sequences of SEQ ID NOs: 286-289, 294, 297 and 313-315 bind HLA-A*02:01. Epitopes having the sequences of SEQ ID NOs: 316-322 bind HLA-A*24:02. Epitopes having the sequences of SEQ ID NOs: 323-326 bind HLA-B*07:02. These epitopes are derived from six different SARS-CoV-2 antigens: ORF1AB (SEQ ID NOs: 287, 289, 297, 314-317, 319 and 326), Spike protein (SEQ ID NOs: 288, 318, 320 and 322), N protein (SEQ ID NOs: 323-325), M protein (SEQ ID NO: 294), 3A protein (SEQ ID NOs: 286 and 321) and E protein (SEQ ID NO: 313). Notably the experiments revealed that a handful of dominant epitopes are emerging and the most reactive epitopes may have assistance by endemic coronavirus, given the level of reactivity to certain epitopes observed in unexposed patient samples. In particular, the N protein-derived, B*07:02-restricted epitope SPRWYFYYL (SEQ ID NO: 323) showed reactivity with all convalescent patient samples tested and almost half of unexposed patients, indicating it is a dominant T cell epitope.

Accordingly, in certain embodiments, the SARS-CoV-2 T cell epitope is a peptide having the amino acid sequence shown in any of SEQ ID NOs: 286-289, 294, 297 and 313-326. In certain embodiments, the SARS-CoV-2 T cell epitope is a peptide having the amino acid sequence shown in any of SEQ ID NOs: 286-289, 294, 297 and 313-315. In certain embodiments, the SARS-CoV-2 T cell epitope is a peptide having the amino acid sequence shown in any of SEQ ID NOs: 316-322. In certain embodiments, the SARS-CoV-2 T cell epitope is a peptide having the amino acid sequence shown in any of SEQ ID NOs: 287, 289, 297, 314-317, 319 and 326. In certain embodiments, the SARS-CoV-2 T cell epitope is a peptide having the amino acid sequence shown in any of SEQ ID NOs: 288, 318, 320 and 322. In certain embodiments, the SARS-CoV-2 T cell epitope is a peptide having the amino acid sequence shown in any of SEQ ID NOs: 323-325. In certain embodiments, the SARS-CoV-2 T cell epitope is a peptide having the amino acid sequence shown in SEQ ID NO: 294. In certain embodiments, the SARS-CoV-2 T cell epitope is a peptide having the amino acid sequence shown in SEQ ID NO: 286 or 321. In certain embodiments, the SARS-CoV-2 T cell epitope is a peptide having the amino acid sequence shown in SEQ ID NO: 313. In certain embodiments, the SARS-CoV-2 T cell epitope is a peptide having the amino acid sequence shown in SEQ ID NO: 323.

In certain embodiments, a peptide disclosed herein that comprises a T cell epitope binds a cognate MHC corresponding to the T cell epitope. In certain embodiments, the complex of the peptide and the cognate MHC is capable of stimulating a T cell immune response. For example, where the cognate MHC is a Class I MHC (e.g., HLA-A, HLA-B, or HLA-C), the complex is capable of stimulating a CD8⁺ T cell immune response, such as proliferation, activation, and/or memory formation of CD8⁺ T cells. Where the cognate MHC is a Class II MHC (e.g., HLA-DP, HLA-DQ, or HLA-DR), the complex is capable of stimulating a CD4+ T cell immune response, such as proliferation, activation, and/or memory formation of CD4⁺ T cells. Such complex can be presented as a soluble complex, immortalized on a solid surface (e.g., beads or nanoparticles), or presented on the surface of an APC. Clonal T cell proliferation can be assessed by methods known in the art such as carboxyfluorescein succinimidyl ester (CFSE) dilution assay. T cell activation can be assessed by methods known in the art such as staining for cell surface markers (e.g., upregulation of CD69, CD27, CD137, CD154 or downregulation of CD62L or CCR7) or cytokines (e.g., IFNγ or TNFα) and quantifying secretion of cytokine proteins (e.g., IFNγ or TNFα). Memory T cell formation can be assessed by methods known in the art such as staining for cell surface markers (e.g., CD45RO).

The SARS-CoV-2 T cell epitopes and the peptide comprising such epitopes disclosed herein can be used to stimulate a T cell immune response in vitro, ex vivo, or in vivo. Accordingly, the disclosure provides a method of stimulating a T cell immune response to SARS-CoV-2, or a cell infected thereby, by contacting a population of T cells with a T cell epitope presented by an MHC to permit activation of one or more T cells in the population for reactivity to a SARS-CoV-2 infected cell.

In certain embodiments, the T cell immune response can stimulated in vitro or ex vivo. A T cell epitope can be presented by an MHC in vitro or ex vivo by forming a complex, such as a complex immobilized on a solid surface (e.g., beads or nanoparticles) or presented on the surface of an APC. In certain embodiments, the disclosure provides a method of producing activated T cells, the method comprising contacting a population of T cells in vitro with the complex or APC to permit activation of one or more T cells in the population for reactivity to a SARS-CoV-2 infected cell. In certain embodiments, where the epitope-MHC complex is a class I complex, the one or more T cells in the population activated by this method are CD8⁺ T cells. In certain embodiments, where the epitope-MHC complex is a class II complex, the one or more T cells in the population activated by this method are CD4⁺ T cells. The method may optionally further comprise culturing the T cells to permit T cell amplification. Suitable conditions for T cell amplification include but are not limited to cell culture medium containing cytokines that support T cell survival and proliferation, such as IL-2 and IL-15. In certain embodiments, soluble anti-CD3 or anti-CD³/anti-CD28 beads are present in the culture media.

### IV. Peptide Libraries

In another embodiment, a composition of the disclosure is a SARS-CoV-2 T cell epitope library, e.g., a library comprising at least 100, at least 200, at least 300, at least 400 or at least 500 peptide moieties, wherein the peptide moieties within the library are included based on certain characteristics. For example, the library can comprise peptide moieties containing identified mutations in SARS-Co-V2 spike protein and optionally peptide moieties from at least one, and preferably multiple, of the following categories:
(a) 8mer-12mer peptides (e.g., 9mer peptides) of SARS-CoV-2 full proteome (e.g., peptide having an IC50, measure or predicted, of less than 500 nM for one or multiple MHC alleles);
(b) peptides of SARS-CoV comprising a sequence at least 90% (or at least 95%, 96%, 97%, 98% or 99%) identical to homologous SARS-CoV-2 sequences;
(c) peptides from common cold coronaviruses;
(d) peptides comprising immunodominant epitopes of SARS-CoV (e.g., identified from the Immune Epitope Database (IEDB);
(e) SARS-Co-V2 peptides with predicted glycosylation sites;
(f) peptide highly conserved across multiple coronavirus species or strains;
(g) peptides of non-structural proteins with low observed mutation rates;
(h) peptides against which T cell reactivity has been detected in abundance in patients with mild disease but not severe disease (e.g., patient that perished or required ventilation);
(i) peptides against which T cell reactivity has been detected in abundance in asymptomatic patients but not symptomatic patients; and
(j) peptides that show T cell reactivity with broad clonal diversity in recovered patients.

In other embodiments, the peptide library comprising peptide moieties from at least two, at least three, or at least four, or at least five, at least six, at least seven, at least eight, at least nine, at least ten or all eleven of the categories set forth in (a)-(j).

In another embodiment, the library include peptides that are predicted to load with IC50<500 nM across the top five, or top ten or top twenty Class I MHC alleles. In another embodiment, the peptide library can incorporate peptides from new viral strains that are shifting in prevalence within a population. In another embodiment, the peptide library can incorporate peptides that are altered by key mutations shown to alter viral function. In another embodiment, peptide can be specifically designed with respect to the MHC allele(s) to be used for peptide loading, e.g., peptides can be designed for a panel of five MHC alleles (e.g., five MCH Class I alleles).

A non-limiting example of a SARS-CoV-2 T cell epitope library is the 596-member library described in detail in Example 18 for MHC class I peptides or in TABLE 4 for MHC class II peptides.

In another embodiment, a composition of the disclosure is an MHC multimer library (e.g., MHC tetramer library). The MHC multimer library can comprise MHC multimers loaded with a SARS-CoV-2 T cell epitope library of the disclosure. In one embodiment, the MHC multimer library comprises MHC Class I multimers. Suitable MHC Class I alleles/sequences for preparation of multimers are described further below. In another embodiment, the MHC multimer library comprises MHC Class II multimers. Suitable MHC Class II alleles/sequences for preparation of multimers are described further below. Methods of preparing MHC multimers and loading them with a peptide epitope library are described further below. A non-limiting example of an MHC multimer library is MHC Class I tetramers loaded with the 596-member T cell epitope library described in detail in Example 18.

In one embodiment, the multimerization domain of the multimer is streptavidin or avidin. In one embodiment, the MHC multimer comprises four MHC monomers covalently conjugated to the streptavidin or avidin molecule at sites other than the biotin-binding site of streptavidin or avidin. In one embodiment, the four MHC monomers each comprise (i.e., are loaded with) a SARS-CoV-2 peptide, wherein each monomer comprises the same peptide. In one embodiment, the MHC multimer further comprises a biotinylated oligonucleotide barcode bound to the biotin-binding site of streptavidin or avidin.

In another embodiment, a composition of the disclosure is a kit for identifying a T cell reactive to a SARS-CoV-2 T cell epitope. The kit can comprise, for example an MHC multimer library of the disclosure (i.e., loaded with SARS-CoV-2 T cell epitope peptides) packaged with instructions for use of the library to identify a T cell reactive to a SARS-CoV-2 T cell epitope. Methods of using an MHC multimer library to identify T cells reactive to SARS-CoV-2 T cell epitopes are described further below.

In one embodiment, the kit comprises a plurality of MHC multimers. In one embodiment, the multimerization domain of each multimer is streptavidin or avidin. In one embodiment, each multimer comprises four MHC monomers covalently conjugated to the streptavidin or avidin molecule at sites other than the biotin-binding site of streptavidin or avidin. In one embodiment, the four MHC monomers each comprise an MHC-binding peptide, wherein each MHC monomer within each single MHC multimer comprises (i.e., is loaded with) the same SARS-CoV-2 peptide and wherein each MHC multimer within the plurality comprises (i.e., is loaded with) a different SARS-CoV-2 peptide, thereby forming a library of SARS-CoV-2 peptides. In one embodiment, each MHC multimer within the plurality further comprises a biotinylated oligonucleotide barcode bound to the biotin-binding site of streptavidin or avidin.

### MHC Polypeptides

(a) MHC Class I Polypeptides

The Class I histocompatibility ternary complex consists of three parts associated by noncovalent bonds. The MHC class I heavy chain is a polymorphic transmembrane glycoprotein of about 45 kDa consisting of three extracellular domains, each containing about 90 amino acids (α1 at the N-terminus, α2 and α3), a transmembrane domain of about 40 amino acids and a cytoplasmic tail of about 30 amino acids. The α1 and α2 domains of the MHCI heavy chain contain two segments of alpha helix that form a peptide-binding groove or cleft. A short peptide of about 8-10 but up to 11 amino acids binds noncovalently (“fits”) into this groove between the two alpha helices. The α3 domain of the MHCI heavy chain is proximal to the plasma membrane. The MHCI heavy chain is non-covalently bound to a β2 microglobulin (β2m) polypeptide, forming a ternary complex. In MHCI, the binding groove is closed at both ends by conserved tyrosine residues leading to a size restriction of the bound peptides to usually 8-10 residues but up to 11 residues with its C-terminal end docking into the F-pocket.

The disclosure provides a multimeric protein comprising a two or more MHCI or MHCI-like polypeptides. The MHCI molecule can suitably be a vertebrate MHC molecule such as a human, a mouse, a rat, a porcine, a bovine or an avian MHC molecule.

In some embodiments, the multimeric MHCI multimers described herein, the MHC molecule is a human MHC class I protein: HLA-A, HLA-B of HLA-C. In some embodiments, the multimer comprises MHC Class I like molecules (including non-classical MHC Class I molecules) including, but not limited to, CD1d, HLA E, HLA G, HLA F, HLA H, MIC A, MIC B, ULBP-1, ULBP-2, and ULBP-3. The amino acid sequences of the MHCI heavy chains, β2m polypeptides and of MHC Class I like molecules from a variety of vertebrate species are known in the art and publicly available.

In some embodiments, the MHCI heavy chain alpha domain is human, and comprise, for example, an MHCI heavy chain alpha domain(s) from a human MHC Class I molecule(s) selected from the group consisting of HLA-A*01:01, HLA-A*03:01, HLA-A*11:01, HLA-A*24:02, HLA-B*07:02, HLA-C*04:01, HLA-C*07:02, HLA-B*08:01, HLA-B*35:01, HLA-B*57:01, HLA-B*57:03, HLA-E, HLA-C*16:01, HLA-C*08:02, HLA-C*07:01, HLA-C*05:01, HLA-B*44:02, HLA-A*29:02, HLA-B*44:03, HLA-C*03:04, HLA-B*40:01, HLA-C*06:02, HLA-B*15:01, HLA-C*03:03, HLA-A*30:01, HLA-B*13:02, HLA-C*12:03, HLA-A*26:01, HLA-B*38:01, HLA-B*14:02, HLA-A*33:01, HLA-A*23:01, HLA-A*25:01, HLA-B*18:01, HLA-B*37:01, HLA-B*51:01, HLA-C*14:02, HLA-C*15:02, HLA-C*02:02, HLA-B*27:05, HLA-A*31:01, HLA-A*30:02, HLA-B*42:01, HLA-C*17:01, HLA-B*35:02, HLA-B*39:06, HLA-C*03:02, HLA-B*58:01, HLA-A*33:03, HLA-A*68:02, HLA-C*01:02, HLA-C*07:04, HLA-A*68:01, HLA-A*32:01, HLA-B*49:01, HLA-B*53:01, HLA-B*50:01, HLA-A*02:05, HLA-B*55:01, HLA-B*45:01, HLA-B*52:01, HLA-C*12:02, HLA-B*35:03, HLA-B*40:02, HLA-B*15:03 and/or HLA-A*74:01. The full-length amino acid sequences (including signal sequence and transmembrane domain) of these MHCI molecules are shown in SEQ ID NOs: 28-93, respectively. The amino acid sequences of soluble forms of these MHCI molecules (lacking signal sequence and transmembrane domain) are shown in SEQ ID NOs: 94-159, respectively.

In some embodiments, the pMHCI multimers described herein comprises the α1 and α2 domains of an MHCI heavy chain. In some embodiments, the compound described herein comprises the α1, α2, and α3 domains of an MHCI heavy chain.

In some embodiments, the two or more pMHCI or pMHCI-like polypeptides in the multimer comprises a β2-microglobulin polypeptide, e.g., a human β2-microglobulin. In some embodiments, the β2-microglobulin is wild-type human β2-microglobulin. In some embodiments, the β2-microglobulin comprises an amino acid sequence that is at least 80, 85, 90, 95, or 99% identical to the amino acid sequence of the human β2 microglobulin, the full-length sequence of which is shown in SEQ ID NO: 160 (UniProt Id. No. P61769). Alternatively, the human β2-microglobulin polypeptide used in the pMHCI multimer can comprise or consist of the amino acid sequence shown in SEQ ID NO: 2.

In some embodiments, the multimeric protein comprises a soluble MHCI polypeptide. In some embodiments the MHC-multimeric protein comprises a soluble MHCI α domain and a β2-microglobulin polypeptide. In some embodiments, the soluble MHCI protein comprises the MHCI heavy chain α1 domain and the MHCI heavy chain α2 domain.

Alternatively, in some embodiments, the MHCI monomer is a fusion protein comprising a β2m polypeptide or functional fragment thereof covalently linked to the MHCI heavy chain or functional fragment thereof. In some embodiments the carboxy (—COOH) terminus of β2m is covalently linked to the amino (−NH₂) terminus of the MHCI heavy chain.

In some embodiments, the MHC monomers comprise one or more linkers between the individual components of the MHCI monomer. In some embodiments, the MHCI monomer comprises a heavy chain fused with β2m through a linker. In some embodiments, the linker between the heavy chain and β2m is a flexible linker, e.g., made of glycine and serine. In some embodiments, the flexible linker between the heavy chain and β2m is between 5-20 residues long. In other embodiments, the linker between the heavy chain and β2m is rigid with a defined structure, e.g. made of amino acids like glutamate, alanine, lysine, and leucine. In one embodiment, the linker is a (G4S)₄ linker (SEQ ID NO: 181, wherein n=4).

The amino acid sequences of a number of MHC Class I proteins are known, and the genes have been cloned, therefore, the heavy chain monomers can be expressed using recombinant methods. Methods for the expression and purification of MHCI molecules have been extensively described (e.g., Altman et al., Curr. Protoc. Enz. 17.3.1-17.2-44, 2016). For example, the MHCI heavy chain and β2-microglobulin can be expressed in separate cells, and isolated by purification and then refolded in vitro. For example, the MHC polypeptide chains can be expressed in E. coli, where MHC polypeptide chains accumulate as insoluble inclusion bodies in the bacterial cell. In vitro refolding occurs in a refolding buffer where the polypeptides are added by e.g. dialysis or dilution. Refolding buffers can be any buffer wherein the MHC polypeptide chains and peptide are allowed to reconstitute the native trimer fold. The buffer may contain oxidative and/or reducing agents thereby creating a redox buffer system helping the MHC proteins to establish the correct fold. Examples of suitable refolding buffers include but are not limited to Tris-buffer, CAPS buffer, TAPs buffer, PBS buffer, other phosphate buffer, carbonate buffer and Ches buffer. Chaperone molecules or other molecules improving correct protein folding may also be added and likewise agents increasing solubility and preventing aggregate formation may be added to the buffer. Examples of such molecules include but is not limited to Arginine, GroE, HSP70, HSP90, small organic compounds, DnaK, CIpB, proline, glycinbetaine, glycerol, tween, salt, PLURONIC™.

Once expressed the MHCI complexes can be purified directly as whole MHCI or MHCI-peptide monomers from MHCI expressing cells. The MHCI monomers may be expressed on the surface of cells, and are then isolated by disruption of the cell membrane using, e.g., detergent followed by purification of the MHCI. In some embodiments, MHC monomers are expressed into the periplasm and expressing cells are lysed and released MHCI monomers purified. Alternatively, MHC monomers may be purified from the supernatant of cells secreting expressed proteins into culture supernatant. Methods for purifying MHCI monomers are well known in the art, for example, via the use of affinity tags together with affinity chromatography, beads coated with ant-tag and/or other techniques involving immobilization of MHCI protein to affinity matrix; size exclusion chromatography using, e.g., gel filtration, ion exchange or other methods able to separate MHC molecules from cells and/or cell lysates.

In some embodiments, recombinant expression of MHCI polypeptides allow a number of modifications of the MHC monomers. For example, recombinant techniques provide methods for carboxy terminal truncation which deletes the hydrophobic transmembrane domain. The carboxy termini can also be arbitrarily chosen to facilitate the conjugation of ligands or labels, for example, by introducing cysteine and/or lysine residues into the molecule. The synthetic gene will typically include restriction sites to aid insertion into expression vectors and manipulation of the gene sequence. The genes encoding the appropriate monomers are then inserted into expression vectors, expressed in an appropriate host, such as E. coli, yeast, insect, or other suitable cells, and the recombinant proteins are obtained. For example, the production of MHC class I polypeptides includes bacterial expression and folding of the MHC class I light chain, β2-microglobulin (β2m), as well as the formation of a complex consisting of the MHC class I heavy chain, β2m, and a placeholder peptide.

In some embodiments, the MHCI monomers are biotinylated on either their heavy chain or β2m. In some embodiments, the MHCI monomers are biotinylated before loading of the peptide either by refolding or peptide exchange. Biotinylation of the MHC monomers can be achieved as known in the art, e.g. by attaching biotin to a specific attachment site which is the recognition site of a biotinylating enzyme. In some embodiments, the biotinylating enzyme is BirA. In some embodiments, biotinylation is carried out on the desired protein chain in vivo as a post translational modification during protein expression.

(b) MHC Class II Polypeptides

MHC class II molecules are heterodimers composed of an a chain and a β chain, both of which are encoded by the MHC. The alpha chain is comprised of α1 and α2 domains. The beta chain is comprised of β1 and β 2 domains. The α1 and β1 domains of the chains interact noncovalently to form a membrane-distal peptide-binding domain, whereas the α2 and β2 domains form a membrane-proximal immunoglobulin-like domain. The antigen binding groove, where a peptide epitope binds, is made up of two α-helices and a β-sheet. Since the antigen binding groove of MHC class II molecules is open at both ends, the groove can accommodate longer peptide epitopes than MHC class I molecules. Peptide epitopes presented by MHC class II molecules can be 13-25 amino acids in length but typically are about 15-24 amino acid residues in length.

The disclosure provides a multimeric protein comprising two or more MHCII or MHCII-like polypeptides. The MHCII molecule can suitably be a vertebrate MHCII molecule such as a human, a mouse, a rat, a porcine, a bovine or an avian MHCII molecule.

In some embodiments, the multimeric MHCII multimers described herein, the MHC molecule is a human MHC class II protein: HLA-DR, HLA-DQ, HLA-DX, HLA-DO, HLA-DZ, and HLA-DP. The amino acid sequences of the MHCII a and 13 chains from a variety of vertebrate species, including humans, are known in the art and publicly available.

In some embodiments, the human MHCII molecule is of an allotype selected from the group consisting of DRB1*0101 (see, e.g., Cameron et al. (2002) J Immunol. Methods, 268:51-69; Cunliffe et al. (2002) Eur. J. Immunol., 32:3366-3375; Danke et al. (2003)J. Immunol., 171:3163-3169), DRB1*1501 (see, e.g., Day et al. (2003)J Clin. Invest, 112:831-842), DRBS*0101 (see, e.g., Day et al., ibid), DRB1*0301 (see, e.g., Bronke et al. (2005) Hum. Immunol., 66:950-961), DRB1*0401 (see, e.g., Meyer et al. (2000) PNAS, 97:11433-11438; Novak et al. (1999) J. Clin. Invest, 104:R63-R67; Kotzin et al. (2000) PNAS, 97:291-296), DRB1*0402 (see, e.g., Veldman et al. (2007) Clin. Immunol., 122:330-337), DRB1*0404 (see, e.g., Gebe et al. (2001)) Immunol. 167:3250-3256), DRB1*1101 (see, e.g., Cunliffe, ibid; Moro et al. (2005) BMC Immunol., 6:24), DRB1*1302 (see, e.g., Laughlin et al. (2007) Infect. Immunol. 75:1852-1860), DRB1*0701 (see, e.g., Danke, ibid), DQA1*0102 (see, e.g., Kwok et al. (2000) J Immunol., 164:4244-4249), DQB1*0602 (see, e.g., Kwok, ibid), DQA1*0501 (see, e.g., Quarsten et al. (2001) J Immunol., 167:4861-4868), DQB1*0201 (see, e.g., Quarsten, ibid), DPA1*0103 (see, e.g., Zhang et al. (2005) Eur. J. Immunol, 35:1066-1075; Yang et al. (2005) J Clin. Immunol., 25:428-436), and DPB1*0401 (see, e.g., Zhang, ibid; Yang, ibid).

In some embodiments, the MHCII molecule is human, and comprise, for example, an MHCII alpha and beta chains selected from the group consisting of HLA-DRA*01:01, HLA-DRB1*01:01, HLA-DRB1*01:02, HLA-DRB1*03:01, HLA-DRB1*04:01, HLA-DRB1*04:04, HLA-DRB1*07:01, HLA-DRB1*08:01, HLA-DRB1*10:01, HLA-DRB1*11:01, HLA-DRB1*11:04, HLA-DRB1*13:01, HLA-DRB1*13:02, HLA-DRB1*14:01, HLA-DRB1*15:01, HLA-DRB1*15:03, HLA-DQA1*01:01, HLA-DQB1*05:01, HLA-DQA1*01:02, HLA-DQB1*06:02, HLA-DQA1*03:01, HLA-DQB1*03:02, HLA-DQA1*05:01, HLA-DQB1*02:01, HLA-DQB1*03:01, HLA-DQB1*03:03, HLA-DQB1*04:02, HLA-DQB1*05:03, HLA-DQB1*06:03 and HLA-DQB1*06:04. The full-length amino acid sequences (including signal sequence and transmembrane domain) of these MHCII chains are shown in SEQ ID NOs: 194-223, respectively. The amino acid sequences of soluble forms of these MHCII chains (lacking signal sequence and transmembrane domain) are shown in SEQ ID NOs: 224-253, respectively.

In certain embodiments, an additional amino acid sequence can be appended to the C-terminal sequence of the alpha or beta chain of the MHCII molecule, for example for purposes of labeling and/or for attaching a moiety that mediates attachment (e.g., conjugation) to the multimerization domain. For example, an avitag (that mediates binding through the biotin binding site of Say) can be appended, such as an avitag with a Myc tag and a His tag (SEQ ID NO: 254) or an avitag with a Myc tag (SEQ ID NO: 255). In another embodiment, a sortag (that can mediate conjugation of click chemistry moieties through sortase, as described herein) can be appended, such as the sortag shown in SEQ ID NO: 257 or a sortag with a His tag as shown in SEQ ID NO: 256. In another embodiment, a V5 tag (SEQ ID NO: 258) is appended to the C-terminus.

In certain embodiments, heterodimerization pairs can be appended to the C-terminal sequence of the alpha and/or beta chains of the MHCII molecule. Non-limiting examples of such heterodimerization pair sequences include Fos and Jun (e.g., having the amino acid sequences shown in SEQ ID NOs: 259 and 260, respectively), acidic and basic leucine zippers (e.g., having the amino acid sequences shown in SEQ ID NOs: 261 and 262, respectively), knob and hole sequences (e.g., having the amino acid sequences shown in SEQ ID NOs: 263 and 264, respectively) for knobs-into-holes technology or spytab and spycatcher sequences (e.g., having the amino acid sequences shown in SEQ ID NOs: 265 and 266, respectively).

In certain embodiments, an MHCII-binding placeholder peptide is included in the expression construct for one of the MHCII chains, preferably the beta chain, such that the placeholder peptide and a digestible linker are encoded in the construct upstream of (N-terminally) and in operative linkage with the coding sequences for the MHCII chain. For example, the expression construct can encode (from N- to C-terminus): a placeholder peptide, an digestible linker, the MHCII chain (e.g., beta chain) and a C-terminal tag (e.g., encoding the amino acid sequence shown in SEQ ID NO: 192). In certain embodiments, an N-terminal tag is also appended upstream of the placeholder peptide, which allows for removal of non-exchanged peptide species following peptide exchange. Non-limiting examples of such N-terminal tags include a FLAG tag (e.g., having the amino acid sequence shown in SEQ ID NO: 267), a Strep-Tag (e.g., having the amino acid sequence shown in SEQ ID NO: 268) and a Protein C tag (e.g., having the amino acid sequence shown in SEQ ID NO: 269).

In some embodiments, the pMHCII multimers described herein comprise the α1 and α2 domains of an MHCII alpha chain and the β1 and β2 domains of an MHCII beta chain. In some embodiments, the multimer described herein comprises only the α1 and β1 domains of an MHCII heavy chain. In other embodiments, the pMHCII multimers comprise an alpha-chain and a beta-chain combined with a peptide. Other embodiments include an MHCII molecule comprised only of alpha-chain and beta-chain (so-called “empty” MHC II without loaded peptide), a truncated alpha-chain (e.g. the α1 domain) combined with full-length beta-chain, either empty or loaded with a peptide, a truncated beta-chain (e.g. the β1 domain) combined with a full-length alpha-chain, either empty or loaded with a peptide, or a truncated alpha-chain combined with a truncated beta-chain (e.g. α1 and β1 domain), either empty or loaded with a peptide.

In some embodiments, the multimeric protein comprises a soluble MHCII polypeptide. In some embodiments the MHC-multimeric protein comprises a soluble MHCII lacking transmembrane and intracellular domains.

The amino acid sequences of numerous MHC Class II proteins, including human MHCII, are known in the art, and the genes have been cloned. Therefore, the alpha and beta chain monomers can be expressed using recombinant methods. Methods for the expression and purification of MHCII molecules have been extensively described (e.g., Crawford et al. (1998) Immunity, 8:675-682; Novak et al. (1999) J. Clin. Invest., 104:R63-R67; Nepom et al. (2002) Arthrit. Rheum., 46:5-12; Day et al. (2003) J. Clin. Invest., 112:831-842; Vollers and Stern (2008) Immunol., 123:305-313; Cecconi et al. (2008) Cytometry, 73A:1010-1018, the entire contents of each of which is hereby incorporated by reference).

For MHC II molecules the alpha-chain and beta-chain may be expressed in separate cells as individual polypeptides or in the same cell as a fusion protein. The peptide of the MHC II-peptide complex may be produced separately and added following purification of whole MHC complexes or added during in vitro refolding or expressed together with alpha-chain and/or beta-chain connected to either chain through a linker. The genetic material can encode all or only a fragment of MHC class II alpha- and beta-chains. The genetic material may be fused with genes encoding other proteins, including proteins useful in purification of the expressed polypeptide chains (e.g., purification tags), proteins useful in increasing/decreasing solubility of the polypeptide(s), proteins useful in detection of polypeptide(s), proteins involved in coupling of MHC complex to multimerization domains and/or coupling of labels to MHC complex and/or MHC multimer.

In contrast to MHC I complexes, MHC II complexes are not easily refolded after denaturation in vitro. Only some MHC II alleles can be expressed in E. coli and refolded in vitro. Therefore, preferred expression systems for production of MHC II molecules are eukaryotic systems where refolding after expression of protein is not necessary. Preferred expression systems include mammalian expression systems, such as CHO cells, HEK cells or other mammalian cell lines suitable for expression of human proteins. Other expression systems include stable Drosophila cell transfectants, baculovirus infected insect T cells or other mammalian cell lines suitable for expression of proteins.

Stabilization of soluble MHC II complexes is even more important than for MHC I molecules, since both alpha- and beta-chain are participants in formation of the peptide binding groove and tend to dissociate when not embedded in the cell membrane. Accordingly, in one embodiment, MHCII monomers are prepared in which the peptide is covalently linked to the MHCII molecule. For example, one approach is the covalent synthesis of single-chain MHC class II chain-peptide complexes, directed by engineering peptide-specific complementary DNA (cDNA) sequences proximal to the beta-chain cDNA (as described in Crawford et al. (1999) Immunity, 8:675-682). In this strategy, the resulting polypeptide refolds with the peptide sequence extended from the amino terminus of the class II molecule. A tethering linker sequence in the peptide allows enough flexibility for the peptide to occupy the peptide binding groove in the mature class II molecule. A cleavable linker can be used to allow for cleavage of the covalent linkage between the peptide and the MHCII molecule (e.g., as described in Day et al. (2003) J. Clin. Invest., 112:831-842), thereby allowing for peptide exchange and loading of the MHCII molecule with other peptides (e.g., a library of different peptides).

Once expressed, the MHCII complexes can be purified directly as whole MHCII or MHCII-peptide monomers from MHCII expressing cells. The MHCII monomers may be expressed on the surface of cells, and are then isolated by disruption of the cell membrane using, e.g., detergent followed by purification of the MHCII. In some embodiments, MHC monomers are expressed into the periplasm and expressing cells are lysed and released MHCII monomers purified. Alternatively, MHC monomers may be purified from the supernatant of cells secreting expressed proteins into culture supernatant. Methods for purifying MHCII monomers are well known in the art, for example, via the use of affinity tags together with affinity chromatography, beads coated with ant-tag and/or other techniques involving immobilization of MHCII protein to affinity matrix; size exclusion chromatography using, e.g., gel filtration, ion exchange or other methods able to separate MHC molecules from cells and/or cell lysates.

In some embodiments, recombinant expression of MHCII polypeptides allow a number of modifications of the MHC monomers. For example, recombinant techniques provide methods for carboxy terminal truncation which deletes the hydrophobic transmembrane domain. The carboxy termini can also be arbitrarily chosen to facilitate the conjugation of ligands or labels, for example, by introducing cysteine and/or lysine residues into the molecule. The synthetic gene will typically include restriction sites to aid insertion into expression vectors and manipulation of the gene sequence. The genes encoding the appropriate monomers are then inserted into expression vectors, expressed in an appropriate host, such as E. coli, yeast, insect, or other suitable cells, and the recombinant proteins are obtained.

In some embodiments, the MHCII monomers are biotinylated on either their alpha or beta chain. In some embodiments, the MHCII monomers are biotinylated before loading of the peptide either by refolding or peptide exchange. Biotinylation of the MHC monomers can be achieved as known in the art, e.g. by attaching biotin to a specific attachment site which is the recognition site of a biotinylating enzyme. In some embodiments, the biotinylating enzyme is BirA. In some embodiments, biotinylation is carried out on the desired protein chain in vivo as a post translational modification during protein expression.

### Placeholder Peptides

(a) MHC Class I Placeholder Peptides

In the methods provided herein, the MHCI monomers are loaded with a placeholder peptide to facilitate proper folding of the MHCI monomers to produce placeholder-peptide loaded MHCI (p*MHCI) prior to multimerization. Examples of placeholder peptides and methods of inducing folding MHCI heavy chains and β2-microglobulin in vitro in the presence of a placeholder peptide have been described in the art (e.g., Bakker et al. 2008) PNAS 105:3825-3830; Rodenko et al. (2006) Nat. Prot. 1: 1120-1132).

In some embodiments, the placeholder peptide is an HLA-A, HLA-B or HLA-C peptide. In some embodiments, the placeholder peptide is an HLA-A1 peptide (e.g., A1:01 binding peptide). In some embodiments, the placeholder peptide is an HLA-A2 peptide (e.g., A02-01 binding peptide). In other embodiments, the placeholder peptide is an HLA-A3 peptide (e.g., A3:01 binding peptide), an HLA-A11 peptide (e.g., A11:01 binding peptide), an HLA-A24 peptide (e.g., A24:02 binding peptide), an HLA-B7 peptide (e.g., B7:02 binding peptide), an HLA-B8 peptide (e.g., B8:01 binding peptide), an HLA-B15 peptide (e.g., B15:01 binding peptide), an HLA-B35 peptide (e.g., B35:01 binding peptide), an HLA-B40 peptide (e.g., B40:01 binding peptide), an HLA-B58 peptide (e.g., B58:01 binding peptide), an HLA-C3 peptide (e.g., C3:04 binding peptide), an HLA-C4 peptide (e.g., C4:01 binding peptide) an HLA-C7 peptide (e.g., C7:02 binding peptide) or an HLA-C8 peptide (e.g., C8:01 binding peptide). In some embodiments, the placeholder peptide is a synthetic peptide.

In some embodiments, the affinity of the placeholder peptide for the binding groove of MHCI is lower than the rescue peptide(s). In some embodiments, the affinity of the placeholder peptide for the MHCI binding groove is about 10-fold lower than the rescue peptide(s). In some embodiments, the affinity of the place holder peptide for the binding groove of MHCI is higher than the rescue peptide(s); however, the placeholder peptide can still be replaced by the rescue peptide by use of an excess concentration of the rescue peptide.

In some embodiments, the placeholder peptide is thermolabile. Is some embodiments, the placeholder peptide is thermolabile at a temperature between about 30-37° C. In some embodiments, the placeholder peptide is labile at a temperature at or above 30° C., at or above 32° C., at or above 34° C., at or above 35° C., at or above 36° C., or at about 37° C. Thermal labile placeholder peptides and methods of identifying and producing thermal labile placeholder peptides have been described (e.g., WO 93/10220; WO 2005/047902; US 2008/0206789; Luimstra et al. (2019) Curr. Protoc. Immunol. 126(1):e85; Luimstra et al. (2018) J. Exp. Med. 215(5):1493-1504).

In some embodiments the placeholder peptide is labile at an acidic pH. In some embodiments, the placeholder peptide is labile between about pH 2.5 and 6.5. In some embodiments, the placeholder peptide is labile at a pH of about 2.5-6.0, 3.0-6.0, 3.0-6.5, 3.5-6.0 3.5-6.5, 4.0-6.0, 4.0-6.5, 4.5-6.0, 4.5-6.5, 5.0-6.0, 5.0-6.5, 5.0, 5.5, 6.0 or 6.5. In some embodiments, the placeholder peptide is labile at a basic pH. In some embodiments, the placeholder peptide is labile between about pH 9-11. In some embodiments, the placeholder peptide is labile at or above pH 9, at or above pH 9.5, at or about pH 10, at or about pH 10.5, or at or about pH 11. Methods of generating and using pH sensitive placeholder peptides are publicly available, for example, as described in WO 93/10220; US 2008/0206789; and Cameron et al. (2002), 1 Immunol. Meth. 268:51-59.

In some embodiments, the placeholder peptide comprises a cleavable moiety. Various types of cleavable moieties are known in the art and include, for example, moieties that are cleaved by photoirradiation, enzymes, nucleophilic or electrophilic agents, reducing and oxidizing reagents (e.g., reviewed in Leriche et al., Biorg. Med. Chem. 20(2):571-582, 2012).

In some embodiments, the cleavable placeholder peptide comprises one or more photocleavable non-natural β-amino acids. In some embodiments, the placeholder peptide comprises 3-amino-3-(2-nitro-phenyl)-proprionic acid. In some embodiments, the placeholder peptide comprises (2-nitro)phenylglycine. In some embodiments, the placeholder peptide comprises an azobenzene group. In some embodiments, the HLA-A2 placeholder peptide is p*A02:01, KILGFVFJV (SEQ ID NO: 15) or GILGFVFJL (SEQ ID NO: 7), wherein J is 3-amino-3-(2-nitro)phenyl-propionic acid. In some embodiments, the placeholder peptide is selected from the group consisting of p*A1:01, STAPGJLEY (SEQ ID NO: 16); p*A3:01, RIYRJGATR (SEQ ID NO:17); p*A11:01, RVFAJSFIK (SEQ ID NO: 18); p*A24:02, VYGJVRACL (SEQ ID NO: 11); p*B7:02, AARGJTLAM (SEQ ID NO: 14); p*B35:01, KPIVVLJGY (SEQ ID NO: 19); p*C3:04, FVYGJSKTSL (SEQ ID NO: 20), p*B8:01, FLRGRAJGL (SEQ ID NO: 21); p*C7:02, VRIJHLYIL (SEQ ID NO: 22); p*C4:01, QYDJAVYKL (SEQ ID NO: 23); p*B15:01, ILGPJGSVY (SEQ ID NO: 24); p*B40:01, TEADVQJWL (SEQ ID NO: 25); p*B58:01, ISARGQJLF (SEQ ID NO: 26); and p*C8:01, KAAJDLSHFL (SEQ ID NO: 27), wherein J is 3-amino-3-(2-nitro)phenyl-propionic acid. Methods of generating placeholder peptides containing photocleavable amino acids are known in the art and have been previously described (e.g., Toebes et al., Curr. Protoc. Immunol. 87:18.16.1-18.16.20, 2009; Bakker et al., supra, Rodenko et al. supra). In various embodiments, the photocleavable placeholder peptide is cleaved upon exposure to UV-light using previously described methods (e.g., Toebes et al., (2006) Nat Med. 12(2):246-51; Bakker et al. (2008) Proc Natl Acad Sci USA. 105(10):3825-30; Rodenko et al. (2006) Nat Protoc. 1(3):1120-32; Frosig et al., (2015) Cytometry A. 87(10):967-75).

In some embodiments, the placeholder peptide comprises a chemoselective moiety. In some embodiments, the chemoselective moiety comprises a sodium dithionite sensitive azobenzene linker, wherein the azobenzene comprises at least one aromatic group comprising an electron-donor group and is located between two amino acid residues. Azobenzine linkers and methods for chemoselective peptide exchange are known in the art, for example, as described in U.S. Pat. No. 10,400,024.

In some embodiments, the placeholder peptide comprises a cleavable moiety that is cleaved upon exposure to an aminopeptidase. In some embodiments, the cleavage of the amino acid residue occurs via the use of a methionine aminopeptidase. The methionine aminopeptidase can cleave a methionine from a peptide when the amino acid residue at position two is, for example, glycine, alanine, serine, cysteine, or proline. In some embodiments, the cleavable moiety comprises a thrombin cleavage domain.

In some embodiments, the placeholder peptide comprises a cleavable moiety is sensitive to a chemical trigger. In some embodiments, the placeholder peptide comprises periodate-sensitive amino acid. In some embodiments, the periodate-sensitive amino acid comprises a vicinal diol moiety. In some embodiments, the periodate-sensitive amino acid comprises a vicinal amino alcohol. In some embodiments, the periodate-sensitive amino acid is 1,2-amino-alcohol-containing amino acid. In some embodiments, the periodate-sensitive amino acid is α,γ-diamino-β-hydroxybutanoic acid (DAHB). Methods for producing and using peptides containing periodate-sensitive amino acids are publicly available, for example, as described in Rodenko et al. ((2009) J. Am. Chem. Soc. 131:12605-12313) and Amore et al. ((2013) ChemBioChem 14:123-131).

In some embodiments, the placeholder peptide is a dipeptide. In some embodiments, the dipeptide binds to the F pocket of the MHCI binding groove. In some embodiments, the second amino acid of the dipeptide is hydrophobic. In some embodiments, the dipeptide is selected from the group consisting of glycyl-leucine (GL), glycyl-valine (GV), glycyl-methione (GM), glycyl-cyclohexylalanine (GCha), glycyl-homoleucine (GHle) and glycyl-phenylalanine (GF). Methods for producing and using dipeptides as placeholder peptides are publicly available, for example, as described in Saini et al. (PNAS 112:202-207, 2015).

In some embodiments, the placeholder peptide comprises GILGFVFJL (SEQ ID NO:7). In some embodiments, the placeholder peptide consists of GILGFVFJL (SEQ ID NO:7).

In some embodiments, the placeholder peptide further comprises a fluorescent label. In some embodiments, the fluorescent label is attached to a cysteine residue in the placeholder peptide.

In some embodiments, p*MHCI molecules are purified, and stored to serve as a source of stock molecules that can be exchanged with peptide epitopes of interest upon exposure to peptide exchange conditions as described herein.

(b) MHC Class II Placeholder Peptides

In the methods provided herein, the MHCII monomers are loaded with a placeholder peptide to facilitate proper folding of the MHCII monomers to produce placeholder-peptide loaded MHCII (p*MHCII) prior to multimerization. In various embodiments, the placeholder peptide is peptide that binds HLA-DR, HLA-DQ, HLA-DX, HLA-DO, HLA-DZ or HLA-DP. In some embodiments, the placeholder peptide is a synthetic peptide.

In some embodiments, the affinity of the placeholder peptide for the binding groove of MHCII is lower than the rescue peptide(s). In some embodiments, the affinity of the placeholder peptide for the MHCII binding groove is about 10-fold lower than the rescue peptide(s).

In some embodiments, the placeholder peptide is thermolabile. In some embodiments, the placeholder peptide is thermolabile at a temperature between about 30-37° C. In some embodiments, the placeholder peptide is labile at a temperature at or above 30° C., at or above 32° C., at or above 34° C., at or above 35° C., at or above 36° C., or at about 37° C. Thermal labile placeholder peptides and methods of identifying and producing thermal labile placeholder peptides have been described (e.g., WO 93/10220; WO 2005/047902; US 2008/0206789; Luimstra et al., Curr. Protoc. Immunol. 126(1):e85, 2019; Luimstra et al., J. Exp. Med. 215(5):1493-1504, 2018).

In some embodiments the placeholder peptide is labile at an acidic pH. In some embodiments, the placeholder peptide is labile between about pH 2.5 and 6.5. In some embodiments, the placeholder peptide is labile at a pH of about 2.5-6.0, 3.0-6.0, 3.0-6.5, 3.5-6.0 3.5-6.5, 4.0-6.0, 4.0-6.5, 4.5-6.0, 4.5-6.5, 5.0-6.0, 5.0-6.5, 5.0, 5.5, 6.0 or 6.5. In some embodiments, the placeholder peptide is labile at a basic pH. In some embodiments, the placeholder peptide is labile between about pH 9-11. In some embodiments, the placeholder peptide is labile at or above pH 9, at or above pH 9.5, at or about pH 10, at or about pH 10.5, or at or about pH 11. Methods of generating and using pH sensitive placeholder peptides are publicly available, for example, as described in WO 93/10220; US 2008/0206789; and Cameron et al. (2002) J. Immunol. Meth. 268:51-59.

In some embodiments, the placeholder peptide comprises a cleavable moiety. Various types of cleavable moieties are known in the art and include, for example, moieties that are cleaved by photoirradiation, enzymes, nucleophilic or electrophilic agents, reducing and oxidizing reagents (e.g., reviewed in Leriche et al. (2012) Biorg. Med. Chem. 20(2):571-582).

In one embodiment, the placeholder peptide is fused to a degradation tag and peptide exchange is promoted by proteolysis in the presence of a corresponding protease (the digests the degradation tag) along with the presence of the rescue peptide(s).

In some embodiments, the cleavable placeholder peptide is a photocleavable peptide, e.g., cleaved upon exposure to UV light. For example, the placeholder peptide can comprise one or more photocleavable non-natural amino acids. MHCII-binding photocleavable peptides, e.g., that incorporate the UV-sensitive amino acid analog 3-amino-3-(2-nitrophenyl)-propionate have been described (see e.g., Negroni and Stern (2018) PLos One, 13(7):e0199704).

In one embodiment, the MHCII placeholder peptide is a CLIP peptide, such as having the amino acid sequence KPVSKMRMATPLLMQA (SEQ ID NO: 189). In one embodiment, the CLIP peptide is cleavable. In one embodiment, the MHCII monomers are synthesized with the cleavable CLIP peptide covalently attached, such as by synthesis of single-chain MHC class II chain-peptide complexes, directed by engineering peptide-specific complementary DNA (cDNA) sequences proximal to the beta-chain cDNA (see e.g., Day et al. (2003) J. Clin. Invest., 112:831-842). Cleavage of the covalent linkage between the CLIP peptide (as the placeholder peptide) and MHCII thus allows for peptide exchange with other MHCII-binding peptides.

Other MHCII binding peptides have been described in the art that can be used as placeholder peptides, based on appropriate pairing of an MHCII molecule and its known MHCII binding peptide. Non-limiting examples of known MHCII molecule/MHCII binding peptide pairs include: DRA1*0101/DRB1*0401 and the immunodominant peptide of hemagglutinin, HA₃₀₇₋₃₁₉ (see Novak et al. (1999) J. Clin. Invest., 104:R63-R67) and HLA-DR*1101 and tetanus-toxoid (TT)-derived p2 peptide (TT₈₃₀₋₈₄₄) having the amino acid sequence QIYKANSKFIGITEL (SEQ ID NO: 190) (see Cecconi et al. (2008) Cytometry, 73A:1010-1018).

Production of p*MHC Multimers

Multimerization domains for use in producing the pMHC multimers provided herein include proteins, polypeptide or other multimeric moieties suitable for the covalent conjugation of two or more pMHC or p*MHC monomers, which do not interfere with binding of the pMHC polypeptides to cells. In some embodiments, the multimerization domain comprises protein subunits. In some embodiments, the multimerization domain is a homomultimer of protein subunits. In some embodiments, the multimerization domain is a heteromultimer of protein subunits. In some embodiments, the multimer is a dimer, trimer, tetramer, pentamer, hexamer, octamer, decamer or dodecamer. In one embodiment, the pMHC multimer is a tetramer.

Examples of suitable binding entities are streptavidin (SA) and avidin and derivatives thereof, biotin, immunoglobulins, antibodies (monoclonal, polyclonal, and recombinant), antibody fragments and derivatives thereof, leucine zipper domain of AP-1 (jun and fos), hexa-his (metal chelate moiety), hexa-hat GST (glutathione S-transferase) glutathione affinity, Calmodulin-binding peptide (CBP), Strep-Tag®, Cellulose Binding Domain, Maltose Binding Protein, S-Peptide Tag, Chitin Binding Tag, Immuno-reactive Epitopes, Epitope Tags, E2Tag, HA Epitope Tag, Myc Epitope, FLAG Epitope, AU1 and AU5 Epitopes, Glu-Glu Epitope, KT3 Epitope, IRS Epitope, Btag Epitope, Protein Kinase-C Epitope, VSV Epitope, lectins that mediate binding to a diversity of compounds, including carbohydrates, lipids and proteins, e. g., Con A (Canavaliaensi formis) or WGA (wheat germ agglutinin) and tetranectin or Protein A or G (antibody affinity) or coiled-coil polypeptides e.g. leucine zipper. Combinations of such binding entities are also included.

In some embodiments, the multimerization domain is a tetramer of streptavidin (SA or SAv) or a derivative thereof. In some embodiments, the multimerization domain is tetrameric streptavidin. In some embodiments, the tetramer comprises Strep-Tag® or Strep-Tactin®. Strep-Tag® or Strep-Tactin® are described in U.S. Pat. Nos. 5,506,121 and 6,103,493, respectively, and are commercially available from a number of sources. To attach MHC monomers to streptavidin non-covalently via the biotin-binding site of SAv, an avitag (such as having the amino acid sequence shown in SEQ ID NO: 161, which includes a 6×His Tag and a FLAG tag) can be incorporated into MHC monomer, for example at the C-terminal end (see e.g., Example 3).

In the methods provided herein, pMHC multimers are produced by covalent conjugation of each p*MHC monomer to the N- or C-terminal of each subunit of the multimerization domain, resulting in a reaction product referred to herein as a Conjugated Multimer. In one embodiment, the Conjugated Multimer is a pMHC Class I (pMHCI) Conjugated Multimer. In another embodiment, the Conjugated Multimer is a pMHC Class II (pMHCII) Conjugated Multimer.

In some embodiments, pMHCI multimers are produced by covalent conjugation of the multimerization domain to the C-terminus of the MHCI α1 domain. In some embodiments, the pMHCI multimers are produced by covalent conjugation of the multimerization domain to the C-terminus of the MHCI α2 domain. In some embodiments, the pMHCI multimers are produced by covalent conjugation of the multimerization domain to the C-terminus of the MHCI α3 domain. In some embodiments, the pMHCI multimers are produced by covalent conjugation of the multimerization domain to the C-terminus of the β2-microglobulin of each p*MHC monomer.

In a preferred embodiment, pMHCII multimers are produced by covalent conjugation of the multimerization domain to the MHCII α chain. In another embodiment, pMHCII multimers are produced by covalent conjugation of the multimerization domain to the MHCII β chain. In certain embodiments, pMHCII multimers are produced by covalent conjugation of the multimerization domain to the C-terminus of the MHCII α1 domain. In certain embodiments, the pMHCII multimers are produced by covalent conjugation of the multimerization domain to the C-terminus of the MHCII α2 domain. In certain embodiments, the pMHCII multimers are produced by covalent conjugation of the multimerization domain to the C-terminus of the MHCII β1 domain. In certain embodiments, the pMHCII multimers are produced by covalent conjugation of the multimerization domain to the C-terminus of the MHCII β2 domain.

A number of suitable methods for forming covalent bonds between each MHC monomer and the multimerization domain are provided herein.

(a) Chemical Bioconjugation

In some embodiments, the p*MHC multimers are produced by chemical conjugation. In some embodiments, the chemical conjugation is mediated by cysteine bioconjugation of the p*MHC polypeptides to the multimerization domain. In some embodiments, the cysteine bioconjugation is mediated by cysteine alkylation. In some embodiments, the cysteine bioconjugation is mediated by cysteine oxidation. In other embodiments, the cysteine bioconjugation is mediated by a desulfurization reaction. In some embodiments, cysteine bioconjugation is mediated by iodoacetamide. In some embodiments, the cysteine bioconjugation is mediated by maleimide. Methods for utilizing cysteine mediated linkage of two moieties which can be used to produce the pMHC multimers disclosed herein have been described, for example, see Chalker et al. (2009) Chem Asian J. 4(5):630-40; Spicer et al. (2015) Nat Commun. 5:4740.

In some embodiments, the MHC multimers are produced by chemical modification of amino acids other than cysteine, including but not limited to lysine, tyrosine, arginine, glutamate, aspartate, serine, threonine, methionine, histidine and tryptophan side-chains, as well as N-terminal amines or C-terminal carboxyls, as previously described (Basle et al. (2010) M Chem Biol. 17(3):213-27; Hu et al. (2016) Chem Soc Rev. 45(6):1691-719; Lin et al. (2017) Science 355(6325):597-602).

(b) Native Chemical Ligation

In some embodiments, the pMHC multimers are produced by native chemical ligation (NCL), wherein each p*MHC polypeptide comprises a C-terminal thioester, and each subunit of the multimerization domain comprises an N-terminal cysteine residue, or functional equivalent thereof, wherein the reaction between the cysteine side-chain and the thioester irreversibly forms a native peptide bond, thus ligating the p*MHC monomers to the multimerization domain. Methods for NCL have been described (Hejjaoui et al. (2015) M Protein Sci. 24(7):1087-99; Mandal et al. (2012) Proc Natl Acad Sci USA 109(37):14779-84; Torbeev et al. (2013) Proc Natl Acad Sci USA 110(50):20051-6).

In some embodiments, β- and/or γ-thio amino acids are incorporated into the p*MHC monomers. In some embodiments, β- and/or γ-thio amino acids replace the cysteine-like residue at an N-terminal position of each subunit of the multimerization domain, e.g., to provide a reactive thiol for trans-thioesterification. Desulfurization protocols can then produce the desired native side-chain. In some embodiments, NCL is performed at an alanine residue. In other embodiments, NCL is performed at phenylalanine (Crich & Banerjee, 2007), valine (Chen et al. 2008; Haase et al. 2008), leucine (Harpaz et al. 2010; Tan et al. 2010), threonine (Chen et al. 2010b), lysine (El Oualid et al. 2010; Kumar et al. 2009; Yang et al. 2009), proline (Shang et al. 2011), glutamine (Siman et al. 2012), arginine (Malins et al. 2013), tryptophan (Malins et al. 2014), aspartate (Thompson et al. 2013), glutamate (Cergol et al. 2014) and asparagine (Sayers et al. 2015). Ligation/desulfurization approaches that remove purification steps and increase the yield of ligated products have been described (Moyal et al. 2013; Thompson et al. 2014).

(c) Click Chemistry Mediated Bioorthogonal Conjugation

In some embodiments, the p*MHC multimers are produced by bioorthogonal conjugation between the conjugation moiety at the C-terminus of each p*MHC monomer and the conjugation moiety at the N-terminus of each subunit of the multimerization domain. In some embodiments, the biorthogonal conjugation is mediated by “click chemistry.” (see, e.g., Kolb et al. (2001) Angewandte Chemie International Edition 40: 2004-2021). Conjugation moieties suitable for click chemistry, reaction conditions, and associated methods are available in the art (e.g., Kolb et al. (2001) Angewandte Chemie International Edition 40:2004-2021; Evans (2007) Australian Journal of Chemistry 60: 384-395; Lahann, Click Chemistry for Biotechnology and Materials Science, John Wiley & Sons Ltd, ISBN 978-0-470-69970-6, 2009). In some embodiments, a click chemistry moiety may comprise or consist of a terminal alkyne, azide, strained alkyne, diene, dieneophile, alkoxyamine, carbonyl, phosphine, hydrazide, thiol, or alkene moiety. In certain embodiments, the azide is a copper-chelating azide. In one embodiment, the copper-chelating azide is a picolyl azide, such as Gly-Gly-Gly-(PEG)4-Picolyl-Azide. Reagents for use in click chemistry reactions are commercially available, such as from Click Chemistry Tools (Scottsdale, Ariz.) or GenScript (Piscataway, N.J.).

For conjugation of each p*MHC monomer to a subunit of the multimerization domain via click chemistry, the click chemistry moieties of the proteins have to be reactive with each other, for example, in that the reactive group of one of the click chemistry moiety of each p*MHC monomer reacts with the reactive group of the second click chemistry moiety on a subunit of the multimerization domain to form a covalent bond. Such reactive pairs of click chemistry handles are well known to those of skill in the art and include but are not limited to those set forth in FIG. 1.

In some embodiments, each p*MHC conjugation moiety can be covalently conjugated under click chemistry reaction conditions to the conjugation moiety of each subunit of the multimerization domain. In some embodiments a sortase-mediated conjugation is used to install a first click chemistry moiety at the C-terminus of each p*MHC monomer, and a second click chemistry moiety reaction to each subunit of the multimerization domain. In the methods provided herein, two or more p*MHC monomers containing the first click chemistry moiety are conjugated to the second click chemistry moiety at the C-terminus of each subunit of the multimerization domain under click chemistry conditions. Methods of attaching click chemistry moieties utilizing sortase are described, for example, in WO2013/00355, the entire contents of which is hereby incorporated by reference. Non-limiting exemplifications of pMHC multimers prepared using Alkyne-Azide click chemistry in combination with sortase-mediated conjugation are described in detail in Examples 1, 5, 6 and 7.

In some embodiments, an intein-mediated conjugation is used to install a first click chemistry moiety at the C-terminus of each p*MHC monomer, and a second click chemistry moiety reaction to each subunit of the multimerization domain. Methods of utilizing intein-mediated conjugated are described further herein.

In some embodiments, the methods of click chemistry mediated covalent conjugation of the p*MHC monomers to the multimerization domain provided herein comprise native chemical ligation of C-terminal thioesters with β-amino thiols (Xiao J et al. (2009) Org Lett. 11(18):4144-7).

In some embodiments, the click chemistry used to produce the p*MHC multimers comprises 1,3-dipolar cycloaddition (e.g., the Cu(I)-catalyzed stepwise variant, often referred to simply as the “click reaction”; see, e.g., Tornoe et al. (2002) Journal of Organic Chemistry 67: 3057-3064). Copper and ruthenium are the commonly used catalysts in the reaction. The use of copper as a catalyst results in the formation of 1,4-regioisomer whereas ruthenium results in formation of the 1,5-regioisomer.

In some embodiments, the MHC monomers are ligated to an alkynated peptide by expressed protein ligation (EPL) and then conjugated to an azide-labeled multimerization domain by Cu(I)-catalyzed terminal azide-alkyne cycloaddition (CuAAC).

In some embodiments, the click chemistry conjugation comprises a cycloaddition reaction, such as the Diels-Alder reaction. In some embodiments, the MHCI and multimerization domain are conjugated by azide-alkyne 1,3-dipolar cycloaddition (“click chemistry). In some embodiments, the cycloaddition is promoted by the presence of Cu(I)-catalyzed cycloaddition (CuAAC).

In some embodiments, the click chemistry conjugation comprises nucleophilic addition to small strained rings like epoxides and aziridines. In some embodiments, the cycloaddition is promoted by strained cyclooctyne systems, for example, as described in Agard et al. (2004) J. Am Chem Soc. 126(46): 15046-7.

In some embodiments, the click chemistry conjugation comprises nucleophilic addition to activated carbonyl groups.

In some embodiments, the conjugation of the pMHC monomers and multimerization domain occurs by a bioorthogonal reaction. In some embodiments, the MHC and multimerization domain are conjugated by inverse-electron demand Diels-Alder reactions between strained dienophiles and tetrazine dienes, for example, as described in Blackman et al. (2008) J. Am Chem Soc. 130(41):13518-9; and Devaraj et al. (2008) Bioconjug Chem. 19(12):2297-9). In some embodiments, the dienophile is a trans-cyclooctene. In some embodiments, the dienophile is a norbornene.

(d) Sortase Mediated Conjugation

In some embodiments, conjugation between the p*MHC monomers and the multimerization domain is mediated by a cysteine transpeptidase. In some embodiments, the cysteine transpeptidase is a sortase, or enzymatically active fragment thereof. A variety of sortase enzymes have been described and are commercially available (e.g., Antos et al. (2016) Curr. Opin. Struct. Biol. 38:111-118. Sortases recognize and cleave an amino acid motif, referred to as a “sortag”, to produce a peptide bond between the acyl donor and acceptor site on two polypeptides, resulting in the ligation of different polypeptides which contain N- or C-terminal sortags. Non-limiting exemplifications of pMHC multimers prepared using sortase-mediated conjugation (in combination with Alkyne-Azide click chemistry) are described in detail in Examples 1, 5, 6 and 7.

Accordingly, in some embodiments, each p*MHC monomer comprises a C-terminal sortag, and each subunit of the multimerization domain comprises an C-terminal sortag. In some embodiments, the sortase catalyzes the formation of a peptide bond between an MHC polypeptide and each of the subunits of the multimerization domain.

In some embodiments, the recognition motif is added to the C-terminus of each of the pMHC monomers, and an oligo-glycine motif is added to the C-terminus of each of the subunits of the multimerization domain. Upon addition of sortase to the mixture of MHC monomers and multimerization domains, the polypeptides are covalently linked through a native peptide bond to produce a pMHC multimer.

In some embodiments, the MHC monomers and/or multimerization domain are expressed in frame with the sortags. In some embodiments, additional tags may be included, for example, a 6×-His tag (Sinisi et al. (2012) Bioconjug. Chem 23:1119-1126), a nucleophilic fluorochrome (Nair et al. (2013) Immun. Inflamm. Dis. 1:3-13), and/or a FLAG tag (Greineder et al. (2018) Bioconjug. Chem. 29:56-66).

In some embodiments, the sortag contains a modified amino acid suitable for chemical conjugation between the MHC monomers and the multimerization domain. In some embodiments, the sortag contains a C-terminal azidolysine residue to enable oriented click-click chemistry conjugation as described herein.

In some embodiments, the MHC polypeptide and/or multimerization domains comprise a linker between the polypeptide and the sortag. In some embodiments, each MHC polypeptide and each subunit of the multimerization domain comprises a sortag with a linker. Suitable linkers have been described, for example, in Greineder et al. (2018) Bioconjug. Chem. 29:56-66. In some embodiments, the linker is a semi-rigid linker. In some embodiments, the linker comprises (SSSSG)₂SAA (SEQ ID NO: 182). In some embodiments, the linker comprises (G)₅ (SEQ ID NO: 183).

In some embodiments, the sortag contains a fluorophore-modified lysine residue to facilitate measurement of reaction progression and efficiency

In some embodiments, the sortase is Ca2+ dependent. In some embodiments, the sortase is Ca2+ independent.

In some embodiments, the sortag-labeled MHC molecule is a soluble HLA-A2 molecule (HLA-A*02:01) with a C-terminal sortag and 6×His tag, such as having the amino acid sequence shown in SEQ ID NO: 1. In some embodiments, the sortag-labeled multimerization domain is a streptavidin molecule with a C-terminal sortag and 6×His Tag, such as having the amino acid sequence shown in SEQ ID NO: 3. In some embodiments, the sortag label with a 6×His tag has the amino acid sequence shown in SEQ ID NO: 162. Various other sortag sequences are known in the art and are suitable for use in preparing the Conjugated Multimers of the disclosure, non-limiting examples of which are described further below.

In some embodiments, the sortag comprises the amino acid sequence LPXTG (SEQ ID NO: 163), wherein X is any amino acid, and the sortase cleaves between the threonine and glycine backbone within the motif.

In some embodiments, the sortase recognizes a sortag comprising an amino acid sequence selected from IPKTG (SEQ ID NO:164), MPXTG (SEQ ID NO:165), LAETG (SEQ ID NO:166), LPXAG (SEQ ID NO:167), LPELG (SEQ ID NO:168), LPELG (SEQ ID NO:169) or LPEVG (SEQ ID NO:170).

In some embodiments, the sortase is a SrtAstaph mutant. In some embodiments, the SrtAstaph mutant is F40, and the recognition motif is XPKTG (SEQ ID NO: 171) (Piotukh et al. (2011) J. Am. Chem. Soc. 133:17536-17539). In some embodiments, the SrtAstaph mutant is F40 and the recognition motif is APKTG (SEQ ID NO:172), DPKTG (SEQ ID NO:173) or SPKTG (SEQ ID NO:174).

In some embodiments, the SrtAstaph mutant is SrtAstaph pentamutant and the recognition motif is LPXTG (SEQ ID NO:163), wherein X is any amino acid, LPEXG, (SEQ ID NO:175), wherein X is any amino acid, or LAETG (SEQ ID NO:166). In some embodiments, the mutant is SrtAstaph pentamutant and the recognition motif is LPEAG (SEQ ID NO:176), LPECG (SEQ ID NO:177) or LPESG (SEQ ID NO:168). In some embodiments, the SrtAstaph mutant is 2A-9 and the recognition motif is LAETG (SEQ ID NO:166). In some embodiments, the SrtAstaph mutant is 4S-9 and the recognition motif is LPEXG (SEQ ID NO:178), wherein X=A, C or S).

In some embodiments, the sortase is a soluble fragment of the wild-type sortase. In some embodiments, the sortase is a soluble fragment of a modified sortase A (Mao et al. (2004) J Am Chem Soc. 126(9):2670-1 A).

In some embodiments, the sortase is a variant or homolog of S. aureus sortase A (Antos et al. (2016) Curr Opin Struct Biol. 38:111-8; Don et al. (2014) Proc Natl Acad Sci USA. 111(37):13343-8; Glasgow et al. (2016) J Am Chem Soc. (24):7496-9).

Methods of conjugation of sortags into proteins have also been described. (Matsumoto et al. (2016) ACS Synth Biol. 5(11):1284-1289; Williams et al. (2016) PLoS One. 11(4):e0154607; and Witte et al. (2012) Proc Natl Acad Sci USA. 109(30):11993-8; Mao et al. (2004) JAm Chem Soc. 126(9):2670-1; Guimaraes et al. (2012) Nat Protoc. 8(9):1787-99 and Theile et al. (2013) NatProtoc. 8(9):1800-7.)

In some embodiments, the aminoglycine peptide fragment generated by the sortase reaction, is removed by dialysis or centrifugation, e.g., while the reaction is proceeding (Freiburger et al. (2015) Biomol NMR. 63(1):1-8). In some embodiments, affinity immobilization strategies or flow-based platforms are used for the selective removal of reaction components (Policarpo et al. (2014) Angew Chem Int Ed Engl. 53(35):9203-8).

In some embodiments, the equilibrium of the reaction can be controlled by ligation product or by-product deactivation. For example, in some embodiments the reaction is controlled by ligation of a WTWTW (SEQ ID NO: 179) motif added to the donor and acceptor as described in Yamamura et al. (2011) Commun (Camb). 47(16):4742-4). In other embodiments, by-products are deactivated by chemical modification of the acyl donor glycine as described, for example, in Liu et al. (2014) J Org Chem. 79(2):487-92; and Williamson et al. (2014) NatProtoc. 9(2):253-62).

(e) Intein-Mediated Conjugation

Inteins are naturally occurring, self-splicing protein subdomains that are capable of excising out their own protein subdomain from a larger protein structure while simultaneously joining the two formerly flanking peptide regions (“exteins”) together to form a mature host protein. Intein-based methods of protein modification and ligation have been developed. An intein is an internal protein sequence capable of catalyzing a protein splicing reaction that excises the intein sequence from a precursor protein and joins the flanking sequences (N- and C-exteins) with a peptide bond. A non-limiting exemplification of pMHC multimers prepared using intein-mediated conjugation is described in detail in Example 2.

As used herein, the term “split intein” refers to any intein in which one or more peptide bond breaks exists between the N-terminal intein segment and the C-terminal intein segment such that the N-terminal and C-terminal intein segments become separate molecules that cannon-covalently reassociate, or reconstitute, into an intein that is functional for splicing or cleaving reactions. Any catalytically active intein, or fragment thereof, may be used to derive a split intein for use in the systems and methods disclosed herein. For example, in one aspect the split intein may be derived from a eukaryotic intein. In another aspect, the split intein may be derived from a bacterial intein. In another aspect, the split intein may be derived from an archaeal intein. Preferably, the split intein so-derived will possess only the amino acid sequences essential for catalyzing splicing reactions.

As used herein, the “N-terminal intein segment” refers to any intein sequence that comprises an N-terminal amino acid sequence that is functional for splicing and/or cleaving reactions when combined with a corresponding C-terminal intein segment. An N-terminal intein segment thus also comprises a sequence that is spliced out when splicing occurs. An N-terminal intein segment can comprise a sequence that is a modification of the N-terminal portion of a naturally occurring (native) intein sequence. For example, an N-terminal intein segment can comprise additional amino acid residues and/or mutated residues so long as the inclusion of such additional and/or mutated residues does not render the intein non-functional for splicing or cleaving. Preferably, the inclusion of the additional and/or mutated residues improves or enhances the splicing activity and/or controllability of the intein. Non-intein residues can also be genetically fused to intein segments to provide additional functionality, such as the ability to be affinity purified or to be covalently immobilized.

As used herein, the “C-terminal intein segment” refers to any intein sequence that comprises a C-terminal amino acid sequence that is functional for splicing or cleaving reactions when combined with a corresponding N-terminal intein segment. In one aspect, the C-terminal intein segment comprises a sequence that is spliced out when splicing occurs. In another aspect, the C-terminal intein segment is cleaved from a peptide sequence fused to its C-terminus. A C-terminal intein segment can comprise a sequence that is a modification of the C-terminal portion of a naturally occurring (native) intein sequence. For example, a C terminal intein segment can comprise additional amino acid residues and/or mutated residues so long as the inclusion of such additional and/or mutated residues does not render the C-terminal intein segment non-functional for splicing or cleaving.

Expressed protein ligation (EPL) refers to a native chemical ligation between a recombinant protein with a C-terminal thioester and a second agent with an N-terminal cysteine. The C-terminal thioester can readily be introduced onto any recombinant protein (i.e., the targeting ligand) through the use of auto-processing, also known as protein-splicing, mediated by an intein (intervening protein). Inteins are proteins that can excise themselves from a larger precursor polypeptide chain, utilizing a process that results in the formation of a native peptide bond between the flanking extein (external protein) fragments. When an auto-processing protein is cloned downstream of the targeting ligand, thiols (e.g., 2-mercaptoethanesulfonic acid, MESNA) can be used to induce the site-specific cleavage of the auto-processing protein, resulting in the formation of a reactive thioester. The thioester will then react with any agent that has an N-terminal cysteine. EPL operates in a site-specific manner, and the reaction is known to be very efficient if both functional groups are in high concentrations. (reviewed in Elias et al. (2010) Small 6:2460-2468).

Accordingly, in some embodiments, the MHC monomers are ligated to an alkynated peptide by expressed protein ligation (EPL) and then conjugated to an azide-labeled multimerization domain by Cu(I)-catalyzed terminal azide-alkyne cycloaddition (CuAAC).

In some embodiments, the MHC monomers are conjugated to the multimerization domain by an intein peptide tag. In some embodiments, the MHC polypeptide comprises a C-terminal thioester, the multimerization domain comprises an N-extein fused to a modified intein lacking the ability to perform trans-esterification and trans-esterification occurs by the addition of exogenous thiol.

A number of inteins have now been described including, but not limited to MxeGyrA (Frutos et al. (2010); Southworth et al. (1999); SspDnaE (Shah et al. (2012); Wu et al. (1998); NpuDnaE (Shah et al. (2012); Vila-Perello et al. (2013); AvaDnaE (David et al. (2015); Shah et al. (2012); Cfa (consensus DnaE split intein) (Stevens et al. (2016)); gp41-1 and gp41-8 (Carvajal-Vallejos et al. (2012)); NrdJ-1 (Carvajal-Vallejos et al. (2012)); IMPDH-1 (Carvajal-Vallejos et al.) and AceL-TerL (Thiel et al. (2014). The properties and use of these inteins are summarized in TABLE 7.

TABLE 7
Inteins used for creating protein conjugates

| Intein | Temperature (° C.) | t_{1/2}* | |
|---|---|---|---|
| MxeGyrA | 25 | 10 | h |
| SspDnaE | 37 | 76 | min |
| NpuDnaE | 37 | 19 | s |
| AvaDnaE | 37 | 23 | s |
| Cfa (consensus DnaE split intein) | 30 | 20 | s |
| gp41-1 | 45 | 4 | s |
| gp41-8 | 37 | 15 | s |
| NrdJ-1 | 37 | 7 | s |
| IMPDH-1 | 37 | 8 | s |
| AceL-TerL | 8 | 7.2 | min |

In some embodiments, the intein is the 198-residue gyrase A intein from Mycobacterium xenopi (Mxe GyrA) (Southworth et al. (1999) Biotechniques. 27(1):110-4, 116, 118-20). In some embodiments, the intein is from cyanobacterium Synechocystis sp. strain PCC6803 (Ssp).

In some embodiments, the intein is a split intein pair. In some embodiments, the split intein pair is an orthogonal split intein pair (Carvajal-Vallejos et al. (2012) J Biol Chem. 287(34):28686-96; Shah et al. (2011) Angew Chem Int Ed Engl. 50(29):6511-5).

In some embodiments, the split intein pair is an artificially split intein pair that are as short as six or eleven residues (Appleby et al. (2009) J Biol Chem. 284(10):6194-9; Ludwig et al. (2006) Angew Chem Int Ed Engl. 45(31):5218-21).

In some embodiments, the intein is a DnaE intein. In some embodiments, the DnaE intein is from Nostoc punctiforme (Npu). In some embodiments, the intein is the gp41-1 intein. In some embodiments, the intein is the gp41-8 intein. In some embodiments, the intein is the IMPDH-1 intein. In some embodiments, the intein is the NrdJ Intein.

In some embodiments, the split intein pair is AceL-TerL (Thiel et al. (2014) Angew Chem Int Ed Engl. 53(5): 1306-10).

In some embodiments, the intein comprises consensus split intein sequence (Cfa) (Stevens et al. (2016) Journal of the American Chemical Society 138(7):2162-2165).

A number of protocols for intein mediated conjugation are available and an exemplary method is provided herein in Example 2. Suitable intein sequences and protocols for use in protein conjugation have been described in the art, such as in Stevens et al. (2016) J Am. Chem. Soc., 138, 2162-2165; Shah et al. (2012) J. Am. Chem. Soc., 134, 11338-11341; and Vila-Perello et al., J. Am. Chem. Soc., 135, 286-292; Batjargal et al. (2015) J. Am Chem Soc. 137(5):1734-7; and Guan et al. (2013) Biotechnol Bioeng. 110(9):2471-81, the entire contents of each of which is hereby incorporated by reference.

In some embodiments, the intein-labeled MHC molecule is a soluble HLA-A2 molecule (HLA-A*02:01) with an N-intein tag, such as having the amino acid sequence shown in SEQ ID NO: 4. In some embodiments, the intein-labeled multimerization domain is a streptavidin molecule with a C-intein tag and FLAG Tag, such as having the amino acid sequence shown in SEQ ID NO: 5. In some embodiments, the N-intein tag, including a FLAG tag, has the amino acid sequence shown in SEQ ID NO: 180. Various other N-intein and C-intein sequences are known in the art and are suitable for use in preparing the Conjugated Multimers of the disclosure, non-limiting examples of which are described in the references cited above.

(f) Peptide Linkers

In other embodiments, the p*MHC multimers comprises a peptide linker. The term “peptide linker” denotes a linear amino acid chain of natural and/or synthetic origin. The linker has the function to ensure that polypeptides conjugated to each other can perform their biological activity by allowing the polypeptides to fold correctly and to be presented properly. The peptide linker may contain repetitive amino acid sequences or sequences of naturally occurring polypeptides. In some embodiments, the peptide linker has a length of from 2 to 50 amino acids. In some embodiments, the peptide linker is between 3 and 30 amino acids, between 5 to 25 amino acids, between 5 to 20 amino acids, or between 10 and 20 amino acids.

In some embodiments, the peptide linker is rich in glycine, glutamine, and/or serine residues. These residues are arranged e.g. in small repetitive units of up to five amino acids. This small repetitive unit may be repeated for one to five times. At the amino- and/or carboxy-terminal ends of the multimeric unit up to six additional arbitrary, naturally occurring amino acids may be added. Other synthetic peptidic linkers are composed of a single amino acid, which is repeated between 10 to 20 times and may comprise at the amino- and/or carboxy-terminal end up to six additional arbitrary, naturally occurring amino acids. All peptidic linkers can be encoded by a nucleic acid molecule and therefore can be recombinantly expressed. As the linkers are themselves peptides, the polypeptide connected by the linker are connected to the linker via a peptide bond that is formed between two amino acids.

Suitable peptide linkers are well known in the art, and are disclosed in, e.g., US2010/0210511, US2010/0179094, and US2012/0094909, which are herein incorporated by reference in its entirety. Other linkers are provided, for example, in U.S. Pat. No. 5,525,491; Alfthan et al. (1995) Protein Eng., 8:725-731; Shan et al. (1999) J. Immunol. 162:6589-6595; Newton et al. (1996) Biochemistry 35:545-553; Megeed et al. (2006) Biomacromolecules 7:999-1004; and Perisic et al. (1994) Structure 12:1217-1226; each of which is incorporated by reference in its entirety.

In some embodiments, the polypeptide linker is synthetic. As used herein, the term “synthetic” with respect to a polypeptide linker includes peptides (or polypeptides) which comprise an amino acid sequence (which may or may not be naturally occurring) that is linked in a linear sequence of amino acids to a sequence (which may or may not be naturally occurring) to which it is not naturally linked in nature. For example, the polypeptide linker may comprise non-naturally occurring polypeptides which are modified forms of naturally occurring polypeptides (e.g., comprising a mutation such as an addition, substitution or deletion) or which comprise a first amino acid sequence (which may or may not be naturally occurring). Polypeptide linkers may be employed, for instance, to ensure that the binding portion (TCR or MHC), the multimerization domain and the Igg-Framework of each multimeric fusion polypeptide is juxtaposed to ensure proper folding and formation of a functional multimeric protein complex. Preferably, a polypeptide linker will be relatively non-immunogenic and not inhibit any non-covalent association among monomer subunits of a binding protein.

In some embodiments, the linker is a Gly-Ser polypeptide linker, i.e., a peptide that consists of glycine and serine residues. One exemplary Gly-Ser polypeptide linker comprises the amino acid sequence (Gly4Ser)n, wherein n=1-6 (SEQ ID NO: 181). In certain embodiments, n=1. In certain embodiments, n=2. In certain embodiments, n=3. In certain embodiments, n=4. In certain embodiments, n=5. In certain embodiments, n=6. Another exemplary Gly-Ser polypeptide linker comprises the amino acid sequence Ser(Gly4Ser)n, wherein n=1-10 (SEQ ID NO: 184). In certain embodiments, n=1. In certain embodiments, n=2. In certain embodiments, n=3, i.e., Ser(Gly4Ser)3. In certain embodiments, n=4, i.e., Ser(Gly4Ser)4. In certain embodiments, n=5. In certain embodiments, n=6. In certain embodiments, n=7. In certain embodiments, n=8. In certain embodiments, n=9. In certain embodiments, n=10.

Other exemplary linkers include GS linkers (i.e., (GS)n), GGSG linkers (i.e., (GGSG)n) (SEQ ID NO: 185), GSAT linkers (SEQ ID NO: 186), SEG linkers, and GGS linkers (i.e., (GGSGGS)n) (SEQ ID NO: 187), wherein n is a positive integer (e.g., 1, 2, 3, 4, or 5). Other suitable linkers for use in multimeric fusion proteins can be found using publicly available databases, such as the Linker Database (ibi.vu.nl/programs/linkerdbwww). The Linker Database is a database of inter-domain linkers in multi-functional enzymes which serve as potential linkers in novel multimeric fusion proteins (see, e.g., George et al. (2002) Protein Engineering 15:871-9).

Polypeptide linkers can be introduced into polypeptide sequences using techniques known in the art. Modifications can be confirmed by DNA sequence analysis. Plasmid DNA can be used to transform hos T cells for stable production of the polypeptides produced.

(g) Additional Peptide Linkers and Tags

Additional tags suitable for use in the methods and compositions provided herein include affinity tags, including but not limited to enzymes, protein domains, or small polypeptides which bind with high specificity to a range of substrates, such as carbohydrates, small biomolecules, metal chelates, antibodies, etc. to allow rapid and efficient purification of proteins. Solubility tags enhance proper folding and solubility of a protein and are frequently used in tandem with affinity tags.

Small-size tags which include, but are not limited to, 6×His, FLAG, Strep II and Calmodulin-binding peptide (CBP) tag, have the benefits of minimizing the effect on structure, activity and characteristics of the MHC polypeptide (Zhao et al. (2013) J Anal. Chem. 581093).

In some embodiments, the tag is a FLAG tag. The FLAG tag is a hydrophilic octapeptide epitope tag that binds to several specific anti-FLAG monoclonal antibodies such as M1, M2, and M5 with different recognition and binding characteristics (Einhauer et al. (2001) J. Biochem. Biophys. 49:455-465: Hopp et al. (1996) Mol. Immunol. 33:601-608). FLAG fusion proteins can be recognized by monoclonal antibody with calcium-dependent (e.g., M2) or calcium-independent manner. In particular, the tag appended to the N-terminus of the fusion protein is necessary for the immunoaffinity purification with M1 monoclonal antibody, while M2 is position-insensitive.

### MHC Peptide Epitopes

(a) Peptide Epitope Selection

Various processes have been developed for identifying new MHC binding peptides that may be T cell epitopes and many experimental methods start with constructing an overlapping library of peptide fragments from a given protein sequence, by synthesizing a constant length (n-mer) amino acid sequences which are offset from one another along the protein sequence by fixed number of amino acids. The MHC binding properties and potential for activating T cells of each sequence can then be assessed in a number of assays.

Existing MHC binding peptides that have been identified with the methods outlined above and other methods, such as crystallographic analysis of the conformation of and charge distribution in the MHC binding groove has led to binding motifs being defined for the most common MHC alleles, setting rules for what type of putative MHC binding peptide can actually bind well to MHC molecules of a given allele. These motifs have been translated into predictive computer algorithms for predicting peptide binding to MHC molecules such as the SYFPEITHI algorithm (Rammensee H.-G., et al. (1995) Immunogenetics 41:178-228).

Protein sequences for the desired antigen are analyzed for potential HLA specific antigens by using the SYFPEITHI algorithm (Rammensee et al. (1999) Immunogenetics 50:213-219), and the artificial neural network (ANN) and stabilized matrix method (SMM) algorithms from IEDB (Peters et al. (2005) PLoS Biol. 3:e91). Peptides are selected based on a predicted binding value of either >21 for SYFPEITHY, <6000 for ANN, or <600 for SMM. Selected peptides are synthesized.

Binding assays can be performed using a fluorescence polarization (FP) assay as previously described (e.g., Buchi et al. (2004) Biochemistry 43:14852-14863; Sette et al. (1994)Mol. Immunol. 31:813-822). To determine binding capacity of the peptides, percentage inhibition relative to controls can be determined in an FP competition assay with the placeholder peptide.

In some embodiments, the peptides bound to the pMHC multimers are from an unbiased library of peptides. In some embodiments, the peptides are 9-mers. In some embodiments, the peptides bound to the pMHCI multimers are 9-mers which include an HLA-A2 binding motif with key amino acids at positions 2 and 9 which can include isoleucine (I), valine (V) or leucine (L).

In some embodiments, the library comprises all k-mer peptides produced by transcription and translation of any polynucleotide sequence of interest, for example, in silico production of the transcription and translation products of both the forward and reverse strands of a genome or metagenome in all six reading frames.

In some embodiments, a library of the disclosure comprises all k-mer peptides that can be derived from in silico translation of an exome of interest. In some embodiments, a library of the disclosure comprises all k-mer peptides that can be derived from in silico translation of a transcriptome of interest. In some embodiments, a library of the disclosure comprises all k-mer peptides that can be derived from a proteome of interest. In some embodiments, a library of the disclosure comprises all k-mer peptides that can be derived from in silico translation of an ORFeome of interest. In some embodiments, an algorithm can be used to select peptides in a peptide library. For example, an algorithm can be used to predict peptides most likely to fold or dock in an MHC/HLA binding pocket, and peptides above a certain threshold value can be selected for inclusion in the library.

In some embodiments, a library of the disclosure comprises all peptides that can be derived from in silico transcription and translation or translation of a group of genomes, proteomes, transcriptomes, ORFeomes, or any combination thereof. In some embodiments, the peptides are derived from in silico transcription and translation or translation of polynucleotide sequences from a group of samples, for example, clinical samples from a patient population, or a group of pathogen genomes.

In some embodiments, the peptides are derived from a differential genome, proteome, transcriptome, ORFeome, or any combination thereof, where two or more genomes, proteomes, transcriptomes, ORFeomes, or a combination thereof are compared to identify sequences that are differential sequences (e.g., that differ between them). In some embodiments, the peptide sequences are identified by comparing tissues of interest. In some embodiments, the peptide sequences are identified by comparing cells of interest. In some embodiments, the peptide sequences are identified by comparing diseased versus healthy cells or tissues. In some embodiments, the diseased cells or tissues are cancer cells or tissues. In some embodiments, the diseased cells are derived from an individual with an autoimmune disorder.

In some embodiments, the peptides are derived from homologous sequences of genomes, proteomes, transcriptomes, ORFeomes, or any combination thereof, where two or more genomes, proteomes, transcriptomes, ORFeomes, or a combination thereof are compared to identify sequences that are homologous sequences.

In some embodiments, the peptides are derived from mutations in a sequence of interest, for example, all 9-mer peptides that can be generated from single nucleotide mutations in a polynucleotide sequence encoding an antigen or epitope.

In some embodiments, the peptides an overlapping peptide library, comprising overlapping peptides from a template sequence (e.g., in silico translated genome), wherein overlapping peptides of a set length are offset by a defined number of residues.

In some embodiments, selection of peptides comprises prioritizing peptides based on predicted binding affinity for a certain HLA type.

In some embodiments, selection of peptides for a library of the disclosure prioritizes HLA types or alleles based on prevalence in a population, e.g., a human population.

In some embodiments, the library comprises all k-mer peptides produced by transcription and translation of any polynucleotide sequence of interest, for example, in silico production of the transcription and translation products of both the forward and reverse strands of a genome or metagenome in all six reading frames. In some embodiments, a library of the disclosure comprises all k-mer peptides that can be derived from in silico transcription and translation of a mammalian genome, for example, a mouse genome, a human genome, a patient genome, an autoimmune patient genome, or a cancer genome. In some embodiments, a library of the disclosure comprises all k-mer peptides that can be derived from in silico transcription and translation of a microorganism genome, for example, a bacterial genome, a viral genome, a protozoan genome, a protist genome, a yeast genome, an archaeal genome, or a bacteriophage genome. In some embodiments, a library of the disclosure comprises all k-mer peptides that can be derived from in silico transcription and translation of a pathogen genome, for example, a bacterial pathogen genome, a viral pathogen genome, a fungal pathogen genome, an opportunistic pathogen genome, a conditional pathogen genome, or a eukaryotic parasite genome. In some embodiments, a library of the disclosure can be derived from a plant genome or a fungal genome. In some embodiments, a library of the disclosure comprises k-mer peptides derived from in silico transcription and translation of a genome, wherein the genome is modified during in silico transcription and translation, for example, in silico mutated to produce k-mer peptides comprising mutations (e.g. substitutions, insertions, deletions).

In some embodiments, a library of the disclosure comprises all k-mer peptides that can be derived from in silico translation of an exome of interest, for example, a mammalian exome, a human exome, a mouse exome, a patient exome, an autoimmune patient exome, a cancer exome, a viral exome, a protozoan exome, a protist exome, a yeast exome, a pathogen exome, a eukaryotic parasite exome, a plant exome, or a fungal exome. In some embodiments, a library of the disclosure comprises k-mer peptides derived from in silico translation of a exome, wherein the exome is modified during in silico translation, for example, in silico mutated to produce k-mer peptides comprising mutations (e.g. substitutions, insertions, deletions).

In some embodiments, a library of the disclosure comprises all k-mer peptides that can be derived from in silico translation of a transcriptome of interest, for example, a mammalian transcriptome, a human transcriptome, a mouse transcriptome, a patient transcriptome, an autoimmune patient transcriptome, a cancer transcriptome, a microorganism transcriptome, a bacterial transcriptome, a viral transcriptome, a protozoan transcriptome, a protist transcriptome, a yeast transcriptome, an archaeal transcriptome, a bacteriophage transcriptome, a pathogen transcriptome, a eukaryotic parasite transcriptome, a plant transcriptome, a fungal transcriptome, a transcriptome derived from RNA sequencing, a microbiome transcriptome, or a transcriptome derived from metagenomic RNA-sequencing. In some embodiments, a library of the disclosure comprises k-mer peptides derived from in silico translation of a transcriptome, wherein the transcriptome is modified during in silico translation, for example, in silico mutated to produce k-mer peptides comprising mutations (e.g. substitutions, insertions, deletions).

In some embodiments, a library of the disclosure comprises all k-mer peptides that can be derived from a proteome of interest, for example, a mammalian proteome, a human proteome, a mouse proteome, a patient proteome, an autoimmune patient proteome, a cancer proteome, a microorganism proteome, a bacterial proteome, a viral proteome, a protozoan proteome, a protist proteome, a yeast proteome, an archaeal proteome, a bacteriophage proteome, a pathogen proteome, a eukaryotic parasite proteome, a plant proteome or a fungal proteome. In some embodiments, a library of the disclosure comprises k-mer peptides derived from a proteome wherein the k-mer peptides are modified from the proteome sequence, for example, k-mer peptides comprising mutations (e.g. substitutions, insertions, deletions).

In some embodiments, a library of the disclosure comprises all k-mer peptides that can be derived from in silico translation of an ORFeome of interest, for example, a mammalian ORFeome, a human ORFeome, a mouse ORFeome, a patient ORFeome, an autoimmune patient ORFeome, a cancer ORFeome, a microorganism ORFeome, a bacterial ORFeome, a viral ORFeome, a protozoan ORFeome, a protist ORFeome, a yeast ORFeome, an archaeal ORFeome, a bacteriophage ORFeome, a pathogen ORFeome, a eukaryotic parasite ORFeome, a plant ORFeome or a fungal ORFeome, an ORFeome derived from next-gen sequencing, a microbiome ORFeome, or an ORFeome derived from metagenomic sequencing. In some embodiments, a library of the disclosure comprises k-mer peptides derived from in silico translation of an ORFeome, wherein the ORFeome is modified during in silico translation, for example, in silico mutated to produce k-mer peptides comprising mutations (e.g. substitutions, insertions, deletions).

In some embodiments, a library of the disclosure comprises all k-mer peptides that can be derived from in silico transcription and translation or translation of a group of genomes, proteomes, transcriptomes, ORFeomes, or any combination thereof. In some embodiments, a library of the disclosure comprises all k-mer peptides that can be derived from in silico transcription and translation or translation of polynucleotide sequences from a group of samples, for example, clinical samples from a patient population, or a group of pathogen genomes. In some embodiments, a library of the disclosure comprises all k-mer peptides that can be derived from in silico transcription and translation of a group of viral genomes, for example, the human virome. In some embodiments, a library of the disclosure comprises all k-mer peptides that can be derived from in silico transcription and translation of a group of genomes, proteomes, transcriptomes, ORFeomes, or any combination thereof, wherein the source sequences are modified during in silico translation, for example, in silico mutated to produce k-mer peptides comprising mutations (e.g. substitutions, insertions, deletions).

In some embodiments, a library of the disclosure comprises all k-mer peptides that can be derived from a differential genome, proteome, transcriptome, ORFeome, or any combination thereof, where two or more genomes, proteomes, transcriptomes, ORFeomes, or a combination thereof are compared to identify sequences that are differential sequences (e.g., that differ between them), for example, differing in nucleotide sequence, amino acid sequence, nucleotide abundance, or protein abundance. In some embodiments, differential sequences of a genome, proteome, transcriptome, or ORFeome are generated by comparing tissues of interest. In some embodiments, differential sequences of a genome, proteome, transcriptome, or ORFeome are generated by comparing sequences from cells of interest (e.g., a healthy cell versus a cancer cell). In some embodiments, differential sequences of a genome, proteome, transcriptome, or ORFeome are generated by comparing sequences of organisms of interest. In some embodiments, differential sequences of a genome, proteome, transcriptome, or ORFeome can be generated by comparing subjects of interest (e.g., diseased versus healthy subjects).

In some embodiments, a library of the disclosure comprises all k-mer peptides that can be derived from homologous sequences of genomes, proteomes, transcriptomes, ORFeomes, or any combination thereof, where two or more genomes, proteomes, transcriptomes, ORFeomes, or a combination thereof are compared to identify sequences that are homologous sequences (e.g., that share a degree of homology), for example, homologous nucleotide sequences, homologous amino acid sequences, homologous nucleotide abundance, or homologous protein abundance. In some embodiments, homologous sequences of genomes, proteomes, transcriptomes, or ORFeomes are generated by comparing tissues of interest. In some embodiments, homologous sequences of genomes, proteomes, transcriptomes, or ORFeomes are generated by comparing sequences from cells of interest (e.g., a healthy cell versus a involved in autoimmunity cell (e.g., a cell that induces autoimmunity or a cell that is targeted during autoimmunity). In some embodiments, homologous sequences of genomes, proteomes, transcriptomes, or ORFeomes are generated by comparing sequences of organisms of interest. In some embodiments, homologous sequences of genomes, proteomes, transcriptomes, or ORFeomes are generated by comparing subjects of interest (e.g., diseased versus healthy subjects).

In some embodiments, a library of the disclosure comprises all k-mer peptides that can be derived from a polypeptide sequence of interest, for example, all possible 9-mer peptides covering the complete protein sequence of a viral protein. In some embodiments, a library of the disclosure comprises k-mer peptides that can be generated from a polypeptide sequence of interest, wherein the polypeptide sequence of interest is modified, e.g. in silico mutated to produce k-mer peptides comprising mutations (e.g. substitutions, insertions, deletions).

In some embodiments, a library of the disclosure comprises all k-mer peptides that can be derived from mutations in a sequence of interest, for example, all 9-mer peptides that can be generated from single nucleotide mutations in a polynucleotide sequence encoding an antigen or epitope. For example, a library of the disclosure comprises all 9-mer peptides that can be generated from two, three, four, five, six, seven, eight, or nine nucleotide mutations in a polynucleotide sequence encoding an antigen or epitope. In some embodiments, a library of the disclosure comprises all k-mer peptides that can be derived from alanine substitutions, for example, alanine substitutions at any position in any of the sequences described herein (e.g., a protein, a group of proteins, a proteome, an in silico transcripted and translated genome). In some embodiments, a library of the disclosure comprises a positional scanning library, wherein selected amino acid residues are sequentially substituted with all other natural amino acids. In some embodiments, a library of the disclosure comprises a combinatorial positional scanning library, wherein selected amino acid residues are sequentially substituted with all other natural amino acids, two or more positions at a time. In some embodiments, a library of the disclosure comprises an overlapping peptide library, comprising overlapping peptides from a template sequence (e.g., in silico translated genome), wherein overlapping peptides of a set length are offset by a defined number of residues. In some embodiments, a library of the disclosure comprises a T cell truncated peptide library, wherein each replicate of the library comprises equimolar mixtures of peptides with truncations at one terminus (e.g., 8-mers, 9-mers, 10-mers and 11-mers that can be derived from C-terminal truncations of a nominal 11-mer). In some embodiments, a library of the disclosure comprises a customized set of peptides, wherein the customized set of peptides are provided in a list.

(b) Peptide Production

Peptides suitable for use in the pMHC multimers are generated according to methods known in the art, or synthetically produced by a commercial vendor or using a peptide synthesizer according to manufacturer's instructions. For example, in some embodiments, peptides suitable for use in the pMHC multimers can be made by in silico production methods.

In other embodiments, peptides can be synthesized via chemical methods, for example, tea bag synthesis, digital photolithography, pin synthesis, and SPOT synthesis. For example, an array of peptides can be generated via SPOT synthesis, where amino acid chains are built on a cellulose membrane by repeated cycles of adding amino acids, and cleaving side-chain protection groups.

In other embodiments, peptides can be expressed using recombinant DNA technology, for example, introducing an expression construct into bacterial cells, insect T cells, or mammalian cells, and purifying the recombinant protein from cell extracts.

In some embodiments, peptides can be synthesized by in vitro transcription and translation, where synthesis utilizes the biological principles of transcription and translation in a cell-free context, for example, by providing a nucleic acid template, relevant building blocks (e.g., RNAs, amino acids), enzymes (e.g., RNA polymerase, ribosomes), and conditions.

In some embodiments, in vitro transcription and translation can include cell-free protein synthesis (CFPS). Obtaining a high yield by CFPS requires the usage of bacterial systems, in which the first amino acid of the translated sequence is N-formylmethionine (fMet). This residue differs from methionine by containing a neutral formyl group (HCO) instead of a positively charged amino-terminus (NH₃⁺). Constructs are engineered to include genes encoding an enzymatic cleavage domain and a library polypeptide as described in U.S. Provisional Application No. 62/791,601, hereby incorporated by reference in its entirety.

Removal of at least the initial methionine amino acid allows successful peptide folding and loading onto MHC protein. In addition, removal of the initial methionine amino acid provides a greater upper limit of peptide library diversity, e.g., 20^{x}, where x is the length of the peptide, while inclusion of this residue will restrict the library diversity to 20^{(x-1)}.

In some embodiments, the peptides are synthesized utilizing an in vitro transcription/translation (IVTT) system that can both transcribe, for example, a DNA construct into RNA, and then translate the RNA into a protein. For example, the methods of the present disclosure comprise a method for performing in vitro transcription/translation (IVTT) to produce a high diversity peptide library and allow for correct folding of proteins. IVTT can allow for protein production in a cell-free environment directly from a DNA or RNA template.

An IVTT method used herein can be performed using, for example, a PCR product, a linear DNA plasmid, a circular DNA plasmid, or an mRNA template with a ribosome-binding site (RBS) sequence. After the appropriate template has been isolated, transcription components can be added to the template including, for example, ribonucleotide triphosphates, and RNA polymerase. After transcription has been completed, translation components can be added, which can be found in, for example, rabbit reticulocyte lysate, or wheat germ extract. In some methods, the transcription and translation can occur during a single step, in which purified translation components found in, for example, rabbit reticulocyte lysate or wheat germ extract are added at the same time as adding the transcription components to the nucleic acid template.

In some embodiments, nucleotide sequence encoding a methionine residue at the N-terminus of the peptide and a cleavable moiety can be encoded in the DNA construct or RNA construct. The cleavable moiety is situated such that at least one N-terminus amino acid residue of the peptide is before or within the cleavable moiety. In some embodiments, the method comprises encoding a cleavable moiety that is situated such that one N-terminus amino acid residue of the peptide is before or within the cleavable moiety. In some embodiments, the one N-terminus amino acid residue is a methionine residue. The cleavable moiety can be cleaved using an enzyme, e.g., a protease, specific to the cleavable moiety, which can also cleave off the cleavable moiety from the remainder of the peptide.

An example of a cleavable moiety that can be encoded in a DNA or RNA construct as described herein includes any cleavable moiety cleaved by an enzyme. In some embodiments, a cleavable moiety can be cleaved by a protease. The cleavage moiety can be cleaved off of the peptide using an enzyme specific for the cleavage moiety. The enzyme can be, for example, Factor Xa, human rhinovirus 3C protease, AcTEV™ Protease, WELQut Protease, Genenase™, small ubiquitin-like modifier (SUMO) protein, Ulp1 protease, or enterokinase. The Ulp1 protease can cleave off a cleavage moiety in a specific manner by recognizing the tertiary structure, rather than an amino acid sequence. Enterokinase (enteropeptidase) can also be used to cleave the cleavage moiety from the candidate peptide.

Enterokinase can cleave after lysine at the following cleavage site: DDDDK (SEQ ID NO.: 188). Enterokinase can also cleave at other basic residues, depending on the sequence and conformation of the protein substrate.

In some embodiments, the cleavable moiety can be a small ubiquitin-like modifier (SUMO) protein. The SUMO domain can be cleaved off of the peptide using a protease specific to SUMO. In some embodiments, the cleavable moiety can be an enterokinase cleavage site: DDDDK (SEQ ID NO.: 188). The protease can be, for example, Ulp1 protease or enterokinase. The Ulp1 protease can cleave off SUMO in a specific manner by recognizing the tertiary structure of SUMO, rather than an amino acid sequence. Enterokinase (enteropeptidase) can also be used to cleave after lysine at the following cleavage site: DDDDK (SEQ ID NO.: 188). Enterokinase can also cleave at other basic residues, depending on the sequence of the protein substrate.

During or after translation of the construct encoding the peptide, the N-terminus amino acid residue(s) (e.g., a SUMO domain) can be efficiently cleaved to produce the properly folded peptide. In some embodiments, at least one N-terminus amino acid residue is cleaved to produce the peptide. In some embodiments, one, two, three, four, five six, seven, eight, nine, ten or more N-terminus amino acid residues are cleaved to produce the peptide. The N-terminus amino acid can be any amino acid residue. The N-terminus amino acid residue can be a methionine amino acid residue. This properly folded peptide is thus not constrained to have an N-terminus methionine, and can be part of a high diversity peptide library produce by cell-free in vitro methods.

After translation of the construct encoding the peptide, an N-terminus amino acid residue can be cleaved to produce the peptide for the high diversity peptide library. In some embodiments, at least one N-terminus amino acid residue is cleaved to produce the peptide. In some embodiments, one or more N-terminus amino acids are cleaved, such as 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 105, 110, 115, 120, 125, 130, 140, 150, 160, 170, 180, 190, 200, 250 or more, N-terminus amino acid residues are cleaved to produce the peptide. The N-terminus amino acid can be any amino acid residue. The N-terminus amino acid residue can be a methionine amino acid residue.

In some embodiments, a DNA or RNA construct comprises a puromycin. In some embodiments, a DNA or RNA construct comprises a spacer sequence lacking a stop codon. In some embodiments, the peptides are purified by affinity tag purification (e.g., with a FLAG-tag). In some embodiments, the peptides comprise a HaloTag enzymatic sequence. In some embodiments, peptides comprise an avidin or streptavidin.

For mammalian expression, a construct encoding the CMV peptide was designed with a C-terminal Flag-tag with and without a C-terminal His-tag in a mammalian expression vector. Peptides were expressed by transient transfection in Expi293F or ExpiCHO-S cells (Life Technologies) according to the manufacturer's recommendations.

Peptides were purified from cell culture supernatants with anti-Flag affinity chromatography (Genscript) or by Ni-affinity chromatography. Size exclusion chromatography (SEC) can be performed on a hydrophilic resin (GE Life Sciences) pre-equilibrated in 20 mM HEPES, 150 mM NaCl, pH 7.2.

Alternatively, peptides are purified by Ni-affinity chromatography without SEC purification, using a column buffer of 23 mM sodium phosphate, 500 mM sodium chloride, 500 mM imidazole, pH 7.4.

Peptides produced in mammalian cells can be quantitated by UV at 280 nm, whereas CFPS-produced peptides were quantitated by a sandwich ELISA relative to a standard protein.

### Peptide Exchange

p*MHC multimers are used to generate a library of or microarray of pMHC multimers loaded with a diversity of unique peptide epitopes by in situ or in vitro peptide exchange reactions as described herein. In some embodiments, the peptide exchange reactions are performed in multiwell formats and under native conditions. Binding is determined by a number of techniques, such as ELISA, which monitors the stability of the MHC structure, or by biophysical techniques that monitor peptide binding, such as fluorescence polarization. Non-limiting exemplifications of peptide exchange via dipeptide exchange or UV-mediated exchange are described in detail in Example 4.

In some embodiments, to measure the dissociation efficiency of placeholder peptides or peptide fragments a fluorescently labeled placeholder peptide is used in exchange reactions in the presence of unlabeled exchange peptides. Aliquots of fluorescently labeled p*MHC multimers are either left untreated or exposed to peptide exchange conditions (e.g., UV exposure) for different time periods. The amount of remaining p*MHC-containing the placeholder peptide is monitored by fluorescence analysis to monitor the reduction in p*MHC complexes.

In some embodiments, the placeholder peptide has a lower affinity for the MHC peptide binding groove than the exchanged peptide epitope, and wherein step (d) comprises contacting the p*MHC monomer with an excess of peptide epitope in a competition assay. In some embodiments, the placeholder peptide has a KD that is about 10-fold lower than the exchanged peptide epitope.

Peptides that bind to the peptide binding groove of the MHC molecule can be a naturally occurring peptide but can also be synthetically created using the knowledge of the binding specificity of the B and F pocket of the particular MHC molecule or the supertype family it belongs to. Suitable ligands can be generated using the available 3D structures of MHC complexes and the knowledge on the binding pocket specificity of the respective MHC molecules.

Peptide binding specificity of MHC I polypeptides is primarily governed by the physiochemical properties of the B and F binding pockets in a coupled fashion. The B and F binding pockets typically bind to “anchor residues” in the peptide that define the binding of the peptide in the peptide binding groove of the MHC. The observed diversity in the amino acid residues of the peptide binding groove of the MHC molecules defines the peptide-binding and the presentation repertoire of the individual MHC molecule (Chang et al. (2011) Frontiers in Bioscience, Landmark Edition, Vol. 16:3014-3035). The specificity of the pockets for anchor residues has been elucidated for a large number MHC molecules, for example, as described in Sidney et al. (2008) BMC Immunology Vol. 9:1).

The disclosure further provides a method of producing a p*MHC multimer comprising: producing an p*MHC multimer in which the peptide in the binding groove is a placeholder peptide; contacting the p*MHC multimer with a reducing agent to remove the placeholder peptide; and contacting the p*MHC multimer with an MHC peptide epitope under conditions sufficient for binding of the peptide epitope in the MHC peptide binding groove.

The two contacting steps are preferably performed by providing a sample comprising the MHC molecule with the MHC peptide epitope and the reducing agent. Preferably the MHC peptide epitope is present when the reducing agent is added. In some embodiments, one MHC peptide epitope is added per reaction. In some embodiments, two or more peptide epitopes are added to the reaction.

In some embodiments, peptide exchange is induced by elevating the temperature of the mixture to between about 30°−37° C. In some embodiments, the mixture is elevated to 31°, 32°, 33°, 34°, 35°, 36° or 37°.

In some embodiments, peptide exchange is induced by reducing the pH of the mixture to between about pH 2.5-5.5. In some embodiments, peptide exchange is induced by increasing the pH of the mixture to about pH 9-11.

In some embodiments, the placeholder peptide comprises a photocleavable moiety to form pMHC complexes as described (e.g., Toebes et al. (2006) Nat. Med. 12:246-251;

Bakker et al. (2008) PNAS 105:3825-383; Frosig et al. (2015) Cytometry Part A, 87A:967-975; Chang et al. (2013) Eur. J. Immunol. 43:1109-1120). In some embodiments, the placeholder peptide comprises a non-natural amino acid that contains a (2-nitro)phenyl side chain. In some embodiments, the amino acid is the UV-sensitive β-amino acid comprising 3-amino-3-(2-nitro)phenyl-propionic acid. In some embodiments, the UV-sensitive amino acid is (2-nitro)phenylglycine.

In some embodiments, the placeholder peptide is an HLA-A2 peptide. In some embodiments, the HLA-A2 placeholder peptide is p*A2, KILGCVFJV (SEQ ID NO:15) or GILGFVFJL (SEQ ID NO: 7), wherein J is 3-amino-3-(2-nitro)phenyl-propionic acid.

In some embodiments, the placeholder peptide is an HLA-A1, -A3, A11 or -B7 peptide containing a photocleavable moiety. In some embodiments, the placeholder peptide is selected from the group consisting of p*A1:01, STAPGJLEY (SEQ ID NO: 16); p*A3:01, RIYRJGATR (SEQ ID NO:17); p*A11:01, RVFAJSFIK (SEQ ID NO: 18); p*A24:02, VYGJVRACL (SEQ ID NO: 11); p*B7:02, AARGJTLAM (SEQ ID NO: 14); p*B35:01, KPIVVLJGY (SEQ ID NO: 19); p*C3:04, FVYGJSKTSL (SEQ ID NO: 20), p*B8:01, FLRGRAJGL (SEQ ID NO: 21); p*C7:02, VRIJHLYIL (SEQ ID NO: 22); p*C4:01, QYDJAVYKL (SEQ ID NO: 23); p*B15:01, ILGPJGSVY (SEQ ID NO: 24); p*B40:01, TEADVQJWL (SEQ ID NO: 25); p*B58:01, ISARGQJLF (SEQ ID NO: 26); AND p*C8:01, KAAJDLSHFL (SEQ ID NO: 27), wherein J is 3-amino-3-(2-nitro)phenyl-propionic acid.

In some embodiments, the placeholder peptide further comprises a fluorescent label. In so embodiments, the fluorescent label is attached to a cysteine residue in the placeholder peptide.

Upon irradiation with long-wavelength UV, the peptide is cleaved and dissociates from the MHC complex in the presence of one or more peptides to facilitate the formation of stable pMHC monomers or multimers. Typically, MHC peptide exchange is performed in multiwell format for high-throughput screening of peptide ligands as described herein. Only peptide candidates that can effectively bind and stabilize the peptide-receptive MHC molecules prevent dissociation of the MHC complexes. Peptide exchange can be monitored by a number of techniques such as ELISA or fluorescence polarization, for example, as generally described in Rodenko et al. ((2006) Nat. Protocol. 1:1120-1132).

The resulting pMHC multimers are subsequently analyzed by gel-filtration HPLC and MHC ELISA to determine three parameters: the efficiency of MHC refolding, the stability of the pMHC complex in the absence of UV exposure, and the UV-sensitivity of the complex.

Certain di-peptides can assist folding and peptide exchange of MHC class I molecules. Di-peptides bind specifically to the F pocket of MHC class I molecules to facilitate peptide exchange and have so far been described and validated for peptide exchange in HLA-A*02:01, HLA-B*27:05, and H-2Kb molecules (Saini et al. (2013) Proc Natl Acad Sci USA. 110(38):15383-8).

Accordingly, in some embodiments, peptide exchange of the placeholder peptide with a peptide or peptides of interest are catalyzed by a dipeptides which catalyze rapid peptide exchange on MHC class I molecules (see, e.g., Saini et al. (2015) Proc Natl Acad Sci USA. 112(1):202). Suitable dipeptides are those with a hydrophobic second residue. In some embodiments, the dipeptide is glycyl-leucine (GL), glycyl-valine (GV), glycyl-methione (GM), glycyl-cyclohexylalanine (GCha), glycyl-homoleucine (GHle) or glycyl-phenylalanine (GF).

Production of pMHC Libraries

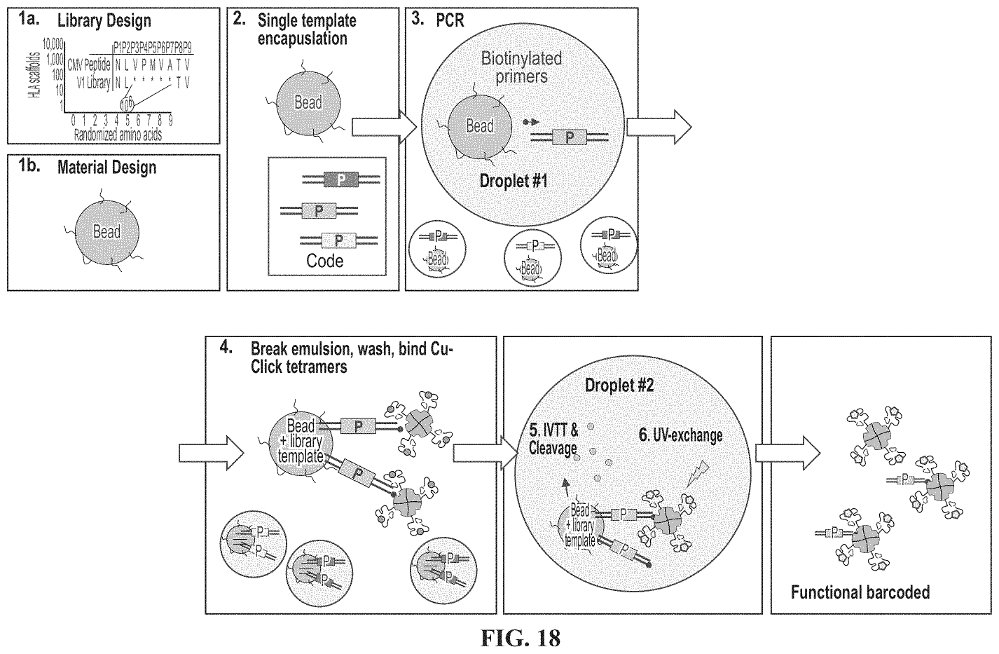

In one aspect, provided herein are methods of producing a library of pMHC multimers comprising a diversity of loaded peptide epitopes. Various steps in the preparation of peptide-exchanged, barcoded pMHC libraries are illustrated schematically in FIG. 18. These steps use standard methods known in the art for preparing barcoded libraries, including use of single-cell sequencing, use of porous hydrogels, use of single template PCR to generate peptide-encoding amplicons (barcodes) and use of in-drop in vitro transcription/translation (IVTT).

A non-limiting exemplification of single-cell sequencing with pooled, barcoded, UV-peptide exchanged MHC tetramers is described in Example 9. A non-limiting exemplification of production of porous hydrogels for high throughput production of barcoded, UV-peptide exchanged MHC tetramer pools is described in detail in Example 10. A non-limiting exemplification of use of single template PCR to generate peptide-encoding amplicons is described in detail in Example 11. A non-limiting exemplification of loading of barcodable, exchange-ready MHC tetramers onto hydrogel is described in Example 12. A non-limiting exemplification of in-drop in vitro transcription/translation (IVTT) of peptide and UV exchange into loaded MHC tetramers is described in detail in Example 13. A non-limiting exemplification of release of UV-peptide exchanged, barcoded pMHC tetramers from hydrogels is described in detail in Example 14.

In some embodiments, the method comprises (a) providing a plurality of placeholder peptide loaded MHCI (p*MHCI) monomers each comprising (i) an MHCI heavy chain polypeptide, or a functional fragment thereof, (ii) a β2-microglobulin polypeptide or functional fragment thereof, (iii) a conjugation moiety, and (iv) a placeholder peptide bound in the peptide binding groove of each MHCI monomer; (b) providing a plurality of multimerization domains, wherein each subunit of the multimerization domain comprises a conjugation moiety; (c) combining the p*MHCI monomers and the multimerization domains under conditions sufficient for covalent conjugation between the two or more p*MHCI monomers and a multimerization domain to produce p*MHCI multimers; and (d) replacing the placeholder-peptide in the plurality of p*MHCI multimers with a peptide library comprising plurality of unique MHCI peptide epitopes to produce a plurality of peptide loaded MHCI (pMHCI) multimers.

In some embodiments, the method comprises (a) providing a plurality of placeholder peptide loaded MHCI (p*MHCI) monomers each comprising (i) an MHCI heavy chain polypeptide, or a functional fragment thereof, (ii) a β2-microglobulin polypeptide or functional fragment thereof, (iii) a conjugation moiety, and (iv) a placeholder peptide bound in the peptide binding groove of each MHCI monomer; (b) providing a plurality of multimerization domains, wherein each subunit of the multimerization domains comprises a conjugation moiety and the multimerization domain comprises at least one non-covalent binding site; (c) combining the plurality of p*MHCI monomers and the plurality of multimerization domain under conditions sufficient for covalent conjugation between the two or more p*MHCI monomers and a multimerization domain to produce a plurality of p*MHCI multimers; (d) replacing the placeholder peptide bound in the peptide binding groove of the p*MHCI multimers with a plurality of unique rescue peptide epitopes to produce a plurality of pMHCI multimers; and (e) binding an oligonucleotide barcode to the non-covalent binding site on the multimerization domain.

In some embodiments, the method comprises (a) providing a plurality of placeholder peptide loaded MHCI (p*MHCI) monomers each comprising (i) an MHCI heavy chain polypeptide, or a functional fragment thereof, (ii) a β2-microglobulin polypeptide or functional fragment thereof, (iii) a peptide linker comprising a conjugation moiety at the C-terminus of (i) or (ii); and (iv) a placeholder peptide bound in the peptide binding groove of each MHCI monomer; (b) providing a plurality of multimerization domains comprising a peptide linker comprising a conjugation moiety at the N-terminus of each subunit of the multimerization domain; (c) combining the plurality of p*MHCI monomers and the plurality of multimerization domains under conditions sufficient for covalent conjugation between two or more p*MHCI monomers to a multimerization domain to produce a plurality of p*MHCI multimers; and (d) replacing the placeholder peptide bound in the peptide binding groove of the p*MHCI multimers with a plurality of unique rescue peptide epitopes to produce a plurality of pMHCI multimers.

### Labeling

pMHC multimers can be conjugated with a fluorescent label, allowing for identification of T cells that bind the peptide-MHC multimer, for example, via flow cytometry or microscopy. T cells can also be selected based on a fluorescence label through, e.g., fluorescence activated cell sorting.

In some embodiments, one or more detectable labels are conjugated to a linker. According to this invention, a “detectable label” is any molecule or functional group that allows for the detection of a biological or chemical characteristic or change in a system, such as the presence of a target substance in the sample.

Examples of detectable labels which may be used include fluorophores, chromophores, electro chemiluminescent labels, bioluminescent labels, polymers, polymer particles, bead or other solid surfaces, gold or other metal particles or heavy atoms, spin labels, radioisotopes, enzyme substrates, haptens, antigens, Quantum Dots, aminohexyl, pyrene, nucleic acids or nucleic acid analogs, or proteins, such as receptors, peptide ligands or substrates, enzymes, and antibodies (including antibody fragments).

Examples of polymer particles labels which may be used include micro particles, beads, or latex particles of polystyrene, PMMA or silica, which can be embedded with fluorescent dyes, or polymer micelles or capsules which contain dyes, enzymes or substrates. Examples of metal particles which may be used include gold particles and coated gold particles, which can be converted by silver stains. Examples of haptens that may be conjugated in some embodiments are fluorophores, myc, nitrotyrosine, biotin, avidin, streptavidin, 2,4-dinitrophenyl, digoxigenin, bromodeoxy uridine, sulfonate, ace tylaminoflurene, mercury trintrophonol, and estradiol.

Examples of enzymes which may be used comprise horseradish peroxidase (HRP), alkaline phosphatase (AP), beta-galactosidase β3-GAL), glucose-6-phosphate dehydrogenase, beta-N-acetylglucosaminidase, βglucuronidase, invertase, Xanthine Oxidase, firefly luciferase and glucose oxidase (GO). Examples of commonly used substrates for HRP include 3,3′-diaminobenzidine (DAB), diaminobenzidine with nickel enhancement, 3-amino-9-ethylcarbazole (AEC), Benzidine dihydrochloride (BDHC), Hanker-Yates reagent (HYR), Indophane blue (IB), tetramethylbenzidine (TMB), 4-chloro-1-naphtol (CN), alpha-naphtol pyronin (α-NP), o-dianisidine (OD), 5-bromo-4-chloro-3-indolylphosphate (BCIP), Nitroblue tetrazolium (NBT), 2-(p-iodophenyl)-3-p-nitrophenyl-5-phenyltetrazolium chloride (INT), tetranitro blue tetrazolium (TNBT), and Δ-bromo-chloro-S-indoxyl-beta-D-galactoside/ferro-ferricyanide (BCIG/FF). Examples of commonly used substrates for AP include Naphthol-AS-B 1-phosphate/fast red TR (NABP/FR), Naphthol-AS-MX-phosphate/fast red TR (NAMP/FR), Naphthol-AS-B1-phosphate/fast red TR (NABP/FR), Naphthol-AS-MX-phosphate/fast red TR (NAMP/FR), Naphthol-AS-B1-phosphate/new fuschin (NABP/NF), bromochloroindolylphosphate/nitroblue tetrazolium (BCIP/NBT), b-Bromo-chloro-S-indolyl-beta-delta-galactopyranoside (BCIG).

Examples of luminescent labels which may be used include luminol, isoluminol, acridinium esters, 1,2-dioxetanes and pyridopyridazines. Examples of electrochemiluminescent labels include ruthenium derivatives. Examples of radioactive labels which may be used include radioactive isotopes of iodide, cobalt, selenium, hydrogen, carbon, sulfur, and phosphorous.

Some “detectable labels” also include “color labels,” in which the biological change or event in the system may be assayed by the presence of a color, or a change in color. Examples of “color labels” are chromophores, fluorophores, chemiluminescent compounds, electrochemiluminescent labels, bioluminescent labels, and enzymes that catalyze a color change in a substrate.

“Fluorophores” as described herein are molecules that emit detectable electro-magnetic radiation upon excitation with electro-magnetic radiation at one or more wavelengths. A large variety of fluorophores are known in the art and are developed by chemists for use as detectable molecular labels and can be conjugated to the pMHC multimers provided herein. Examples include FLUORESCEIN™ or its derivatives, such as FLUORESCEIN®-5-isothiocyanate (FITC), 5-(and 6)-carboxyFLUORESCEIN®, 5- or 6-carboxyFLUORESCEIN®, 6-(FLUORESCEINO)-5-(and 6)-carboxamido hexanoic acid, FLUORESCEIN® isothiocyanate, rhodamine or its derivatives such as tetramethyl rhodamine and tetramethylrhodamine-5-(and -6) isothiocyanate (TRITC). Other fluorophores include: coumarin dyes such as (diethyl-amino)coumarin or7-amino-4-methylcoumarin-3-acetic acid, succinimidyl ester (AMCA); sulforhodamine 101 sulfonyl chloride (TexasRed® or TexasRed® sulfonyl chloride; 5-(and -6)-carboxyrhodamine 101, succinimidyl ester, also known as 5-(and -6)-carboxy-X-rhodamine, succinimidyl ester (CXR); lissamine or lissamine derivatives such as lissamine rhodamine B sulfonyl Chloride (LisR); 5-(and -6)-carboxyFLUORESCEIN®, succinimidyl ester (CFI); FLUORESCEIN®5-isothiocyanate (FITC); 7-diethylaminocoumarin-3-carboxylic acid, succinimidyl ester (DECCA); 5-(and -6)-carboxytetramethyl-rhodamine, succinimidyl ester (CTMR); 7-hydroxycoumarin-3-carboxylic acid, succinimidyl ester (HCCA); 6->FLUORESCEIN®-5-(and -6)-carboxamidolhexanoic acid (FCHA); N-(4,4-difluoro-5,7-dimethyl-4-bora-3a,4a-diaza-3-indacenepropionic acid, succinimidyl ester; also known as 5,7-dimethylBODIPY® propionic acid, succinimidyl ester (DMBP); “activated FLUORESCEIN® derivative” (FAP), available from Probes, Inc.; eosin-5-isothiocyanate (EITC); erythrosin-5-isothiocyanate (Er1TC); and Cascade® Blue acetylazide (CBAA) (the O-acetylazide derivative of 1-hydroxy-3,6,8-pyrene-trisulfonic acid). Yet other potential fluorophores useful in the invention include fluorescent proteins such as green fluorescent protein and its analogs or derivatives, fluorescent amino acids such as tyrosine and tryptophan and their analogs, fluorescent nucleosides, and other fluorescent molecules such as Cy2, Cy3, Cy3.5, CY5™, CY5.5™, Cy7, IR dyes, Dyomics dyes, phycoerythrine, Oregon green 488, pacific blue, rhodamine green, and Alexa dyes. Yet other examples of fluorescent labels include conjugates of R-phycoerythrin orallophycoerythrin, inorganic fluorescent labels such as particles based on semiconductor material like coated CdSe nanocrystallites.

A number of the fluorophores above, as well as others, are available commercially, from companies such as Probes, Inc. (Eugene, Oreg.), Pierce Chemical Co. (Rockford, Ill.), or Sigma-Aldrich Co. (St. Louis, Mo.).

The detectable label can be detected by numerous methods, including, for example, reflectance, transmittance, light scatter, optical rotation, and fluorescence or combinations hereof in the case of optical labels or by film, scintillation counting, or phosphorimaging in the case of radioactive labels. See, e.g., Larsson, 1988, Immunocytochemistry: Theory and Practice, (CRC Press, Boca Raton, Fla.); Methods in Molecular Biology, vol. 80 1998, John D. Pound (ed.) (Humana Press, Totowa, N.J.). In some embodiments, more than one detectable labels employed.

### Identifiers and Barcoding

In certain embodiments, a Conjugated Multimer of the disclosure comprises an identifier tag or label, such as an oligonucleotide barcode, that facilitates identification of the Conjugated Multimer. Typically, the identifier tag, e.g., oligonucleotide barcode, is attached to the multimerization domain of the Conjugated Multimer, such as through a binding moiety on the identifier tag, e.g., oligonucleotide barcode, that binds to a binding site on the multimerization domain. For example, when the multimerization domain is streptavidin or avidin, since the pMHCI monomers are conjugated to the multimerization domain at a site other than the biotin-binding site, the Conjugated Multimer can be labeled with an identifier tag, e.g., oligonucleotide barcode, using a biotinylated form of the identifier tag, e.g., a biotinylated oligonucleotide barcode. Labeling of the Conjugated Multimer is then easily achieved by incubation of the Conjugated Multimer with the biotinylated identifier tag, e.g., biotinylated oligonucleotide barcode. A non-limiting exemplification of barcoding of Conjugated Multimers using biotinylated oligonucleotides is described in detail in Example 8.

In another embodiment, the Conjugated Multimer is labeled with an identifier tag, e.g., oligonucleotide barcode, in the peptide portion of the multimer. That is, barcode-labeled MHC-binding peptides can be used in an exchange reaction as described herein to the load the Conjugated Multimers with barcode-labeled peptides.

Typically, an oligonucleotide barcode is a unique oligonucleotide sequence ranging for 10 to more than 50 nucleotides. The barcode has shared amplification sequences in the 3′ and 5′ ends, and a unique sequence in the middle. This sequence can be revealed by sequencing and can serve as a specific barcode for a given molecule.

In one embodiment, the nucleic acid component of the barcode (typically DNA) has a special structure. Thus, in one embodiment, the at least one nucleic acid molecule is composed of at least a 5′ first primer region, a central region (barcode region), and a 3′ second primer region. In this way the central region (the barcode region) can be amplified by a primer set. The length of the nucleic acid molecule may also vary. Thus, in other embodiments, the at least one nucleic acid molecule has a length in the range 20-100 nucleotides, such as 30-100, such as 30-80, such as 30-50 nucleotides. In one embodiment, the nucleic acid identifier is from 40 nucleotides to 120 nucleotides in length. The coupling of the oligonucleotide barcode to the Conjugated Multimer may also vary. Thus, in one embodiment, the at least one oligonucleotide barcode is linked to said Conjugated Multimer via a biotin binding domain interacting with streptavidin or avidin within the Conjugated Multimer. Other coupling moieties may also be used, depending on the availability of an appropriate binding site with the Conjugated Multimer (e.g., within the multimerization domain of the Conjugated Multimer) and an appropriate corresponding binding domain that can be attached to the oligonucleotide barcodes molecules to facilitate attachment.

In a further embodiment, the at least oligonucleotide barcode molecule comprises or consists of DNA, RNA, and/or artificial nucleotides such as PLA or LNA. Preferably DNA, but other nucleotides may be included to e.g. increase stability.

The use of barcode technology is well known in the art, see for example Shiroguchi et al. (2012) Proc. Natl. Acad. Sci. USA., 109(4):1347-52; and Smith et al. (2010) Nucleic Acids Research 38(13)11:e142. Further methods and compositions for using barcode technology include those described in U.S. 2016/0060621. Use of barcode technology specifically to label MHC multimers also has been described, see for example Bentzen et al. (2016) Nature Biotech. 34:10: 1037-1045; Bentzen and Hadrup (2017) Cancer Immunol. Immunotherap. 66:657-666. Standard methods for preparing barcode oligonucleotides, including conjugating them with a suitable binding moiety (e.g., biotinylation) that can bind the Conjugated Multimer, are known in the art and can be applied to preparing barcode oligonucleotides for labeling the Conjugated Multimers.

Methods for generating customizable DNA barcode libraries are publicly available. Programs include Generator and nxCode, consisting of 96-587 barcodes, respectively, as well as The DNA Barcodes Package and TagD software (reporting generating libraries consisting of 100,000 barcodes).

Preparation of a variety of large-scale barcode libraries have been described in the art, which approaches can be used to obtain barcode libraries for labeling pMHC Conjugated Multimer libraries. For example, Xu et al. describe a set of 240,000 unique 25-mer oligonucleotides with sequences that have similar amplifications properties while maintaining maximum diversity of their identification motifs (Xu et al. (2008) PNAS 106:2289-2294). Wang et al. describe construction of barcode sets using particle swarm optimization (Wang et al. (2008) IEEE/ACM Trans. Comput. Biol. Bioinform. 15:999-1002). Lyons describes generation of large-scale libraries of DNA barcodes of up to one million members (Lyons (2017) Sci. Reports 7:13899).

In some cases, the unique molecular identifier barcode is encoded by a contiguous sequence of nucleotides tagged to one end of a target nucleic acid. In other cases, the unique molecular identifier (UMI) barcode is encoded by a non-contiguous sequence. Non-contiguous UMIs can have a portion of the barcode at a first end of the target nucleic acid and a portion of the barcode at a second end of the target nucleic acid. In some cases, the UMI is a non-contiguous barcode containing a variable length barcode sequence at a first end and a second identifier sequence at a second end of the target nucleic acid. In some cases, the UMI is a non-contiguous barcode having a variable length barcode sequence at a first end and a second identifier sequence at a second end of the target nucleic acid, wherein the second identifier sequence is determined by a position of a transposase fragmentation event, e.g., a transposase fragmentation site and transposon end insertion event.

In some cases, the barcode is a “variable length barcode.” As used herein, a variable length barcode is an oligonucleotide that differs from other variable length barcode oligonucleotides in a population, by length, which can be identified by the number of contiguous nucleotides in the barcode. In some cases, additional barcode complexity for the variable length barcode can be provided by the use of variable nucleotide sequence, as described in the paragraphs above, in addition to the variable length.

In an exemplary embodiment, a variable length barcode can have a length of from 0 to no more than 5 nucleotides. Such a variable length barcode can be denoted by the term “[0-5].” In such an embodiment, it is understood that a population of target nucleic acids that are attached to such a variable length barcode is expected to include at least one target nucleic acid attached to a variable length barcode that has at least 1 nucleotide (e.g., attached to a variable length barcode having only 1, only 2, only 3, only 4, or only 5 nucleotides). In such an embodiment, it is further understood that a population of target nucleic acids that are attached to such a variable length barcode can include at least one target nucleic acid that contains no variable length barcode (i.e., a variable length barcode having a length of 0), and/or at least one target nucleic acid that contains a variable length barcode having only 1 nucleotide, and/or at least one target nucleic acid that contains a variable length barcode having only 2 nucleotides, and/or at least one target nucleic acid that contains a variable length barcode having only 3 nucleotides, and/or at least one target nucleic acid that contains a variable length barcode having only 4 nucleotides, and/or and at least one target nucleic acid that contains a variable length barcode having only 5 nucleotides. In such an embodiment, the [0-5] variable length barcode can uniquely identify (differentiate), by itself, 5 different target nucleic acid molecules of the same sequence. Further, in such an embodiment, the [0-5] variable length barcode can uniquely identify (differentiate) 5 different target nucleic molecules of a first sequence, 5 different target nucleic acid molecules of a second sequence, etc. for each different target nucleic acid sequence. Furthermore, barcode labelled MHC-multimers can be used in combination with single-cell sorting and TCR sequencing, where the specificity of the TCR can be determined by the co-attached barcode. This will enable us to identify TCR specificity for potentially 1000+different antigen responsive T cells in parallel from the same sample, and match the TCR sequence to the antigen specificity. The future potential of this technology relates to the ability to predict antigen responsiveness based on the TCR sequence.

The complexity of the barcode labeled MHC multimer libraries will allow for personalized selection of relevant TCRs in a given individual.

The barcode is co-attached to the multimer and serves as a specific label for a particular peptide-MHC complex. In this way at least 1000 to 10,000 or more different peptide-MHC multimers can be mixed, allow specific interaction with T cells from blood or other biological specimens, wash-out unbound MHC-multimers and determine the sequence of the DNA-barcodes. When selecting a cell population of interest, the sequence of barcodes present above background level, will provide a fingerprint for identification of the antigen responsive cells present in the given cell-population. The number of sequence-reads for each specific barcode will correlate with the frequency of specific T cells, and the frequency can be estimated by comparing the frequency of reads to the input-frequency of T cells.

The DNA-barcode serves as a specific labels for the antigen specific T cells and can be used to determine the specificity of a T cell after e.g. single-cell sorting, functional analyses or phenotypical assessments. In this way antigen specificity can be linked to both the T cell receptor sequence (that can be revealed by single-cell sequencing methods) and functional and phenotypical characteristics of the antigen specific cells.

Barcode labeled MHC multimer libraries can be used for the quantitative assessment of MHC multimer binding to a given T cell clone or TCR transduced/transfected cells. Since sequencing of the barcode label allow several different labels to be determined simultaneously on the same cell population, this strategy can be used to determine the avidity of a given TCR relative to a library of related peptide-MHC multimers. The relative contribution of the different DNA-barcode sequences in the final readout is determined based on the quantitative contribution of the TCR binding for each of the different peptide-MHC multimers in the library. Using titration based analyses it is possible to determine the quantitative binding properties of a TCR in relation to a large library of peptide-MHC multimers, all merged into a single sample. For this particular purpose the MHC multimer library may specifically hold related peptide sequences or alanine-substitution peptide libraries.

In some embodiments, unique identifiers can be used for each sample of a plurality of samples. In some embodiments, identifiers can be shared between two or more samples. In some embodiments, identifiers can comprise some sequences that are shared between all samples, and other sequences that are unique to one sample. In some embodiments, an identifier can comprise a sequence shared between all samples, and a sequence unique to one sample. In some embodiments, a sequence shared between samples can be used for identifier amplification (e.g., PCR amplification with suitable primers). In some embodiments, a sequence unique to one sample or shared between a subset of samples can be used for detection or quantification via qPCR (e.g., sequences for hydrolysis probes, such as TaqMan probes). In some embodiments, a sequence unique to one sample or shared between a subset of samples can be used for detection or quantification via sequencing.

In some embodiments, an identifier can comprise a unique, in sitico-generated sequence; each identifier sequence can be assigned to a sample of a plurality of samples and the identifier-sample assignment can be stored in a database. In some embodiments, an identifier can comprise a nucleotide sequence that codes for all or part of a peptide or protein.

In some embodiments, an identifier can comprise a nucleotide sequence that codes for an open reading frame. In some embodiments, an identifier can comprise a nucleotide sequence that includes a promoter sequence. In some embodiments, an identifier can comprise a nucleotide sequence that includes a binding site for a DNA-binding protein, e.g. a transcription factor or polymerase enzyme. In some embodiments, an identifier can comprise one or more sequences targeted by a nuclease, e.g. a restriction enzyme. In some embodiments, an identifier can comprise all sequence elements necessary for in vitro transcription and translation of a sequence. In some embodiments, an identifier does not comprise all sequence elements necessary for in vitro transcription and translation of a sequence.

In some embodiments, an identifier can comprise a biotinylated nucleotide sequence. In some embodiments, an identifier can be biotinylated by PCR amplification with a biotinylated primer(s). In some embodiments, an identifier can be biotinylated by enzymatic incorporation of a biotinylated label, e.g. a biotin dUTP label, by use of Klenow DNA polymerase enzyme, nick translation or mixed primer labeling RNA polymerases, including T7, T3, and SP6 RNA polymerases. In some embodiments, an identifier can be biotinylated by photobiotinylation, e.g. photoactivatable biotin can be added to the sample, and the sample irradiated with UV light.

In some embodiments, an identifier can be generated from a template polynucleotide, e.g. via PCR amplification of a template DNA. In some embodiments, a template polynucleotide can comprise a nucleotide sequence that codes for an open reading frame. In some embodiments, a template polynucleotide can comprise a nucleotide sequence that includes a promoter sequence. In some embodiments, a template polynucleotide can comprise a nucleotide sequence that includes a binding site for a DNA-binding protein, e.g. a transcription factor or polymerase enzyme. In some embodiments, a template polynucleotide can comprise one or more sequences targeted by a nuclease, e.g. a restriction enzyme. In some embodiments, a template polynucleotide can comprise all sequence elements necessary for in vitro transcription and translation of a sequence. In some embodiments, a template polynucleotide does not comprise all sequence elements necessary for in vitro transcription and translation of a sequence.

pMHC multimers with attached identifiers (e.g., oligonucleotide barcodes) can be incubated with a plurality of T cells, followed by sorting of T cells into single-cell compartments. T cells are lysed, and nucleic acids from lysed T cells comprising identifiers are produced. Nucleic acids are pooled and sequenced. Identifiers allow matching of peptide identifiers to T cell sequences from the same compartment. TCR-antigen specificity profiles are determined by identifying a TCR sequence (e.g., variable region, hypervariable region, or CDR) from a compartment, and quantifying peptide identifier reads from the same compartment.

Multiple TCRs can be identified that exhibit binding affinity for peptides of the peptide library, and multiple peptides can be identified that exhibit binding affinity for specific TCRs.

Epitope mutations in an antigen of an identified TCR-antigen pair can be identified that result in increased or TCR binding affinity.

Peptides and TCR sequences can be identified that are associated with control of disease associated protein, and can be used to design vaccines and cell therapies.

For assessing response to therapy, for each peptide identifier sequenced, corresponding TCR sequences are identified. Multiple TCRs are identified that exhibit binding affinity for some peptides of the peptide library, and multiple peptides are identified that exhibit binding affinity for some TCRs. Subjects are followed longitudinally and results of assays are compared to identify peptides and TCR sequences that are associated with successful response to immunotherapy.

### V. Peptide Synthesis

Peptides can be are generated according to methods known in the art, or synthetically produced by a commercial vendor or using a peptide synthesizer according to manufacturer's instructions. It is understood that the T cell epitopes described herein can be produced by a variety of approaches using synthetic chemistries and recombinant methodologies. Methods for making recombinant proteins, using recombinant technologies, e.g., recombinant DNA technologies, cloning vectors, expression vectors, transfection methodologies, host cells, and culture conditions are known in the art. See, e.g., US 2020/0207849, US 2021/0101955, US 2021/0101975 and US 2021/013043.

It is contemplated that a T cell epitope can be expressed using recombinant DNA technology, for example, introducing an expression construct into bacterial cells, insect cells, or mammalian cells, and purifying the recombinant protein from cell extracts.

General techniques for nucleic acid manipulation are described in, for example, Sambrook et al., Molecular Cloning. A Laboratory Manual, 2nd Edition, Vols. 1-3, Cold Spring Harbor Laboratory Press (1989), or Ausubel, F. et al., Current Protocols in Molecular Biology, Green Publishing and Wiley-Interscience, New York (1987) and periodic updates, herein incorporated by reference. Generally, the DNA encoding the polypeptide is operably linked to suitable transcriptional or translational regulatory elements derived from mammalian, viral, or insect genes. Such regulatory elements include a transcriptional promoter, an optional operator sequence to control transcription, a sequence encoding suitable mRNA ribosomal binding site, and sequences that control the termination of transcription and translation. The ability to replicate in a host, usually conferred by an origin of replication, and a selection gene to facilitate recognition of transformants is additionally incorporated.

Both expression and cloning vectors contain a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the 2 micron plasmid origin is suitable for yeast, and various viral origins (SV40, polyoma, adenovirus, VSV or BPV) are useful for cloning vectors in mammalian cells. Generally, the origin of replication component is not needed for mammalian expression vectors (the SV40 origin may typically be used only because it contains the early promoter).

Expression and cloning vectors may contain a selection gene, also termed a selectable marker. Typical selection genes encode proteins that (a) confer resistance to antibiotics or other toxins, e.g., ampicillin, neomycin, methotrexate, or tracycline, (b) complement auxotrophic deficiencies, or (c) supply critical nutrients not available from complex media, e.g., the gene encoding D-alanine racemase for Bacilli.

Expression and cloning vectors usually contain a promoter that is recognized by the host organism and is operably linked to the nucleic acid encoding the protein described herein, e.g., a fibronectin-based scaffold protein. Promoters suitable for use with prokaryotic hosts include the phoA promoter, beta-lactamase and lactose promoter systems, alkaline phosphatase, a tryptophan (trp) promoter system, and hybrid promoters such as the tan promoter. However, other known bacterial promoters are suitable. Promoters for use in bacterial systems also will contain a Shine-Dalgarno (S.D.) sequence operably linked to the DNA encoding the protein described herein. Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Transcription from vectors in mammalian host cells can be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and most preferably Simian Virus 40 (SV40), from heterologous mammalian promoters, e.g., the actin promoter or an immunoglobulin promoter, from heat-shock promoters, provided such promoters are compatible with the host cell systems.

Transcription of a DNA encoding protein described herein by higher eukaryotes is often increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv (1982) Nature, 297:17-18 on enhancing elements for activation of eukaryotic promoters. The enhancer may be spliced into the vector at a position 5′ or 3′ to the peptide-encoding sequence, but is preferably located at a site 5′ from the promoter.

Expression vectors used in eukaryotic hos T cells (e.g., yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of mRNA encoding the protein described herein. One useful transcription termination component is the bovine growth hormone polyadenylation region. See WO 94/11026 and the expression vector disclosed therein.

The expression construct is introduced into the host cell using a method appropriate to the host cell, as will be apparent to one of skill in the art. A variety of methods for introducing nucleic acids into host cells are known in the art, including, but not limited to, electroporation; transfection employing calcium chloride, rubidium chloride, calcium phosphate, DEAE-dextran, or other substances; microprojectile bombardment; lipofection; and infection (where the vector is an infectious agent).

Suitable host cells include prokaryotes, yeast, mammalian cells, or bacterial cells. Suitable bacteria include gram negative or gram positive organisms, for example, E. coli or Bacillus spp. Yeast, preferably from the Saccharomyces species, such as S. cerevisiae, may also be used for production of polypeptides. Various mammalian or insect cell culture systems can also be employed to express recombinant proteins. Baculovirus systems for production of heterologous proteins in insect cells are reviewed by Luckow et al. (1988) Bio/Technology, 6:47. Examples of suitable mammalian host cell lines include endothelial cells, COS-7 monkey kidney cells, CV-1, L cells, C127, 3T3, Chinese hamster ovary (CHO), human embryonic kidney cells, HeLa, 293, 293T, and BHK cell lines. Purified polypeptides are prepared by culturing suitable host/vector systems to express the recombinant proteins. For many applications, the small size of many of the polypeptides described herein would make expression in E. coli as the preferred method for expression. The protein is then purified from culture media or cell extracts.

Alternatively, a given peptide can be synthesized by in vitro transcription and translation, where synthesis utilizes the biological principles of transcription and translation in a cell-free context, for example, by providing a nucleic acid template, relevant building blocks (e.g., RNAs, amino acids), enzymes (e.g., RNA polymerase, ribosomes), and conditions.

Alternatively, a given peptide can be produced using synthetic chemistries. Methods of chemical synthesis of peptides, such as Fmoc-polyamide mode of solid-phase peptide synthesis, are well known in the art and are described in, for example, Lukas et al., (1981) Proc. Natl. Acad. Sci. US.A 78:2791-95. In certain embodiments, the peptide is present in the form of a salt, for example, a pharmaceutically acceptable salt. The peptides can then be purified by one or a combination of techniques such as re-crystallization, size exclusion chromatography, ion-exchange chromatography, hydrophobic interaction chromatography and reverse-phase high performance liquid chromatography using, e.g., acetonitrile/water gradient separation. In certain embodiments, the peptide is formulated as a salt, such as a pharmaceutically acceptable salt. In certain embodiment, the pharmaceutically acceptable salt comprises one or more anions selected from PO₄³⁻, SO₄²⁻, CH₃COO⁻, Cl⁻, Br⁻, NO₃⁻, ClO₄⁻, I⁻, and SCN⁻ and/or one or more cations selected from NH₄⁺, Rb⁺, K⁺, Na⁺, Cs⁺, Li⁺, Zn₂₊, Mg₂⁺, Ca₂+, Mn₂⁺, Cu₂⁺ and Ba₂⁺.

Peptides and proteins can be purified by isolation/purification methods for peptide and proteins generally known in the field of protein chemistry. Non-limiting examples include extraction, recrystallization, salting out (e.g., with ammonium sulfate or sodium sulfate), centrifugation, dialysis, ultrafiltration, adsorption chromatography, ion exchange chromatography, hydrophobic chromatography, normal phase chromatography, reversed-phase chromatography, get filtration, gel permeation chromatography, affinity chromatography, electrophoresis, countercurrent distribution or any combinations of these. After purification, polypeptides may be exchanged into different buffers and/or concentrated by any of a variety of methods known to the art, including, but not limited to, filtration and dialysis. The resulting peptide is preferably at least 85% pure, or preferably at least 95% pure, and most preferably at least 98% pure. Regardless of the exact numerical value of the purity, the peptide should be sufficiently pure for its intended use.

### VI. Antigen Presenting Cells

The peptide or composition disclosed herein can be used to load an antigen-presenting cell (APC) in complex with a MHC. MHC class I, composed of an alpha heavy chain and beta-2-microglobulin, is found on most nucleated cells. They present peptides that result from proteolytic cleavage of predominantly endogenous proteins, defective ribosomal products (DRIPs) and larger peptides. However, peptides derived from endosomal compartments or exogenous sources are also frequently found on MHC class I molecules. This non-classical way of class I presentation is referred to as cross-presentation (see Brossart and Bevan (1997) Blood 90:1594-99). MHC Class II molecules, composed of an alpha and a beta chain, is found predominantly on professional APCs such as dendritic cells, macrophages, and B lymphocytes. They primarily present peptides of exogenous or transmembrane proteins that are taken up by APCs during endocytosis and are subsequently processed.

In certain embodiments, the disclosure provides a composition comprising an isolated APC that presents on an outer cell surface of the APC a peptide comprising a SARS-CoV-2 T cell epitope (e.g., a CD8+ T cell epitope) comprising an amino acid sequence set forth in TABLE 1, wherein the peptide is no more than 100 amino acids in length, and an optional pharmaceutically acceptable carrier. Alternatively, the APC may present an immunodominant T cell epitope, for example, as set forth in TABLE 2. Alternatively or in addition, the T cell epitope is specific for a subject infected with SARS-CoV-2 as noted in TABLE 2. In each case, it the T cell epitope is presented by a MHC class I molecule at the surface of the APC. In certain embodiments, the T cell epitope in the composition comprises at least 8 continuous amino acids of an epitope sequence set forth in TABLE 1 or 2. Furthermore, the composition may comprises a plurality of APCs, each APC presenting a different T cell epitope. For example, the composition can comprise a second, different APC that presents on its outer cell surface of the APC a second, different peptide comprising a SARS-CoV-2 T cell epitope, wherein the second, different epitope optionally comprises an amino acid sequence set forth in any one of TABLES 1-4, and wherein the peptide is no more than 100 amino acids in length.

In certain embodiment, the disclosure provides a composition comprising an isolated APC that presents on at its outer cell surface (e.g., via MHC class II molecule) a SARS-CoV-2 T cell epitope (a CD4+ epitope) comprising an amino acid sequence set forth in TABLE 3 or 4, wherein the peptide is no more than 100 amino acids in length, and an optional pharmaceutically acceptable carrier. In certain embodiments, the T cell epitope comprises at least 13 continuous amino acids of an epitope sequence set forth in TABLE 3 or 4.

Furthermore, the composition may comprises a plurality of APCs, each APC presenting a different T cell epitope. For example, the composition can comprise a second, different APC that presents on its outer cell surface of the APC a second, different peptide comprising a SARS-CoV-2 T cell epitope, wherein the second, different epitope optionally comprises an amino acid sequence set forth in any one of TABLES 1-4, and wherein the peptide is no more than 100 amino acids in length.

In certain embodiments of each of the foregoing aspects, the peptide is no more than 50, 45, 40, 35, 30, 25 or 20 amino acids in length. Alternatively or in addition, the T cell epitope is synthetic. In certain embodiments of each of the foregoing aspects, the APC is a cell of the myeloid lineage, a cell of the lymphoid lineage, or an artificial APC.

Methods for making APCs are well known in the art and disclosed, for example, in International Application Publication Nos. WO/2020/055931 and WO/2020/198366 and U.S. Patent Application Publication No. 2019/0264176.

In certain embodiments, the APC is a cell of the myeloid lineage, for example, a dendritic cell (DC), monocyte, macrophage, or Langerhans cell. In certain embodiments, the APC is an immature dendritic cell. In certain embodiments, the APC is a mature DC. In certain embodiments, the APC is a myeloid dendritic cell (mDC), e.g., a CD1c/BDCA-1⁺CD11c^{hi}CD123⁻ or CD141/BDCA-3⁺CD11c^{lo} dendritic cell. In certain embodiments, the APC is a plasmacytoid dendritic cell (pDC), e.g., CD11c⁻CD123⁺BDCA-2/CD303⁺.

In certain embodiments, the dendritic cell can be prepared in vitro from monocyte-derived DCs (moDCs), which can be generated in vitro from peripheral blood mononuclear cells (PBMCs). In some embodiments, the monocytes can be acquired by elutriating PBMCs into at least a lymphocyte-rich fraction and a monocyte-rich fraction, wherein preferably the PBMCs are from a patient in need of a therapy for SARS-CoV-2. Plating of PBMCs in a tissue culture flask permits adherence of monocytes. Treatment of these monocytes with interleukin 4 (IL-4) and granulocyte-macrophage colony stimulating factor (GM-CSF) leads to differentiation to immature DCs. Subsequent treatment with tumor necrosis factor (TNF), IL6, IL1B, and PGE2 further differentiates the immature DCs into mature DCs.

T cell epitopes and/or peptides disclosed herein can be loaded on an APC in vitro at various stages of differentiation.

In a conventional loading process, the APC is a DC generated by briefly (typically for 1-3 hours) pulsing mature DCs with one or more T cell epitope or peptide disclosed herein. This method loads peptides directly onto MHC I and MHC II on the cell surface.

In a preloading method, monocytes, immature DCs, or cells prior to becoming mature DC are contacted with one or more T cell epitope or peptide disclosed herein. The cells are induced to internalize and proteolytically process the peptides into shorter fragments for subsequent loading onto MHC class I and/or MHC class II. The processed peptides may be stored by the monocytes and/or immature DCs during the differentiation and/or maturation process and subsequently loaded onto the MHC by the resulting mature DCs. Without wishing to be bound by theory, it is believed that when loaded with 15-mers, most peptides are processed to 8-11 amino acids in length and presented on DCs. Preloading uses intracellular processing of peptides to present peptides that are MHC I allele-specific and thus, can result in a more robust stimulation of a physiologically relevant CD8+ T cell repertoire that can bind peptide:MHC complexes better and more effectively. Furthermore, using preloading, the peptides may be customized by the cell via proteolysis (which may be different across patients), so that the most biologically preferred peptides are loaded regardless of MHC allele.

In certain embodiments, the present disclosure provides a composition comprising a mixture of conventionally loaded DCs and preloaded DCs, and methods for making and using the same. In certain embodiments, the method of preparing APC comprises a preloading process followed by a conventional loading process after cell differentiation, wherein one or more T cell epitopes and/or peptides disclosed herein are used in the preloading process, the conventional loading process, or both.

In another embodiment, the APC is a cell of the lymphoid lineage, for example, B cell.

Also provided herein is a population of APCs presenting the T cell epitopes and/or peptides disclosed herein. In certain embodiments, the population comprises cells of the myeloid lineage. In certain embodiments, the population comprises cells of the lymphoid lineage. In certain embodiments, the population comprises cells of the myeloid lineage and cells of the lymphoid lineage.

In certain embodiments, the disclosure provides a method of making an APC with a T cell epitope on the surface of an APC. The method comprises contacting the APC in vitro with a peptide or composition disclosed herein. In certain embodiments, the composition comprises an agent (e.g., liposome or lipid nanoparticle) to deliver the peptide into the cytoplasm, thereby allowing the peptide to be presented by a MHC Class I protein. In other embodiments, the composition does not comprise an agent that delivers the peptide into the cytosol. In one embodiment, the peptide consists of or consists essentially of an MHC Class I-restricted epitope. Such peptide can be loaded directly on the MHC Class I on the cell surface. In another embodiment, the peptide comprises an MHC Class II-restricted epitope. Such peptide can be internalized into the endosome, then processed and presented by an MHC Class II protein. In certain embodiments, the APC expresses an MHC cognate to the epitope in the peptide.

In certain embodiments, the disclosure provides a method of presenting a T cell epitope on the surface of an APC. The method comprises transfecting the APC in vitro with a nucleic acid (e.g., mRNA) encoding a peptide disclosed herein. In certain embodiments, the peptide is expressed in the cytosol and presented by MHC Class I. In certain embodiments, the peptide is secreted into the extracellular space and is presented by MHC Class I or Class II as described above in connection with contacting the APC in vitro with a peptide. In certain embodiments, the APC expresses an MHC cognate to the epitope in the peptide encoded by the nucleic acid.

In addition, it is contemplated that the APCs can be included in a pharmaceutical composition that further comprises a pharmaceutically acceptable carrier or excipient.

### VII. T Cell Receptors

Also disclosed herein are recombinant T cell receptors (TCRs) reactive to SARS-CoV-2 T cell epitopes and fragments of the TCRs that bind the SARS-CoV-2 T cell epitopes. Methods for making and using engineered TCRs (e.g., soluble and membrane bound forms) and T cells (e.g., CD4+ T cells and CD8+ T cells) that express on their cell surface engineered TCRs are known in the art. See, e.g., US 2020/0207849, US 2021/0101955, US 2021/0101975 and US 2021/013043.

In certain embodiments, the recombinant TCR or the fragment thereof comprises an alpha chain variable domain (Vα) and a beta chain variable domain (Vβ), wherein the Vα and the Vβ comprise an alpha chain CDR3 and an beta chain CDR3 having the amino acid sequences set forth in the same line of TABLE 5. In certain embodiments, the Vα comprises the CDR1 and CDR2 sequences of the alpha V gene in the same line of TABLE 5, and the Vβ comprises the CDR1 and CDR2 sequences of the beta V gene in the same line of TABLE 5. In certain embodiments, the Vα comprises an amino acid sequence at least 90% (e.g., at least 91%, at least 92%, at least 93%, at least 94%, at least 95%, at least 96%, at least 97%, at least 98%, at least 99%, or 100%) identical to the Vα portion of an amino acid sequence encoded by the corresponding alpha chain nucleotide sequence in TABLE 6, and the Vβ comprises an amino acid sequence at least 90% (e.g., at least 91%, at least 92%, at least 93%, at least 94%, at least 95%, at least 96%, at least 97%, at least 98%, at least 99%, or 100%) identical to the VP portion of an amino acid sequence encoded by the corresponding beta chain nucleotide sequence in TABLE 6.

The present disclosure further provides proteins comprising the TCR fragments, such as soluble TCRs, bispecific T-cell engagers, and TCR mimetics (see Chandran and Klebanoff (2019) Immunol. Rev. 290:127-47; Goebeler and Bargou (2020) Nat. Rev. Clin. Oncol. 17:418-34). These proteins are useful for therapeutic as well as diagnostic purposes.

### VIII. T Cells

The peptide or compositions disclosed herein can be used to produce T cells sensitized to the T cell epitope presented to the T cell via an APC.

In one embodiment, the disclosure provides a population of activated and/or expanded T cells produced by a method disclosed herein. The activated T cells can be isolated or enriched.

In certain embodiments, the disclosure provides a composition comprising an isolated T cell (e.g., CD8+ T cell) that binds a peptide comprising a SARS-CoV-2 T cell epitope (CD8+ epitope) comprising an amino acid sequence set forth in TABLE 1, wherein the peptide is no more than 100 amino acids in length, and an optional pharmaceutically acceptable carrier. Alternatively or in addition, the composition comprises an immunodominant T cell epitope set forth in TABLE 2. Alternatively or in addition, the T cell epitope is specific for a subject infected with SARS-CoV-2 as denoted in TABLE 2 as the T cell epitope is present in convalescent patients but not in patients not exposed to SARS-CoV-2. In certain embodiments, the T cell epitope comprises at least 8 continuous amino acids of an epitope sequence set forth in TABLE 1 or 2. The composition may comprises a plurality of different T cells. For example, the composition can comprise a second, different T cell that binds a second, different peptide comprising a SARS-CoV-2 T cell epitope, wherein the second, different epitope optionally comprises an amino acid sequence set forth in any one of TABLES 1-4, and wherein the peptide is no more than 100 amino acids in length.

In certain embodiments, the disclosure provides a composition comprising an isolated T cell (e.g., CD4+ T cell) that binds a peptide comprising a SARS-CoV-2 T cell epitope (CD4+ epitope) comprising an amino acid sequence set forth in TABLE 3, wherein the peptide is no more than 100 amino acids in length, and an optional pharmaceutically acceptable carrier. In certain embodiments, the T cell epitope comprises at least 13 continuous amino acids of an epitope sequence set forth in TABLE 3. The composition may comprises a plurality of different T cells. For example, the composition can comprise a second, different T cell that binds a second, different peptide comprising a SARS-CoV-2 T cell epitope, wherein the second, different epitope optionally comprises an amino acid sequence set forth in any one of TABLES 1-4, and wherein the peptide is no more than 100 amino acids in length.

In each of the foregoing embodiments, the peptide is no more than 50, 45, 40, 35, 30, 25 or 20 amino acids in length. Alternatively or in addition, the T cell epitope is synthetic. In each of the foregoing embodiments, the APC is a dendritic cell, monocyte, macrophage, B cell or an artificial APC.

Methods for making T cells are well known in the art and disclosed, for example, in International Application Publication Nos. WO/2020/055931 and WO/2020/198366 and U.S. Patent Application Publication No. 2019/0264176.

T cells can be obtained from various sources such as PBMCs. It is understood that for activation and expansion, the T cells need not be isolated or purified from PBMCs. Rather, crude PBMCs or a lymphocyte-rich fraction thereof can be stimulated by APCs. Alternatively, T cells can be isolated or enriched using one or more T cell markers (e.g., CD3). In certain embodiments, a subset of T cells (e.g., CD4+ helper T cells such as T_{H}1 cells, CD8+ cytotoxic T cells, regulatory T cells) are isolated or enriched. In further embodiments, a subset of T cells reactive to SARS-CoV-2 can be isolated or enriched using an MHC multimer comprising a peptide and its cognate MHC disclosed herein. It is contemplated that other than APCs, T cells can also be stimulated by immobilized peptides or soluble peptides in complex with the cognate MHCs.

In certain embodiments, the activated T cell population is prepared by co-culturing a lymphocyte-rich fraction of the PBMCs with the APCs disclosed herein (e.g., at a ratio between about 40:1 to about 1:1, e.g., about 20:1 or 10:1) to expand the T cells that are reactive to SARS-CoV-2 epitopes. The cells can be co-cultured in the presence of one or more of IL-2, IL-6, IL-7, IL-12, IL-15 and IL-21. In some embodiments, the cells are co-cultured in the presence of IL-15, IL-12 and optionally one or more of IL-2, IL-21, IL-7 and IL-6. Advantageously, using methods and compositions disclosed herein, the entire process time from PBMCs to T cells can be shortened to 10-20 days, whereas conventional methods typically require at least 20 days (see, e.g., Putz et al. (2005) Methods Mol Med. 109:71-82, incorporated herein by reference in its entirety). The resulting T cells can be used in various T-cell therapies as further disclosed herein.

In certain embodiments, PBMCs can be stimulated directly with one or more T cell epitopes and/or peptides disclosed herein to activate antigen-specific T cells by the APCs in the PBMCs. This method does not require a separate step of preparing APCs presenting SARS-CoV-2 epitopes in a separate population. In certain embodiments, PBMCs are cultured in the presence of the one or more T cell epitopes and/or peptides. In certain embodiments, PBMCs are transduced with one or more nucleic acids encoding the one or more T cell epitopes and/or peptides.

In certain embodiments, the T cells are stimulated by the APCs more than once (e.g., twice, three times, or more). Progressive expansion can be achieved with weekly restimulation. The cell culture can include cytokines that promote proliferation and/or inhibiting cell death, for example, IL-2, IL-7, and/or IL-4. In certain embodiments, the T cells are cultured ex vivo in the presence of IL-7 and IL-4. In certain embodiments, the T cells are expanded in cell culture for 9 days, 10 days, 11 days, 12 days, 13 days, 14 days, 15 days, 16 days, 17 days, 18 days, 19 days, or 20 days of culture.

In certain embodiments, the T cells reactive to SARS-CoV-2 epitopes are further loaded with a cytokine on the cell surface. Suitable cytokines, which increases T cell survival, activity, or memory formation, include but are not limited to IL-15, IL-2, IL-7, IL-10, IL-12, IL-18, IL-21, IL-23, IL-4, IL-6, IL-7, IL-27, IL-1α, IL-1β, IL-5, IFNγ, TNFα, IFNα, IFNβ, GM-CSF, GCSF, and variants thereof. In certain embodiments, the cytokine is linked to a moiety that binds a T cell antigen (see International Application Publication No. WO/2019/010219). In certain embodiments, the cytokine is cross-linked into a protein nanogel (see International Application Publication No. WO/2019/050978). In certain embodiments, the T cells are loaded with two or more cytokines (see International Application Publication No. WO/2020/205808).

In another embodiment, provided is a method of activating a T cell for reactivity to a SARS-CoV-2 antigen. The method comprises contacting a T cell with at least one SARS-CoV-2 T cell epitope of the disclosure, complexed with an MHC molecule, such that the T cell is activated for reactivity to the SARS-CoV-2 T cell epitope. The T cell epitope-MHC molecule complex is such that it effectively presents the SARS-CoV-2 T cell epitope to the T cell. In one embodiment, the T cell epitope-MHC molecule complex is an MHC multimer loaded with the T cell epitope. In another embodiment, the T cell epitope-MHC molecule complex is displayed on a cell surface for presentation of the antigen to the T cells.

In addition, it is contemplated that the T cells can be included in a pharmaceutical composition that further comprises a pharmaceutically acceptable carrier or excipient. For example, the disclosure also provides isolated T cells activated in vitro for reactivity to at least one SARS-CoV-2 T cell epitope using methodologies described herein, and a pharmaceutically acceptable carrier. The resulting T cells can be used in a method for treating a subject with COVID-19. The method comprises administering to the subject isolated cells that have been activated in vitro for reactivity to at least one SARS-CoV-2 T cell epitope.

### IX. Pharmaceutical Compositions and Therapeutic Methods

The present invention also features pharmaceutical compositions that contain a therapeutically effective amount of one or more T cell epitopes, peptides, APCs, or T cells described herein. The composition can be formulated for use in a variety of drug delivery systems. One or more physiologically acceptable excipients or carriers can also be included in the composition for proper formulation.

### Vaccines

The SARS-CoV-2 T cell epitopes can be used to design prophylactic or therapeutic vaccines comprising such composition (e.g., pharmaceutical compositions) for immunizing subjects at risk of contracting, or subjects having already contacted, SARS-CoV-2. In certain embodiments, the vaccine is a subunit vaccine. In certain embodiments, the vaccine elicits a protective immune reaction against a plurality of viruses (e.g., SARS-CoV-1, HCoV-OC43, HCoV-HKU1, HCoV-229E, HCoV-NL63, CMV, EBV, and/or Influenza).

A vaccine composition of the disclosure can comprise a peptide composition(s) comprising the T cell epitope(s). Alternatively, a vaccine composition of the invention can comprise a nucleic acid composition, e.g., an RNA composition or DNA composition, encoding the T cell epitope(s). For such nucleic acid vaccines, suitable regulatory sequences are included such that the peptide epitope is expressed from the nucleic acid (RNA or DNA) in cells of the subject being immunized.

In certain embodiments, the vaccine of the disclosure comprises at least one SARS-CoV-2 T cell epitope peptide such that the vaccine stimulates a T cell immune response when administered to a subject. In various embodiments, the vaccine comprises, e.g., at least one SARS-CoV-2 T cell epitope peptide(s), e.g., comprising a sequence shown in any of TABLES 1-4, and/or combinations thereof. In certain embodiments, the composition comprises two or more (e.g., three or more, four or more, five or more, six or more, seven or more, eight or more, nine or more, ten or more, 11 or more, 12 or more, 13 or more, 14, or more, 15 or more, 16 or more, 17 or more, 18 or more, 19 or more, or 20 or more) of the peptides disclosed herein (e.g., set forth in TABLES 1-4). In certain embodiments, the two or more peptides are derived from the same SARS-CoV-2 antigen. In certain embodiments, the two or more peptides are derived from at least two different SARS-CoV-2 antigens. In certain embodiments, the vaccine comprises two or more SARS-CoV-2 T cell epitope peptides derived from the same SARS-CoV-2 antigen. In certain embodiments, the vaccine comprises two or more SARS-CoV-2 T cell epitope peptides derived from at least two different SARS-CoV-2 antigens. In certain embodiments, the vaccine comprises one or more, or two or more, SARS-CoV-2 T cell epitope peptides derived from one or more, or at least two or more, SARS-CoV-2 antigens selected from the group consisting of ORF1AB, Spike protein, N protein, M protein, 3A protein and E protein. In certain embodiments, the two or more peptides collectively recognize MHC molecules in at least 50%, at least 60%, at least 70%, at least 80%, at least 90%, at least 95%, or at least 99% of the human population. In certain embodiments, the vaccine contains individualized components according to the personal need (e.g., MHC variants) of the particular patient.

In one embodiment, the vaccine comprises one or more SARS-CoV-2 T cell epitope peptides in addition to one or more conformational epitopes recognized by anti-SARS-CoV-2 antibodies.

A vaccine composition of the disclosure can comprise one or more short (e.g., 8-35 amino acids) peptides as the immunostimulatory agent. In certain embodiments, a T cell epitope sequence is incorporated into a larger carrier polypeptide or protein, to create a chimeric carrier polypeptide or protein that comprises the T cell epitope(s). This chimeric carrier polypeptide or protein can then be incorporated into the vaccine composition.

It is understood that a peptide can be expressed from a nucleic acid (e.g., an mRNA) in a cell of the subject. Exemplary methods of producing peptides by translation in vitro or in vivo are described in U.S. Patent Application Publication No. 2012/0157513 and He et al., J. Ind. Microbiol. Biotechnol. (2015) 42(4):647-53. The present disclosure provides a composition (e.g., pharmaceutical composition) comprising one or more nucleic acids (e.g., mRNAs) encoding one or more peptides disclosed herein, optionally further comprising a pharmaceutically acceptable carrier or excipient. In certain embodiments, the composition comprises nucleic acid sequences encoding two or more (e.g., three or more, four or more, five or more, six or more, seven or more, eight or more, nine or more, ten or more, 11 or more, 12 or more, 13 or more, 14, or more, 15 or more, 16 or more, 17 or more, 18 or more, 19 or more, or 20 or more) of the peptides disclosed herein. In certain embodiments, the two or more peptides are derived from the same SARS-CoV-2 antigen. In certain embodiments, the two or more peptides are derived from at least two different SARS-CoV-2 antigens. In certain embodiments, the composition comprises a nucleic acid sequence encoding one or more of the T cell epitopes set forth in TABLES 1-4. In certain embodiments, the two or more peptides collectively recognize MHC molecules in at least 50%, at least 60%, at least 70%, at least 80%, at least 90%, at least 95%, or at least 99% of the human population. In certain embodiments, the vaccine contains individualized components according to the personal need (e.g., MHC variants) of the particular patient. In certain embodiments, each of the nucleic acids further comprises one or more expression control sequences (e.g., promoter, enhancer, translation initiation site, internal ribosomal entry site, and/or ribosomal skipping element) operably linked to one or more of the peptide coding sequences.

The compositions (e.g., pharmaceutical compositions) disclosed herein may be formulated for delivery into cells (e.g., APCs, such as dendritic cells, monocytes, macrophages, or artificial APCs). In certain embodiments, the composition comprises an agent that facilitate transfection in vitro or in vivo, such as a liposome or a nanoparticle (e.g., lipid nanoparticle). In certain embodiments, the liposome or nanoparticle further comprises a binding moiety (e.g., an antibody or an antigen-binding fragment thereof) for delivering the liposome or nanoparticle to a target T cell (e.g., a professional APC). Another delivery method employs virus particles (e.g., adenovirus, adeno-associated virus, vaccinia virus, fowlpox virus, self-replicating alphavirus, marabavirus, or lentivirus). In certain embodiments, the composition comprises a pharmaceutically acceptable carrier or excipient, such as a diluent, an isotonic solution, water, etc. Excipients also can be selected for enhancement of delivery of the composition.

APCs are also useful as vaccines. Such vaccines may be advantageous over peptide vaccines in avoiding immune tolerance (see Toes et al. (1998) J. Immunol. 160, 4449-56; Monzavi et al. (2021) Cellular Immunity 367: 104398). Accordingly, in certain embodiments, the composition or vaccine comprises one or more of the APCs disclosed herein.

In certain embodiments, the composition or vaccine comprises at least one immunogenicity enhancing adjuvant. Adjuvants included in the vaccine preparation are selected to enhance immune responsiveness to the T cell epitope(s) while maintaining suitable pharmaceutical delivery and avoiding detrimental side effects. Numerous adjuvants and excipients known in the art for use in T cell epitope vaccines can be evaluated for inclusion in the vaccine composition. Suitable adjuvants include any substance that, for example, activates or accelerates the immune system to cause an enhanced antigen-specific immune response. Examples of adjuvants that can be used in the present invention include mineral salts, such as calcium phosphate, aluminum phosphate and aluminum hydroxide; immunostimulatory DNA or RNA, such as CpG oligonucleotides; proteins, such as antibodies or Toll-like receptor binding proteins; saponins (e.g., QS21); cytokines; muramyl dipeptide derivatives; LPS; MPL and derivatives including 3D-MPL; GM-CSF (Granulocyte-macrophage colony-stimulating factor); imiquimod; colloidal particles; complete or incomplete Freund's adjuvant; Ribi's adjuvant or bacterial toxin e.g. cholera toxin or enterotoxin (LT). More adjuvants are disclosed by U.S. Pat. No. 10,772,915. The amounts and concentrations of adjuvants useful in the context of the present invention can be readily determined by the skilled artisan without undue experimentation.

A T cell immune response can be stimulated in vivo. In certain embodiments, the T cell immune response is stimulated in vivo in a patient, wherein the method comprises administering to the patient a peptide, nucleic acid, or composition (e.g., pharmaceutical composition) disclosed herein. It is understood that the peptide, nucleic acid, or composition can be given as a vaccine for therapeutic or prophylactic uses. Accordingly, in certain embodiments, the disclosure provides a method of stimulating an anti-SARS-CoV-2 T cell immune response in a subject, the method comprising administering a vaccine of the disclosure to the subject. In one embodiment, the patient is at risk of infection by SARS-CoV-2. In another embodiment, the patient has an acute infection by SARS-CoV-2. In another embodiment, the patient has a chronic or latent infection by SARS-CoV-2.

Suitable routes of administration and dosages for vaccines are known in the art and can be determined by a person of medical skill. In certain embodiments, the vaccine is administered parenterally, e.g., by intramuscular, intradermal, subcutaneous, intravenous, topical, nasal, or local administration. In certain embodiments, the vaccine comprising peptide(s) is administered via skin scarification. In certain embodiments, the vaccine comprising peptide(s) is administered at a dosage of 0.1-10 mg, e.g., 0.1-0.5 mg, 0.5-1 mg, 1-3 mg, 1-5 mg, or 5-10 mg of total amount per human patient. In certain embodiments, the vaccine comprises a plurality of different peptides, wherein each peptide is provided at a dosage of 0.01-0.05 mg, 0.05-0.1, or 0.1-0.5 mg per human patient. Stimulation of an anti-SARS-CoV-2 T cell immune response in a subject by the vaccine can be monitored by methods established in the art, e.g., by isolating T cells from the subject and measuring reactivity of the T cells to the SARS-CoV-2 T cell epitope(s) contained within the vaccine (see, e.g., Section X below).

### T Cell Therapies

The disclosure facilitates the use of SARS-CoV-2 T cell epitopes described herein for designing T cell-mediated therapies to treat COVID-19. In certain embodiments, the T cells described herein (e.g., obtained by contacting with APCs) are useful as T cell therapy. In certain embodiments, the T cell therapy comprises a plurality of (e.g., 2, 3, 4, 5, 6, 7, 8, 9, 10, or more) clonally different T cells. In certain embodiments, the T cell therapy comprises T cells reactive to a plurality of (e.g., 2, 3, 4, 5, 6, 7, 8, 9, 10, or more) SARS-CoV-2 T cell epitopes.

Alternatively, a TCR disclosed herein can be used as part of a therapeutic intervention. For example, a TCR sequence, TCR variable region sequence, or CDR sequence can be transfected or transduced into T cells to generate modified T cells of the same antigenic specificity. The modified T cells can be expanded, polarized to a desired effector phenotype (e.g., T_{H}1, Tc1, Treg), and infused into a subject. In some embodiments, multiple TCRs identified using compositions and methods disclosed herein are used in an oligoclonal therapy.

In certain embodiments, T cells are engineered to express one or more recombinant TCRs reactive to one or more SARS-CoV-2 T cell epitopes. For example, the SARS-CoV-2 T cell epitopes identified by the methods described herein can be used in designing recombinant TCRs for use in TCR-T or CAR-T technology. In the CAR-T technology, a chimeric antigen receptor (CAR) is used to confer T cells the ability to target a specific epitope, e.g., a SARS-CoV-2 T cell epitope, identified by the methods described herein. In the TCR-T technology, a TCR is used to confer T cells the ability to target a specific epitope, e.g., a SARS-CoV-2 T cell epitope. Methods for expressing a TCR in T cells are known in the art (see, e.g., U.S. Pat. No. 11,033,584). Methods for preparing TCR-transgenic T cells are known in the art and disclosed, for example, by Rath and Arber (2020) Cells 9:1485 and Xu et al. (2020) J. Cellular Immunol. 2(6):284-88). In certain embodiments, the T cell therapy comprises T cells having a plurality of (e.g., 2, 3, 4, 5, 6, 7, 8, 9, 10, or more) of different TCRs disclosed herein.

In certain embodiments, the T cells are transfected with one or more nucleic acids encoding a TCR disclosed herein (see, e.g., Section VII above). In certain embodiments, each of the nucleic acids further comprises one or more expression control sequences (e.g., promoter, enhancer, translation initiation site, internal ribosomal entry site, and/or ribosomal skipping element) operably linked to one or more of the TCR coding sequences.

The T cell therapy is particularly useful for treating patients who are not able to generate sufficient T cells by the vaccination methods disclosed herein. Such patients include but are not limited to immunocompromised individuals, lymphopenic individuals, and patients with low pre-existing COVID-specific T cells.

The T cells disclosed herein, expressing an anti-SARS-CoV-2 TCR either recombinantly or from the genome, can be genetically modified for increased survival, increased and/or prolonged activity, and reduced interference from other therapies commonly used for COVID-19 treatment. For example, in certain embodiments, the T cells can be genetically engineered to inactivate or reduce the expression level of an immune suppressor such as an immune checkpoint protein. Exemplary immune checkpoint proteins expressed by wild-type T cells include but are not limited to PD-1, CTLA-4, A2AR, B7-H3, B7-H4, BTLA, KIR, LAG3, TIM-3, TIGIT, VISTA, PTPN6 (SHP-1), and FAS. In certain embodiments, the T cells can be genetically modified to inactivate or reduce the expression level of the glucocorticoid receptor gene (NR3C1), thereby rendering the T cells insensitive to corticosteroid, which is useful for managing severe COVID-19 (see Basar et al. (2021) Cell Reports 36:109432).

The T cells disclosed herein have therapeutic or prophylactic uses as adoptive cell therapies. For example, they can be used for treating SARS-CoV-2 infection. Accordingly, the present invention provides a method of stimulating a T cell immune response to SARS-CoV-2 in a subject, the method comprising administering to the subject a composition comprising a population of activated T cells disclosed herein. The disclosure also provides a method of stimulating a T cell immune response to SARS-CoV-2 in a subject, the method comprising administering to the subject a composition comprising T cells disclosed herein (e.g., T cells engineered to express a TCR). Where the subject has been infected with SARS-CoV-2, the method can be used to ameliorating a symptom of SARS-CoV-2 infection in the subject.

In certain embodiments, the patient has an acute infection by SARS-CoV-2. In certain embodiments, the patient has a chronic or latent infection by SARS-CoV-2. In certain embodiments, the subject is at risk of infection by SARS-CoV-2. The cell therapy can be provided in a MHC matching manner. For example, in certain embodiments, the T cells are reactive to an epitope-MHC complex wherein the patient has the same MHC allele. In certain embodiments, the T cells are generated by contacting with an epitope-MHC complex wherein the patient has the same MHC allele. In certain embodiments, the cell therapy is autologous, i.e., the T cells were obtained from the same subject. In other embodiments, the cell therapy is allogeneic, i.e., the T cells are obtained from another subject (e.g., a healthy donor).

Suitable routes of administration and dosages for T cell therapies are known in the art and can be determined by a person of medical skill. In certain embodiments, the T cell therapy is administered by intravenous infusion. In certain embodiments, the T cell therapy is administered at a dosage of 10⁴ to 10⁹ cells/kg body weight, e.g., 10⁵ to 10⁶ cells/kg body weight. In certain embodiments, the T cell therapy is administered at a dosage of 10⁶ to 10⁸ cells/m² of body surface area, e.g., 5×10⁶ to 5×10⁷ cells/m² of body surface area. Anti-SARS-CoV-2 T cell immune response in a subject by the vaccine can be monitored by methods established in the art, e.g., by isolating T cells from the subject and measuring reactivity of the T cells to the SARS-CoV-2 T cell epitope(s) contained within the vaccine (see, e.g., Section X below).

### X. Diagnostic Methods

It is contemplated that the T cell epitopes and their corresponding APCs and T cells can be used in a variety of diagnostic and prognostic approaches. For example, information about a given T cell epitope or group of T cell epitopes and corresponding T cells can be used to determine whether a subject has been infected with SARS-CoV-2, and, if infected, whether the subject is likely to have an acute response to the infection, which may impact patient treatment. In some embodiments, the compositions and methods disclosed herein are used to guide clinical decision making, e.g. treatment selection, identification of prognostic factors, monitoring of treatment response or disease progression, or implementation of preventative measures. For example, the sequences identified as COVID-specific in TABLE 2 can be used to determine if a subject or patient has COVID-19. In certain embodiments, a cutoff of frequency can be established in which a patient is diagnosed as having COVID-19 if a certain number of COVID-19-specific T cells are detected from a patent sample.

Furthermore, information about a given T cell epitope or group of T cell epitopes and corresponding T cells can be used to determine whether a subject may elicit a more desirable immune response to one therapeutic agent over another. For example, the information (e.g., sequences) described herein and associated clinical data from a patient can permit the identification of features, for example, biomarkers, that indicated whether a person is likely to be asymptomatic or if they will develop symptoms of COVID-19, e.g., severe symptoms.

Using the information provided herein, it is possible to identify a T cell immune response in a COVID-19 patient. The method comprises contacting a sample of T cells from the COVID-19 patient with a MHC multimer library described herein and identifying a T cell within the sample that binds to at least one member of the MHC multimer library to thereby identify a T cell immune response in the COVID-19 patient. The method can further comprise determining the sequence of the peptide(s) loaded onto the MHC multimer(s) to which the T cell binds to thereby determine the antigenic specificity of the T cell response in the COVID-19 patient. The method can further comprise selecting a treatment regimen for the COVID-19 patient based on the antigenic specificity of the T cell response in the COVID-19 patient. It is contemplated that such a method can be conducted on a plurality of COVID-19 patients, and the resulting information can be used to identify a patient subpopulation having an antigen-specific T cell response of interest.

### T Cell Detection

It is contemplated that a given T cell population, e.g., a T cell population described herein (see, e.g., Tables 1-4), can be detected using a variety of approaches.

a. Nucleic Acid Amplification and Sequencing

In some embodiments, the identity and quantification of a TCR on a selected T cell or population of T cells can be determined by nucleic acid amplification and sequencing (e.g., sequencing a variable, hypervariable region or complementarity determining region (CDR) of a TCR, e.g., alpha and/beta chain CDR3 sequences). Methods of nucleic acid amplification are known in the art and include, for example, PCR, qPCR, nicking endonuclease amplification reaction (NEAR), transcription mediated amplification (TMA), loop-mediated isothermal amplification (LAMP), helicase dependent amplification (HAD), and strand displacement amplification (SDA). In some embodiments, following nucleic acid amplification, the identity of the peptide of the pMHC that binds to a TCR is determined by sequencing (e.g., using an identifier as disclosed herein). Sequencing can be performed, for example, using any suitable sequencing method or instrument known in the art, including an Illumina NextSeq550 instrument (San Diego, Calif., USA). Sequencing data can be processed using any suitable software (e.g., the Cell Ranger Software Suite).

b. Flow Cytometry

MHC multimers using the peptides disclosed herein can be used for detection of individual T cells in fluid samples using flow cytometry or flow cytometry-like analysis.

MHC multimers can be used to identify antigen-specific T cells of interest, for example by screening a plurality of T cells with a library of MHC multimers. In various embodiments, the library comprises MHC multimers loaded with a diversity of more than 10, more than 100, more than 500, more than 1000, more than 2,000, more than 5,000, more than 10,000, more than 10⁶, more than 10⁷, more than 10⁸, more than 10⁹, or more than 10¹⁰ unique peptides. The identification approach can comprise compartmentalizing a cell of the plurality of cells bound to a MHC multimer of the library in a single compartment, wherein the MHC multimer comprises a unique identifier; and determining the unique identifier for each MHC multimer bound to the compartmentalized cell. A compartment can be a separate space, e.g., a well, a plate, a divided boundary, a phase shift, a vessel, a vesicle, a cell, etc.

Liquid cell samples can be analyzed using a flow cytometer, able to detect and count individual cells passing in a stream through a laser beam. For identification of specific T cells using MHC multimers, cells are stained with fluorescently labeled MHC multimer by incubating cells with MHC multimer and then forcing the cells with a large volume of liquid through a nozzle creating a stream of spaced cells. Each cell passes through a laser beam and any fluorochrome bound to the cell is excited and thereby fluoresces. Sensitive photomultipliers detect emitted fluorescence, providing information about the amount of MHC multimer bound to the cell. By this method, MHC multimers can be used to identify individual T cells and/or specific T cell populations in liquid samples.

Cell samples capable of being analyzed by MHC multimers in flow cytometry analysis include, but are not limited to, blood samples or fractions thereof, T cell lines (hybridomas, transfected cells) and homogenized tissues like spleen, lymph nodes, tumors, brain or any other tissue comprising T cells.

When analyzing blood samples, whole blood can be used with or without lysis of red blood cells prior to analysis on a flow cytometer. Lysing reagent can be added before or after staining with MHC multimers. When analyzing blood samples without lysis of red blood cells, one or more gating reagents may be included to distinguish lymphocytes from red blood cells. Preferred gating reagents are marker molecules specific for surface proteins on red blood cells, enabling subtraction of this cell population from the remaining cells of the sample. As an example, a fluorochrome labelled CD45 specific marker molecule e.g., an antibody, can be used to set the trigger discriminator to allow the flow cytometer to distinguish between red blood cells and stained white blood cells.

Alternative to analysis of whole blood, lymphocytes can be purified before flow cytometry analysis, e.g., using standard procedures like a FICOLL®-Hypaque gradient. Another possibility is to isolate T cells from the blood sample, for example, by adding the sample to antibodies or other T cell-specific markers immobilized on solid support. Marker specific T cells will then be attached to the solid support, and following washing, specific T cells can be eluted. This purified T cell population can then be used for flow cytometry analysis together with MHC multimers.

T cells may also be purified from other lymphocytes or blood cells by rosetting. Human T cells form spontaneous rosettes with sheep erythrocytes also called E-rossette formation. E-rossette formation can be carried out by incubating lymphocytes with sheep red erythrocytes followed by purification over a density gradient, e.g., a FICOLL® Hypaque gradient.

Instead of actively isolating T cells, unwanted cells like B-cells, NK cells or other cell populations can be removed prior to the analysis. A method for removing unwanted cells is to incubate the sample with marker molecules specific or one or more surface proteins on the unwanted cells immobilized unto solid support. An example includes use of beads coated with antibodies or other marker molecule specific for surface receptors on the unwanted cells, e.g., markers directed against CD19, CD56, CD14, CD15 or others. Briefly, beads coated with the specific surface marker(s) are added to the cell sample. Non-T cells with appropriate surface receptors will bind the beads. Beads are removed by, e.g., centrifugation or magnetic withdrawal (when using magnetic beads) and the remaining cells are enriched for T cells.

Another example is affinity chromatography using columns with material coated with antibodies or other markers specific for the unwanted cells.

Alternatively, specific antibodies or markers can be added to the blood sample together with complement, thereby killing cells recognized by the antibodies or markers.

Various gating reagents can be included in the analysis. Gating reagents can be labeled antibodies or other labelled marker molecules identifying subsets of cells by binding to unique surface proteins or intracellular components or intracellular secreted components. Preferred gating reagents when using MHC multimers are antibodies and marker molecules directed against CD2, CD3, CD4, and CD8 identifying major subsets of T cells. Other preferred gating reagents are antibodies and markers against CD11a, CD14, CD15, CD19, CD25, CD30, CD37, CD49a, CD49e, CD56, CD27, CD28, CD45, CD45RA, CD45RO, CD45RB, CCR7, CCR5, CD62L, CD75, CD94, CD99, CD107b, CD109, CD152, CD153, CD154, CD160, CD161, CD178, CDw197, CDw217, Cd229, CD245, CD247, Foxp3, or other antibodies or marker molecules recognizing specific proteins unique for different lymphocytes, lymphocyte populations or other cell populations. Also included are antibodies and markers directed against interleukins, e.g., IL-2, IL-4, IL-6, IL-10, IL-12, and IL-21; interferons e.g., INFγ, TNFα, and TNFβ, and other cytokines or chemokines.

Gating reagents can be added before, after or simultaneously with the addition of an MHC multimer to the sample. Following labelling with an MHC multimer and before analysis on a flow cytometer, stained cells can be treated with a fixation reagent (e.g., formaldehyde, ethanol or methanol) to cross-link bound MHC multimer to the cell surface. Stained cells can also be analyzed directly without fixation.

The flow cytometer can in one embodiment be equipped to separate and collect particular types of cells. This is called cell sorting. MHC multimers in combination with sorting on a flow cytometer can be used to isolate antigen specific T cell populations. Gating reagents as described above can be including further specifying the T cell population to be isolated. Isolated and collected specific T cell populations can then be further manipulated as described elsewhere herein, e.g., expanded in vitro.

Direct determination of the concentration of MHC-peptide specific T cells in a sample can be obtained by staining blood cells or other cell samples with MHC multimers and relevant gating reagents followed by addition of an exact amount of counting beads of known concentration. In general, the counting beads are microparticles with scatter properties that put them in the context of the cells of interest when registered by a flow cytometer. They can be either labelled with antibodies, fluorochromes or other marker molecules or they may be unlabeled. In some embodiments, the beads are polystyrene beads with molecules embedded in the polymer that are fluorescent in most channels of the flow cytometer. In connection with this assay, the terms “counting bead” and “microparticle” are used interchangeably.

Beads or microparticles suitable for use include those which are used for gel chromatography, for example, gel filtration media such as SEPHADEX®. Suitable microbeads of this sort include, but are not limited to, SEPHADEX® G-10 having a bead size of 40-120 μm (Sigma Aldrich catalogue number 27, 103-9), SEPHADEX® G-15 having a bead size of 40-120 μm (Sigma Aldrich catalogue number 27, 104-7), SEPHADEX® G-25 having a bead size of 20-50 μm (Sigma Aldrich catalogue number 27, 106-3), SEPHADEX® G-25 having a bead size of 20-80 μm (Sigma Aldrich catalogue number 27, 107-1), SEPHADEX® G-25 having a bead size of 50-150 μm (Sigma Aldrich catalogue number 27, 109-8), SEPHADEX® G-25 having a bead size of 100-300 μm (Sigma Aldrich catalogue number 27, 110-1), SEPHADEX® G-50 having a bead size of 20-50 μm (Sigma Aldrich catalogue number 27, 112-8), SEPHADEX® G-50 having a bead size of 20-80 μm (Sigma Aldrich catalogue number 27, 113-6), SEPHADEX® G-50 having a bead size of 50-150 μm (Sigma Aldrich catalogue number 27, 114-4), SEPHADEX® G-50 having a bead size of 100-300 μm (Sigma Aldrich catalogue number 27, 115-2), SEPHADEX® G-75 having a bead size of 20-50 μm (Sigma Aldrich catalogue number 27, 116-0), SEPHADEX® G-75 having a bead size of 40-120 μm (Sigma Aldrich catalogue number 27, 117-9), SEPHADEX® G-100 having a bead size of 20-50 μm (Sigma Aldrich catalogue number 27, 118-7), SEPHADEX® G-100 having a bead size of 40-120 μm (Sigma Aldrich catalogue number 27, 119-5), SEPHADEX® G-150 having a bead size of 40-120 μm (Sigma Aldrich catalogue number 27, 121-7), and SEPHADEX® G-200 having a bead size of 40-120 μm (Sigma Aldrich catalogue number 27, 123-3).

Other preferred particles for use in the methods and compositions described here comprise plastic microbeads. While plastic microbeads are usually solid, they may also be hollow inside and could be vesicles and other microcarriers. They do not have to be perfect spheres in order to function in the methods described here. Plastic materials such as polystyrene, polyacrylamide and other latex materials may be employed for fabricating the beads, but other plastic materials such as polyvinylchloride, polypropylene and the like may also be used.

The counting beads are used as reference population to measure the exact volume of analyzed sample. The sample(s) are analyzed on a flow cytometer and the amount of MHC-specific T cell is determined using, e.g., a predefined gating strategy and then correlating this number to the number of counted counting beads in the same sample.

Detection of specific T cells in a sample combined with simultaneous detection of activation status of T cells can also be measured using marker molecules specific for up- or down-regulated surface exposed receptors together with MHC multimers. The marker molecule and MHC multimer can be labelled with the same label or different labelling molecules and added to the sample simultaneously or sequentially or separately.

c. Microscopy

Another method of detecting individual T cells in fluid samples uses microscopy. Microscopy comprises any type of microscopy including optical, electron and scanning probe microscopy, bright field microscopy, dark field microscopy, phase contrast microscopy, differential interference contrast microscopy, fluorescence microscopy, confocal laser scanning microscopy, X-ray microscopy, transmission electron microscopy, scanning electron microscopy, atomic force microscope, scanning tunneling microscope and photonic force microscope.

In an exemplary approach, a suspension of T cells are added to MHC multimers. The sample is washed and then the amount of MHC multimer bound to each cell is measured. Bound MHC multimers may be labelled directly or measured through addition of labelled marker molecules. The sample is then spread out on a slide or similar in a thin layer able to distinguish individual cells and labelled cells identified using a microscope. Depending on the type of label different types of microscopes may be used, e.g. if fluorescent labels are used a fluorescent microscope is used for the analysis. For example, MHC multimers can be labeled with a fluorochrome or bound MHC multimer detected with a fluorescent antibody. Cells with bound fluorescent MHC multimers can then be visualized using e.g. an immunofluorescence microscope or a confocal fluorescence microscope.

d. Immunohistochemistry (IHC)

IHC is a method where MHC multimers can be used to directly detect specific T cells e.g. in sections of solid tissue. In some embodiments, sections of fixed or frozen tissue sample are incubated with MHC multimer allowing MHC multimer to bind specific T cells in the tissue. The MHC multimer may be labelled with a fluorochrome, chromophore, or any other labelling molecule that can be detected. The labeling of the MHC multimer may be directly or through a second marker molecule. As an example, the MHC multimer can be labelled with a tag that can be recognized by e.g. a secondary antibody, optionally labelled with horseradish peroxidase (HRP) or another label. The bound MHC multimer is then detected by its fluorescence or absorbance (for fluorophore or chromophore), or by addition of an enzyme-labelled antibody directed against this tag, or another component of the MHC multimer (e.g. one of the protein chains, a label on the one or more multimerization domain). The enzyme can be, e.g. HRP or alkaline phosphatase (AP), both of which convert a colorless substrate into a colored reaction product in situ. This colored deposit identifies the binding site of the MHC multimer and can be visualized under, e.g., a light microscope. The MHC multimer can also be directly labelled with, e.g., HRP or AP, and used in IHC without an additional antibody.

In some embodiments, the detection of T cells in solid tissue includes use of tissue embedded in paraffin, from which tissue sections are made and fixed in formalin before staining. Antibodies are standard reagents used for staining of formalin-fixed tissue sections; these antibodies often recognize linear epitopes. In contrast, most MHC multimers are expected to recognize a conformational epitope on the TCR. In this case, the native structure of TCR needs to be at least partly preserved in the fixed tissue.

e. Immunofluorescence Microscopy

In some embodiments, MHC multimers can be used to identify specific T cells in sections of solid tissue. Instead of visualization of bound MHC multimer by an enzymatic reaction, MHC multimers are labelled with a fluorochrome or bound MHC multimer are detected by a fluorescent antibody. Cells with bound fluorescent MHC multimers can be visualized in an immunofluorescence microscope or in a confocal fluorescence microscope. This method can also be used for detection of T cells in fluid samples using the principles described for detection of T cells in fluid sample described elsewhere herein.

f. Microchip MHC Multimer Technology

A microarray of MHC multimers can be formed by immobilization of different MHC multimers on solid support, to form a spatial array where the position specifies the identity of the MHC-peptide complex or specific empty MHC immobilized at this position. When labelled cells are passed over the microarray (e.g. blood cells), the cells carrying TCRs specific for MHC multimers in the microarray will become immobilized. The label will thus be located at specific regions of the microarray, which will allow identification of the MHC multimers that bind the cells, and thus, allows the identification of, e.g., T cells with recognition specificity for the immobilized MHC multimers. Alternatively, the cells can be labelled after they have been bound to the MHC multimers. The label can be specific for the type of cell that is expected to bind the MHC multimer, or the label can stain cells in general (e.g., a label that binds DNA). Alternatively, cytokine capture antibodies can be co-spotted together with MHC on the solid support and the cytokine secretion from bound antigen specific T cells analyzed. This is possible because T cells are stimulated to secrete cytokines when recognizing and binding specific MHC-peptide complexes.

The MHC multimers, and libraries thereof, can be used in a number of screening methods that allow for the convenient detection and quantification of antigen-specific binding to immune cell receptors. Such MHC multimer libraries can allow, for example, detection of T cells specific for a given antigen, multiplex detection of T cell specificities in a given sample, matching of TCR sequence with specificity (e.g., via single cell sequencing), comparative TCR affinity determination, determination of a consensus specificity sequence of a given TCR, or mapping of antigen responsiveness of T cells against sequences of interest. MHC multimer libraries may be used in T cell screens to determine antigen-reactive T cells as described, for example, in Simon et al. (2014) Cancer Immunol Res 2(12):1230-1244.

A non-limiting example of the method of identifying reactive T cells to SARS-CoV-2 T cell epitopes using an MHC multimer-based approach is described in further detail in Example 18. In another embodiment, T cells reactive to SARS-CoV-2 T cell epitopes are identified using an MCR system for Membrane Epitope Display, described in further detail below and exemplified in Example 17.

g. Indirect Detection of T Cells Using pMHC Multimers

T cells in a sample may also be detected indirectly using MHC multimers. In indirect detection, the number or activity of T cells is measured by detecting events that are the result of TCR-MHC-peptide complex interaction. Interaction between an MHC multimer and a T cell may stimulate the T cell, resulting in activation of the T cell, cell division and proliferation of T cell populations. Alternatively, interaction between an MCH multimer and a T cell may result in inactivation of a T cell.

Activation can be assessed by, for example, measuring the secretion of specific soluble factors (e.g., cytokines) using, e.g., flow cytometry as described herein; measurement of expression of activation markers, e.g., measurement of expression of CD27 and CD28 and/or other receptors by e.g. flow cytometry and/or ELISA or ELISA-like methods; and measurement of T cell effector function, e.g., using a CD8 T cell cytotoxicity assay to measure, e.g., chromium release, as is known by persons skilled in the art. In certain embodiments, activation of a T cell is measured using an Activation Induced Marker (AIM) assay, in which expression of activation markers, e.g., CD27 and CD28 and/or other receptors, are measured by, e.g., flow cytometry. In certain embodiments, activation of a T cell is assessed using an Enzyme Linked Immuno Spot Assay (ELISpot), which detects cytokine-secreting cells at the single cell level using a sandwich assay similar to ELISA.

Proliferation of T cell populations can be assessed by measuring mRNA, measuring incorporation of thymidine or incorporation of other molecules like bromo-2′-deoxyuridine (BrdU).

Inactivation of T cells can be assessed by measuring the effect of blockade of specific TCRs or measuring apoptosis.

When contacted with a diverse population of T cells, such as is contained in a sample of the peripheral blood lymphocytes (PBLs) of a subject, those tetramers containing pMHCs that are recognized by a T cell in the sample will bind to the matched T cell. The contents of the reaction are analyzed using fluorescence flow cytometry to determine, quantify and/or isolate those T cells having an MHC tetramer bound thereto.

h. Detection of T Cells in Solid Tissue In Vivo

MHC multimers may also be used to detect T cells in solid tissue in vivo. To detect T cells in vivo, labeled MHC multimers are injected into the body of the subject to be investigated. The MHC multimers may be labeled with, e.g., a paramagnetic isotope. Using a magnetic resonance imaging (MRI) scanner or electron spin resonance (ESR) scanner, MHC multimer binding T cells can then be measured and localized. In general, any conventional method for diagnostic imaging visualization can be utilized. Usually gamma and positron emitting radioisotopes are used for camera and paramagnetic isotopes for MRI.

i. Detection of T Cells Immobilized on Solid Support

In a number of applications, it may be advantageous to immobilize the T cell onto a solid or semi-solid support. Such support may be any which is suited for immobilization, separation, etc. Non-limiting examples include particles, beads, biodegradable particles, sheets, gels, filters, membranes (e.g., nylon membranes), fibers, capillaries, needles, microtiter strips, tubes, plates or wells, combs, pipette tips, microarrays, chips, slides, or indeed any solid surface material. The solid or semi-solid support may be labelled, if desired. The support may also have scattering properties or sizes, which enable discrimination among supports of the same nature, e.g., particles of different sizes or scattering properties, color or intensities.

An example of a method in which MHC multimers can be used for detection of immobilized T cells is an ELISA (Enzyme-Linked Immunosorbent Assay). ELISA is a binding assay originally used for detection of antibody-antigen interaction. Detection is based on an enzymatic reaction, and commonly used enzymes are, e.g., HRP and AP. MHC multimers can be used in ELISA-based assays for analysis of purified TCR's and T cells immobilized in wells of a microtiter plate. The bound MHC multimers can be labelled either by direct chemical coupling of, e.g., HRP or AP to the MHC multimer (e.g. the one or more multimerization domain or the MHC proteins), or e.g. by an HRP- or AP-coupled antibody or other marker molecule that binds to the MHC multimer. Detection of the enzyme-label occurs when a substrate (e.g. colorless) is added and turned into a detectable product (e.g. colored) by the HRP or AP enzyme.

The solid support may be made of, e.g., glass, silica, latex, plastic or any polymeric material. The support may also be made from a biodegradable material. Generally speaking, the nature of the support is not critical and a variety of materials may be used. The surface of support may be hydrophobic or hydrophilic. Non-magnetic polymer beads may also be applicable. Such are available from a wide range of manufactures, e.g., Dynal Particles AS, Qiagen, Amersham Biosciences, Serotec, Seradyne, Merck, Nippon Paint, Chemagen, Promega, Prolabo, Polysciences, Agowa, and Bangs Laboratories.

Another example of a suitable support is magnetic beads or particles. The term “magnetic” as used herein is intended to mean that the support is capable of having a magnetic moment imparted to it when placed in a magnetic field, and thus is displaceable under the action of that magnetic field. In other words, a support comprising magnetic beads or particles may readily be removed by magnetic aggregation, which provides a quick, simple and efficient way of separating out the beads or particles from a solution. Magnetic beads and particles may suitably be paramagnetic or superparamagnetic. Superparamagnetic beads and particles are e.g. described in EP 0 106 873. Magnetic beads and particles are available from several manufacturers, e.g., Dynal Biotech ASA (Oslo, Norway, previously Dynal AS, e.g., DYNABEADS).

### XI. Methods of Identifying T Cell Epitopes

### Chimeric MHC/T Cell Receptor System

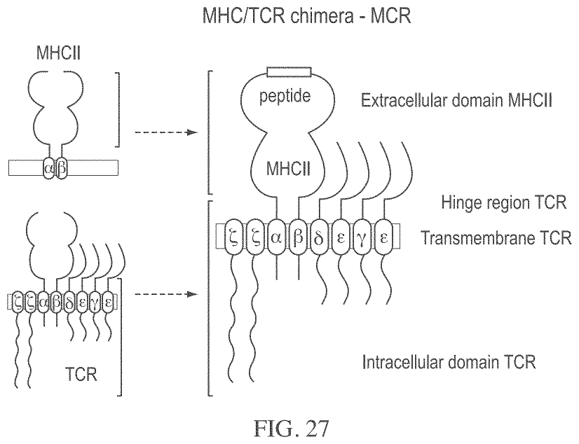

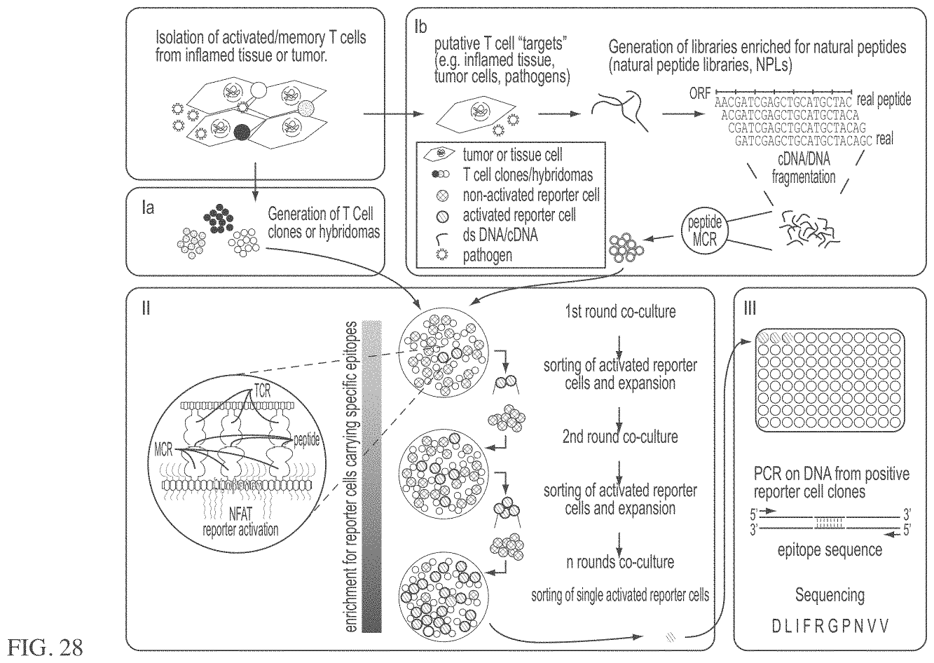

One approach for identifying SARS-CoV-2 T cell epitopes is through the use of the MCR™ system, which is described further in WO2020/142720, WO2020/142722, WO2020/142724, WO2016/097334, WO2019/197671, and WO2020/079264, as well as Kisielow et al. (2019) Nat. Immunol. 20:652-662. In the MCR™ system, chimeric MHC/TcR receptors (MCR) are expressed on mammalian cells to display epitopes to T cells, wherein epitope binding triggers expression of a reporter gene in the cell expressing the chimeric MHC/TcR receptor. Cells are sorted based on fluorescence into multiple gates and higher scores are assigned to cells that preferentially get sorted into higher-fluorescence gates. FIG. 27 shows a schematic diagram of the chimeric MHC/TcR receptor used in the MCR™ system. FIG. 28 shows a schematic diagram of the steps of the MCR™ system for identifying T cell epitopes.

A non-limiting example of use of the MCR™ system to identify SARS-CoV-2 T cell epitopes is described in detail in Example 17. The MCR™ system can also be used to validate T cell epitopes. For example, the epitopes shown in SEQ ID NOs: 271-278 were assessed by this approach.

### Screening of Peptide-MHC Tetramers

Various approaches for preparing MHC multimers (e.g., tetramers) have been described in the art, which can be applied to the identification of SARS-CoV-2 T cell epitopes. MHC multimers have been used for detection of antigen-responsive T cells since Altman et al. (Science (1996) 274:94-96) showed that tetramerization of peptide-loaded MHC class I (pMHCI) molecules provided sufficient stability to T cell receptor (TCR)-pMHC interactions, allowing detection of fluorescently-labeled MHC multimer-binding T cells using flow cytometry. However, since MHC Class I molecules are largely unstable when they are not part of a complex with peptide, pMHCI-based technologies were initially restricted by the tedious production of molecules in which each peptide required an individual folding and purification procedure (Bakker et al. (2005) Curr. Opin. Immunol. 17:428-433).

More recently, a variety of MHCI molecules with covalently linked peptides have been reported (e.g., reviewed by Goldberg et al. (2011) J Cell. Mol. Med. 15:1822-1832). Several types of pMHCI microarrays systems also have been developed, but most work has focused on optimizing the supporting surface and modifying the conditions applied during binding and/or washing. The use of these systems is also limited due to poor detection limits and low reproducibility compared to existing cytometry-based analyses. For example, a general limitation to such array-based strategies is the propensity of a given T cell to pursue all potential pMHCI interactions displayed on a given array. As a consequence, the frequency of antigen-responsive T cells in the cell preparations typically needs to be >0.1% to allow a robust readout.

MHCI multimers, and libraries thereof, have been prepared using biotinylated peptide-MHCI monomers that then associate with the biotin-binding site on streptavidin to form tetramers (see e.g., Leisner et al. (2008) PLoS One 3(2):e1678). For the creation of MHC Class I libraries, approaches have been described in which oligonucleotide barcode labels have been conjugated to the streptavidin. However, existing strategies involve complex and/or costly approaches that limit the facile production of large libraries. For example, in one approach, individual streptavidin precursors must be barcoded individually by overlap extension PCR prior to tetramerization of biotinylated peptide-HLA monomers (Zhang et al. (2018) Nature Biotech. doi:10.1038.nbt.4282). In another approach, streptavidin-conjugated dextran, which is a costly reagent, is used to create a dextramer to which both the biotinylated peptide-HLA monomers and the biotinylated barcode oligonucleotide are complexed (Bentzen et al. (2016) Nature Biotech. 34:10: 1037-1045) via the streptavidin conjugated to the dextran backbone.

Similar to the approach with pMHCI tetramers, soluble MHC class II molecules also have been used to prepare pMHCII tetramers, which have been used in the study of the antigenic specificity of CD4+ T helper cells (as reviewed in, for example, Nepom et al. (2002) Arthrit. Rheumat. 46:5-12; Vollers and Stern (2008) Immunol. 123:305-313; Cecconi et al. (2008) Cytometry 73A:1010-1018). Typically, to prepare pMHCII multimers, soluble biotinylated MHCII α/β dimers are recombinantly expressed and then tetramerized by binding to streptavidin or avidin through their biotin-binding sites. Fluorescent labeling of the streptavidin or avidin then allows for isolation of T cells that bind the pMHCII multimers by flow cytometry. With regard to antigenic peptide loading of the MHCII molecules, in one approach, a peptide is attached to the MHCII α/β dimers covalently. Some groups have generated pMHCII loaded with a covalent but cleavable “stuffer” peptide that can be exchanged with a peptide of interest under acidic conditions (Day et al., (2003) J. Clin Invest. 112(6):831-842).

In an alternative approach, “empty” MHCII α/β dimers are prepared and then loaded with soluble MHCII-binding peptides (see e.g., Novak et al. (1999) J Clin. Invest. 104:63-67; Nepom et al. (2002) Arthrit. Rheumat. 46:5-12; Macaubus et al. (2006) J. Immunol. 176:5069-5077). While this approach allows for greater diversity of peptide loading onto the MHCII α/β dimers, the ability to recombinantly express stable “empty” MHCII α/β dimers is limited, thus again hampering the preparation of large scale pMHCII multimer libraries. For example, production of “empty” MHCII α/β dimers by refolding from E. coli inclusion bodies or by insect T cell or mammalian cell expression has been reported, but with yields that are too low to support high throughput methods (reviewed in Vollers and Stern (2008) Immunology 123: 305-313).

Additionally, this disclosure provides an alternative method for preparing MHC multimers, which method provides for the high-throughput generation of libraries containing peptide-loaded MHC (pMHC) multimers containing a plurality of unique peptides in the MHC binding groove and having oligonucleotide barcode labeling to facilitate identification of library members. In the methods provided herein, all of the challenging and potentially inefficient chemistry steps for generation of pMHC multimers are done in a single bulk reaction including chromatographic cleanup and purification, followed by highly efficient peptide exchange and oligonucleotide barcoding. In particular, pMHC monomers are linked to the multimerization domain through the use of conjugation moieties on the monomers and the multimerization domain that react to form a stable chemical linkage (i.e., covalent bond) between the monomers and the multimerization domain, thereby forming a pMHC Conjugated Multimer, such as a pMHC Conjugated Tetramer. Various conjugation moieties and reactions are suitable for use in forming the Conjugated Multimers, as described herein, including use of bioorthogonal chemistry, such as click chemistry, that allow for ease and efficiency of the reactions. Moreover, when the multimerization domain is streptavidin, since the biotin-binding site is not being used for attaching the pMHC monomers, this biotin-binding site is thus available for convenient attachment of biotinylated oligonucleotide barcodes, to thereby label the multimers easily and efficiently.

The libraries of pMHC multimers provided herein are useful in a range of therapeutic, diagnostic, and research applications, essentially in any situation in which pMHC multimers are useful. For example, pMHC multimers as described herein can be used in a variety of methods, for example, to identify and isolate specific T cells in a wide array of applications. In one embodiment, the pMHC multimers are pMHC Class I multimers, which are useful for determining the antigenic specificity of CD8+ T cells (e.g., cytotoxic T cells). In another embodiment, the pMHC multimers are pMHC Class II multimers, which are useful for determining the antigenic specificity of CD4+ T cells (e.g., helper T cells).

In another embodiment, the disclosure provides a method of identifying a T cell reactive to a SARS-CoV-2 T cell epitope. The method comprises contacting a sample of T cells (e.g., a sample of T cells from a COVID-19 patient) with a MHC multimer library, for example, a MHC multimer library disclosed herein, and identifying a T cell within the sample that binds to at least one member of the MHC multimer library to thereby identify a T cell reactive with a SARS-CoV-2 T cell epitope. The MHC multimer library can be an MHC class I multimer library and the T cells are CD8+ T cells. Alternatively or in addition, the MHC multimer library is an MHC class II multimer library and the T cells are CD4+ T cells. The binding can be detected by amplifying a barcode region of the oligonucleotide barcode linked to the MHC multimer, as described herein.

In another embodiment, the disclosure provides a method of identifying a SARS-CoV-2 T cell epitope. The method comprises contacting a T cell sample with a MHC multimer library, for example, a MHC multimer library disclosed herein, identifying a T cell that binds to at least one member of the MHC multimer library, and determining the sequence of the peptide loaded onto the MHC multimer to which the T cell binds to thereby identify a SARS-CoV-2 T cell epitope. The MHC multimer library can be an MHC class I multimer library and the T cells are CD8+ T cells. Alternatively or in addition, the MHC multimer library is an MHC class II multimer library and the T cells are CD4+ T cells. A non-limiting example of the method of identifying a SARS-CoV-2 T cell epitopes using an MHC multimer-based approach is described in further detail in Example 18. In another embodiment, SARS-CoV-2 T cell epitopes are identified using an MCR system for Membrane Epitope Display, described in further detail below and exemplified in Example 17.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context. Similarly, the use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

It should be understood that the order of steps or order for performing certain actions is immaterial so long as the present invention remain operable. Moreover, two or more steps or actions may be conducted simultaneously.

### EXAMPLES

Below are examples of specific embodiments for carrying out the present invention. The examples are offered for illustrative purposes only and are not intended to limit the scope of the present invention.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al, Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3rd Ed. (Plenum Press) Vols A and B (1992).

Unless otherwise stated, all reagents and chemicals were obtained from commercial sources and used without further purification.

### Example 1: Generation of Exchangeable Peptide MHC Class I Multimers with Sortase Tag

In this example, MHC I heavy chains were expressed and complexed with 02-microglobulin (β2m) and an exchangeable peptide. The MHC heavy chain, having the amino acid sequence of SEQ ID NO: 1, contained a C-terminal sortase tag that enables post-translational coupling to Streptavidin (SAv) to form barcodable exchangeable MHC I tetramers. The SAv, having the amino acid sequence of SEQ ID NO: 3, was also expressed with a C-terminal sortase tag. A sortase enzyme having the amino acid sequence shown in SEQ ID NO: 6 was then used to conjugate a GGG-X click handle peptide to MHC I or a GGG-Y click handle peptide to SAv, where a click handle peptide contains a click moiety such as an alkyne (X) or an azide (Y), or vice versa. Subsequent chemical conjugation of MHC I to SAv by copper-assisted alkyne-azide cycloaddition or copper-free alkyne-azide cycloaddition then resulted in exchangeable-peptide-loaded MHC I tetramers.

HLA and β2m Expression and Refolding. Bacterial expression plasmids encoding _{HLA-A*02:01} linked to a Sorttag, referred to herein as HLA-A2-Sorttag (containing a C-terminal Sortase tag, 6×-His-tag) (the amino acid sequence of which is shown in SEQ ID NO: 1) and β2m (the amino acid sequence of which is shown in SEQ ID NO: 2) were generated. HLA-A2-Sorttag and β2m were expressed in E. coli in inclusion bodies. Inclusion bodies were purified and solubilized in urea buffer (20 mM MES, pH 6.0, 8 M urea, 10 mM EDTA) containing 1 mM or 0.1 mM DTT for HLA-A2-Sorttag or 0.1 mM DTT for β2m. UV-labile placeholder peptide (GILGFVFJL (SEQ ID NO: 7), where J is 3-amino-3-(2-nitro)phenylpropionic acid) was chemically synthesized. HLA-A2 was refolded with β2m and placeholder peptide according to previously described protocols (Garboczi, et al., PNAS, 89: 3429-3433, 1992; Rodenko, et al., Nat Protoc., 1:1120-32, 2006) with minor modifications. Briefly, the following components were added with stirring to pre-chilled refold buffer (100 mM Tris, pH 8.0, 0.4 M Arginine-HCl, 2 mM EDTA, 5 mM reduced glutathione, 0.5 mM oxidized glutathione, 0.2 mM PMSF) in the following order with final concentration indicated: Peptide (45 uM), β2m (3 uM) and then HLA-A2-Sorttag (1.5 uM) solubilized inclusion bodies. The refold reaction was incubated with stirring overnight at 4° C. On the next day, β2m and HLA-A2-Sorttag solubilized inclusion bodies were added to the refold reaction for 6 μM and 3 μM final concentrations, respectively. On Day 4, the refold reaction was clarified of any precipitation by centrifugation followed by filtration through a 0.2 um filter. The refold reaction was then concentrated using a Minimate Tangential Flow Filtration System (Pall) with a 10 kDa Minimate TFF Capsule (Pall) and Amicon Ultra-15 Centrifugal filters with 10,000 Da molecular weight cutoff membranes (Millipore). The concentrated refold reaction was purified by size exclusion chromatography (SEC) on a HiLoad 26/600 Superdex 200 prep grade (GE Life Sciences) pre-equilibrated in SEC buffer (20 mM HEPES pH 7.2, 150 mM NaCl). Purified fractions corresponding to the monomeric HLA-A2-Sorttag/β2m/peptide complex were pooled and concentrated. A similar procedure was followed for HLA-A2, β2m, and NLVPMVATV (SEQ ID NO: 8) peptide (abbreviated NLV) refolding and purification.

Conjugation of Click-Handle peptide to HLA-A2-Sorttag using Sortase. HLA-A2 was modified enzymatically with a Click-Handle peptide using the transpeptidase Sortase. Sortase enzyme containing 5 enhancing mutations (Chen, PNAS 2011 108(28) 11399-11404) (the amino acid sequence of which is shown in SEQ ID NO: 6) was expressed in E. coli and purified according to (Antos, Curr Protoc Protein Sci, 2009doi:10.1002/0471140864.ps1503s56). Click-Handle Peptides containing an N-terminal triglycine followed by a PEG linker (PEG₄ or PEG₅) were linked synthetically to: 1) Propargylglycine (referred to as GGG-Alkyne, Alkyne or Alk), 2) Sulfo-DBCO (referred to as GGG-DBCO or DBCO), or 3) Picolyl azide (referred to as GGG-Azide, Azide or Az). GGG-PEG₅-Alkyne peptide with C-terminal amidation was synthesized by GenScript (Piscataway, N.J.). GGG-PEG₄-Azide peptide with C-terminal amidation and GGG-PEG4-DBCO peptide were synthesized by Click Chemistry Tools (Scottsdale, Ariz.).

HLA-A2/β2m/peptide monomer (100-150 uM), Click Handle Peptide (GGG-Alkyne, GGG-DBCO, or GGG-Azide at 6-10 mM), Sortase (5-6 uM) and 10 mM CaCl2 were mixed and incubated at 4° C. for up to 4 hrs to generate an HLA-Click-Handle fusion. The reaction mixture was purified by SEC as described above to remove residual Sortase and Click-Handle-Peptide. Purified fractions corresponding to the monomeric HLA-Click-Handle/β2m/peptide complex were pooled and concentrated.

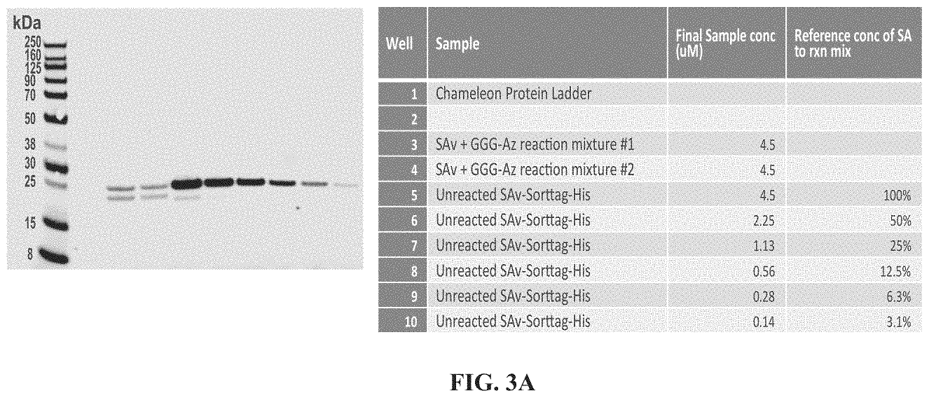

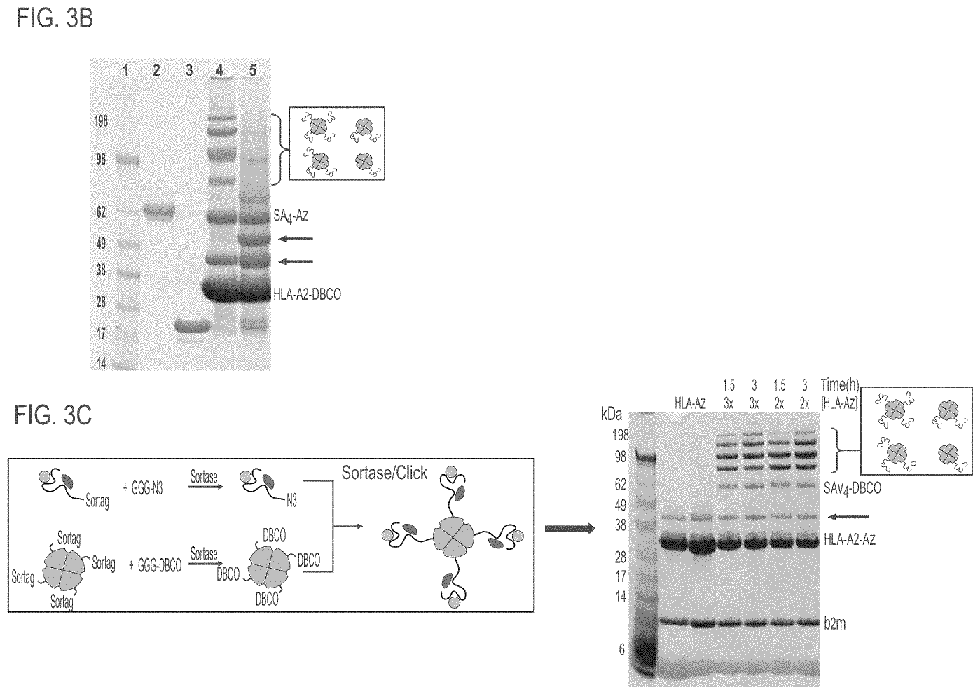

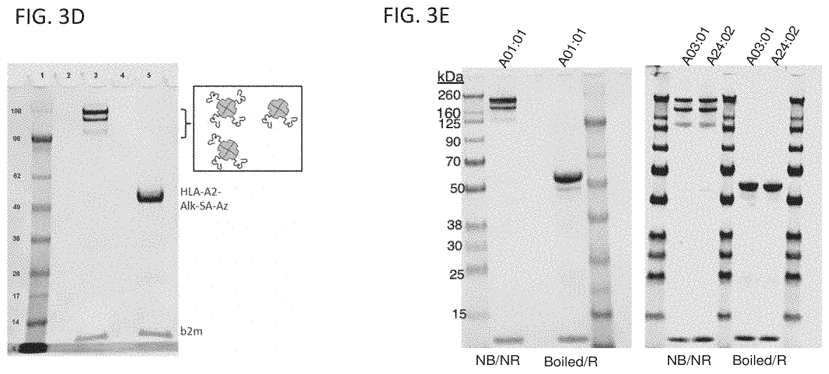

SAv expression, purification and Conjugation of Click-Handle peptide to SAv using Sortase. Full length SAv containing a C-terminal Sortase-tag and 6×HisTag (the amino acid sequence of which is shown in SEQ ID NO: 3) was expressed in BL21(DE3) cells by standard methods. SAv was purified from the soluble fraction by immobilized metal affinity chromatography (IMAC) and SEC as described above. SAv forms a native tetramer and migrates as a stable tetramer on SDS-PAGE (Waner M. J., et al., 2004, doi:10.1529/biophysj.104.047266). Purified fractions corresponding to Tetrameric SAv were pooled and concentrated. SAv-Click-Handle fusions were generated by mixing SAv (70-150 uM), Click Handle Peptide (GGG-DBCO or GGG-Azide at 3-10 mM), Sortase (6 μM) and CaCl2 (10 mM) at 4° C. for up to 4 hrs. The reaction mixture was purified by SEC to remove residual sortase and peptide, and purified fractions corresponding to the SAv-Click-Handle fusion were pooled and concentrated. The extent of conjugation to SAv was assessed by Anti-His Western blot analysis by determining the degree of loss of anti-6×His reactive band intensity relative to varying amounts of the untreated SAv sample (FIG. 3A).

Generation of clicked Peptide/MHC Class I-SAv multimers. The generation of clicked HLA-Streptavidin fusions is described herein using several different click chemistry formats (e.g., click chemistry that is described in Agard N J, Prescher J A, Bertozzi C R J. Am Chem Soc. 2004 Nov. 24; 126(46):15046-7; and Hong, V., et al., Angew Chem Int Ed Engl. 2009; 48(52): 9879-9883. doi:10.1002/anie.200905087). Because SAv forms an SDS-resistant tetramer, SDS-PAGE can be employed to monitor the extent of reaction and determine the valency of HLA on SAv (Waner M. J., et al., 2004, doi: 10.1529/biophysj.104.047266).

Formation of the clicked multimer by copper-free alkyne-azide cycloaddition was performed by mixing HLA-A2-DBCO/NLV (150 μM) with SAv-Az (50 μM with respect to SA-monomer) and incubating on ice for 3 hrs. SDS-PAGE analysis confirmed the formation of tetrameric SA with 1, 2, 3, and 4 HLA molecules attached (FIG. 3B). Side-products were observed that were attributed to undesired side-reactions of DBCO with Cysteine residues on β2m or HLA-A2 (van Geel, R, Bioconjugate Chem. 2012, 23(3): 392-398. doi.org/10.1021/bc200365k).

Covalently conjugated multimeric HLA was also prepared by mixing different ratios of HLA-A2-Az/NLV and SA-DBCO (3:1 and 2:1) at room temperature or on ice for 1.5-3.0 hr. SDS-PAGE analysis shows the formation of tetramer, trimer, dimer and monomer HLA-A2-Az-SAv-DBCO species, with a reduced level of undesirable side-reaction products compared to HLA-A2-DBCO-SAv-Az. (FIG. 3C).

An additional method to generate covalently linked HLA-A2 and SAv was through copper-assisted alkyne-azide cycloaddition. HLA-A2-Alk-SAv-Az was generated by mixing the following reaction components on ice: HLA-A2-Alk/GILGFVFM (SEQ ID NO: 7)/β2m (100-130 μM), SAv-Az (70-80 μM with respect to SA-monomer), Copper Sulfate (0.5 mM), BTTAA (2.5 mM) and Ascorbic Acid (5 mM). The reaction was monitored by SDS-PAGE and after 4 hrs the reaction mixture was purified by SEC to separate unreacted HLA, SAv, and other reaction components from purified HLA-A2-Alkyne-SAv-Az multimer. SEC fractions were analyzed by SDS-PAGE and fractions corresponding to majority tetramer/trimer species were pooled and concentrated. The peptide/HLA-A2-Alkyne-SAv-Az/□2m sample was analyzed by SDS-PAGE, which showed apparent tetramer and trimer species and very small amount of monomer for the non-boiled/non-reduced samples, while boiled and reduced gel analysis confirms the covalent linkage of HLA-A2-Alk and SAv-Az monomer at approximately 53 kDa (FIG. 3D). Mass spectrometry under denaturing conditions also confirmed the formation of an azide-alkyne fusion between HLA-A2 and SAv (not shown). HLA-Alkyne-SAv-Az formats were also generated for HLA-A01:01, HLA-A*03:01 and HLA-A*24:02, as shown in FIG. 3E.

### Example 2: Generation of Exchangeable Peptide MHC Class I Multimers with Intein Tag

In this example, MHCI heavy chain was expressed with a C-terminal N-intein tag, and streptavidin (SA) was expressed with an N-terminal C-intein tag, followed by intein-mediated conjugation to create the exchangeable-peptide-loaded MHC I tetramers. Sequences for inteins and use thereof to conjugate proteins are described further in, for example, Stevens, et al. (2016) J. Am. Chem. Soc., 138, 2162-2165, 2016; Shah et al. (2012) J. Am. Chem. Soc., 134, 11338-11341, 2012; and Vila-Perello et al. (2013) J. Am. Chem. Soc., 135, 286-292. HLA-A2 (HLA-A*02:01) was expressed in BL21(DE3) as a fusion to the Npu N-intein fragment at the C-terminus (the amino acid sequence of which is shown in SEQ ID NO: 4). Streptavidin was expressed in BL21(DE3) with an N-terminal fusion to the Npu-C-intein fragment and a C-terminal Flag tag (the amino acid sequence of which is shown in SEQ ID NO: 5). HLA-A2-N-intein and C-intein-SAv expressed in bacterial inclusion bodies. Inclusion bodies were isolated and solubilized in Urea buffer (25 mM MES, 8 M urea, 10 mM EDTA, 0.1 mM DTT, pH 6.0). HLA-A2-N-intein was refolded with β2m and UV-labile placeholder peptide (GILGFVFJL (SEQ ID NO: 7), where J is 3-amino-3-(2-nitro)phenylpropionic acid). The following components were added with stirring to pre-chilled refold buffer as described in Example 1. The refold reaction was concentrated using an Amicon Stir Cell with 10000 Da MWCO, Millipore Biomax Ultrafiltration Discs (Millipore) and Amicon Ultra-15 Centrifugal Filter Units 10,000 MWCO (Millipore). The concentrated refold reaction was purified by size exclusion chromatography (SEC) on a HiLoad 26/600 Superdex 200 prep grade (GE Life Sciences) pre-equilibrated in SEC buffer (20 mM HEPES pH 7.2, 150 mM NaCl). Purified fractions corresponding to the monomeric HLA-A2-N-intein/β2m/peptide complex were pooled and concentrated to 100-200 uM. C-intein-SAv was refolded by the same approach: briefly, urea-solubilized C-intein-SAv was injected into prechilled refold buffer and refolded according to the protocol described in Example 1, concentrated in Amicon stir cell with a 10K MWCO membrane as described and purified by size exclusion chromatography as described above. SEC purified C-intein-SAv was concentrated to 100-200 μM.

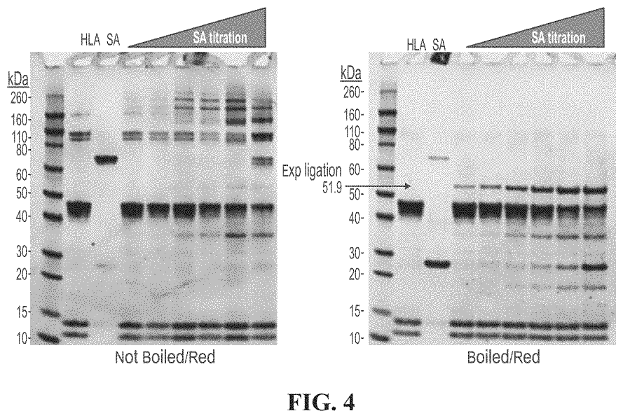

Splicing reactions between HLA-A2-N-intein/β2m/peptide complex and C-intein-SAv were carried out by adding Tris(2-carboxyethyl)phosphine hydrochloride (TCEP) to a final concentration of 0.5 mM to both the HLA-A2-int and the C-int-SA components. All components were kept on ice. To favor formation of tetrameric species, streptavidin was added in 5 increments over a 16 hrs period until an equimolar amount to HLA-A2-intein was achieved. SDS-PAGE analysis of the reaction under non-reducing/non-boiled conditions shows the formation of higher MW species, while the boiled/reduced samples showed a species at approximately 52 kDa, consistent with the expected size for an HLA-A2-SAv fusion (FIG. 4).

### Example 3: Production of Exchangeable MHCI Tetramers via Biotinylation and Coupling to Streptavidin

HLA-A*02 heavy chain with a C-terminal Avitag was expressed in E. coli in inclusion bodies. The amino acid sequence of the Avitag is shown in SEQ ID NO: 161. Purified inclusion bodies were solubilized in urea and refolded with β-2-microglobulin and the peptide NLVPMVATV (SEQ ID NO:8) or the conditional ligand GILGFVFJL (SEQ ID NO:7), where J is a 2-nitrophenylamino acid residue, according to literature methods (Rodenko et. al., (2006) Nat. Protoc. 1(3):1120-32). SEC-purified MHC monomers comprising the heavy chain, β-2-microglobulin and peptide were then biotinylated using biotin ligase and then SEC-purified once again. Streptavidin was added to biotinylated MHC monomers in 10 separate aliquots to achieve a slight molar excess of biotin sites over MHC monomers. Peptide exchanges (as described in Example 4) are executed on either the biotin-mediated streptavidin tetramer or on the biotinylated HLA monomer. In the case of the latter, monomers are tetramerized with streptavidin after exchange.

### Example 4: Peptide Exchange Via Dipeptide or UV Exchange

HLA-A*02:01-Alk-SAv-Az Conjugated Tetramers produced as described above in Example 1, as well as biotin-mediated HLA-A*02 tetramers produced as in Example 3, were exchanged by either of two methods. For dipeptide exchange, 5 μM MHC tetramers loaded with a place-holder peptide (e.g., GILGFVFJL (SEQ ID NO:7)) were incubated with a 30-fold excess of NLVPMVATV (SEQ ID NO:8) peptide in the presence or absence of 10 mM GM dipeptide for 3 hours at room temperature (Saini et al. (2006) PNAS 112(1):202-206). For UV-exchange, 2-10 μM MHC monomers or 0.5-2.5 μM MHC tetramers loaded with a place-holder peptide (GILGFVFJL (SEQ ID NO:7)) were incubated with a 30-100-fold molar excess of NLVPMVATV (SEQ ID NO:8) (or other peptide) for 1 hour on ice, followed by 30 minutes exposure to 365 nm UV light from a lamp held 2-5 cm from the sample. The UV exposure was sometimes followed by 30 minutes incubation at 30° C. to allow complete exchange. Efficiency of peptide exchange was monitored by Differential Scanning Fluorimetry (DSF), ELISA and cell staining/flow cytometry.

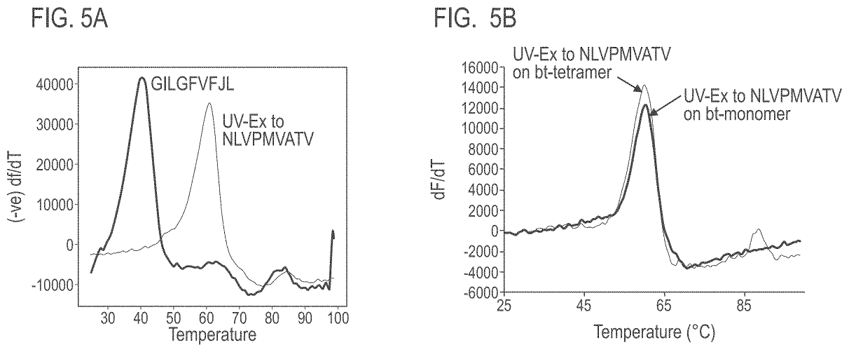

For DSF, 0.25 mg/ml HLA-A*02 tetramers were mixed with an equal volume of 20× Sypro Orange (Invitrogen S6650), and subjected to a 0.05° C./s ramp from 25° C. to 99° C. in a qPCR instrument (e.g., Applied Biosystems Quant Studio 3). A peak in the first derivative of the melt curve indicates the Tm of the pMHC. As seen in FIG. 5A, the Tm of HLA-A*02:01-Alk-SAv-Az Conjugated Tetramers produced as in Example 1, shifts from 40° C. to 61° C. upon UV-exchange from the placeholder GILGFVFJL (SEQ ID NO:7) peptide to NLVPMVATV (SEQ ID NO:8). The Tm after UV exchange is identical to that observed for NLVPMVATV (SEQ ID NO:8) exchanged into biotinylated monomers followed by tetramerization (industry standard) or exchanged directly into biotin-mediated tetramers (FIG. 5B). These data confirm that multimeric state has no impact on the efficiency of UV-exchange, and that such Conjugated Tetramers described herein have the same stability as the industry standard pMHC.

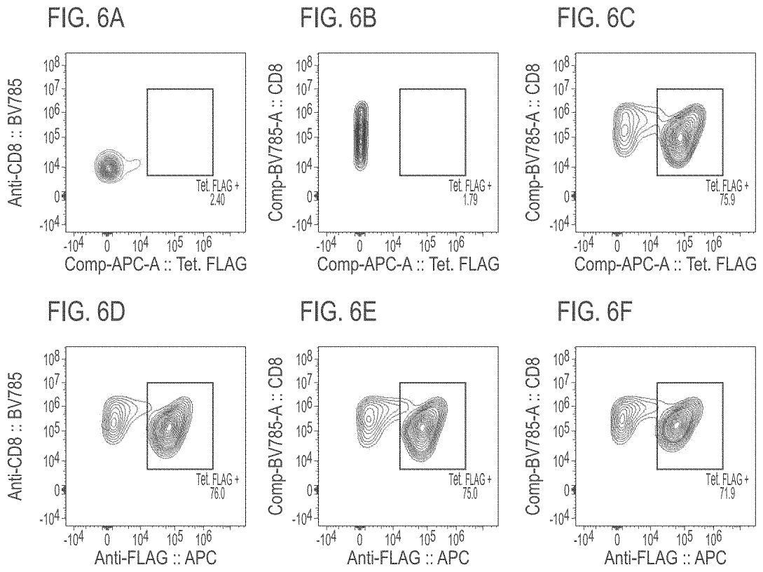

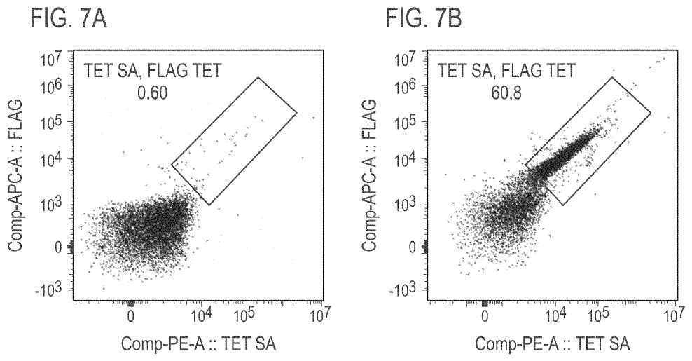

For flow cytometry, 10⁵ donor T cells that had been expanded with NLVPMVATV (SEQ ID NO:8) (or other peptide) were stained with pMHC tetramers produced as above. All pMHC were diluted in PBS plus 10% FBS, and stained with anti-CD8-BV785, and anti-Flag-APC or anti-streptavidin-PE (Biolegend) was used as secondary. As seen in FIG. 6A-F, either dipeptide exchange or UV exchange executed on the biotin-mediated tetrameric form produces HLA-A*02 tetramers that display the same level of binding to expanded T cells as those produced by industry-standard methods (tetramerization post refolding or post UV exchange of biotinylated monomers). FIG. 7 illustrates the high affinity binding of HLA-A*02:01-Alk-SAv-Az Conjugated Tetramers that were UV-exchanged to the NLVPMVATV (SEQ ID NO:8) peptide to expanded T cells.

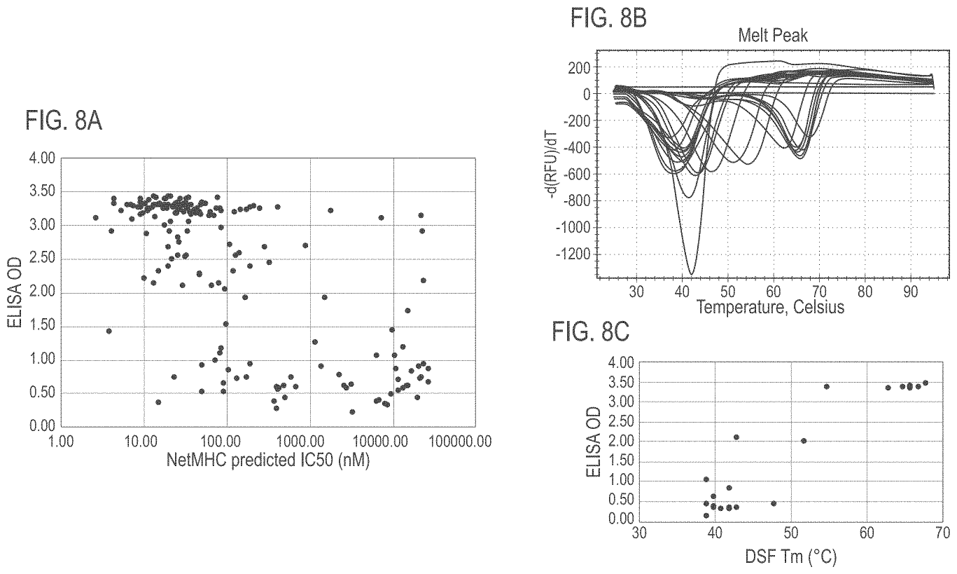

ELISA were also used to monitor exchange on tetramers and is another indicator of pMHC stability. Plates were first coated with anti-streptavidin antibody, followed by capture of tetramers in Citrate-phosphate buffer at pH 5.4, and then read out using HRP-conjugated anti-β2-microglobulin (Biolegend). As seen in FIG. 8A, a panel of NLVPMVATV (SEQ ID NO:8) mutant peptides can be effectively UV-exchanged into HLA-A*02:01-Alk-SAv-Az Conjugated Tetramers, generating a span of ELISA signals. A smaller panel of similar peptides UV-exchanged into biotin-mediated HLA-A*02 tetramers also generated a range of ELISA signals (FIG. 8C), which positively correlated with Tm measured by DSF (FIG. 8B).

### Example 5: Conjugated Tetramers Produced with HLA-A*01:01

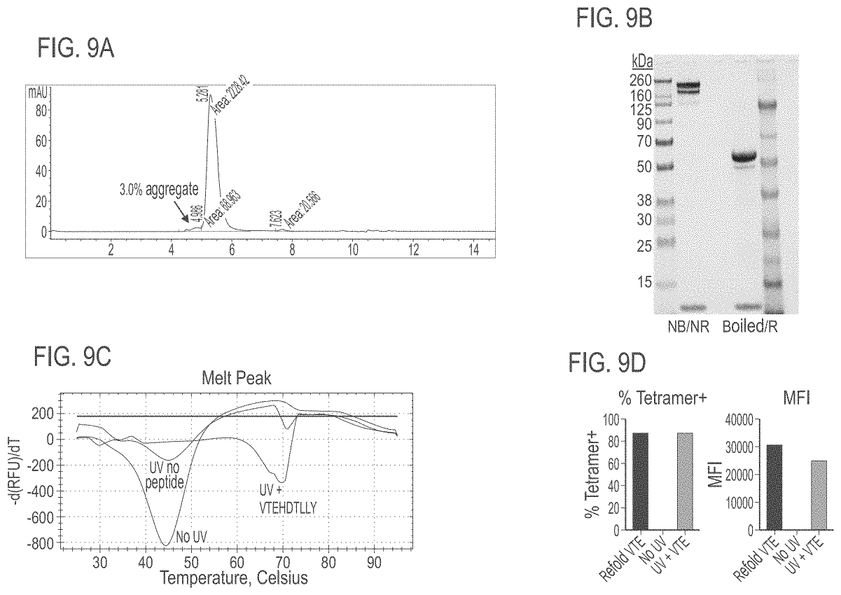

HLA-A*01:01 monomers refolded with the peptide STAPGJLEY (SEQ ID NO: 16) were used for construction of HLA-A*01:01-Alk-SAv-Az Conjugated Tetramers and characterized as described in Example 1. As seen in FIG. 9A and FIG. 9B, HLA-A*01:01-Alk-SAv-Az Conjugated Tetramers were highly multimeric with a low percentage of aggregates (3%). UV treatment in the presence of a cognate peptide VTEHDTLLY (SEQ ID NO: 10) resulted in a characteristic shift in the DSF melt curve, indicating effective peptide exchange (FIG. 9C). The exchanged HLA-A*01:01-Alk-SAv-Az conjugated tetramers bound strongly to PBMCs expanded with the VTEHDTLLY peptide (SEQ ID NO: 10), similar to HLA-A*01:01 refolded with VTEHDTLLY peptide (SEQ ID NO: 10) that was conjugated to streptavidin via biotin (FIG. 9D). As expected, no binding was observed in the absence of UV exchange.

### Example 6: Conjugated Tetramers Produced with HLA-A*24:02

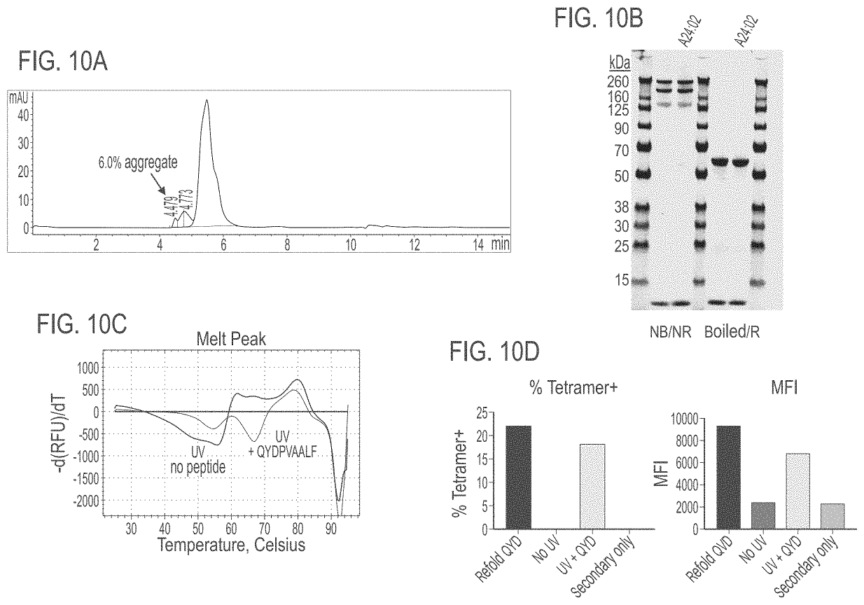

HLA-A*24:02 monomers refolded with the peptide VYGJVRACL (SEQ ID NO: 11) were used for construction of HLA-A*24:02-Alk-SAv-Az Conjugated Tetramers and characterized as described in Example 1. As seen in FIG. 10A and FIG. 10B, HLA-A*24:02-Alk-SAv-Az Conjugated Tetramers were highly multimeric with a low percentage of aggregates (6%). UV treatment in the presence of a cognate peptide QYDPVAALF (SEQ ID NO: 12) resulted in a characteristic shift in the DSF melt curve, indicating effective peptide exchange (FIG. 10C). The exchanged HLA-A*24:02-Alk-SAv-Az conjugated tetramers bound strongly to PBMCs expanded with the QYDPVAALF peptide (SEQ ID NO: 12), similar to HLA-A*24:02 refolded with QYDPVAALF peptide (SEQ ID NO: 12 that was conjugated to streptavidin via biotin (FIG. 10D). As expected, no binding was observed in the absence of UV exchange.

### Example 7: Conjugated Tetramers Produced with HLA-B*07:02

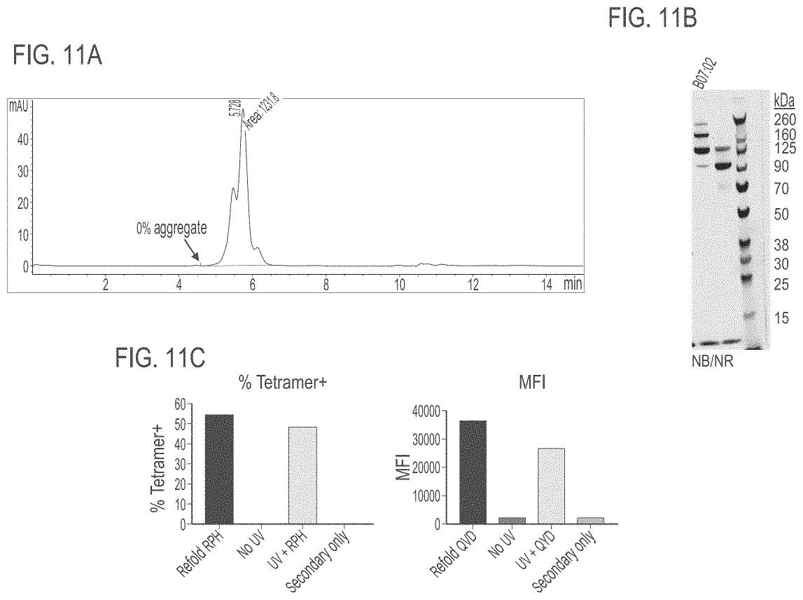

HLA-B*07:02 monomers refolded with the peptide AARGJTLAM (SEQ ID NO: 14) were used for construction of HLA-B*07:02-Alk-SAv-Az Conjugated Tetramers and QC′d as described in Example 1 above. As seen in FIG. 11A and FIG. 11B, HLA-B*07:02-Alk-SAv-Az Conjugated Tetramers were multimeric with no detectable aggregates. After UV treatment in the presence of a cognate peptide RPHERNGFTVL (SEQ ID NO: 13), exchanged HLA-B*07:02-Alk-SAv-Az conjugated tetramers bound strongly to PBMCs expanded with the RPHERNGFTVL peptide (SEQ ID NO: 13), similar to HLA-B*07:02 refolded with RPHERNGFTVL peptide (SEQ ID NO: 13) that was conjugated to streptavidin via biotin (FIG. 11C). As expected, no binding was observed in the absence of UV exchange.

### Example 8: Barcoding and Pooling of UV-Exchanged Tetramers

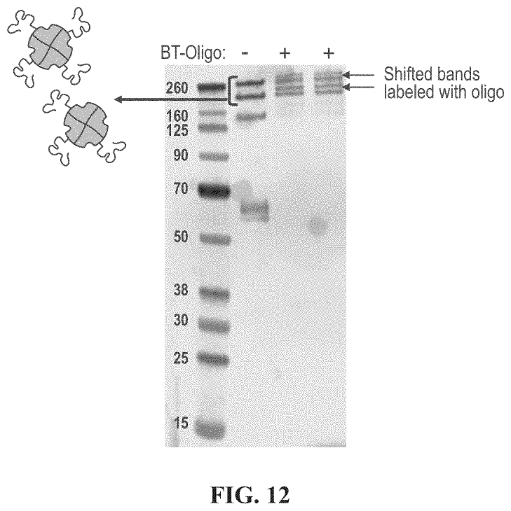

Exchanged HLA-A*02:01-Alk-SAv-Az Conjugated Tetramers were easily labeled with an identifying oligonucleotide tag (barcode) due to the fact that the biotin binding sites on streptavidin were empty. 5′ biotinylated oligonucleotides were added at a 2:1 oligo:tetramer molar ratio, and incubated for 30 mins at 4° C., followed by quench with biotin at 400:1 biotin:tetramer molar ratio for 30 min at 4° C. Barcoding was confirmed by electrophoresis on a 4-12% bis-Tris gel, followed by blotting to nitrocellulose and staining with anti-Flag antibody (Invitrogen #MA1-91878-D800). As seen in FIG. 12, a gel shift relative to the tetramer starting material indicates proper labeling with the oligonucleotide barcode.

### Example 9: Single Cell Sequencing with Pooled Barcoded UV-Exchanged Tetramers

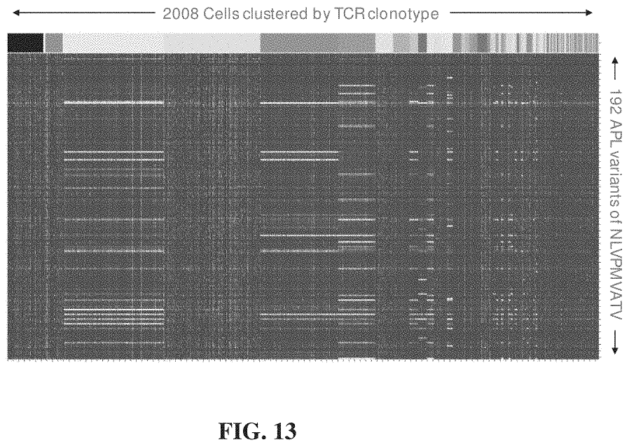

After confirmation of oligonucleotide labeling, individual HLA-A*02:01-Alk-SAv-Az Conjugated Tetramer samples that were UV-exchanged for 192 different APL variants of NLVPMVATV (SEQ ID NO:8) were pooled, stained on NLVPMVATV (SEQ ID NO: 8)-expanded T cells, and subjected to single cell sequencing. The analyzed results are shown in a heatmap in FIG. 13, indicating clonotype-specific binding of a subset of APL variants.

### Example 10: Production of a Porous Hydrogels for High Throughput Production of Barcoded UV-Exchanged Tetramer Pools

Hydrogel beads were produced by mixing acrylamide monomer units and bis-acrylamide crosslinker units at a variety of relative concentrations along with a mixture of acrylated oligonucleotide primers, encapsulating in droplets using a microfluidic drop-maker, and incubating the mixture until crosslinking was complete. In this Example, the pre-crosslinked aqueous mix included 0.75% bis-acrylamide, 3% acrylamide, 25 μM 5′-acrylated forward primer, 0.5% ammonium persulfate, in 10% TEBST (Tris-EDTA-buffered saline plus Tween-20). All reagents of the aqueous mixture were combined and stirred. The mixture was supplemented with 1.5% TEMED and 1% of 008-FluoroSurfactant, encapsulated in droplets, incubated at room temperature for 1 hour, and then transferred into an oven at 60° C. for overnight incubation, thus forming the hydrogels. The hydrogel beads were washed once with 20% 1H,1H,2H,2H-perfluoro-1-octanol (PFO), then washed three times with TEBST, and then washed three times with low TE (1 mM Tris-Cl pH 7.5, 0.1 mM EDTA). Hydrogel beads were stored in TEBST at 4° C. until use.

### Example 11: Single Template PCR to Generate Peptide-Encoding Amplicons

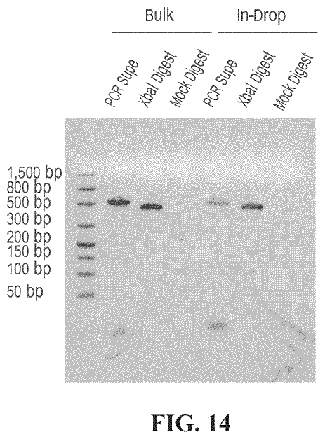

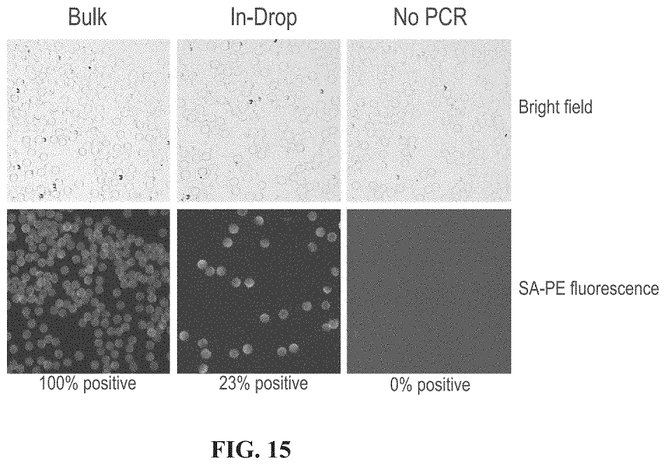

Linear DNA templates encoding a SUMO domain-peptide fusion were PCR-amplified onto hydrogel beads in drops under single template conditions, where each drop gets at most a single DNA template. 1.4 mL hydrogel beads produced in Example 10 were mixed together with PCR components as follows in a 2 mL reaction volume: 400 μL Q5 reaction buffer (New England Biolabs), 40 μL 10 mM dNTP, 40 μL 1 uM μforward primer, 40 μL 25 μM 5′-biotinylated reverse primer, 40 μL 0.1 pg/μL linear DNA template (or mix of templates), 8 μL 20% IGEPAL, and 20 μL Q5 DNA polymerase (New England Biolabs). The mixture was encapsulated in drops and subjected to 35 cycles of PCR. After drop lysis by addition of an equal volume of 100% perfluorooctanol (PFO), hydrogels were washed with 10 volumes of low TE five times. Aliquots (10 μL) of hydrogel beads were digested with Xbal, which cuts within the amplicon, for 1 hour at 37° C. and run on a 1.2% agarose gel along with PCR supernatant to quantify yield and quality of amplicons (FIG. 14). That single template conditions were in effect was demonstrated by labeling hydrogels with streptavidin-PE, where only 23% of drop-amplified hydrogels were stained, compared to 100% of bulk-amplified hydrogels (FIG. 15).

### Example 12: Loading of Barcodable Exchange-Ready Conjugated Tetramers onto Hydrogels

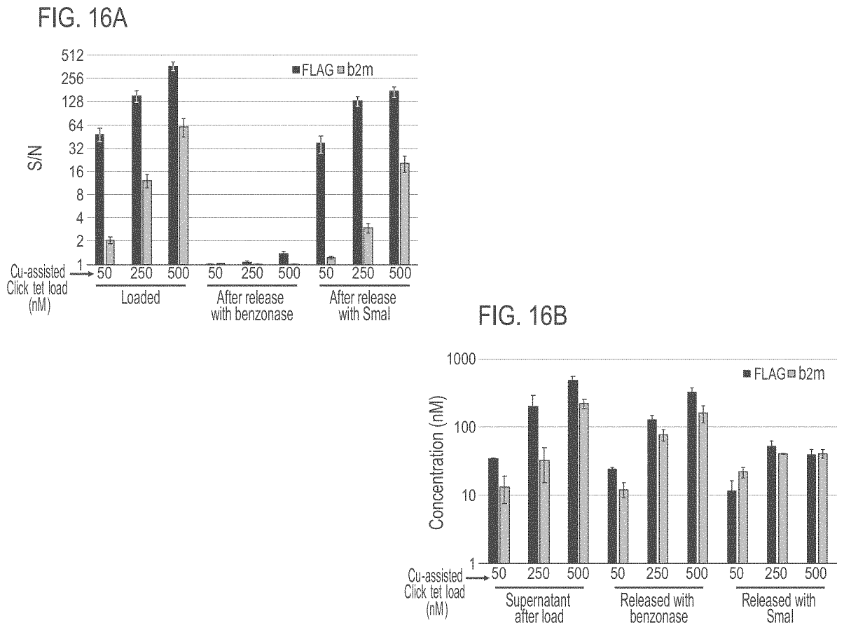

PCR-amplified hydrogels were mixed 1:1 by volume with 50 to 500 nM HLA-A*02:01-Alk-SAv-Az Conjugated Tetramers loaded with the UV-labile peptide (e.g., GILGFVFJL (SEQ ID NO:7), protected from ambient light, and incubated on ice for 2 hours. Loading of HLA-A*02:01-Alk-SAv-Az Conjugated Tetramers was confirmed by washing and staining with anti-Flag-APC or anti-β2M-Alexa488 as seen in FIG. 16A. The quantity of tetramers loaded was quantified by releasing with benzonase or SmaI, which cuts within the amplicon, followed by ELISA with anti-streptavidin capture and either anti-Flag-HRP or anti-β2M-HRP detection, as shown in FIG. 16B.

### Example 13: In-Drop In Vitro Transcription/Translation (IVTT) of Peptide and UV Exchange into Loaded Tetramers

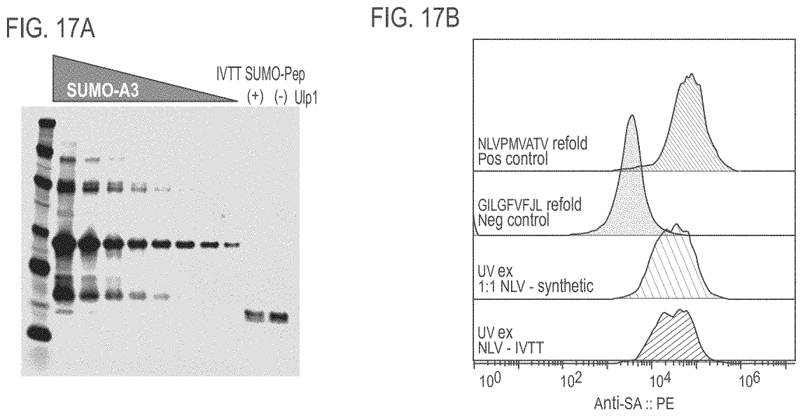

120 μL of hydrogel beads are co-encapsulated in drops with 240 μL of IVTT master mix, including 120 μL PURExpress solution A (New England Biolabs), 90 μL PURExpress solution B (NEB), 6 μL RNAse OUT (Invitrogen), and 1.2U Ulp1 protease (Invitrogen). Drops were incubated at 30° C. for 4 hours, without shaking, then UV-exchanged by 30-minute exposure to 365 nm UV light. The UV exposure was followed by 30 minutes incubation at 30° C. to allow complete exchange. D-Biotin was added to the IVTT reactions to a final concentration of 500 μM prior to breaking drops, which was then accomplished by addition of an equal volume of 100% PFO. Hydrogel beads were washed five times with 10 volumes of PBS plus 2% BSA. Sufficient peptide can be produced from a PCR amplicon to generate functional exchanged tetramers, as shown in FIGS. 17A and 17B.

### Example 14: Release and Analysis Of Single Chain Multimeric Peptide-MHC

UV-exchanged pMHC were released from washed hydrogels by digestion with SmaI, which cuts within the amplicon upstream of the peptide-encoding region, such that the tetramers were released with a self-identifying oligonucleotide tag (barcode) as indicated in FIG. 16B and summarized in FIG. 18.

### Example 15: Generation of Conjugated Peptide/MHC Class II-SAv Multimers

Conjugation of Click-Handle peptide to MHC II-Sorttag using Sortase. The sequences of MHC II α- and β-chains were recombinantly expressed as follows: the α-chain extracellular domain sequence was expressed with a C-terminal sortase tag that enables post-translational coupling to Streptavidin (SAv) to form barcodable exchangeable MHC II multimers The α-chain also contained a Myc tag for diagnostic purposes. The amino acid sequence of the α-chain extracellular domain with sortag and Myc tag is shown in SEQ ID NO: 191. The β-chain was recombinantly expressed with an N-terminal low-affinity placeholder peptide (CLIP peptide, the sequence of which is shown in SEQ ID NO:189) followed by a flexible linker, the β-chain extracellular domain and a Histidine purification tag. The amino acid sequence of the β-chain extracellular domain with placeholder peptide, flexible linker and His Tag is shown in SEQ ID NO:192. The flexible linker contained a cleavage site that permitted breaking the connection between the peptide and the 0-chain by a specific protease, thus facilitating subsequent peptide exchange. MHCII molecules with a covalent placeholder peptide loaded therein are referred to herein as p*MHCII.

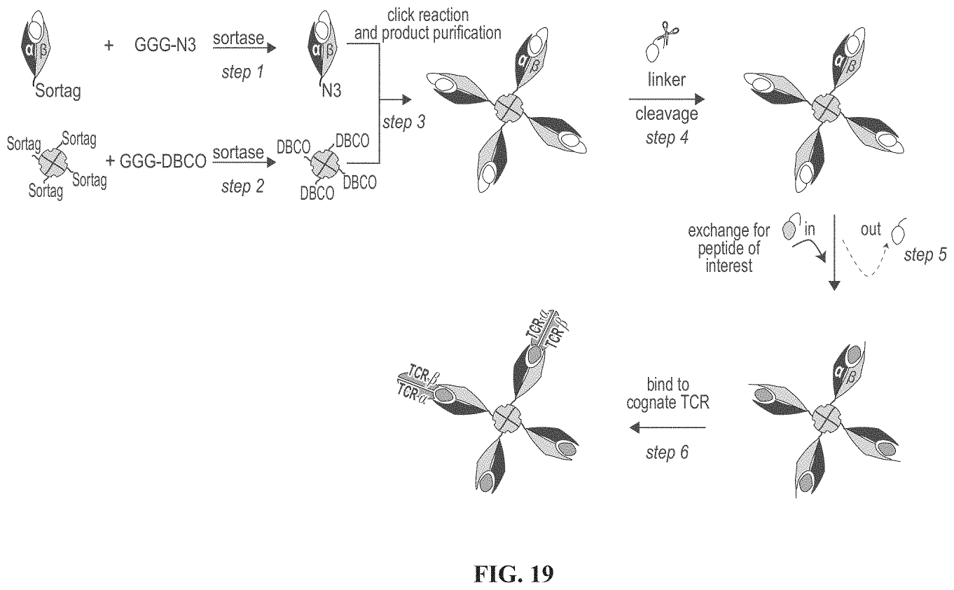

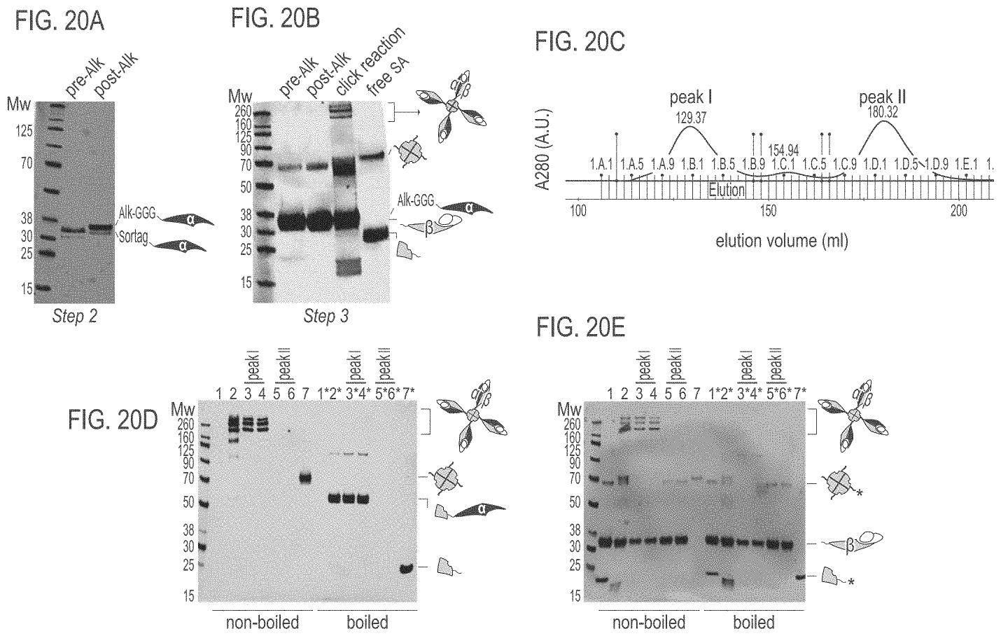

p*MHCII α- and β-chains were co-expressed in CHO cells and secreted into the expression medium as a stable heterodimer. Following CHO expression, p*MHCII was purified by immobilized metal ion affinity chromatography and size exclusion chromatography (SEC). Sortase enzyme was then used to conjugate a GGG-X peptide to the p*MHCII α-chain (FIG. 19, step 1) where X can be an azide, an alkyne, or any clickable chemical moiety. To execute the chemical conjugation reaction p*MHCII (30-50 μM), Click Handle Peptide (GGG-Alkyne, GGG-DBCO, or GGG-Azide at 6-10 mM), Sortase (5-6 μM) and 10 mM CaCl₂) were mixed and incubated at 4° C. for up to 2 hours to generate an p*MHCII-Click-Handle fusion. The reaction mixture was purified by SEC to remove residual Sortase and Click-Handle-Peptide. Purified fractions corresponding to p*MHCII-Click-Handle fusion were pooled and concentrated. Click Handle addition caused a shift in the size of the conjugated protein, validating a successful sortase-mediated ligation (FIG. 20A).

The generation of conjugated p*MHCII-SAv multimers. The expression, purification and conjugation of Click-Handle p*MHCII to SAv using Sortase is illustrated in FIG. 19, step 2, and was carried out essentially as described in Example 1 for MHCI multimers. Copper-assisted alkyne-azide cycloaddition was used to generate covalently linked p*MHCII and SAv (FIG. 19, step 3). p*MHCII-Alk-SAv-Az was generated by mixing the following reaction components on ice: MHC II-Alk (50 uM), SAv-Az (25 uM with respect to SA-monomer), Copper Sulfate (0.5 mM), BTTAA (2.5 mM) and Ascorbic Acid (5 mM). The reaction was monitored by SDS-PAGE (FIG. 20B) and after 4 hours the reaction mixture was purified by SEC to separate unreacted HLA, SAv, and other reaction components from purified p*MHCII-Alk-SAv-Az multimer (FIG. 20C). The SAv and the β-chain contained FLAG and His tags, respectively, enabling to distinguish fractions corresponding to multimer species (FIGS. 20D and 20E). The multimer fractions showed apparent tetramer and trimer species. More importantly, free SAv species were not observed in boiled samples taken from multimer fractions under SDS-PAGE and western blot analysis (FIG. 20D). This indicates that the dominant species is a tetramer, in which each SAv subunit is covalently linked to an p*MHCII subunit.

### Example 16: pMHC II Multimers Are Exchangeable and Bind Cognate Epitope-Specific TCR

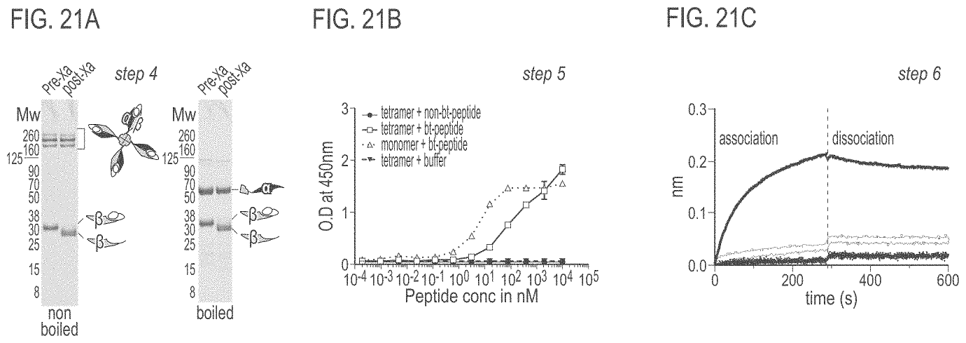

Linker digestion and peptide exchange. p*MHCII-Alk-SAv-Az multimer (henceforth—p*MHCII-SAv) was digested by Factor Xa (NEB) at a ratio of 5:1 (w/w) over night at 4° C. in the presence of 1 mM CaCl₂) (FIG. 19, step 4). Then the protease was irreversibly inactivated by the addition of 1,5-Dansyl-Glu-Gly-Arg Chloromethyl Ketone inhibitor according to the manufacturer's recommendations (Sigma-Aldrich). Digested samples migrated faster than non-digested samples indicating the removal of the freshly cleaved peptide under SDS-PAGE denaturing conditions (FIG. 21A).

To test whether cleaved p*MHCII-SAv (henceforth—p↓MHCII-SAv) bound an exchanged peptide, an ELISA binding assay was performed. In this assay, a biotinylated peptide epitope from Influenza A virus (Hemagglutinin, HA, the amino acid sequence of which is shown in SEQ ID NO:193) was loaded while the cleaved placeholder peptide was removed under mild acidic pH conditions (FIG. 19, step 5). The level of exchange was then determined by monitoring the binding of streptavidin-HRP to the newly swapped biotinylated peptide. Free biotin binding sites on the streptavidin molecules were blocked with an excess of free biotin prior to the exchange reaction. This step ensured that any detected biotinylated peptide can only be bound to the peptide-binding pocket. The exchange-buffer composition was as follows: 100 mM sodium citrate pH 5.5, 50 mM sodium Chloride, 1% octyl glucoside (v/v), lx of SIGMAFAST protease inhibitor cocktail (Sigma-Aldrich) and 0.1 mM DTT. 150 μL of peptide exchange reactions were prepared in a 96-well plate where each well consists of: 1× exchange buffer, 30 nM p↓MHCII-SAv and 5-fold serial dilutions of either HA-biotinylated peptide, HA-non-biotinylated peptide or buffer. Incubation of 6 nM of p↓MHCII monomer with 5-fold serial dilutions of HA-biotinylated peptide was included as a positive control. The exchange reaction was stopped after an over-night incubation at 37° C. by neutralizing the acidic pH with the addition of 1:15 (v/v) of 1 M Tris-HCl, pH 10. Using a 96 channel benchtop pipettor, 100 μL from each well were transferred to an ELISA plate that was pre-coated with (100 ng/well) L243 conformational sensitive antibody (Abcam), washed (3×PBS-T) and blocked with PBS-T supplemented with 2% (v/v) BSA. Following 1 hr incubation at RT, the plate was washed (3×PBS-T), incubated with SA-HRP for 30 mins in the dark, washed again (3×PBS-T) and developed using an HRP substrate and stop solution. A positive correlation between peptide concentrations and the levels of SA-HRP binding was observed for both monomeric p↓MHCII and p↓MHCII-SAv (FIG. 21B). This indicates that both species exchanged the placeholder peptide for biotinylated-HA peptide. Incubation with either non-biotinylated peptide or buffer did not yield a detectable signal implying that binding of the biotinylated epitope was specific. In contrast to monomeric p↓MHCII, the curve for p↓MHCII-SAv was shifted to the right and did not reach saturation at higher peptide concentrations. The multimer is at least 4-fold bigger in size, which might occlude binding to the capturing antibody and/or to the SA-HRP readout probe.

Binding of exchanged p↓MHCII-SAv to soluble TCR. F11, an HA-peptide epitope specific soluble TCR, was fused to an FC domain and produced as described in Wagner et al. (2019) J Biol Chem., 294:5790-5804 (FIG. 19, step 6). Briefly, DNA encoding the F11 extracellular alpha- and beta-chains was cloned into pDT5 plasmids downstream of a mouse IgGk chain leader sequence. The human TCR constant domains contained an additional inter-chain disulfide bond. The C-alpha domain was followed by the upper hinge sequence of human IgG1 (VEPKSC; SEQ ID NO: 270), the core and lower hinge, and then the Fc domain. The native IgG1 light-chain cysteine was inserted at the C-terminus of C-beta to pair with the upper hinge cysteine and further stabilize the TCR heterodimerization. Additional modifications included the removal of N-linked glycosylation sites. Plasmids encoding alpha-Fc and beta domains were expressed in Expi-CHO cells by transient transfection, and the product was purified from clarified supernatants by protein A affinity chromatography.

The exchange reaction was performed as described above in Example 1 with two differences: a single tube was used instead of a 96-well plate and the protein concentrations varied. 1.75 μM of p↓MHCII-SAv were incubated with 100 μM of HA peptide in the presence of exchange buffer. After the reaction was stopped and kept on ice, a Bio-layer interferometry (BLI) assay was carried out using an Octet RED96 instrument (ForteBio) at 30 C in BLI buffer (PBS+0.02% Tween20, 0.1% BSA, 0.05% sodium azide). F11 TCR was loaded onto Anti-hIgG Fc Capture Biosensors (Molecular Devices) to 0.6 nm loading signal. After washing with BLI buffer, biosensors were transferred to wells containing either 14 nM of exchanged p↓MHCII-SAv, 125 nM of non-exchanged p*MHCII-SAv or BLI buffer to measure association kinetics (FIG. 21C). To measure dissociation kinetics, biosensors were transferred back to BLI buffer devoid of multimers. A significant increase in BLI-response signal was observed for HA-exchanged p↓MHCII-SAv suggesting a strong association with F11 TCR (FIG. 21C). In contrast, non-exchanged p*MHCII-SAv showed very little association indicating that the interaction between F11-TCR and an HA displaying multimer is specific. No association was observed when the biosensors were dipped into BLI buffer. HA-exchanged NMHCII-SAv exhibited very slight dissociation from F11-TCR. This result indicates a tight TCR-MHC II binding which is characteristic of high-avidity multimer interaction.

### Example 17: SARS-CoV-2 T Cell Epitope Identification by Membrane Epitope Display

In this example, the MCR™ system was used to identify SARS-CoV-2 T cell epitopes using T cells from SARS-CoV-2 patients. In brief, the MCR™ system uses chimeric MHC/TcR receptors (MCR) expressed on mammalian cells to display epitopes to T cells, wherein epitope binding triggers expression of a reporter gene in the cell expressing the chimeric MHC/TcR receptor. Cells are sorted based on fluorescence into multiple gates and higher scores are assigned to cells that preferentially get sorted into higher-fluorescence gates. This technology is described further in Kisielow et al. (2019) Nat. Immunol. 20:652-662. Additionally, FIG. 27 shows a schematic diagram of the chimeric MHC/TcR receptor used in the MCR™ system and FIG. 28 shows a schematic diagram of the steps of the MCR™ system for identifying T cell epitopes.

Four different HLA Class II molecules were examined: DRB 1*07:01, 1*04:04, 1*15:01, 1*10:01. Peptides from several different SARS-CoV-2 antigenic peptides were examined, including the Spike Protein (S), the Nucleocapsid Protein (NP) and ORF3a. Peptides were specifically presented by one HLA allele, but peptides may also be presented by more than one allele, e.g., all four HLA alleles in this experiment.

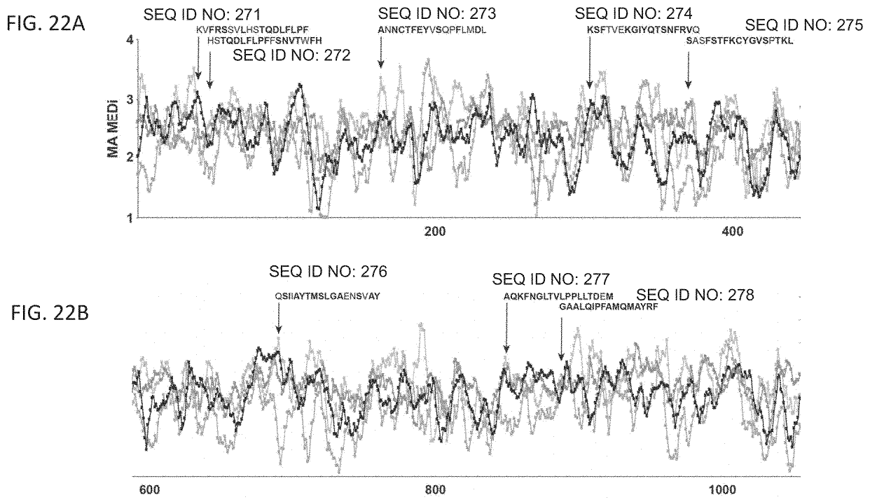

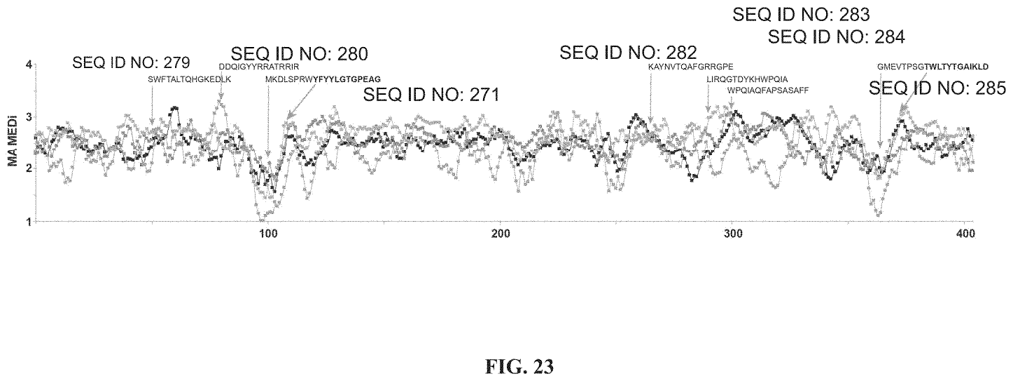

Representative results for the S protein are shown in FIG. 22A-22B, with FIG. 22A showing five different T cell epitopes (the sequences of which are shown in SEQ ID NOs: 271-275) and FIG. 22B showing three different T cell epitopes (the sequences of which are shown in SEQ ID NOs: 276-278). Representative results for the NP protein are shown in FIG. 23, which shows seven different T cell epitopes (the sequences of which are shown in SEQ ID NOs: 276-278). T cell epitopes were also identified in the ORF3a peptides.

Overall, the results indicated that use of the MCR™ system was an effective approach for identifying T cell epitopes from a variety of SARS-CoV-2 antigens.

### Example 18: SARS-CoV-2 T Cell Epitope Identification Using Peptide-MHC Tetramers

In this Example, MHC tetramers loaded with SARS-CoV-2 antigenic peptides were used to identify T cell epitopes using T cells from SARS-CoV-2 patients. MHC tetramers such as those described herein can be used. Additionally or alternatively, other MHC tetramer approaches described in the art can be used, such as described in Altman et al. (1996) Science 274:94-96, Bakker et al. (2005) Curr. Opin. Immunol. 17:428-433, 2005), Goldberg et al., J Cell. Mol. Med. (2011) 15:1822-1832, Leisner et al. (2008) PLoS One 3(2):e1678, Zhang et al., Nature Biotech. (2018); doi:10.1038.nbt.4282, Nepom et al. (2002) Arthrit. Rheumat. 46:5-12; Vollers and Stern (2008) Immunol. 123:305-313; Cecconi et al. (2008) Cytometry 73A:1010-1018, Day et al., (2003) J Clin Invest. 112(6):831-842, Novak et al. (1999) J. Clin. Invest. 104:63-67; and Macaubus et al. (2006) J. Immunol. 176:5069-5077.

A 596 member SARS-CoV-2 peptide library for display on HLA-A*02:01 tetramers was prepared. The library comprised 9mers of the SARS-CoV-2 full proteome with IC₅₀ less than 500 nM, as well as close match sequences between SARS-CoV and SARS-CoV-2, published predicted peptides that were above IC₅₀ less than 500 nM for HLA-A*02:01, epitopes from common cold coronaviruses with evidence of positive T cell assays, immunodominant epitopes to SARS-CoV (IEDB), peptides with predicted glycosylation sites, peptides containing identified mutations in the spike protein, platform control epitopes against which control cells have been expanded, and CEF (CMV, EBV, influenza) epitope controls.

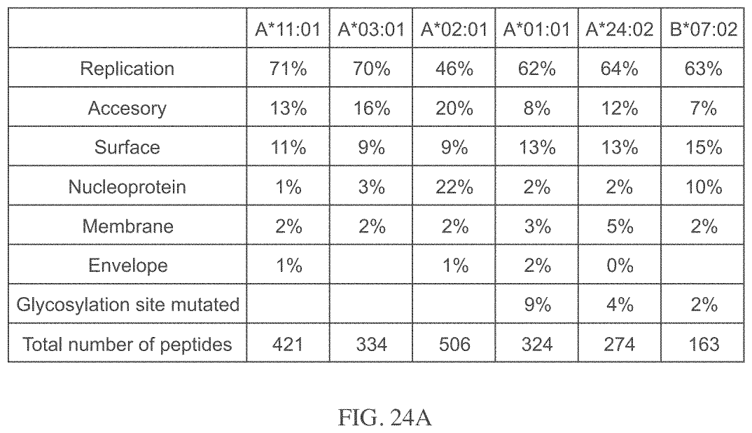

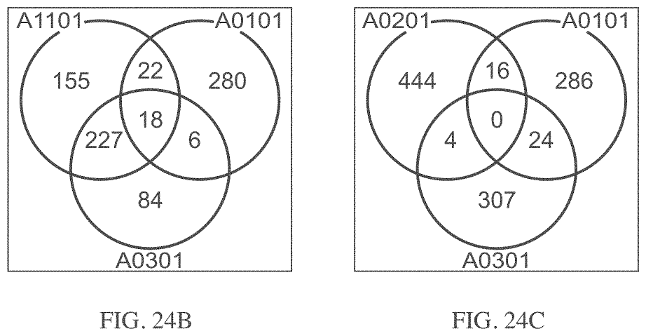

Separate peptide libraries were designed for five additional MHC Class I molecules: HLA-A*11:01, HLA-A*03:01, HLA-A*01:01, HLA-A*24:02 and HLA-B*07:02. The percentage of peptides in the library that bound to each MHC Class I molecule, grouped by types of antigens, is shown in FIG. 24A. The results showed designed library sizes between 163 and 506 peptide epitopes and all alleles had a similar representation of SARS-CoV-2 proteins. The ORF lab antigen was the most represented, occupying 40-70% of the library. Overall, 62% of all peptides identified across HLAs were from ORF lab, while only 11% were from the spike protein, even though the spike protein is the most abundant in the virus itself.

The overlap of peptides in the designed libraries by A1101, A0101 and A0301 is shown in FIG. 24B. The overlap of peptides in the designed libraries by A0201, A0101 and A0301 is shown in FIG. 24C. These overlap analyses showed that there is a significant overlap between the peptides predicted to bind A1101 and A0301. Besides those two alleles, the number of shared peptides between libraries is less than 10% of the total.

To analyze T cell binding to the 596 member peptide-HLA-A*02:01 tetramer library, peripheral blood mononuclear cells (PBMCs) from SARS-CoV-2 patients was obtained and CD8+ T cells were enriched by standard methods. The CD8+ T cells were stained with the tetramer library at 1 nM per tetramer. Cells were washed, stained with anti-FLAG-PE (tetramers each contained a FLAG peptide), washed again, stained with TCR-ADT, washed a final time and then all tetramer+ cells were sorted on a standard cell sorter.

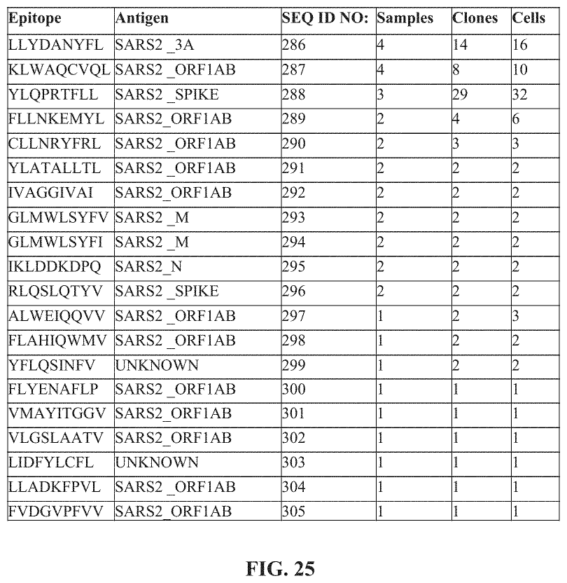

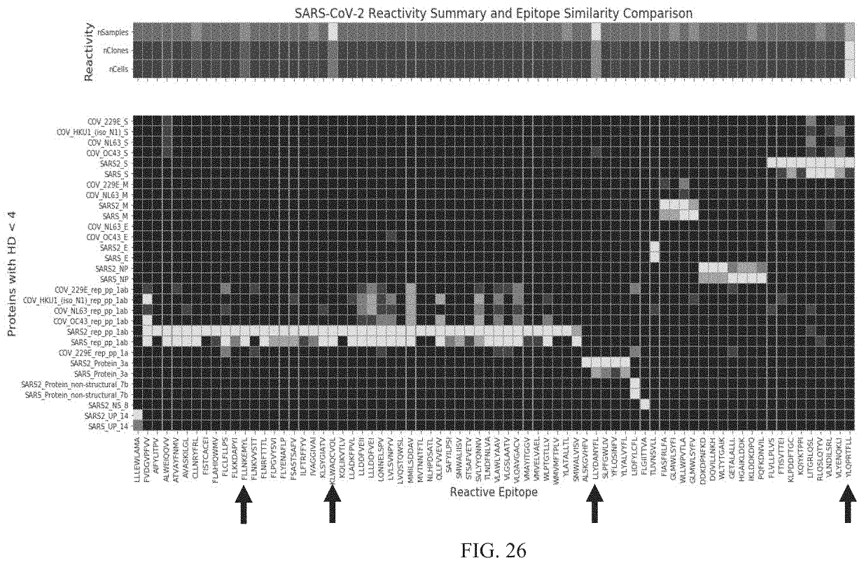

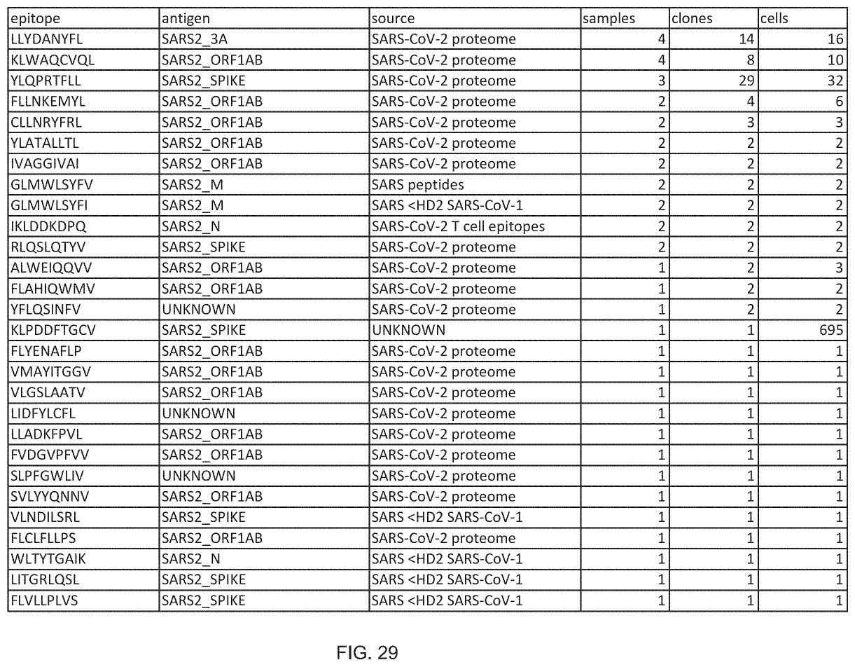

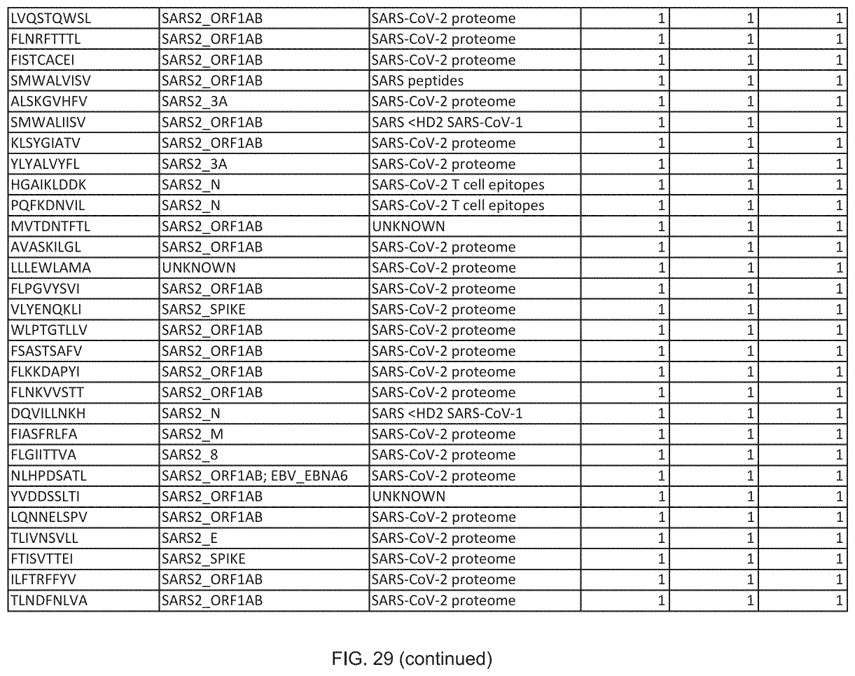

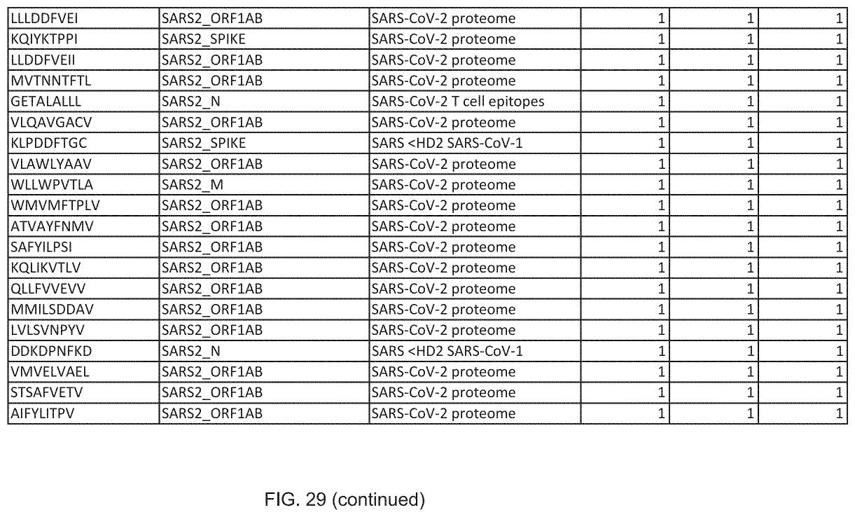

Representative results for A*02:01 samples are shown in FIG. 25, which shows the top 20 different peptide epitopes from the indicated SARS-CoV-2 antigens, along with the number of samples, number of clones and number of cells reactive with each peptide. The sequences of the peptide epitopes shown in FIG. 25 are shown in SEQ ID NOs: 286-305. Additional peptide epitope sequence that showed T cell reactivity are shown in FIG. 29.

The results showed that T cell reactivity was observed across the entire SARS-CoV-2 proteome. The top three epitope hits showed up across at least three different samples. Multiple epitope hits showed diverse clonality. The highest T cell reactivity observed was 9 cells/2000 cells. Thus, there is an estimated T cell reactivity of up to about 0.05% per epitope when taking into account enrichment by sorting. The antigen reactivity prevalence, from highest to lowest, was ORF lab, Spike, 3A and N (equal), M. This antigen reactivity trend was similar across samples, clones and cells.

For further analysis, reactive epitopes were mapped across related viruses, including SARS-Co-V, COV-229E, COV-NL63, COV-OC43 and COV-HKU1. The results are summarized in FIG. 26, with the four most prevalent epitope hits from the library screen (the epitopes shown in SEQ ID NOS: 286-289) highlighted. The results indicated that most of the reactive epitopes are similar or identical in SARS-CoV but are not conserved across endemic coronaviruses. The top epitope from the library screen (SEQ ID NO: 286) shows less similarity to SARS-CoV, and thus was largely unique to SARS-CoV-2.

Overall, the results indicated that screening of the SARS-CoV-2 peptide-MHC tetramer library was an effective approach for identifying T cell epitopes from a variety of SARS-CoV-2 antigens.

### Example 19: SARS-CoV-2 T Cell Epitope Identification by Membrane Epitope Display

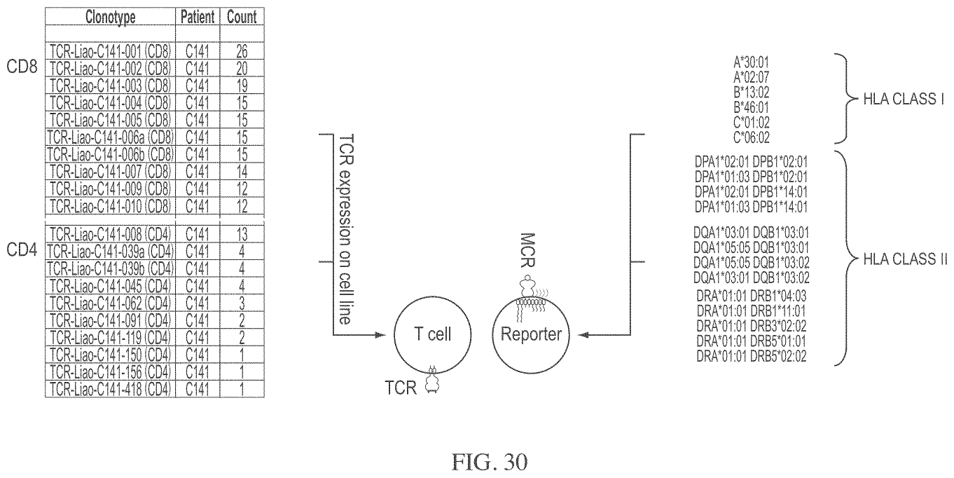

In this example, the MCR™ system schematically illustrated in FIG. 27 and FIG. 28 and described further in Example 17 was used to identify SARS-CoV-2 T cell epitopes. Published T cell receptor sequences were used from T cells obtained from the bronchoalveolar lavage fluid (BAL) of acute COVID-19 patients with mild or severe symptoms. FIG. 30 illustrates the T cell clonotypes from a representative patient and the HLA Class I (A*30:01, A*02:07, B*13:02, B*46:01, C*01:02, and C*06:02) and Class II alleles (DPA1*02:01 DPB1*02:01, DPA1*01:03 DPB1*02:01, DPA1*02:01 DPB1*14:01, DPA1*01:03 DPB1*14:01, DQA1*03:01 DQB1*03:01, DQA1*05:05 DQB1*03:01, DQA1*05:05 DQB1*03:02, DQA1*03:01 DQB1*03:02, DRA*01:01 DRB1*04:03, DRA*01:01 DRB1*11:01, DRA*01:01 DRB3*02:02, DRA*01:01 DRBS*01:01, and DRA*01:01 DRBS*02:02) tested in the reporter assay, based on patient-specific HLA selection. The T cell epitope libraries screened were designed to cover the SARS-CoV-2 genome in its entirety in one amino acid shifts, having 23mers for HLA-II screening and 9mers for HLA-I screening.

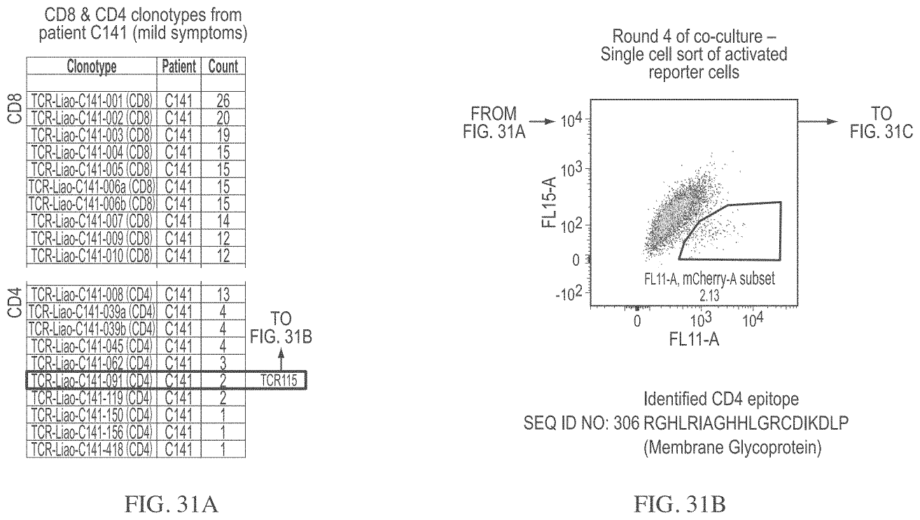

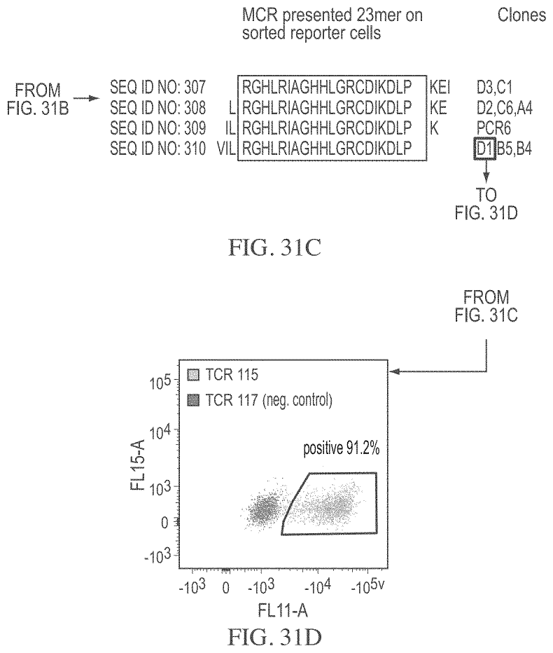

Representative results are shown in FIG. 31A-D.

As shown in FIG. 31A, a representative patient (C141) having mild COVID-19 symptoms exhibited numerous CD8 and CD4 T cell clonotypes. A representative CD4 T cell clonotype was selected for further analysis of the epitope specificity of its T cell receptor (TCR). This TCR (TCR115) was screened in the MCR™ system using a 23mer HLA-II library, with FIG. 31B showing the results of Round 4 of co-culture—single cell sort of activated reporter cells. As shown in FIG. 31C, analysis of the sequences of the 23mer peptides bound by the highest responders uncovered a common 20mer epitope having the amino acid sequence RGHLRIAGHHLGRCDIKDLP (SEQ ID NO: 306), that is found within the four 23mer epitopes shown in SEQ ID NOs: 307-310. FIG. 31D shows results confirming that T cells expressing TCR115 strongly recognized the 20mer epitope, whereas negative control T cells expressing a different receptor (TCR117) did not.

The CD4 helper T cell epitope of SEQ ID NO: 306 recognized by TCR115 is derived from the Membrane Glycoprotein (M protein) of SARS-CoV-2. A nine amino acid subsequence found within this 20mer HLA-II epitope (HLRIAGHHL; SEQ ID NO: 311) has also been reported to be an HLA-I class I epitope (Grifoni et al. (2020) Cell Host & Microbe, 27:671-680). An 18mer epitope that partially overlaps the 20mer of SEQ ID NO: 306, having the amino acid sequence GAVILRGHLRIAGHHLGR (SEQ ID NO: 312), has also been reported to be a CD4 T cell epitope (Peng et al. (2020) Nat. Immunol., published Sep. 4, 2020).

The T cell epitope recognized by TCR115 was further analyzed to compare the peptide presentation capacity of different HLA-II molecules. Five different HLA-II molecules were tested: DRB1*11:01, DRB1*07:01, DRB1*04:04, DRB1*15:01 and DRB1*10:01. Four different 23mer peptides (SEQ ID NOs: 307-310), each containing the 20mer sequence of SEQ ID NO: 306, were tested. Additionally, the overlapping 18mer peptide of SEQ ID NO: 312, reported to be a CD4 epitope, was also tested as a positive control.

The results are shown in FIG. 32, showing the binding of the 23mer epitopes (SEQ ID NOs: 307-310) in grey and the binding of the 18mer epitope (SEQ ID NO: 312) in grey. Additionally, the netMHCIIpanII prediction for binding is shown in grey, with the threshold for positive shown as the horizontal line. The results showed that all five HLA-II molecules tested bound all four 23mers, as well as the 18mer, well about the threshold level for positivity. This is in contrast to the predicted binding from netMHCIIpanII, indicating that the MCR™ system was successful in identifying an (unpredicted) MHCII-restricted CD4 helper T cell epitope with good presentability across a panel of HLA-II alleles.

Overall, these experiments further confirm that use of the MCR™ system is an effective approach for identifying T cell epitopes for T cell clones obtained from symptomatic COVID-19 patients. These experiments further identify epitopes comprising the sequence of SEQ ID NO: 306 (e.g., the sequences of SEQ ID NOs: 307-310) as MHCII-restricted CD4 helper T cell epitopes with good presentability across a panel of HLA-II alleles.

### Example 20: SARS-CoV-2 T cell Epitope Identification Using Peptide-MHC Tetramers

In this Example, peptide-MHC tetramers were loaded with a SARS-CoV-2 9mer epitope library and screened as described in Example 18 for T cell recognition using three different MHC I alleles: A*02:01, A*24:02 and B*07:02. T cell clones obtained from COVID-19 convalescent patients, as well as unexposed controls, were analyzed.

The results showed hits across all three HLA-I alleles tested. 1248 unique clones were tested (200 with high confidence). Hits were obtained for 521 unique epitopes, 20 with high confidence. High confidence hits were further validated by T cell functional assay using an NFAT reporter gene functional assay. The 9mer sequences for the 20 highest confidence hits are shown below in TABLE 8, along with their MHC restriction and the SARS-CoV-2 antigen from which they are derived.

TABLE 8
Exemplary T cell Epitopes

| Epitope | MHC | | SEQ |
|---|---|---|---|
| Sequence | Restriction | SARS-CoV-2 Ag | ID NO: |
| LLYDANYFL | A*02:01 | 3A protein | 286 |
| KLWAQCVQL | A*02:01 | ORF1AB | 287 |
| YLQPRTFLL | A*02:01 | Spike protein | 288 |
| FLLNKEMYL | A*02:01 | ORF1AB | 289 |
| GLMWLSYFI | A*02:01 | M protein | 294 |
| ALWEIQQVV | A*02:01 | ORF1AB | 297 |
| SVLLFLAFV | A*02:01 | E protein | 313 |
| MLDMYSVML | A*02:01 | ORF1AB | 314 |
| KLNEEIAII | A*02:01 | ORF1AB | 315 |
| NYMPYFFTL | A*24:02 | ORF1AB | 316 |
| VYIGDPAQL | A*24:02 | ORF1AB | 317 |
| QYIKWPWYI | A*24:02 | Spike protein | 318 |
| YYTSNPTTF | A*24:02 | ORF1AB | 319 |
| NYNYLYRLF | A*24:02 | Spike protein | 320 |
| YYQLYSTQL | A*24:02 | 3A protein | 321 |
| VYAWNRKRI | A*24:02 | Spike protein | 322 |
| SPRWYFYYL | B*07:02 | N protein | 323 |
| RIRGGDGKM | B*07:02 | N protein | 324 |
| KPRQKRTAT | B*07:02 | N protein | 325 |
| QPGQTFSVL | B*07:02 | ORF1AB | 326 |

The epitope hits showed excellent alignment with published studies, confirming the accuracy of the peptide-MHC tetramer screening approach. Further analysis of the top 20 hits (shown in TABLE 8) is summarized in FIG. 33. This figure shows the breadth/depth for each antigen (% samples, #clones, #cells), as well as the epitope homology to five other coronaviruses (SARS, HKU1, OC43, 229E, NL63). Results are shown for A*02:01, A*24:02 and B*07:02 for convalescent T cells and for B*07:02 for unexposed T cells. The top hits from the screen were detected in 14%-100% of the samples tested with the appropriate HLA-I allele.

Most epitopes were homologous to SARS, but less so to endemic coronaviruses. However, two of the top hits did show strong homology with endemic coronaviruses, VYIGDPAQL (SEQ ID NO: 317), which is from ORF1AB and restricted to A*24:02 and SPRWYFYYL (SEQ ID NO: 323), which is from the N protein and restricted to B*07:02. Notably, reactivity to the epitope of SEQ ID NO: 323 was detected in every convalescent sample tested and in almost half (42%) of unexposed patients.

In summary, these experiments further confirmed that use of the peptide-MHC tetramer system is an effective approach for identifying T cell epitopes for T cell clones obtained from COVID-19 patients. These experiments further identified epitopes comprising the 9mer sequences of SEQ ID NOs: 286-289, 294, 297 and 313-326 as MHCI-restricted T cell epitopes. In particular, SEQ ID NOs: 286-289, 294, 297 and 313-315 were identified as A*02:01-restricted epitopes, SEQ ID NOs: 316-322 were identified as A*24:02-restricted epitopes and SEQ ID NOs: 323-326 were identified as B*07:02-restricted epitopes. Epitopes were identified from six different SARS-CoV-2 antigens: ORF1AB (SEQ ID NOs: 287, 289, 297, 314-317, 319 and 326), Spike protein (SEQ ID NOs: 288, 318, 320 and 322), N protein (SEQ ID NOs: 323-325), M protein (SEQ ID NO: 294), 3A protein (SEQ ID NOs: 286 and 321) and E protein (SEQ ID NO: 313). The experiments revealed that a handful of dominant epitopes are emerging and the most reactive epitopes may have assistance by endemic coronavirus, given the level of reactivity to certain epitopes observed in unexposed patient samples. In particular, the N protein-derived, B*07:02-restricted epitope SPRWYFYYL (SEQ ID NO: 323) showed reactivity with all convalescent patient samples tested and almost half of unexposed patients, indicating it is a dominant T cell epitope.

### Example 21: High Resolution Profiling of MHC-II Peptide Presentation Capacity Reveals SARS-CoV-2 Targets for CD4 T Cells and Mechanisms of Immune-Escape

Understanding peptide presentation by specific MHC alleles is fundamental for controlling physiological functions of T cells and harnessing them for therapeutic use. Currently, two strategies are used: characterization of peptides eluted from purified MHC molecules by mass spectroscopy or in-silico prediction of peptide presentation. However, both approaches have their limitations in sensitivity, precision and throughput, in particular for MHC class II. In this Example, MEDi, a novel mammalian epitope display system which allows an unbiased, affordable, high-resolution mapping of MHC peptide presentation capacity was used. This platform provides a detailed picture by testing every antigen-derived peptide and is scalable to all the MHC alleles. Given the urgent need to understand immune evasion for formulating effective responses to threats like SARS-CoV-2, a comprehensive analysis of the presentability of all SARS-CoV-2 peptides, in the context of several HLA class II alleles was conducted.

This Example demonstrates that some mutations arising in the viral strains expanding globally resulted in reduced peptide presentation by multiple HLA class II alleles, while some increased it, suggesting alteration of MHC-II presentation landscapes as a possible immune escape mechanism.

Decoding antigen presentation in the context of individual HLA alleles is central for understanding immune homeostasis and protection from pathogens and underlies the design of immune medicines. Precise and comprehensive analysis of the short peptides presented by the MHC molecules is therefore of major interest. The main approaches used currently, analysis of MHC-eluted peptides by liquid chromatography with tandem mass spectrometry (LC-MS/MS) and in-silico prediction algorithms, have contributed to the understanding of peptide presentation. However, they do not provide complete presentability landscapes across many HLAs. LC-MS/MS analysis allows the identification of thousands of naturally presented peptides, but it is technically challenging and requires very large numbers of cells (i.e. 10⁸ to 10¹⁰) for good coverage (Sofron, A. et al. (2016) Eur. J. Immunol. 46:319-328, and Kowalewski, D. J. et al. (2015) Proc. Natl. Acad. Sci. 112:E6254-E6256). Moreover, presentation of peptides with proven T cell reactivity can be missed (Kowalewski, D. J. et al. (2015) Proc. Natl. Acad. Sci. 112:E6254-E6256).

The limited sensitivity of LC-MS/MS is especially problematic when working with small tissue samples like human biopsies. Attempting to circumvent these problems, computational prediction methods have been developed and are relatively reliable in identifying strong (IC50<50 nM) MHC I-binders (Röhn, T. A. et al. (2005) Cancer Res. 65:10068-10078). This method is particularly useful for HLA class II, but due to the expression of several HLA alleles on DCs, determination of the individual restriction requires additional experiments. Attempting to circumvent these problems, computational prediction methods have been developed and are relatively reliable in identifying strong (IC50<50 nM) MHC I-binders (Zhao, W. & Sher, X. (2018) PLOS Comput. Biol. 14:e1006457). While for MHC II the algorithms are also improving (Jensen, K. K. et al. (2018) Immunology 154:394-406), the efficiency in predicting MHC-binding peptides is quite variable and limited. In this respect, the recently improved NetMHCIIpan4 shows better performance than conventional binding prediction algorithms but is accurate only for a limited number of alleles, owing to the lack of suitable peptide datasets for training. To circumvent this, a recently published study improved algorithm performance using yeast-display peptide libraries (Reynisson, B. et al. (2020) J. Proteome Res. 19:2304-2315). (Rappazzo, C. G. et al. (2020) Nat. Commun. 11:4414). Still, there is a big gap from the several HLAs with high-quality in-silico prediction scores and the thousands of unique HLA alleles present in the human population.

Predicting antigen presentation by MHC is further complicated by the fact that it is a dynamic process and can change depending on the physiological state of the cell. It is also regulated by tightly controlled chaperones like HLA-DM (Sloan, V. S. et al. (1995) Nature 375:802-806), dysregulation of which has been linked to autoimmune disease progression (Amria, S. et al. (2008) Eur. J. Immunol. 38:1961-1970; Zhou, Z. et al. (2017) Eur. J. Immunol. 47:314-326), while high expression of HLA-DM correlated with improved survival in cancer patients (Oldford, S. A. et al. (2006) Int. Immunol. 18:1591-1602). Thus, an unbiased method, testing pure peptide presentation capacity of the MHC not obscured by other physiological factors, would help getting the complete picture of all possible pMHC ligands present in a given protein. This reductionist approach could provide a basic set of allele-specific peptides (the presentable peptide space) ready for the generation of peptide libraries for screening of T cell reactivities or the generation of pMHC tetramers. Taking this set as a basis, subsets of peptides could be derived by incorporating protein processing and chaperone functions, dependent on cellular state and chaperone expression levels.

Severe acute respiratory syndrome coronavirus 2 (SARS-CoV-2) is the infectious agent responsible for the worldwide COVID-19 pandemic with over two million fatalities (Peiris, J. S. M. et al. (2003) The Lancet 361:7; Matheson, N. J. & Lehner, P. J. (2020) Science 369:510-511). Several companies are now providing vaccines inducing humoral and cellular responses against SARS-CoV-2, but for long lasting protection, generation of T cell memory will be required (Peng, Y. et al. (2020) Nat. Immunol. 21:1336-1345), even if pre-existing T cell immunity to common cold coronavirus might play a role (Nelde, A. et al. (2020) Nat. Immunol. 22:74-85; Grifoni, A. et al. (2020) Cell 181:1489-1501.e15; Sette, A. & Crotty, S. Adaptive immunity to SARS-CoV-2 and COVID-19, 21). Because protection by antibodies is related to protein function (e.g., blocking receptors that are required for viral cell entry), and/or protein localization (surface expression to allow opsonizing antibodies to bind), it has limited target space, increasing selection pressure for pathogen escape. Protection by T cells, on the other hand, relies entirely on TCR recognition of pathogen-derived peptides presented by MHC and is mostly independent of physiological function or localization of the target protein. Consequently, while only particular epitopes of surface proteins allow targeting by neutralizing antibodies, many peptides can serve as T cell targets, providing a much bigger epitope space for therapeutic development. Clearly, a high-resolution map of all SARS-CoV-2 presentable peptides resolved on different HLA alleles would greatly help these efforts.

In this work, utilizing a novel mammalian epitope display system called MEDi, the capacity of several HLA alleles to present SARS-CoV-2 virus peptides was tested. The findings were validated biologically by studying T cell recognition of the SARS-CoV-2 virus in acute COVID-19 patients and analyzed the impact of mutations carried by the novel SARS-CoV-2 strains. The results provided herein suggest immune evasion based on shifting peptide presentation away from well recognized CD4 epitopes. Given the importance of CD4 T cells in controlling B cell and CD8 T cell responses in COVID-19 patients, the results described here may help guide the generation of vaccines or therapeutics designed to elicit efficient cellular immunity.

### Material and Methods

MEDi Procedure and Score Calculation

Libraries carrying 15-amino acid (aa) long peptides, spanning the entire sequences of the SARS-CoV-2 virus, were cloned as oligonucleotides (Twist) into chimeric MHC/TcR receptor (MCR) vectors carrying different HLA alleles. 16.2× reporter cell line was transduced with these libraries and surface expression of the MCR molecules was analyzed by flow cytometry. Four fractions were sorted: Fr.0 (cells expressing no detectable MCRs on the surface), Fr.1 (cells expressing low levels of MCRs), Fr.2 (cells expressing intermediate levels of MCRs), Fr.3 (cells expressing high levels of MCRs). Peptides carried by the MCRs from sorted cells were amplified from cDNA by RT-PCR using the peptide flanking regions and sequenced on a miniSeq (Illumina). Sequences from the Illumina output files were trimmed, merged and translated using the CLC genomic workbench program. Counting and further analysis was done with FilemakerPro18 and Excel (Microsoft).

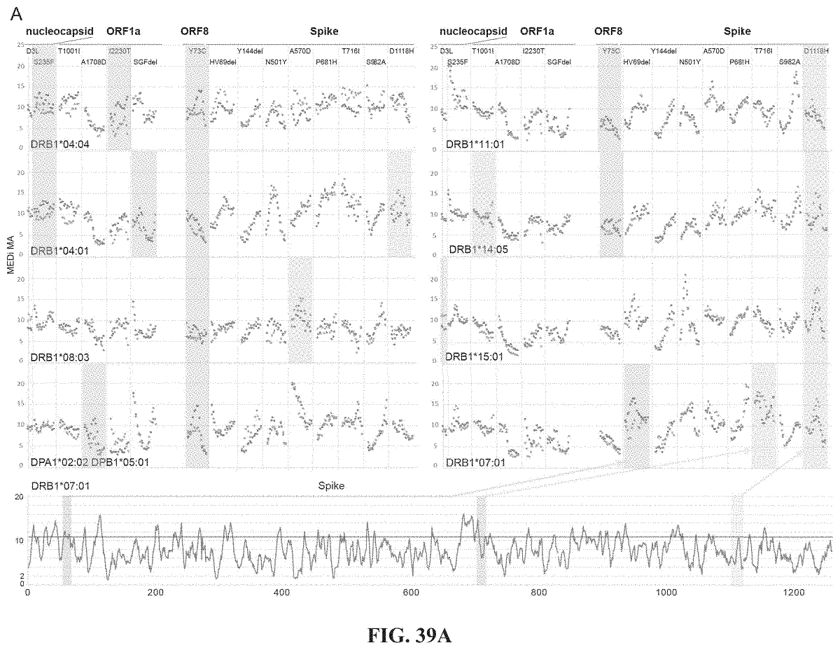

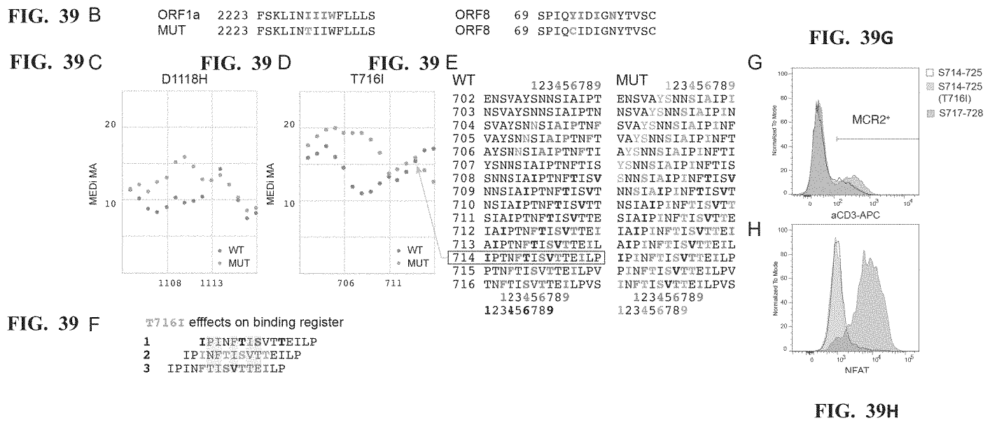

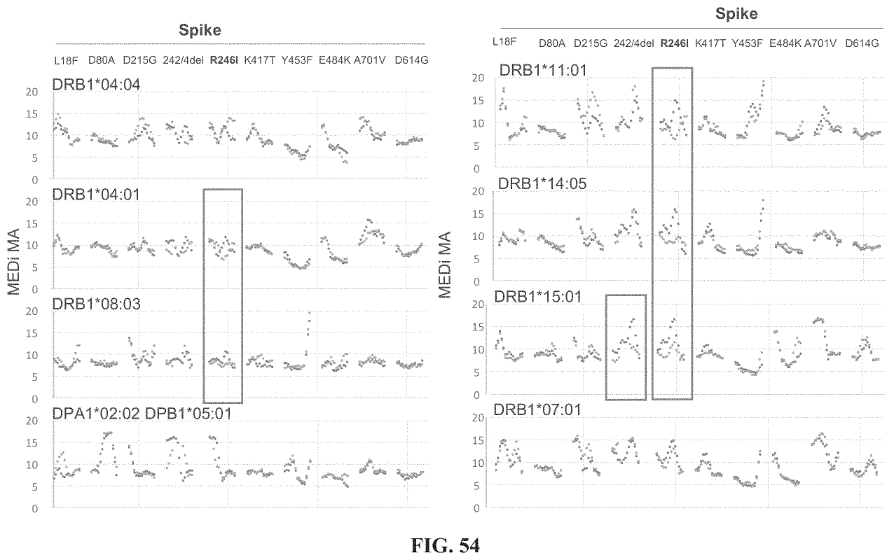

The individual peptide counts in each fraction were normalized to the total counts in the fraction. For each peptide, a MEDi score was calculated with the following formula: sum_i [(Frindex_i*Frcount_i)/sum_i(Frcount_i)]. Fr_indexes: Fr1=1, Fr2=2, Fr3=4, Fr4=28. MEDi-MA was calculated by averaging MEDi scores for 5 peptides (−2/−1/0/1/2) and assigned to the middle(0) peptide, except for FIG. 39 and FIG. 54 where MEDi-MA was calculated by averaging MEDi scores for 3 peptides (−1/0/1). MEDi-MA85 indicates the threshold calculated as the 85th percentile of the MEDi-MA score for the individual protein.

MEDi MA Score Quality Threshold.

MEDi MA score for a given peptide was considered of good quality if at least 40 reads were collected for a peptide and the MEDi MA value had a coefficient of variation (CV=Std.Deviation/Average) lower than 0.75.

Local Maximum of MEDi MA Peak Definition

Local maximum of 7 MEDi MA scores was determined (−3/−2/−1/0/1/2/3) and assigned to the middle(0) peptide.

MCR2 Screening

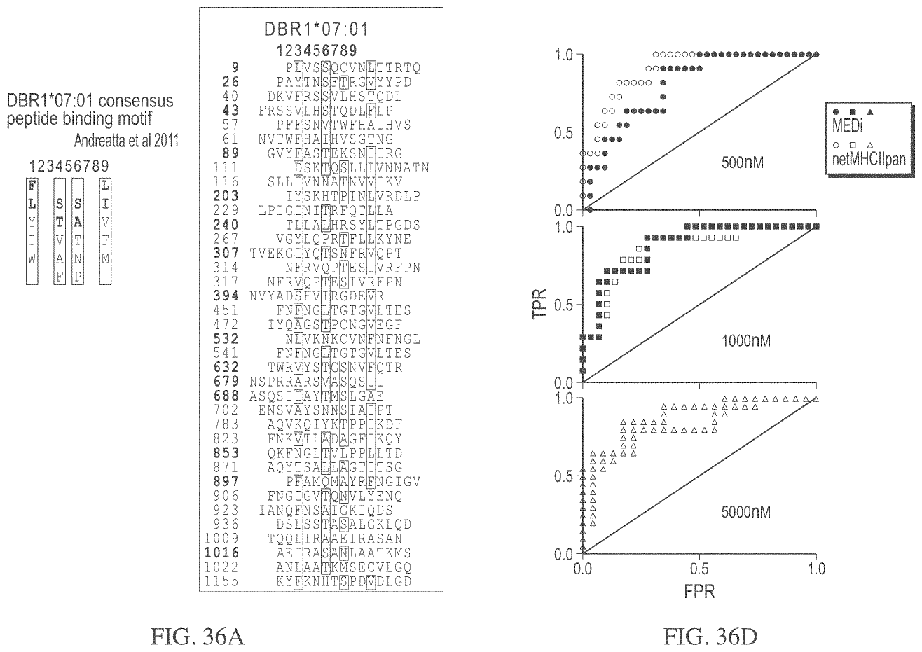

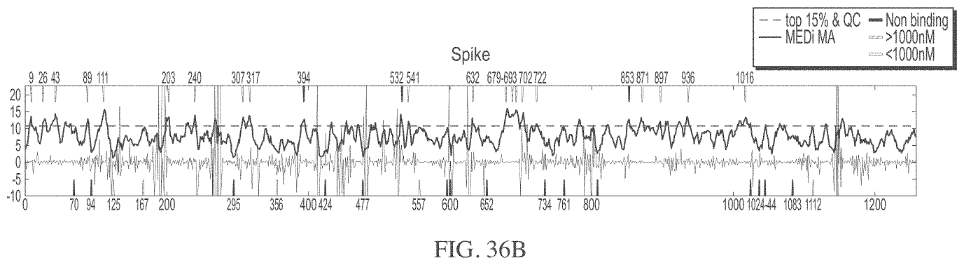

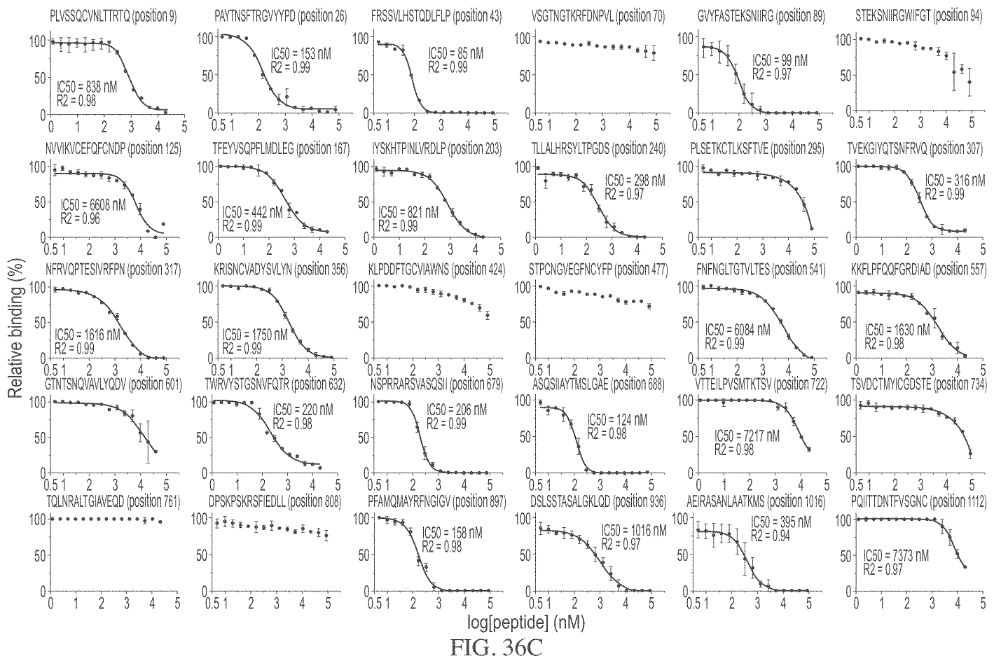

Libraries were generated by cloning all SARS-CoV-2-derived peptides in MCR2 molecules carrying the complete viral genome in 23mers shifted by 1 aa. For screening, the libraries were pooled at equal ratios, generating a combined patient-specific library of roughly 120,000 different peptide-MCR2 combinations. MCR2 screening was performed as described previously (Kisielow, J. et al. (2019) Nat. Immunol. 20:652-662). Briefly, MCR2 expressing 16.2× cells were co-cultured with cell clones expressing one specific TCR selected from Liao et al. (Nat. Med. (2020) 26:842-844) in a ratio of 1:5 to 1:10. Cells were mixed and co-cultured for 8-12 hours in a standard tissue culture medium, in the presence of 13 μg/mL anti-mouse FasL antibodies (BioXcell) to inhibit induction of cell death during incubation. After harvesting, reporter cells positive for NFAT signaling were sorted on a BD FACS Aria Fusion Cell Sorter as bulk or into 96 well plates for further expansion. Expanded single cells were harvested, DNA was isolated (Kapa Express Extract) followed by Sanger sequencing of the MCR2 alpha and beta chain including the linked antigen. When overlapping peptides were found in the screen (e.g., FIG. 36A and TABLE 9), the common part as the specific peptide recognized by the TCR was listed.

TABLE 9 depicts exemplary peptides identified in the screen

TABLE 9

| Sequence | SEQ ID NO: |
|---|---|
| PLVSSQCVNLTTRTQ | 1327 |
| PAYTNSFTRGVYYPD | 1328 |
| DKVFRSSVLHSTQDL | 1329 |
| FRSSVLHSTQDLFLP | 1330 |
| PFFSNVTWFHAIHVS | 1331 |
| NVTWFHAIHVSGTNG | 1332 |
| GVYFASTEKSNIIRG | 1333 |
| DSKTQSLLIVNNATN | 1334 |
| SLLIVNNATNVVIKV | 1335 |
| IYSKHTPINLVRDLP | 1336 |
| LPIGINITRFQTLLA | 1337 |
| TLLALHRSYLTPGDS | 1338 |
| VGYLQPRTFLLKYNE | 1339 |
| TVEKGIYQTSNFRVQPT | 1340 |
| NFRVQPTESIVRFPN | 1341 |
| NFRVQPTESIVRFPN | 1342 |
| NVYADSFVIRGDEVR | 1343 |
| FNFNGLTGTGVLTES | 1344 |
| IYQAGSTPCNGVEGF | 1345 |
| NLVKNKCVNFNFNGL | 1346 |
| FNFNGLTGTGVLTES | 1347 |
| TWRVYSTGSNVFQTR | 1348 |
| NSPRRARSVASQSII | 1349 |
| ASQSIIAYTMSLGAE | 1350 |
| ENSVAYSNNSIAIPT | 1351 |
| AQVKQIYKTPPIKDF | 1352 |
| FNKVTLADAGFIKQY | 1353 |
| QKFNGLTVLPPLLTD | 1354 |
| AQYTSALLAGTITSG | 1355 |
| PFAMQMAYRFNGIGV | 1356 |
| FNGIGVTQNVLYENQ | 1357 |
| IANQFNSAIGKIQDS | 1358 |
| DSLSSTASALGKLQD | 1359 |
| TQQLIRAAEIRASAN | 1360 |
| AEIRASANLAATKMS | 1361 |
| ANLAATKMSECVLGQ | 1362 |
| KYFKNHTSPDVDLGD | 1363 |

Single Chain Trimer (SCT) Screening

Single chain trimers of class I HLAs of all seven patients were generated by linking the leader sequence, epitope, β2m and HLA alpha chain with 3×G4S linkers, transmembrane and intracellular domain were cloned from the CD247 molecules. Each alpha chain was modified/mutated to open the groove of class I by introducing the Y84A mutation in every alpha chain.

For SCT, libraries were used that covered the whole SARS-CoV-2 genome with 10mers shifted by 1 amino acid cloned as oligonucleotides into the SCTz vectors.

Fluorescence Polarization Assay

The MHC II α- and β-chain extracellular domains were recombinantly expressed with C-terminal Myc and His tag sequences, respectively. For DRB1*15:01 the Myc tag was replaced with a V5 tag. The N-terminus of the β-chain was fused to CLIP peptide followed by a flexible Factor Xa-cleavable linker. Both α- and β-chains were co-expressed in CHO cells and secreted into the expression medium as a stable CLIP-loaded heterodimer. Heterodimerization of the α- and β-chains of DRB1*07:01 and DRB1*1501 was forced using a fusion of an engineered human IgG1-Fc protein to each chain (Gunasekaran, K. et al. (2010) J Biol. Chem. 285:19637-19646). Following CHO expression, the heterodimer was purified by immobilized metal ion affinity chromatography and size exclusion chromatography (SEC). The fluorescence polarization assay was performed as described in Yin, L. & Stern, L. J. (2014) Curr. Protoc. Immunol. 106:5.10.1-5.10.12 with few modifications. Following Factor Xa cleavage, 100 nM of HLA were incubated overnight with 25 nM fluorescent probe and various concentrations of the indicated peptide competitor in 100 mM Sodium citrate pH 5.5, 100 mM NaCl, 0.1% octylglucoside and 1× protease inhibitors (SigmaFast) at 30° C. The fluorescent probe for DRB1*04:04, DRB1*07:01 and DRB1*11:01 was PRFV(K/Alexa488)QNTLRLAT. The fluorescent probe for DRB1*15:01 was ENPVVHFF(C/Alexa488Mal)NIVTPR.

### Results

MEDi, a Mammalian Epitope Display Platform Based on MCR

Using the MCR system, an immunogenic, murine leukemia virus envelope protein-derived mutant peptide (MLVenvs126R,D127v, aka envRV) was identified as being efficiently recognized by mouse tumor infiltrating lymphocytes (Kisielow, J. et at (2019) Nat. Immunol. 20:652-662). While MCR2 molecules carrying envRV were expressed well on the surface of the reporter cells, the ones carrying the nonmutated WT peptide (env) could not be detected, consistent with netMHCIIpan affinity predictions. Given that MHC molecules without a bound peptide were unstable (Rabinowitz, J. D. et al. (1998) Immunity 9:699-709 this observation led to the hypothesis that peptides fitting well into the peptide binding groove and therefore being efficiently presented by the MHC, may effectively stabilize the MCR2 molecules on the surface of cells. In contrast, peptides not well presented by the MHC destabilize the MCR2 molecules and therefore little, if any, cell surface expression will be detected. This hypothesis was tested. A number of peptides with biochemically tested I-Ab binding affinity ranging from 7.5 nM to 10,000 nM (TABLE 10) were cloned.

TABLE 10
I-Ab presented peptides cloned in MCR2 vector

| Number | Peptide | SEQ ID NO: | IC50 |
|---|---|---|---|
| 1 | KSAFQSSVASGFIGF | 1417 | 7.56 |
| 2 | ISGYNFSLSAAVKAG | 1418 | 32.3 |
| 3 | IEYAKLYVLSPILAE | 1419 | 282 |
| 4 | FSLSAAVKAGASLID | 1420 | 638 |
| 5 | SLINSMKTSFSSRLL | 1421 | 1700 |
| 6 | LLNNQFGTMPSLTLA | 1422 | 4740 |
| 7 | GLVSQLSVLSSITNI | 1423 | 5280 |
| 8 | YDMFNLLLMKPLGIE | 1424 | 6750 |
| 9 | LIEDYFEALSLQLSG | 1425 | 6760 |
| 10 | IIKYNRRLAKSIICE | 1426 | 8390 |
| 11 | NKVKSLRILNTRRKL | 1427 | 8580 |
| 12 | AWENTTIDLTSEKPA | 1428 | 10000 |

The clones of the peptides were transduced into 16.2× reporter cell line and determined the MCR2 expression by flow cytometry and staining with I-Ab and CD3 specific antibodies. As expected, there was a clear linear correlation between both stainings, but CD3 allowed a better separation of the positive and negative populations. As a result, anti-CD3 staining was used in further MEDi analyses, with the added advantage of being MHC agnostic and therefore universally usable with all mouse H-2 and human HLA haplotypes. MCR2 expression dependence on peptide-I-Ab binding affinity was measured by the mean fluorescence. It was found that the MCR2s carrying peptides with a good I-Ab binding affinity were expressed on the surface at high levels, while MCR2s presenting low affinity peptides showed lower surface expression. Peptides with an affinity below 1 μM (IC₅₀) were considered good MHC-binders and all MCRs carrying such peptides were expressed well on the cell surface. In addition, some peptides with lower MHC binding affinity appeared on the surface, indicating that linking peptides directly to the MHC beta chain stabilizes low-affinity peptide-MHC interactions. Being able to test the presentation of such peptides was important, as self-peptides known as targets in autoimmune diseases often bind MHC with low affinity (Stadinski, B. D. et al. (2010) Proc. Natl. Acad. Sci. 107:10978-10983). Six (6) out of 6 peptides with an I-Ab binding affinity below 5 μM (IC₅₀) stabilized MCR2 surface expression, while for peptides with lower binding affinity, MCR expression was variable and generally much lower. Some of the MCR2s carrying peptides with an apparently low affinity (e.g., 8.39 μM) were expressed on the surface at good levels, suggesting that additional factors apart from pure binding affinity (measured in vitro), regulate peptide-MHC interactions. Similarly, the envRV peptide could stabilize MCR2 expression, even if its I-Ab binding affinity was predicted by netMHCIIpan to be very low at 7.7 μM. As a result, high amounts of envRV peptide were added for in vitro T cell stimulations by dendritic cells (Kisielow, J. et al. (2019) Nat. Immunol. 20:652-662).

Analysis of SARS-CoV-2 Peptides Presentability by Common HLA Alleles.

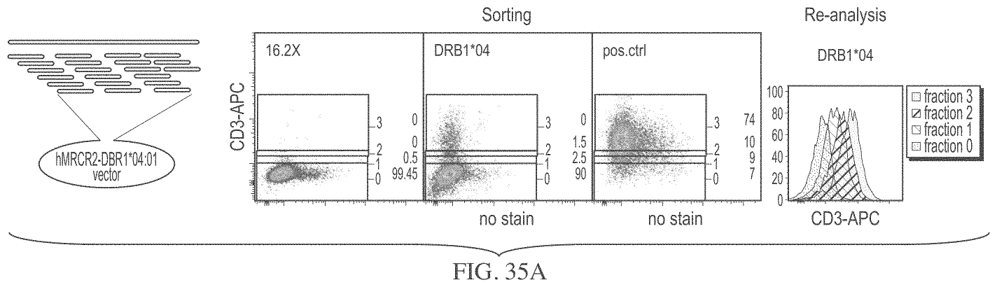

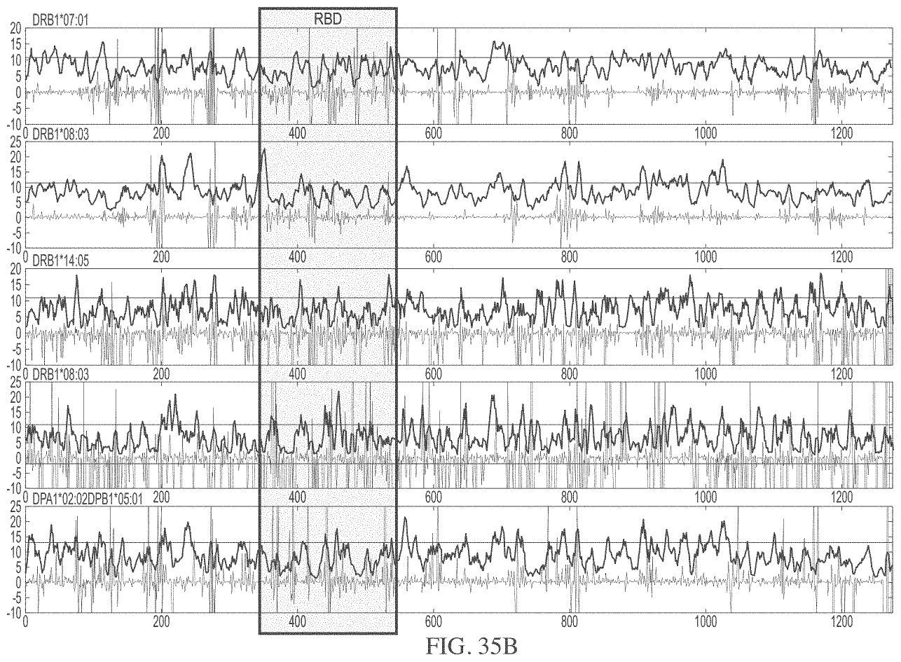

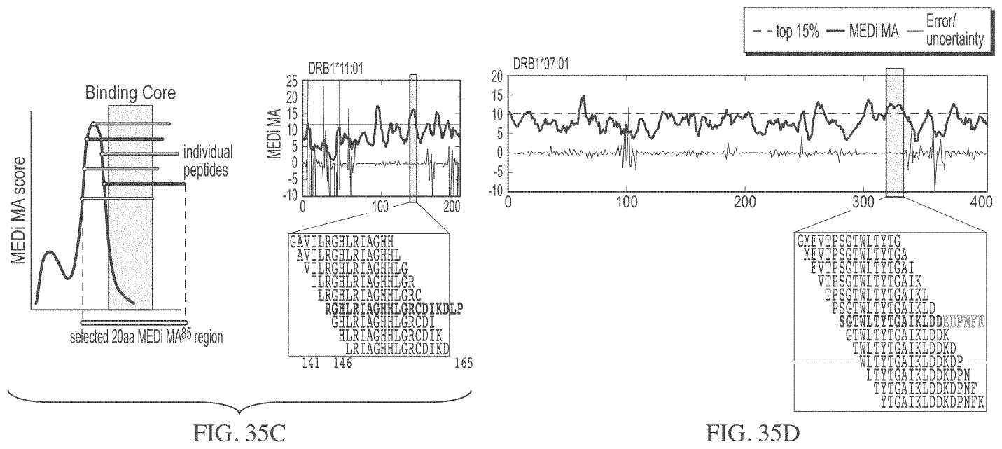

Considering the recent interest in SARS-CoV-2 T cell epitopes effectively presented across the possibly highest number of HLA alleles, MEDi was used to determine the presentability of all peptides encoded in the SARS-CoV-2 genome in the context of some of the most common HLA class II haplotypes. The critical role of CD4 T cell help in supporting B cell and CD8 T cell responses is well known and also crucial for COVID-19 protection (Le Bert, N. et al. (2020) Nature (2020) 584:457-462; Juno, J. A. et al. (2020) Nat. Med. (2020) 26:1428-1434). However, a complete picture of the important MHC class II epitopes was missing, as they are more difficult to predict by computer algorithms than MHC class I ligands. To achieve a good resolution, all possible 15aa peptides derived from the SARS-CoV-2 genome (FIG. 35A), shifted by 1 aa, were cloned into MCR2 vectors containing extracellular domains of the HLAs: DRB1*04:04, DRB1*07:01, DRB1*08:03, DRB1*11:01, DRB1*14:05, DRB1*15:01 and DPA1*02:02/DPB1*05:01 (FIGS. 35A, B, C, and D). These libraries were transduced into the 16.2× reporter cell line, stained for CD3 and sorted the cells into 4 fractions (neg, low, mid and hi) based on the surface expression level of the MCR2 (FIG. 35A). The peptides carried by the MCR2s in the different fractions were determined by RT-PCR and deep sequencing. For each peptide a MEDi score was calculated and plotted against the position of the starting amino acid of the peptide within the protein (see Methods). FIG. 35B shows plots of the MEDi score moving average (MEDi-MA, average of 5 peptides) for the SARS-CoV-2 Spike peptide presentability by a set of 5 HLA alleles. Peptides derived from particular regions of the protein stabilized surface expression of the MCR better than others and so are being better presented by the MHC. Such peptides grouped in regions (“peaks/waves”), indicating that a core MHC-binding epitope was present in a number of peptides starting at several consecutive amino acids (FIGS. 35C and D, and TABLE 11).

TABLE 11 Exemplary peptides and respective HLA alleles identified in the screen. TABLE 11

| Sequence | SEQ ID NO: | HLA |
|---|---|---|
| GAVILRGHLRIAGHH | 1364 | DRB1*11:01 |
| AVILRGHLRIAGHHL | 1365 | DRB1*11:01 |
| VILRGHLRIAGHHLG | 1366 | DRB1*11:01 |
| ILRGHLRIAGHHLGR | 1367 | DRB1*11:01 |
| LRGHLRIAGHHLGRC | 1368 | DRB1*11:01 |
| RGHLRIAGHHLGRCDIKDLP | 1369 | DRB1*11:01 |
| GHLRIAGHHLGRCDI | 1370 | DRB1*11:01 |
| HLRIAGHHLGRCDIK | 1371 | DRB1*11:01 |
| LRIAGHHLGRCDIKD | 1372 | DRB1*11:01 |
| GMEVTPSGTWLTYTG | 1373 | DRB1*07:01 |
| MEVTPSGTWLTYTGA | 1374 | DRB1*07:01 |
| EVTPSGTWLTYTGAI | 1375 | DRB1*07:01 |
| VTPSGTWLTYTGAIK | 1376 | DRB1*07:01 |
| TPSGTWLTYTGAIKL | 1377 | DRB1*07:01 |
| PSGTWLTYTGAIKLD | 1378 | DRB1*07:01 |
| SGTWLTYTGAIKLDDKDPNFK | 1379 | DRB1*07:01 |
| GTWLTYTGAIKLDDK | 1380 | DRB1*07:01 |
| TWLTYTGAIKLDDKD | 1381 | DRB1*07:01 |
| WLTYTGAIKLDDKDP | 1382 | DRB1*07:01 |
| LTYTGAIKLDDKDPN | 1383 | DRB1*07:01 |
| TYTGAIKLDDKDPNF | 1384 | DRB1*07:01 |
| YTGAIKLDDKDPNFK | 1385 | DRB1*07:01 |

This observation is consistent with the fact that, owing to its open peptide-binding groove, MHC class II molecules present peptides of different length (Sofron, A. et al. (2016) Eur. J. Immunol. 46:319-328). Usually the minimal MHC-binding core is composed of 9aa as shown by the commonly described binding motifs (Andreatta, M. et al. (2011) PLoS ONE 6:11)), even if residues outside of it also contribute to the MHCbinding affinity (O'Brien, C. et al. (2008) Immunome Res. 4:6). As expected, MEDi graphs derived from these analyses showed a diverse presentation pattern. Each HLA molecule was unique, with regions of specific and promiscuous peptide presentation.

To account for data quality differences related to sorted cell numbers and sequencing depth, a MEDi-MA quality metric composed of a minimal read count and the coefficient of variation (see Materials & Methods) was applied. Graphs in FIG. 35B show that the best results were obtained for DRB1*07:01 and DRB1*15:01 and DPA1*02:02/DPB1*05:01, while DRB1*14:05 and DRB1*08:03 showed lower quality. As a result, most of the MEDi platform testing was performed on DRB1*07:01 and DRB1*15:01.

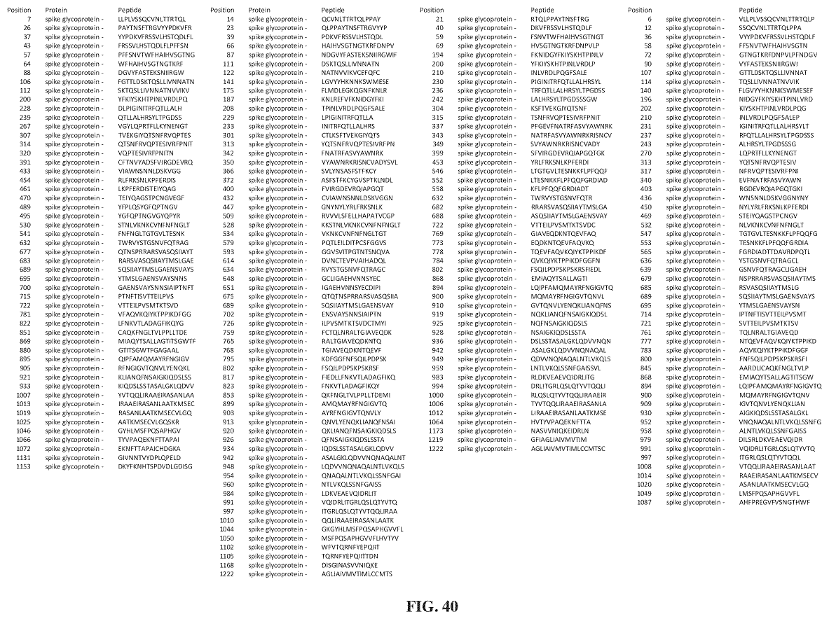

To distill the best HLA-binding epitopes from this data, peptide sequences scoring above the 85th percentile (MEDi-MA85) were selected. As an example, FIG. 40 provides a list of presentable peptides derived from the Spike protein. This analysis was performed on all peptides derived from the SARS-CoV-2 genome in the context of 3 HLAs. Of note, the spike list contains peptides greatly overlapping with the immunogenic peptides described in recent literature (Peng, Y. et al. (2020) Nat. Immunol. 21:1336-1345; Nelde, A. et al. (2020) Nat. Immunol. 22:74-85).

Validation of MEDi by a Competitive Peptide Binding Assay.

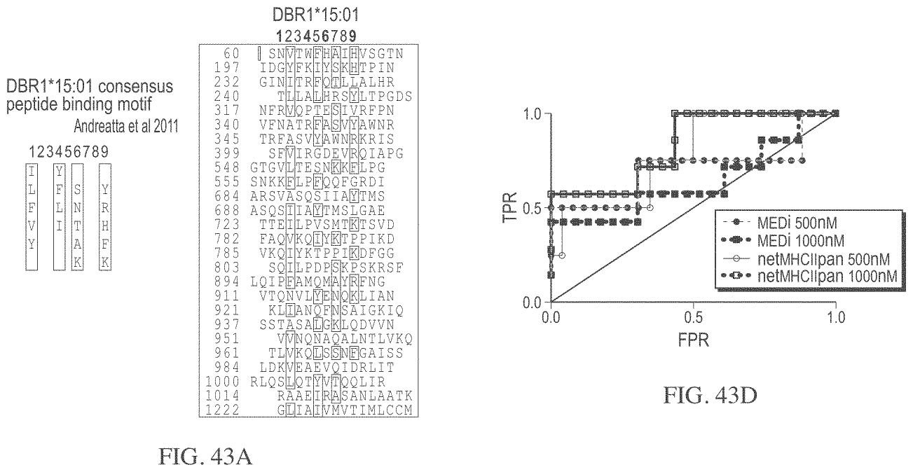

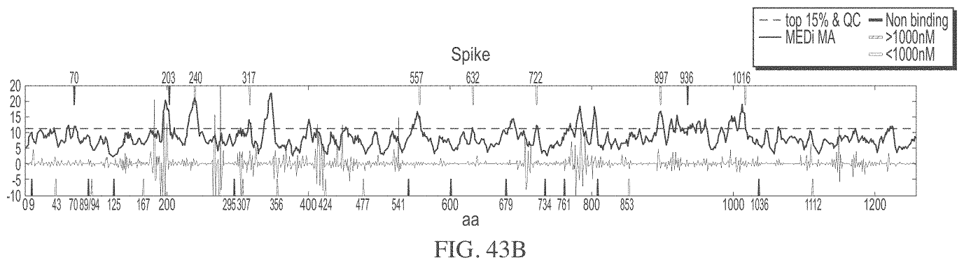

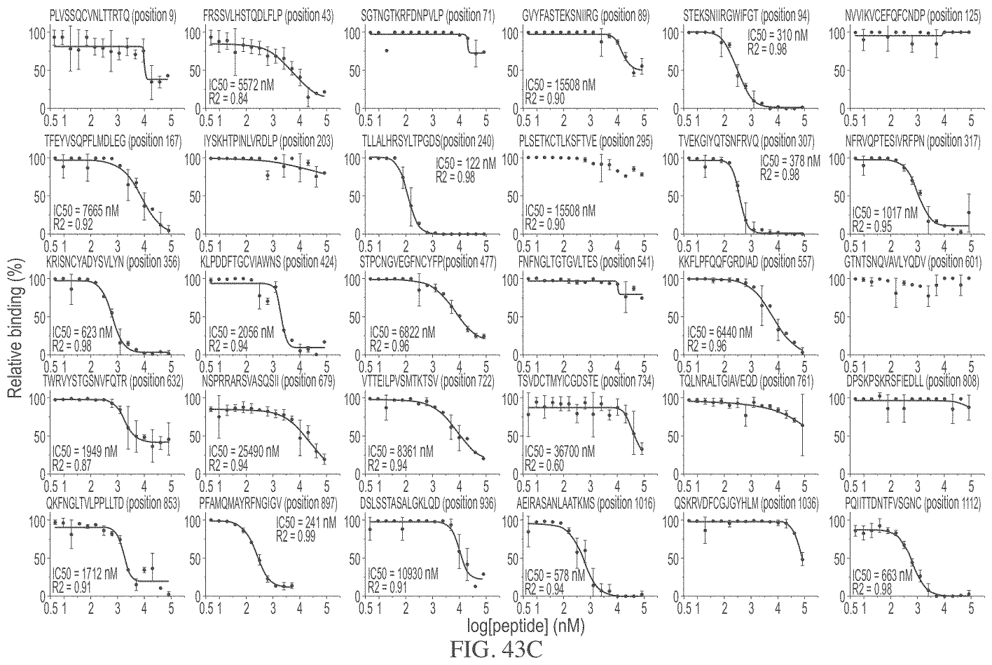

Next, peptides from the Spike protein in major MEDi MA⁸⁵ peaks were analyzed for the presence of a binding motif and an enrichment of known (Andreatta, M. et al. (2011) PLoS ONE 6:11), appropriately spaced anchor residues in most of the selected peptides (FIG. 36A: DRB1*07:01, and TABLE 9; and FIG. 43A: DRB1*15:01) was identified, thus validating the assay. Still, because tethering peptides to the MCR might stabilize some low affinity interactions not efficiently presented in vivo, independent validation and quantification of the HLA binding of the peptides was performed by MEDi. To this end, measurements of competitive peptide binding by fluorescence polarization were performed (Yin, L. & Stern, L. J. (2014) Curr. Protoc. Immunol. 106:5.10.1-5.10.12) fora set of Spike peptides for DRB1*07:01. Thirty-three (33) peptides were selected representing MEDi MA peaks and 10 peptides representing valleys (FIG. 36B) and considered peptides with IC₅₀ below 10 μM as binders. When IC₅₀ calculation was impossible due to very low peptide binding it was set arbitrarily to 20 μM. 20 out of the 23 peptides (87%) corresponding to MEDi-MA85 peaks bound to the HLA with IC₅₀ between 85 nM to 10 μM (FIG. 36C, peptide sequences listed in TABLE 12), 13 of them below 1 μM. From the remaining 10 peaks, three peptides bound to the HLA (IC₅₀ 442 nM, 1,630 nM and 7.3 μM) but missed MEDi-MA85 cut-off by a small margin (FIG. 36C), and for the rest, no binding could be shown. For peptides from the valleys, 2 out of 10 (20%) bound to the HLA with low affinity, while the rest did not bind (FIG. 36C). This data set allowed us to analyze the ability of the MEDi assay to qualify peptides for HLA presentation and compare it to netMHCIIpan. Receiver operating characteristic curves (ROC) were plotted for different IC₅₀ cut-offs and compared MEDi-MA scores to netMHCIIpan EL rank (FIG. 36D). Overall, the performance of both methods was comparable, with MEDi performing better for low affinity peptides (1 μM and 5 μM IC₅₀ cut-offs: AUC 87.5% to 86% and 88% to 82% respectively), while netMHCIIpan was better for the 500 nM IC₅₀ cut-off (AUC 89.8% to 80.2%).

TABLE 12. Exemplary peptide sequences tested for binding to DRB1*07:01.

TABLE 12

| SEQ ID NO: | Sequence |
|---|---|
| 1386 | PLVSSQCVNLTTRTQ |
| 1387 | PAYTNSFTRGVYYPD |
| 1388 | VSGTNGTKRFDNPVL |
| 1389 | STEKSNIIRGWIFGT |
| 1390 | NVVIKVCEFQFCNDP |
| 1391 | TFEYVSQPFLMDLEG |
| 1392 | IYSKHTPINLVRDLP |
| 1393 | TLLALHRSYLTPGDS |
| 1394 | PLSETKCTLKSFTVE |
| 1395 | TVEKGIYQTSNFRVQPT |
| 1396 | NFRVQPTESIVRFPN |
| 1397 | KRISNCVADYSVLYN |
| 1398 | KLPDDFTGCVIAWNS |
| 1399 | STPCNGVEGFNCYFP |
| 1400 | FNFNGLTGTGVLTES |
| 1401 | KKFLPFQQFGRDIAD |
| 1402 | GTNTSNQVAVLYQDV |
| 1403 | TWRVYSTGSNVFQTR |
| 1404 | NSPRRARSVASQSII |
| 1405 | ASQSIIAYTMSLGAE |
| 1406 | VTTEILPVSMTKTSV |
| 1407 | TSVDCTMYICGDSTE |
| 1408 | TQLNRALTGIAVEQD |
| 1409 | DPSKPSKRSFIEDLL |
| 1410 | PFAMQMAYRFNGIGV |
| 1411 | DSLSSTASALGKLQD |
| 1412 | AEIRASANLAATKMS |
| 1413 | PQIITTDNTFVSGNC |
| 1414 | DSLSSTASALGKLQD |
| 1415 | AEIRASANLAATKMS |
| 1416 | PQIITTDNTFVSGNC |

Next, using 30 of these peptides, an unbiased analysis was performed for DRB1*15:01 (FIG. 43). Here, because the peptides were chosen according to MEDi data for DRB1*07:01, most peptides corresponded to MEDi scores below the 85^{th} percentile threshold and were not in major peaks (FIG. 43A and FIG. 43B), in other words they should not be well presented. Indeed, the majority did not bind the HLA with sufficient affinity (FIG. 43C). Nevertheless, 10 of the peptides were in peaks above the threshold and 7 bound to HLA. The two peptides with the highest IC₅₀ (122 nM and 241 nM) corresponded to 2 of the 5 highest MEDi MA85 peaks and were on the top of the MEDi ranking NetMHCIIpan placed them lower at the 3′ and 30′ rank. On the other hand, 4 of 7 HLA binding peptides (IC50 from 3 10 nM to 663 nM) missed the MEDi 85^{th} percentile threshold, two of them by a small margin, possibly due to low quality data in these regions. NetMHCIIpan also did not qualify 3 of the 7 binding peptides as good HLA binders but placed them at slightly higher positions in the overall ranking (FIG. 43D). Both methods performed similarly for this well characterized HLA allele. These results validate the MEDi platform as a means to select peptides highly presentable by an HLA allele.

De-Orphaning TCRs from the Bronchoalveolar Lavages (BAL) of Acute COVID-19 Patients.

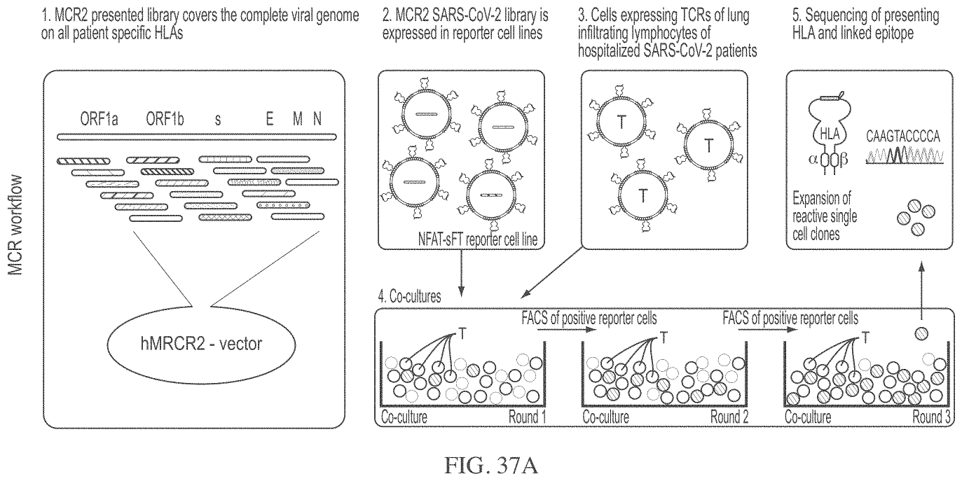

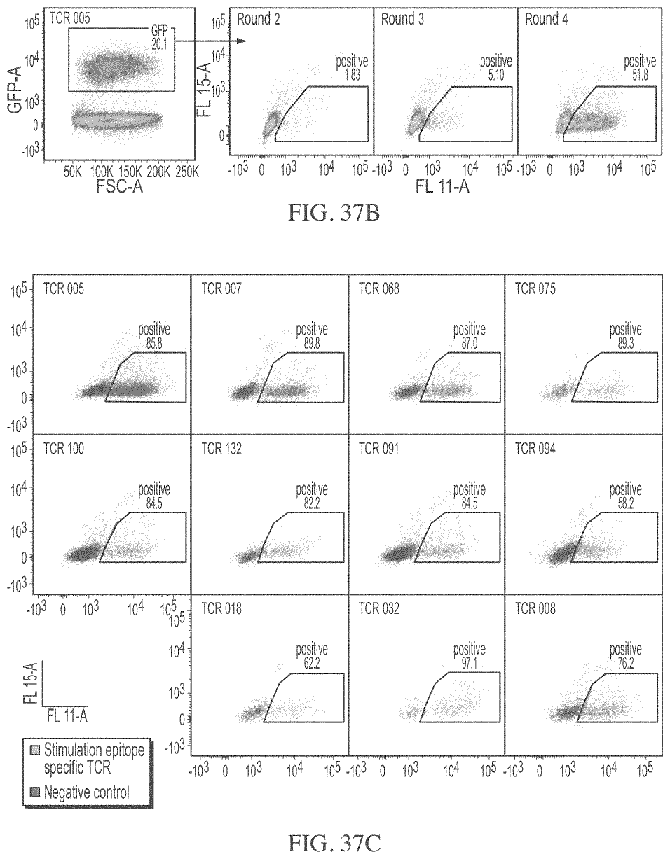

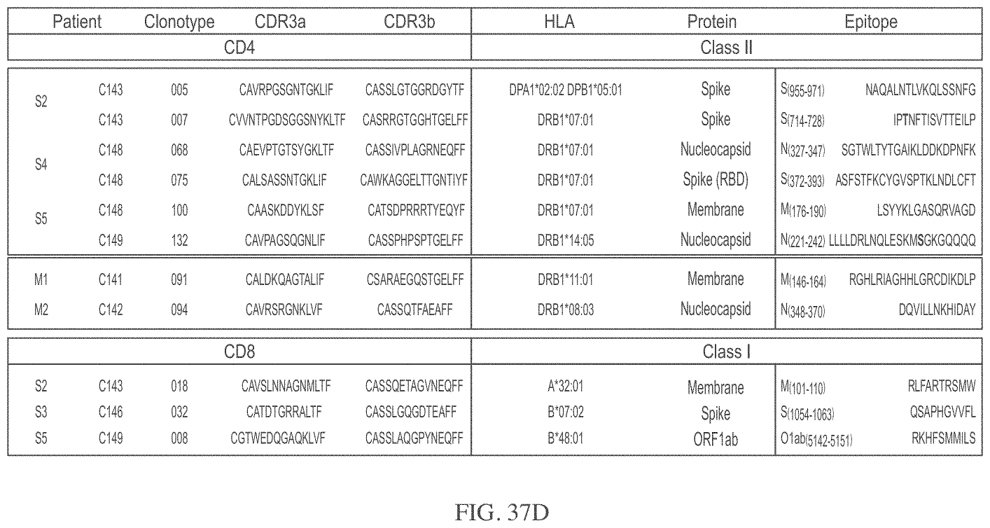

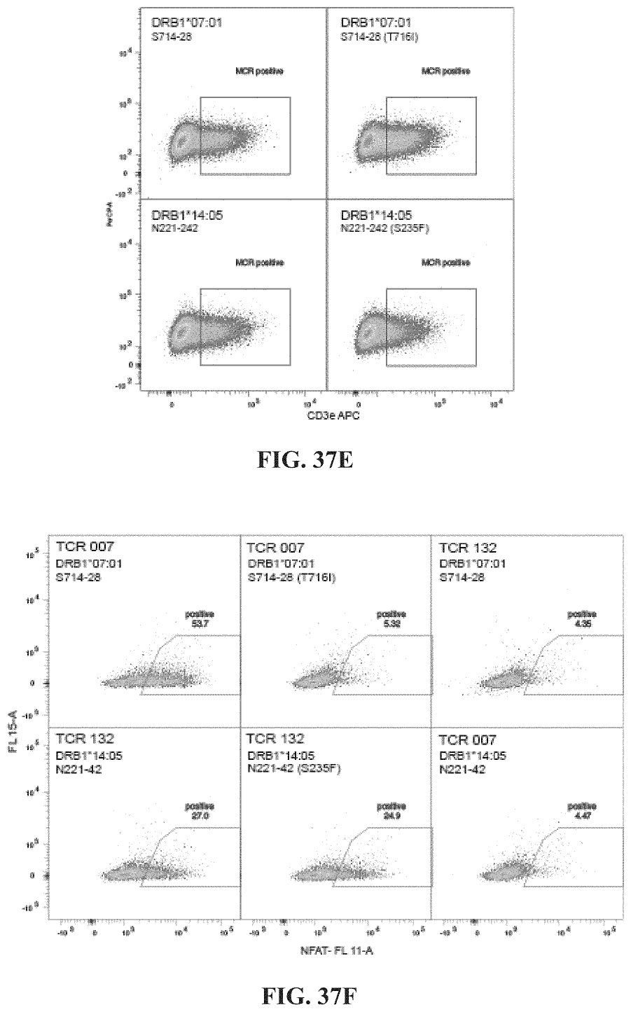

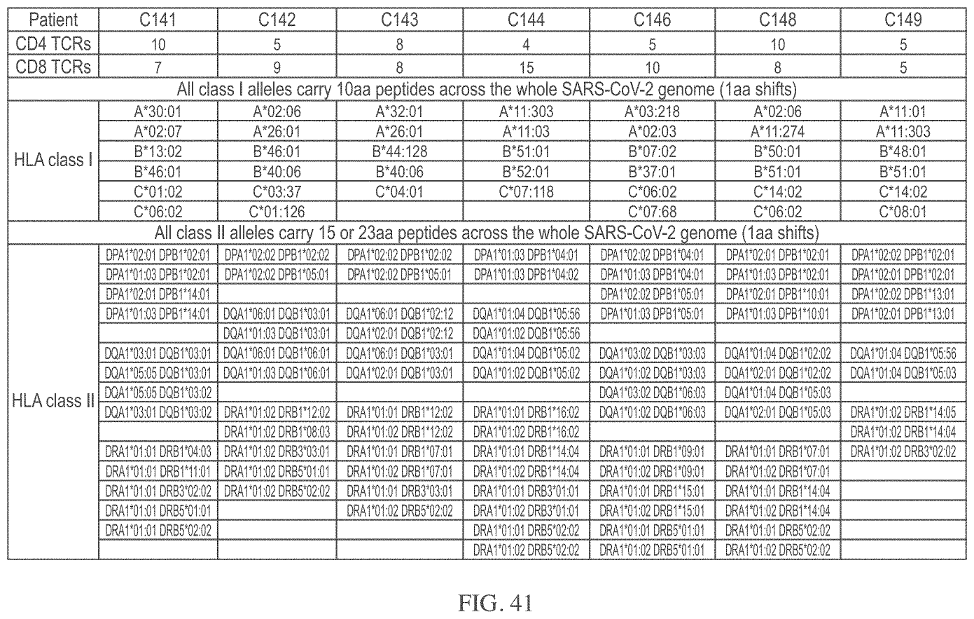

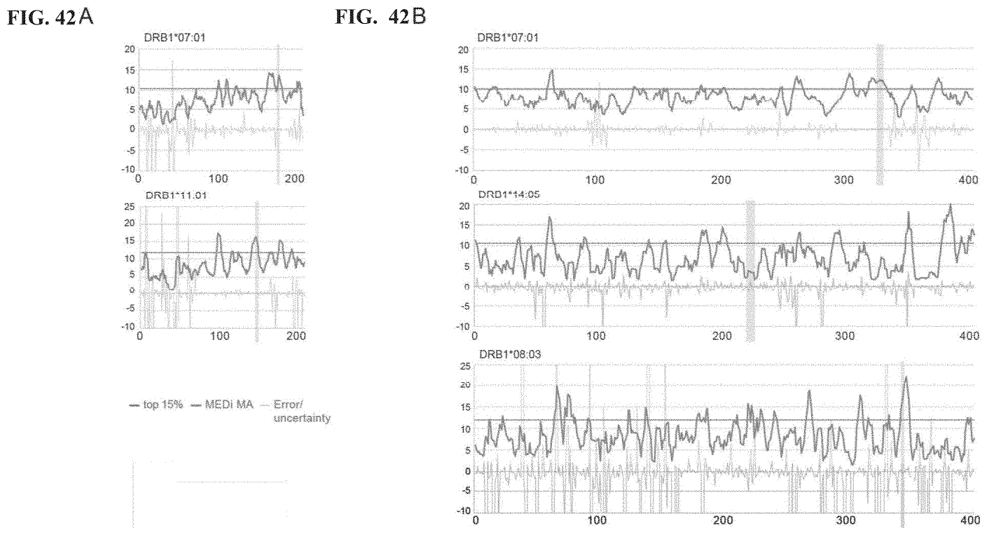

To further test MEDi and the proposed scoring approach for antigen presentability the analysis was extended to natural T cell targets. Although T cell SARS-CoV-2 reactivities against peptides scattered across the viral genome have been reported, analyses that comprehensively decode “immune synapses”, including TCR alpha and beta chain sequences, the recognized peptide and the presenting HLA, are sparse. Thus, the MCR technology (Kisielow, J. et al. (2019) Nat. Immunol. 20:652-662) (FIG. 37A) was used and single chain trimers (Hansen, Ted. H. & Lybarger, L. (2006) Cancer Immunol. Immunother. 55:235-236) (linked to the intracellular domain of the TCR zeta chain (SCTz) (Zhang, T. et al. (2004) FASEB J. 18:600-602; Joglekar, A. V. et al. (2019) Nat. Methods 16:191-198), to de-orphan TCRs of enriched clonotypes from the BALs of COVID-19 patients, described recently by Liao et al. (Nat. Med. (2020)26:842-844). Liao et al., provided high resolution single cell data indicating aberrant cellular responses and identified expanded T cell clonotypes, but they neither decoded their antigenic specificity, nor the HLA restriction. To address this, the 109 most enriched TCRs were cloned, expressed in a T cell line, and subjected to an unbiased epitope screening. This included MCR2 libraries containing all possible 23aa SARS-CoV-2-derived peptides (1aa shifts through all proteins) and libraries containing all possible 10aa SARS-CoV-2-derived peptides presented in the context of SCTz. This setup allowed for an unprecedented, complete screen of all SARS-CoV-2 peptides in the context of all HLAs from every patient (FIG. 41). Screening these patient specific MCR2 libraries of approximately 120,000 different peptide-MCR2 combinations and 60.000 peptide-SCTz combinations required at least 4 rounds of enrichment (FIG. 37B) before single cell clones revealed the specific peptides and the presenting HLA alleles (FIG. 37C). As expected, not all TCRs showed reactivity against SARS-CoV-2 antigens, but the cognate peptides and the HLA restriction for 8 CD4 and 3 CD8 TCRs were identified (FIG. 37C, FIG. 37D).

A variety of peptides presented by several HLA alleles were found. For example, 3 CD4 T cell clones from severely affected patient C148 recognized peptides from the SARS-CoV-2 proteins spike (S), membrane glycoprotein (M) and nucleocapsid (N), all presented by DRB1*07:01. TCR091 from patient C141, reacted with the membrane glycoprotein-derived peptide M₁₄₆₋₁₆₅ presented by DRB1*11:01. In line with a high immunogenicity of this epitope, Peng et al. ((2020) Nat. Immunol. 21:1336-1345). Interestingly, two of the CD4 T cell specific peptides identified in this study (S₇₁₄₋₇₂₈ and N₂₂₁₋₂₄₂) were mutated in the SARS-CoV-2 B1.1.7 variant first identified in Britain (Rambaut, A., Loman, N. & Volz, E. (2020 COVID-19 Genomics Consortium UK). Reporter cells were transduced with MCR2 carrying the WT and mutated S₇₁₄₋₇₂₈ or N₂₂₁₋₂₄₂ peptides (FIG. 37E), and it was discovered that S_{714-728 (T716I)} was not recognized by the TCR₀₀₇ (FIG. 37F). Recognition of the N₂₂₁₋₂₄₂ peptide was unaffected by the mutation, suggesting that Ser236 was not part of the minimal epitope (FIG. 37F), nor did it affect peptide presentation.

MEDi Indicates Efficient Presentation of Immunogenic CD4 T Cell Epitopes

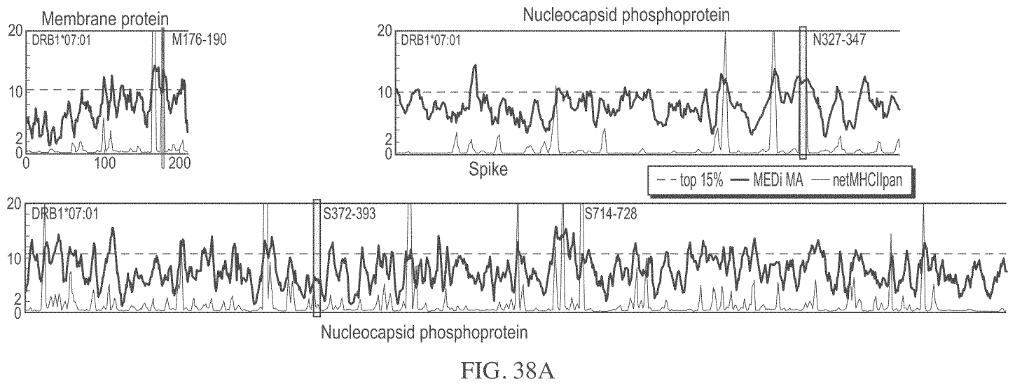

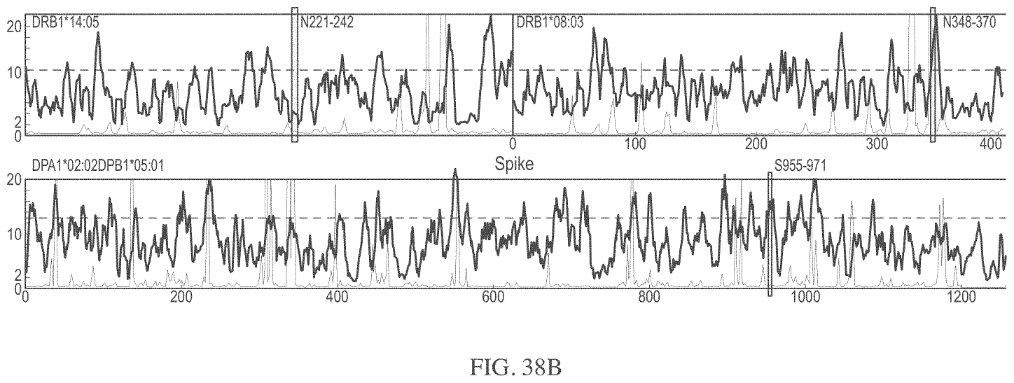

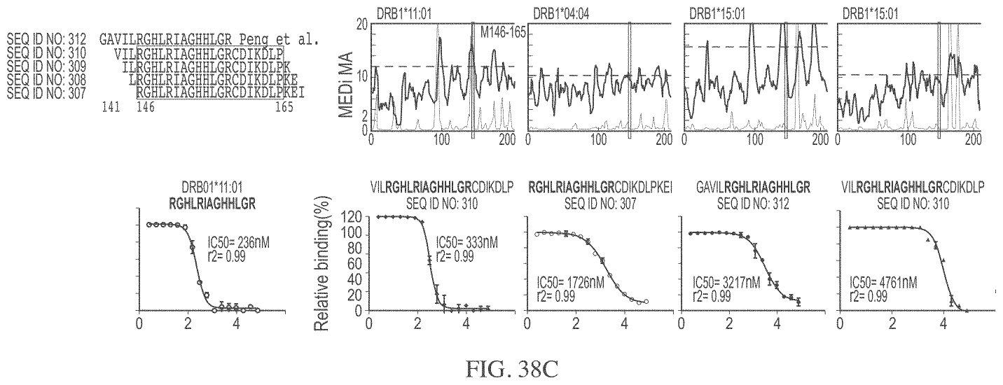

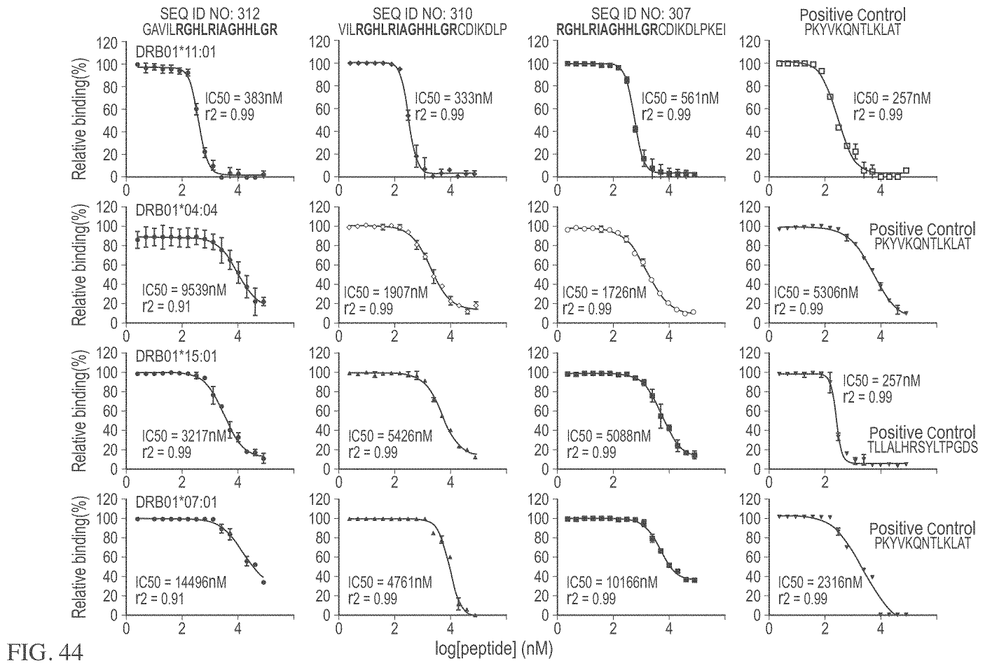

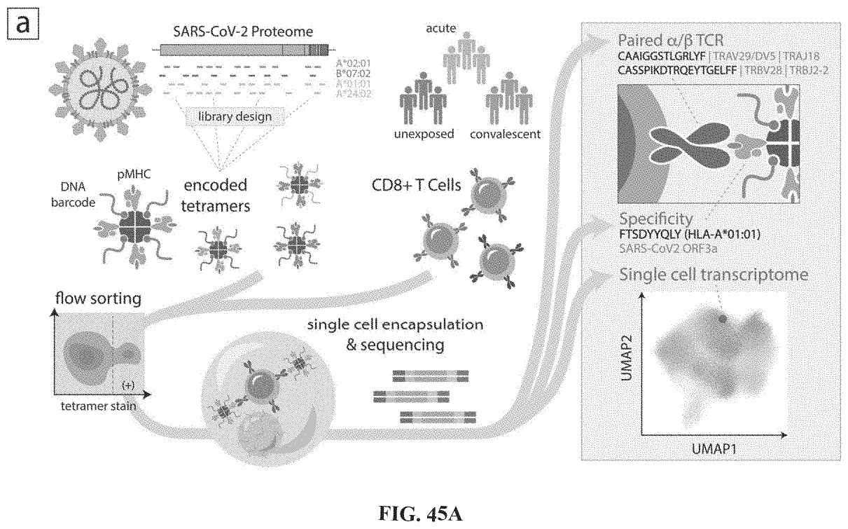

Next, the presentability of the CD4 T cell targets identified in the MCR screens was analyzed. MEDi data indicated good presentability of the TCR091 target peptide region by DRB1*11:01 (FIG. 38C and FIG. 44). Furthermore, consistent with high reactivity among patients, MEDi suggested presentation of this region by other HLA alleles like DRB1*04:04 and DRB1*15:01, and to a lower extent by DRB1*07:01 (FIG. 38). NetMHCIIpan only predicted DRB1*11:01, but the competitive peptide binding assay confirmed the MEDi results: DRB1*11:01 showed the highest IC₅₀ (236 nM-561 nM), followed by DRB1*04:04(1.7-9.5 μM) and DRB1*15:01(3.2-5.4 μM) and the lowest DRB1*07:01 (4.7-14 μM) (FIG. 38C and FIG. 44). Even if these values do not precisely indicate differences in binding affinity, because the competing fluorescent peptides bind the HLAs with different affinity, the results highlight the advantages of MEDi over netMHCIIpan for discovering low-affinity peptide presentation.

Next, MEDi scores of the other immunogenic peptides found in this study were analyzed, and were compared to netMHCIIpan predictions (FIGS. 38A-B). All of the CD4 T cell immunogenic peptides were found in the MEDi peaks, with S₉₅₅₋₉₇₁ presented by DPA1*02:02/DPB1*05:01 and N₂₂₁₋₂₄₂ presented by DRB1*14:05 being uniquely identified by MEDi. Also, 7 of the 8 peptides passed the MEDi-MA85 threshold. Only S₃₇₂₋₃₉₃ showed a peak with lower MEDi scores, suggesting lower affinity HLA binding. Thus, selecting all immunogenic peptides for screening applications may require an adjustment of the MEDi threshold. Taken together, these results indicate that MEDi selected peptides are enriched for immunogenic epitopes and that MEDi has an advantage over in-silico predictions for MHC class II alleles, where no high-quality mass spec results or other training data are available.

MEDi Reveals Candidate Immune-Escape Mutants

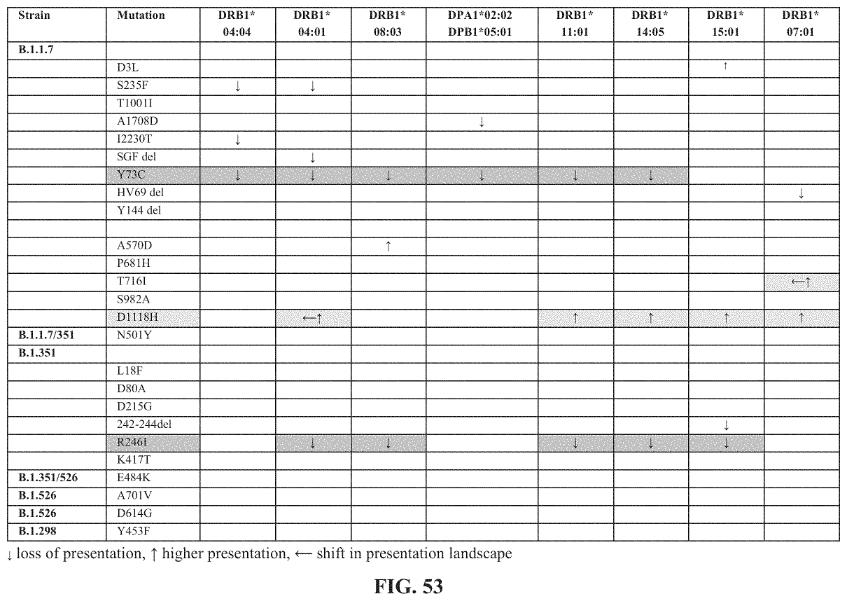

Having established the ability of MEDi to determine presentable peptides, MEDi was used to analyze the effects of 25 mutations present in SARS-CoV-2 variant strains expanding across the globe (FIG. 53). MCR2 libraries were generated containing mutation-overlapping 15mer peptides in the context of 8 different HLA alleles and MEDi analysis was performed. As shown in FIG. 39 and FIG. 54, there was a notable HLA-dependent difference in mutant peptide presentability. ORF8 Y73C and spike R2461 mutations abolished peptide presentation by 6/8 and 5/8 HLA alleles, respectively, suggesting the possibility of immune escape of the virus in patients with these alleles. Several other mutated peptides from nucleocapsid, ORF1a and ORF8 were inefficiently presented by DRB1*04:04 and DRB1*04:01 and DPA1*02:02/DPB1*05:01. For some, the molecular mechanism could be envisioned, e.g. mutations 12230T and Y73C disrupted the N-terminal hydrophobic amino acid stretches constituting a binding motif for DRB1*04:04 (Andreatta et al. supra) (FIG. 39A-B). Also, the spike HV69 deletion reduced presentation by DRB1*07:01. The other alleles showed no difference between WT and mutated peptides, with a few exceptions where presentability of mutated peptides was enhanced. In particular, the spike D1118H mutation stabilized binding of several peptides to DRB1*14:05, DRB1*15:01 and DRB1*07:01 and caused a shift in the peptide presentation landscape of DRB1*04:01 (FIGS. 39A and C). In line, the peptide S1111-1130(D1118H) triggered weaker responses in DRB1*04:01 positive patients (Reynolds et al. (2021) Science, Article eabh1282). Similarly, T716I affected the presentation landscape of DRB1*07:01 and abolished T cell reactivity (FIG. 37F). While FACS staining (FIG. 37E) and MEDi-MA scores showed that the 15mer S_{714-728(T716I)} was presented as well as the WT, they also indicated that mutated peptides starting from Asp₇₀₂ to Asn₇₁₀ would be presented substantially better than WT (FIG. 39D). Indeed, the T716I mutation introduced a perfect P9 anchor residue at position 716, complementing residues Tyr₇₀₇/Ser₇₀₈, Ser₇₁₁ and Ala₇₁₃ to form a good DRB1*07:01 binding motif (FIG. 39D and FIG. 36A). Furthermore, the T716I mutation introduced additional DRB1*07:01-binding motifs potentially allowing three different presentation registers for peptide S_{714-728(T716I)} (FIGS. 39E and F): first, comprising a weak HLA-binding motif starting at Ile714, with Thr₇₁₆ directly facing the TCR; second starting with the mutated Ile₇₁₆ as a new anchor residue; and third, where the T716I mutation would be outside of the minimal epitope for TCR007. Thus, the T716I mutation could abrogate TCR recognition by either of two mechanisms: it could alter peptide presentation on DRB1*07:01, or it could abolish direct TCR007 contacts.

To answer this question, 12mer peptides S₇₁₄₋₇₂₅, S_{714-725(T716}I) and S₇₁₇₋₇₂₈ were cloned into the DRB1*07:01-MCR2 and cocultured MCR2+reporter cells with TCR007 T cells. As shown in FIG. 39G, all constructs were expressed well with S₇₁₇₋₇₂₈ reaching the highest levels indicating best presentation. Intriguingly, TCR007 recognized S₇₁₇₋₇₂₈, but not S₇₁₄₋₇₂₅ (FIG. 39H). This indicates that T716I abrogated TCR recognition of the S₇₁₄₋₇₂₈₁₅mer indirectly.

These results suggest several mechanisms of peptide presentation modulation and highlight the ability of the MEDi platform to decipher molecular details underlaying possible viral immune escape strategies. Comprehensive analyses of the arising viral mutants, studying the relation of presentability and immunogenicity, will be important for the development of future therapeutics.

Discussion

Identifying the specificity of pathogen-reactive lymphocytes is important for the fields of therapeutics and vaccine development. While protection from viral infections is mostly attributed to B cell and CD8 T cell effector functions, the balance between enabling and restricting them decides about life and death of the host. Thus, understanding the CD4 T cell reactivity, which orchestrates these responses, is important, and deep knowledge of epitope presentation by HLA class II would greatly help clinical developments. However, owing to the limited sensitivity of mass spectroscopy and varying accuracy of the in silico methods, it is difficult to generate peptide presentation landscapes across multiple HLA alleles. MEDi, provides a powerful alternative approach, based on functional cell surface expression of the MCR2 molecules. It is HLA agnostic due to the association of MCR2 with the CD3 chains and allows unbiased, fast, and affordable testing of all antigen-derived peptides for their ability to be presented by an HLA. The results described herein indicate that antigenic peptides usually reside within the MEDi high regions (some missed by prediction algorithms), provide a list of presentable SARS-CoV-2 peptides for several different HLA alleles and describe mechanisms for viral immune evasion. The MEDi results were validated biochemically and biologically, through presentation analysis of immunogenic epitopes discovered by “de-orphaning” TCRs from over a hundred T cells enriched in the lungs of acute COVID-19 patients. Highlighting the importance of CD4 T cells, it was discovered that among the enriched TCRs, 8/47 (17.0%) of the CD4-derived ones and 3/63 (4.7%) of the CD8-derived ones recognized SARS-CoV-2 peptides. The appearance of mutated SARS-CoV-2 with higher transmissibility raises important questions about the selective pressure that gave rise to the fitter variants and the role of immune escape in their evolution. While viral escape from antibody-mediated neutralization has been well documented for many diseases, much less is known about a potential selective pressure to evade T cell reactivity. Understanding HLA presentation and TCR recognition of mutant and WT epitopes is important in this regard. Several mutations present in the emerging SARS-CoV-2 variant strains reduced presentability of the affected peptides by several HLA class II alleles. Furthermore, 2 of the 8 immunogenic peptides found in this study were targeted by the arising mutations. Both mutations were just outside of the minimal epitopes, but one still affected TCR recognition. Two different mechanisms of escape were tested and it was determined that Thri6 was not directly bound by the TCR, but that the T716I mutation altered peptide presentation by enabling binding in a different register. This evasion strategy would affect all T cells recognizing this peptide, so the T716I mutation might provide a bigger advantage for the virus than appreciated so far. Furthermore, given the optimal peptide length for MHC class II being 18-20 amino acids (O'Brien, C. et al. (2008) Immunome Res. 4:6), it is very likely that most peptides, comprising the HLA binding core starting at Phe718, will include Thr/Ile716. A significant advantage of MEDi is that it is easily scalable to the thousands of alleles present in humans and enables peptide presentability studies with patient-specific HLA alleles for which no good training data are available. Consistently, the immunogenic spike S₉₅₅₋₉₆₉ peptide presented by DPA1*02:02/DPB1*05:01 and N₂₂₁₋₂₄₂ presented by DRB1*14:05, both MEDi high, were not well predicted by netMHCIIpan. Furthermore, with MEDi it was possible to provide presentability information for any immunogenic peptide across multiple HLA alleles. This is exemplified by the very immunogenic membrane protein peptide M₁₄₆₋₁₆₅, recognized by TCR091 in the context of DRB1*11:01 and shown by MEDi to be also presentable by several other HLAs, not predicted by netMHCIIpan. However, the information gained from MEDi can support further training of predictive models similar to Rappazzo et al. ((2020) Nat. Commun. 11:4414).

The results presented in this study validate the MEDi platform and provide insights into the molecular mechanisms of SARS-CoV-2 peptide presentation and potential escape from T cell recognition. MEDi should help closing the gap in peptide-presentation landscape for thousands of HLA alleles and be useful for the development of novel therapeutical approaches beyond prevention of COVID-19 or treatment of SARS-CoV-2 patients.

### Example 22: Allelic Variation in Class I HLA Determines Pre-Existing Memory Responses to SARS-CoV-2 that Shape the CD8⁺ T Cell Repertoire Upon Subsequent Viral Exposure

Effective presentation of antigens by HLA class I molecules to CD8⁺ T cells is required for viral elimination and generation of long-term immunological memory. In this study, a single-cell, multi-omic technology was applied to generate the first unified ex vivo characterization of the CD8⁺ T cell response to SARS-CoV-2 across 4 major HLA class I alleles. It was found that HLA genotype conditions key features of epitope specificity, TCR a/b sequence diversity, and the utilization of pre-existing SARS-CoV-2 reactive T cell memory pools. Single-cell transcriptomics revealed functionally diverse T cell phenotypes associated with both disease stage and epitope specificity. The results described herein show that HLA variations influence pre-existing immunity to SARS-CoV-2 and shape the immune repertoire upon subsequent viral exposure.

### Introduction

Elicitation of a robust and durable neutralizing antibody response following immunization of large sections of the population with approved SARS-CoV-2 vaccines is limiting viral transmission and decreasing mortality, providing hope that the global threat from the COVID-19 pandemic is diminishing. However, the appearance of new emerging viral variants warrants continued vigilance. A more complete understanding of the underlying cellular mechanisms that regulate host immunity and guarantee long term protection is required. Infection with SARS-CoV-2 leads to an upper respiratory tract infection, which can be benign or even asymptomatic. If not controlled by the immune response, it can evolve into a lethal pneumonia with immunopathology due to excessive amplification of the innate inflammatory response, complicated by several extra-respiratory manifestations (Huang et al. (2020) Lancet 395:497-506). While humoral responses play an important role in immunological control of infection, the generation of effective cellular immunity and expansion of cytotoxic CD8⁺ memory T cells is also required to eliminate virally infected cells (Sette and Crotty (2021) Cell 184:861-880) as shown from the earlier SARS-CoV-1 epidemic, even in the absence of seroconversion (Ng et al. (2016) Vaccine 34:2008-2014; Seow et al. (2020) Nat Microbiol 5:1598-1607; Rydyznski Moderbacher et al. (2020) Cell 183:996-1012 e1019).

Several recent studies have focused on the discovery of relevant SARS-CoV-2 epitopes in both CD4⁺ and CD8⁺ responses, leveraging in silico predictions, stimulation/expansion with peptide pools (Peng et al. (2020) Nat Immunol 21:1336-1345; Braun et al. (2020) Nature 587:270-274; Nelde et al. (2021) Nat Immunol 22:74-85; Sekine et a1. (2020) Cell 183:158-168 e114; Schulien et al. (2021) Nat Med 27:78-85; Mateus et al. (2020) Science 370:89-94; Ferretti et al. (2020) Immunity 53:1095-1107 e1093), and tetramer binding (Kared et al. (2020) bioRxiv; Saini et al (2020) bioRxiv). Collectively, these studies identified a number of immunodominant epitopes derived across the viral proteome including structural and non-structural proteins (Braun et al. (2020) Nature 587:270-274; Nelde et al. (2021) Nat Immunol 22:74-85; Sekine et al. (2020) Cell 183:158-168 e114; Schulien et al. (2021) Nat Med 27:78-85; Mateus et al. (2020) Science 370:89-94; Grifoni et al. (2020) Cell 181:1489-1501 e1415; Weiskopf et al. (2020) Sci Immunol 5; Snyder et al. (2020) medRxiv). Interestingly, some of these specificities were also detected in uninfected individuals, suggesting potential cross-reactivity from endemic human coronaviruses (HCoV) to which the population is routinely exposed (Gorse, G. B. et al. (2010) Clin Vaccine Immunol 17:1875-1880), though a direct connection to pre-existing memory cells has not been established.

The breadth and nature of the cellular immune response to SARS-CoV-2 infection is driven by diversity in both TCR repertoire and human leukocyte antigen (HLA) genetics. Mammalian cells express up to six different HLA class I alleles that shape antigen presentation in disease, and allelic diversity has been associated with both disease susceptibility and outcome of viral infections (MacDonald et al. (2020) J Infect Dis 181:1581-1589; Ochoa et al. (2020) Vivol J17:128). There are divergent reports regarding HLA polymorphism and COVID-19 incidence and severity although the major GWAS studies clearly show no dominant effect of the locus (Severe Covid GWAS Group (2020) N Engl J Med 383:1522-1534; Pairo-Castineira et al. (2021) Nature 591:92-98; Habel et al. (2020) Proc Natl Acad Sci USA 117:24384-24391; Shkurnikov et al. (2021) Front Immunol 12:641900; Nguyen et al. (2020) J Vivol 94: e00510-20). Together with genetic influences on HLA-associated antigen presentation, the clonal selection of T cell receptors (TCRs) that compose an individual's repertoire contributes to the nature and dynamics of the antiviral response, including cellular cytotoxicity and memory formation. Interestingly, despite a potential TCR diversity of 10¹⁵ (Qi et al. (2014) Proc Natl Acad Sci USA 111:13139-13144), several studies have described “public” T cell responses in COVID-19, where complementary determining region (CDR) sequences are conserved within and across individuals (Snyder et al. (2020) medRxiv). The extent to which TCR diversity, especially in the context of epitope specificity restricted to HLA, contributes to response is not well understood.

Here, a unique technology was used to elucidate, at the single-cell resolution, the connection between T cell specificity, HLA variation, conserved features of paired a/b TCR repertoires, and cellular phenotype observed in CD8⁺ T cell responses to SARS-CoV-2 infection. 108,078,030 CD8+ T cells ex vivo were profiled across 76 acute, convalescent, or unexposed individuals and identified T cell specificity to 648 epitopes presented by four HLA alleles across the SARS-CoV-2 proteome, few of which are implicated by the current variants of concern. Epitope-specific TCR repertoires were surprisingly public in nature, though a high degree of pre-existing immunity associated with a clonally diverse response to HLA-B*07:02 was found, which can efficiently present homologous epitopes from SARS-CoV-2 and HCoVs. Transcriptomic analysis and functional validation were used to confirm a central memory phenotype and TCR cross-reactivity in unexposed individuals with HLA-B*07:02. The data provided in this Example suggests a strong association between HLA genotype and the CD8+ T cell response to SARS-CoV-2, which may have important implications for understanding herd immunity and elements of vaccine design that are likely to confer long-term immunity to protect against SARS-CoV-2 variants and related viral pathogens.

### Materials and Methods

Antigen library design. Antigenic peptide libraries were made by scoring all possible 9mer peptides derived from the entire SARS-CoV-2 (NC_045512.2) proteome using netMHC-4.0 (29) in the HLA-A*02:01, HLA-A*01:01, HLA-A*24:02 or HLA-B*07:02 alleles. SARS-CoV-1 peptides that had evidence of T cell positive assays, obtained from the Immune Epitope Database and Oh et al. (2011) J Virol 85:10464-10471, and that were highly homologous to their SARS-CoV-2 counterparts within Hamming-distance of 2 were converted to 9-mers. Additionally, SARS-CoV-2 peptides predicted to raise immunogenic responses by others were also included (Campbell et al. (2020) bioRxiv; Grifoni et al. (2020) Cell Host Microbe 27:671-680 e672). Finally, libraries included a set of well-defined viral epitopes from Cytomegalovirus, Epstein-Barr virus, and Influenza viruses (CEF peptide pool) that elicit T cell responses in the population at large. Antigenic peptides with 500 nM affinity or lower were then selected.

Production of tetramer library pools. HLA-A*01:01, -A*02:01, -A*24:02 and HLA-B*07:02 extracellular domains were expressed in E. coli and refolded along with beta-2-microglobulin and UV-labile place-holder peptides STAPGJLEY (SEQ ID NO: 16), KILGFVFJV (SEQ ID NO: 15), VYGJVRACL (SEQ ID NO: 11) and AARGJTLAM (SEQ ID NO: 14), respectively (Altman and Davis (2016) Curr Protoc Immunol 115:171311-171344). The MHC monomer was then purified by size exclusion chromatography (SEC). MHC tetramers were produced by mixing alkylated MHC monomers and azidylated streptavidin in 0.5 mM copper sulfate, 2.5 mM BTTAA and 5 mM ascorbic acid for up to 4 h on ice, followed by purification of highly multimeric fractions by SEC. Individual peptide exchange reactions containing 500 nM MHC tetramer and 60 uM peptide were exposed to long-wave UV (366 nm) at a distance of 2-5 cm for 30 min at 4° C., followed by 30 min incubation at 30° C. A biotinylated oligonucleotide barcode (Integrated DNA Technologies) was added to each individual reaction followed by 30 minute incubation at 4° C. Individual tetramer reactions were then pooled and concentrated using 30 kDa molecular weight cut-off centrifugal filter units (Amicon).

Cell Staining. Peripheral blood mononuclear cells (PBMCs) from convalescent COVID-19 positive donors or unexposed donors were obtained from Precision 4 Medicine (USA), the Massachusetts Consortium on Pathogen Readiness (MassCPR), or CTL (USA), all under appropriate informed consent. PBMCs were thawed, and CD8+ T cells were enriched by magnetic-activated cell sorting (MACS) using a CD8+ T cell Isolation Kit (Miltenyi) following the manufacturers protocol. The CD8+ T cells were then stained with 1 nM final concentration tetramer library in the presence of 2 mg/mL salmon sperm DNA in PBS with 0.5% BSA solution for 20 minutes. Cells were then labeled with anti-TCR ADT (IP26, Biolegend) for 15 minutes followed by washing. Tetramer bound cells were then labeled with PE conjugated anti-Flag antibody (BioLegend) followed by dead cell discrimination using 7-amino-actinomycin D (7-AAD). The live, tetramer positive cells were sorted using a Sony MA900 Sorter (Sony).

Single-cell Sequencing. Tetramer positive cells were counted by Nexcelom Cellometer (Lawrence, Mass., USA) using AOPI stain following manufacturer's recommended conditions. Single-cell encapsulations were generated utilizing 5′ v1 Gem beads from 10× Genomics (Pleasanton, Calif., USA) on a 10× Chromium controller and downstream TCR, and Surface marker libraries were made following manufacturer recommended conditions. All libraries were quantified on a BioRad CFX 384 (Hercules, Calif., USA) using Kapa Biosystems (Wilmington, Mass., USA) library quantified kits and pooled at an equimolar ratio. TCRs, surface markers, and tetramer generated libraries were sequenced on Illumina (San Diego, Calif., USA) NextSeq550 instruments. Sequencing data were processed using the Cell Ranger Software Suite (Version 3). Samples were demultiplexed and unique molecular identifier (UMI) counts were quantified for TCRs, tetramers, and gene expression.

Single-cell Transcriptomic Analysis. Hydrogel-based RNA-seq data were analyzed using the Cell Ranger package from 10× Genomics (v3.1.0) with the GRCh38 human expression reference (v3.0.0). Except where noted, Scanpy (v1.6.0,(52)) was used to perform the subsequent single cell analyses. Any exogenous control cells identified by TCR clonotype were removed before further gene expression processing. Hydrogels that contain UMIs for less than 300 genes were excluded. Genes that were detected in less than 3 cells were also excluded from further analysis. Several additional quality control thresholds were also enforced. To remove data generated from cells likely to be damaged, upper thresholds were set for percent UMIs arising from mitochondrial genes (13%). To exclude data likely arising from multiple cells captured in a single drop, upper thresholds were set for total UMI counts based on individual distributions from each encapsulation (from 1500 to 3000 UMIs). A lower threshold of 10% was set for UMIs arising from ribosomal protein genes. Finally, an upper threshold of 5% of UMIs was set for the MALAT1 gene. Any hydrogel outside of any of the thresholds was omitted from further analysis. A total of 15,683 hydrogels were carried forward. Gene expression data were normalized to counts per 10,000 UMIs per cell (CP10K) followed by log 1p transformation: ln(CP10K+1).

Highly variable genes were identified (1,567) and scaled to have a mean of zero and unit variance. They were then provided to scanorama (v1.7) (Hie et al. (2019) Nat Biotechnol 37:685-691) to perform batch integration and dimension reduction. These data were used to generate the nearest neighbor graph which was in turn used to generate a UMAP representation that was used for Leiden clustering. The hydrogel data (not scaled to mean zero, unit variance, and before extraction of highly variable genes) were labeled with cluster membership and provided to SingleR (v1.4.0) (Aran et al. (2019) Nat Immunol 20:163-172) using the following references from Celldex (v1.0.0) (Aran et al. (2019) Nat Immunol 20:163-172), Monaco Immune Data, Database Immune Cell Expression Data, and Blueprint Encode Data. SingleR was used to annotate the clusters with their best-fit match from the cell types in the references. Clusters that yielded cell types other than types of the T cell lineage were removed from consideration and the process was repeated starting from the batch integration step. The best-fit annotations from SingleR after the second round of clustering and annotation were assigned as putative labels for each Leiden cluster. Further clustering of transcriptomic data was performed across the genes shown in FIG. 5 using KMeans in sklearn (v0.24) with n_clusters set to 8. As the method has a preference to assign like-sized clusters, further consolidation of two central memory clusters was performed.

In order to provide corroboration for the SingleR best-fit annotations and further evidence as to the phenotype of the clusters, gene panels representing functional categories (Naïve, Effector, Memory, Exhaustion, Proliferation) were used to score each hydrogel's expression profiles using scanpy's “score_genes” function (Wolf et al. (2018) Genome Biol 19:15), which compares the mean expression values of the target gene set against a larger set of randomly chosen genes that represent background expression levels. The gene panels for each class were: Naïve—TCF7, LEF1, CCR7; Effector—GZMB, PRF1, GNLY; Memory—AQP3, CD69, GZMK; Exhaustion—PDCD1, TIGIT, LAG3; Proliferation—MKI67, TYMS. The gene expression matrix for all hydrogels were first imputed using the MAGIC algorithm (v2.0.4) (van Dijk et al. (2018) Cell 174:716-729 e727). These functional scores were the only data generated from imputed expression values.

Scoring pMHC-TCR interactions. Tetramer data analysis was performed using Python (v3.7.3). For each single-cell encapsulation, tetramer UMI counts (columns) were matrixed by cell (rows) and log-transformed. Duplicates of this matrix were independently Z-score transformed by row or column, and subsequently median-centered by the opposite axis (column or row), respectively. For each pMHC-cell interaction, this provided two scores—inter-tetramer (Sₜₑₜ) and inter-cell (S_{cell}), which were used to calculate a classifier for unique CDR3 a/b clonotypes across N cells as N*Sₜₑₜ*Sₚₑₗₗ. A classifier threshold of 40 for positive interactions.

TCR Network Analysis. TCR motif analysis was performed using scirpy (v0.6.1) with receptor_arms=“any,” metric=“alignment,” and default cutoff of 10. Once clusters were identified, sequence alignment was performed using the pairwise2 module in Biopython (v1.78) and visualized using logomaker (v0.8).

Recombinant TCR validation. Recombinant TCRs identified from patient samples were ordered from TWIST Biosciences in the pLVX-EF1a lentiviral backbone (Takara) as a bicistronic TCRb-T2A-TCRa vector. Viral supernatants from transfected HEK 293 T cells were collected 48 and 72 hours after transfection and added to the parental TCRab^{-/-} Jurkat J76 cell like (Jutz et al. (2016) J Immunol Methods 430:10-20) expressing CD8 and an NFAT-GFP reporter, referred to as J76-CD8-NFAT-GFP. Recombinant TCR surface expression was confirmed through flow cytometry by staining transduced J76-CD8-NFAT-GFP cells with anti-CD3-PE (Clone UCHT1) and anti-TCRab-APC (Clone IP26) antibodies.

To assess functional activity of recombinant TCRs, J76-CD8-NFAT-GFP expressing recombinant TCRs were incubated at a 1:1 ratio with the HLA-A*02:0 rand HLA-B*07:02⁺HCC 1428 BL (ATCC CRL-2327) lymphoblastic cell line, with a final concentration of 0.5% DMSO (vehicle) or 50 mM of cognate peptide (New England Peptide, >95% pure). Cell mixtures were incubated in the Sartorius IncuCyte at 37° C., 5% CO2 overnight and analyzed for NFAT-GFP expression measured as total integrated intensity (GCU×um²/image) at 12 hours after assay setup. At 16 hours, cells were removed from the IncuCyte and subsequently washed and blocked with BD Staining Buffer (BD 554656), stained with anti-CD3-PE-Cy7 (Clone UCHT1) and anti-CD69-APC (Clone FN50) antibodies, and analyzed using the Intellicyt iQue Screener Plus and FlowJo v10. CD69 activity was measured as percent positive of CD3⁺ cells.

### Results

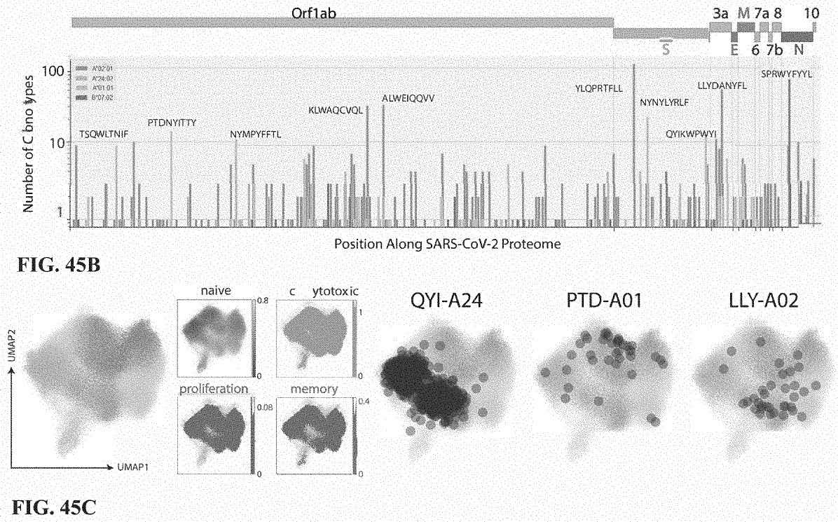

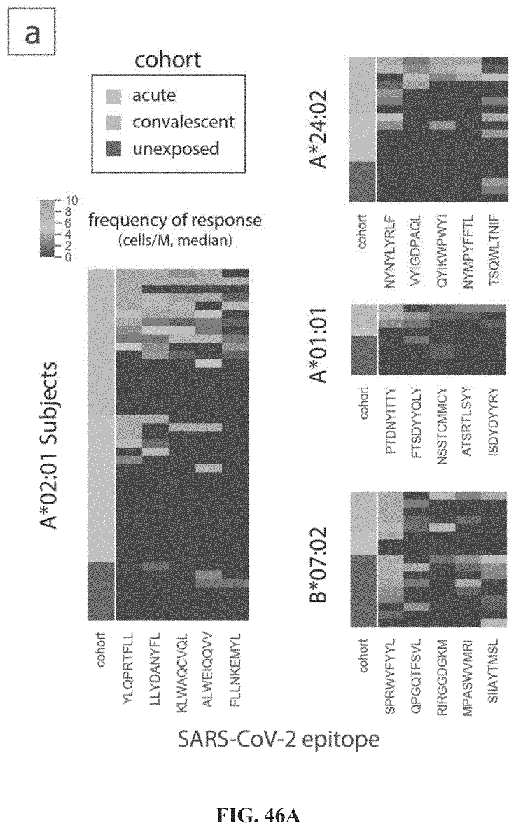

Direct ex-vivo detection and decoding of SARS-CoV-2-specific CD8+ T cells: Single-cell RNA sequencing with DNA-encoded peptide-HLA tetramers was used to characterize CD8⁺ T cell responses to SARS-CoV-2 across multiple Class I alleles in subjects with varying degrees of disease severity. The technology illustrated in FIG. 45A simultaneously determines the specificity of paired a/b TCR sequences for HLA-restricted epitopes and provides transcriptomic phenotype at single-cell resolution. Peptide-HLA tetramer libraries were created to ensure comprehensive coverage of SARS-CoV-2 and related betacoronaviruses across four class I HLA alleles prevalent in North America (A*02:01, B*07:02, A*01:01, and A*24:02, hereafter A*02, B*07, A*01, and A*24). Library inclusion was determined computationally using predicted HLA binding (NetMHC-4.0)(Andreatta et al. (2016) Bioinformatics 32:511-517) of candidate peptides from a set of all possible 9-mers from the SARS-CoV-2 proteome (40% from structural, 60% from non-structural proteins), potentially immunogenic neopeptides from known SARS-CoV-2 variants, and immunogenic epitopes from SARS-CoV-1. A total of 1,355 SARS-CoV-2 related epitopes were included in the libraries in addition to well-characterized epitopes from common endemic viruses (CMV, EBV, and influenza).

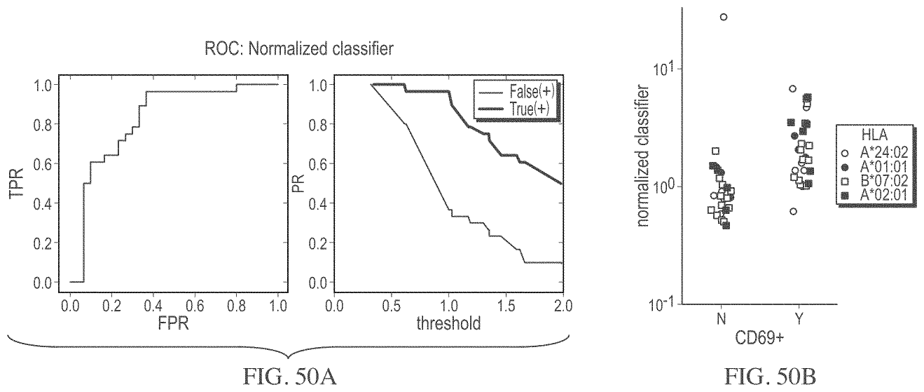

The peptide-HLA tetramer libraries were used to interrogate PBMCs from individuals who had been infected with SARS-CoV-2 (N=28 convalescent, N=27 with acute disease that required hospitalization), or who were unexposed (N=23). For each sample, CD8⁺ cells were isolated from PBMCs, incubated with HLA-matched tetramer libraries, and sorted by flow cytometry to enrich viable, tetramer positive cells. Sorted single cells were encapsulated with DNA-encoded hydrogel beads to provide cell-specific barcodes and unique molecular identifiers (UMIs) that could be used to unify reads across independent sequencing libraries for TCR, peptide-HLA tetramer, and mRNA (FIG. 45A). The specificity of TCRs was determined using a classification method that identified UMI counts for TCR-peptide-HLA interactions that were outliers when Z-score transformed within and across cells for each sample. The resulting classifier was evaluated against functional assay data for each allele by receiver-operator curve (ROC) analysis to identify thresholds, which were then used for normalization. The normalized classifier evaluated by ROC analysis provided an area under the curve (AUC) of 0.82 (FIG. 50), and at a threshold of 1, which was applied to the entire data set, yielded a true positive rate of 93% and a false positive rate of 32%.

From the 55,956,215 CD8⁺ cells interrogated from acute and convalescent COVID-19 patients, high-confidence TCR-peptide-HLA interactions were identified across 434 immunogenic SARS-CoV-2-derived epitopes and 1,163 independent a/b TCR clonotypes (FIG. 45B). The immunodominant epitopes discovered ex vivo were consistent with those measured by other means, but many epitopes were identified with less dominant representation (yet observed with two or more reactive clonotypes), 188 of which had not been previously reported as minimal epitopes. Importantly, specificity to SARS-CoV-2 antigens was observed across the entire proteome, generally distributed in a manner consistent with protein lengths, as summarized below in TABLE 13:

TABLE 13

| Antigen | A*01:01 | A*02:01 | A*24:02 | B*07:02 | Summary |
|---|---|---|---|---|---|
| SARS2_ORF1AB | 51 | 419 | 77 | 26 | 573 |
| SARS2_SPIKE | 11 | 158 | 46 | 6 | 221 |
| SARS2_N | — | 45 | — | 72 | 117 |
| SARS2_3A | 6 | 73 | 6 | 1 | 86 |
| SARS2_M | 6 | 24 | 11 | 2 | 43 |
| SARS2_7A | — | 12 | — | 1 | 13 |
| SARS2_E | 2 | 10 | — | — | 12 |
| SARS2_9B | — | 9 | — | — | 9 |
| SARS2_10 | — | 6 | 1 | — | 7 |
| SARS2_14 | — | 6 | — | — | 6 |
| SARS2_7B | — | 5 | — | — | 5 |
| SARS2_8 | — | 5 | — | — | 5 |
| SARS2_6 | 1 | 3 | — | — | 4 |

Of relevance, 85 of these epitopes were derived from the Spike protein currently used in vaccines, but only six of them (a total of 20 CD8⁺ T cell clonotypes in the study) would be affected by the recent SARS-CoV-2 variants (B.1.1.7, B.1.351, P.1).

Dimensionality reduced projections of mRNA expression for 224,780 CD8⁺ T cells revealed the broad phenotypic variance observed within this study, spread across 8 clusters (FIG. 45C). The phenotypic features of clusters were determined using gene signatures generally associated with various CD8⁺ T cell states, including those with naïve, memory, effector, and proliferative status (FIG. 45C). In this space, cells from convalescent patients that recognized different dominant epitopes were commonly associated with divergent phenotypes, as shown for representative epitopes in FIG. 45D. For example, T cells specific to QYIKWPWYI (SEQ ID NO: 318) in A*24 (QYI-A24) were clustered in regions with high effector scores while those specific for PTDNYITTY (SEQ ID NO: 327) in A*01 (PTD-A01) and LLYDANYFL (SEQ ID NO: 286) in A*02 (LLY-A02) resided at opposite ends of memory-rich regions. Thus, and as will be further detailed below, the different immunoreactive epitopes of SARS-CoV-2 elicit distinct CD8⁺ T cell phenotypes.

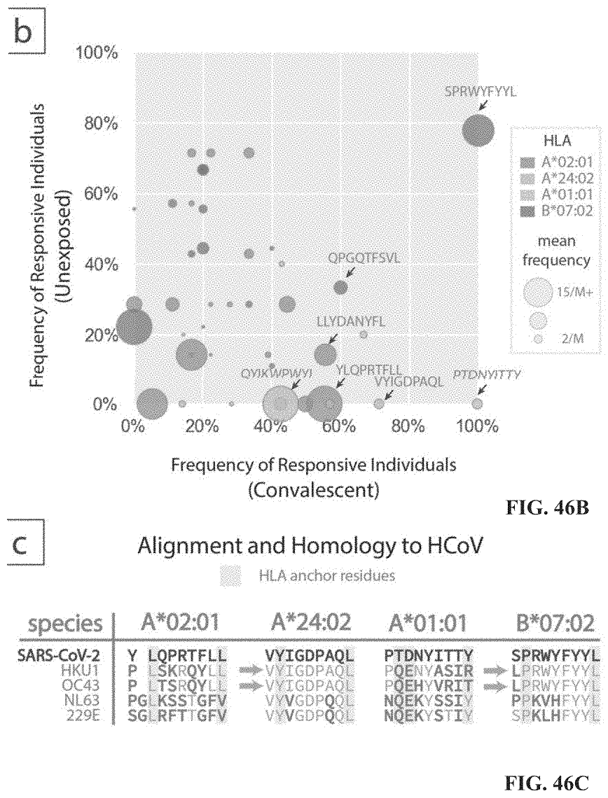

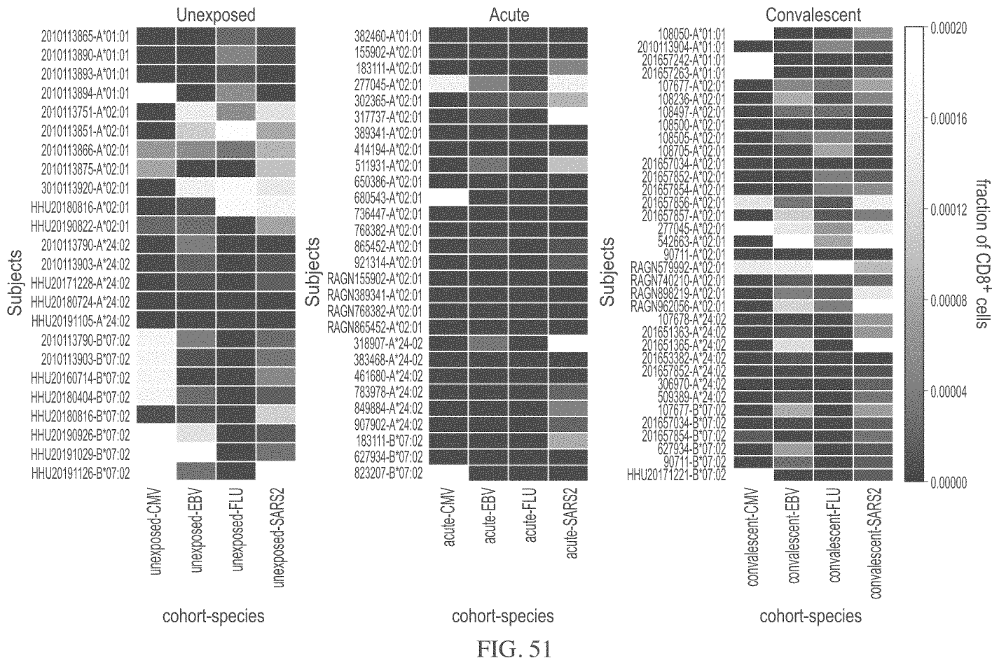

Evolution of immunoreactivity through COVID-19 disease progression: Having established a broad landscape of SARS-CoV-2-reactive CD8⁺ T cells, a study was designed to determine how TCR repertoires evolve over the course of infection and recovery. As this approach does not require cell expansion to determine TCR specificity, it was possible to directly quantify the frequency of epitope-specific CD8⁺ T cells in the blood of convalescent, acute, and unexposed individuals. FIG. 46A shows the frequency, for each subject, of T cells reactive to the top five epitopes detected across each of the four HLA variants analyzed. Notably, markedly fewer SARS-CoV-2-specific T cells were observed in patients with acute disease compared to those in convalescence (p=6.0e-5 for A*02, Wilcoxon rank sum); dramatic reduction also applied to memory T cells from prior antiviral responses in these patients, including influenza and EBV, but potentially less to the CMV-specific pool in multiple acute subjects (FIG. 51). The paucity of virus reactive T cells is consistent with the severe T cell lymphopenia that has previously been reported to occur in patients with acute COVID-19 (Huang et al. (2020) Lancet 395:497-506; Chen et al. (2020) J Clin Invest 130:2620-2629).

The frequencies of SARS-CoV-2-specific T cells in unexposed individuals varied markedly with the HLA allele (FIG. 46A). While several dominant epitopes in HLA-A*02, A*24, and A*01 were associated with high-frequency responses in >40% of convalescent subjects (FIG. 46B) the depth of the overall response was significantly lower in unexposed compared to convalescent subjects (p=2.3e-5, 2.2e-4, 1.1e-6 by by Wolcoxon rank sum, respectively). In stark contrast, there was no discernible difference in response frequency detected across the most immunodominant epitopes in B*07:02 indivisuals (p=0.2). In fact, CD8⁺ T cells recognizing nucleocapsid-derived SPRWYFYYL (SEQ ID NO: 323) in B*07 (SPR-B07) were found in almost over 80% of unexposed subjects with a mean frequency of 4 cells/M cells screened (FIG. 46B), presaging the immunodominance of this epitope in convalescent COVID-19 patients, where reactivity was detected in >100% of the samples.

The broad presence of SARS-CoV-2-specific T cells in unexposed B*07 subjects could originate from fortuitous cross-reactivity of a public specificity, or from priming via previous exposure to a highly related endemic human coronavirus (HCoV). Indeed, SPR-B07 shows marked homology to the corresponding segments of the nucleocapsid proteins from multiple prevalent HCoVs, including HKU1 and OC43, with only a single amino acid residue mismatched at the N-terminus (FIG. 46C). The nature of the homology preserves internal TCR-contact residues as well as the P and L anchors for HLA binding in peptide positions 2 and 9. Accordingly, the HCoV epitope (LPR-B07) is predicted to bind with high affinity to HLA-B*07 and could reasonably be expected to cross-react with SPR-B07-specific TCRs. Broader sequence alignment with HCoVs revealed very little homology to the immunodominant epitopes of A*02 and A*01, but did identify a perfect match to VYIGDPAQL (SEQ ID NO: 317) for A*24 (VYI-A24). Surprisingly, T cell specificity to VYI-A24 was not detected in a single unexposed subject. This likely reflects the lower frequency of response elicited by this epitope or an insufficient commitment to memory following exposure to HCoVs. Overall, it was found that the response to SARS-CoV-2 is sharply distinguished by HLA genotype, as can be seen clearly in the case of A*02 and B*07, where it appears that highly specific CD8⁺ responses are either generated de novo or amplified from an abundant pre-existing pool, respectively.

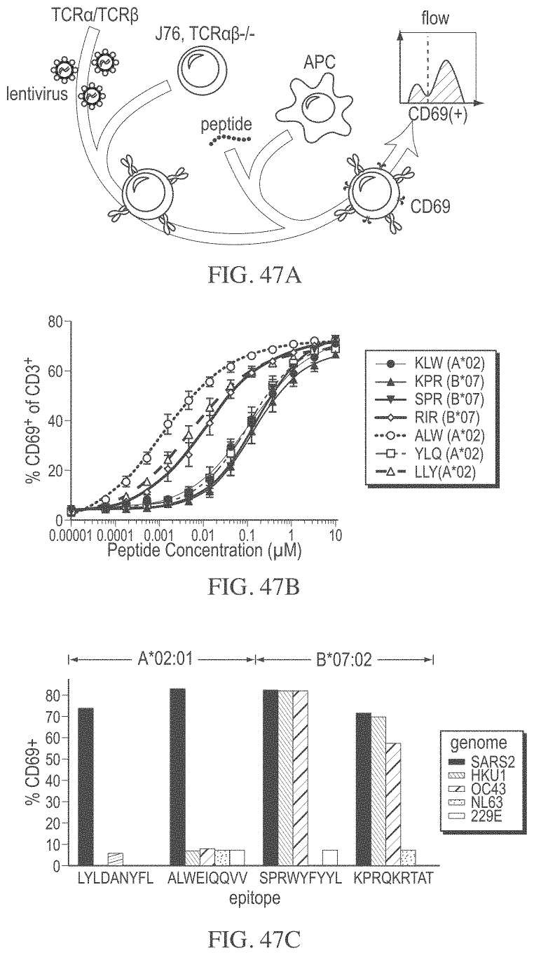

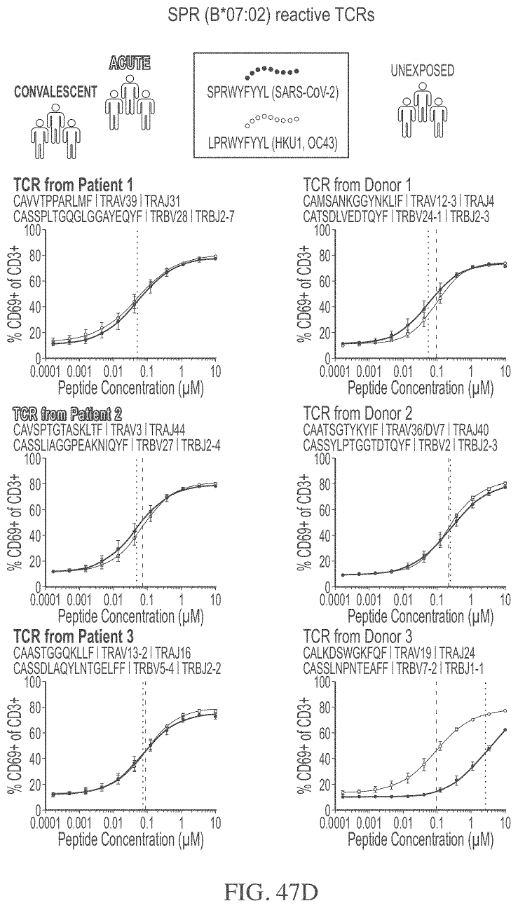

Functional reactivity and cross-reactivity of SARS-CoV-2-specific clonotypes: To confirm the specificity and functionality of TCR-peptide-HLA interactions identified in this study, several of the discovered a/b TCRs clonotypes were cloned and expressed in a TCR-null Jurkat J76 cell line (Jutz et al. (2016) J Immunol Methods 430:10-20). Activation of these transductants upon stimulation by SARS-CoV-2 peptides, presented by an HLA-matched lymphoblast T cell line, was evaluated by measuring the induction of surface CD69 (FIG. 47A). Altogether, 28 interactions were observed for epitopes derived from Orflab, Spike, Nucleocapsid, Membrane, and ORF3a proteins, spanning high confidence interactions observed across multiple cells as well as interactions observed exclusively in a single cell. Dose-response curves for a subset of interactions in A*02 and B*07 are shown in FIG. 47B. The EC50s measured for these interactions ranged from 1 to 100 nM, with no particular relationship to epitope immunodominance or clonotype frequency measured ex vivo from the respective subject. These values are consistent with interactions measured for CMV-specific epitopes in A*02 using the same system. The recombinant TCR expressing rTCR cell lines were used to compare the functional reactivity elicited by homologous epitopes from HCoVs (FIG. 47C). Activation was insignificant for the closest homologs of Orf3a-derived LLY-A02 and Orflab-derived ALW-A02, all of which actually originated from HCoV spike proteins. In contrast, HKU1 and OC43 homologs of nucleocapsid-derived SPR-B07 and KPR-B07 epitopes drove substantial T cell activation (FIG. 47C).

The sensitivity of B*07 interactions was assessed, comparing the reactivity of SPR-B07-specific clonotypes identified from COVID-19 patients or unexposed subjects to SARS-CoV-2-derived SPR-B07 or HCoV-derived LPR-B07 (FIG. 47D). The three TCRs identified from COVID-19 individuals yielded EC50s that were essentially identical for the two epitopes, all falling between 50-100 nM (FIG. 47D, left). Two of the TCRs from unexposed individuals yielded EC50s in the same range, again comparable for the HCoV and SARS-CoV-2 variants, while a third showed a >10-fold preference for the HCoV epitope (even though it was originally detected as binding to the SARS-CoV-2 peptide). Aside from providing validation that the specificities detected in the barcoded tetramer technology indeed correspond to antigen-reactive T cells, these findings support that the homologies between SARS-CoV-2 and HCoV epitopes are functionally relevant, and that pre-existing cellular reactivity to SARS-CoV-2 in B*07 subjects likely results from previous exposure to HCoVs like HKU1 or OC43.

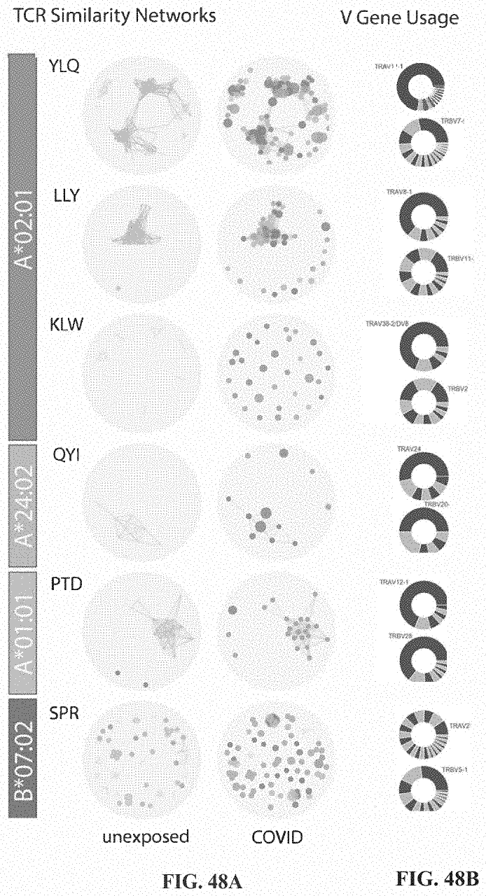

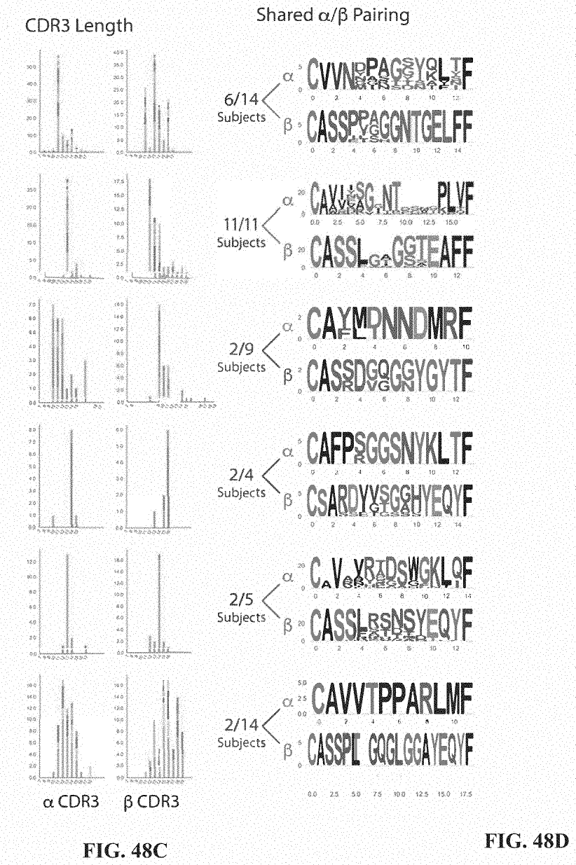

HLA Restricted Epitopes Impact V(D)J Gene Usage: Given the comprehensive landscape of TCR specificity determined with this approach, a study was designed to elucidate the extent to which TCR usage is shared within and across subjects. The linkage between paired TCR α/β sequences and their epitope specificity was examined to determine if any features are implicated in the CD8⁺ T cell response to SARS-CoV-2. TCRs from 2,469 SARS-CoV-2-specific T cells were used to perform network mapping of epitope-specific subsets across several immunodominant epitopes identified (FIG. 48A). Importantly, because it is known that during development a TCR β-chain can be paired with many different α-chains, the network analysis allowed clonotype linkages with α or β CDR3 sequences (indicated by edges), identifying conserved motifs based on physicochemical similarity (via BLOSUM matrices) within in the epitope specific T cell population (Dash et al. (2017) Nature 547:89-93). T cells from COVID-19 patients that recognize the most dominant A*02-, A*24-, and A*01-restricted epitopes, which have no counterpart in unexposed repertoires, showed a high degree of motif sharing with the exception of KLW-A02 (FIG. 48A). Interestingly, all of these epitopes, including KLW-A02, show dominant usage of a single TCR alpha variable (TRAV) region, and in the cases of QYI-A24 and PTD-A01, dominant usage both TRAV and TCR beta variable (TRBV) regions (FIG. 48B). In marked contrast, SPR-B07-specific T cells, including those that also recognize homologs from HCoV, were far more diverse in CDR3 across subjects (FIG. 48A), also using 8 TRAV and 3 TRBV regions to cover 50% of the clonotypes represented. Interestingly, two instances of CDR3 homology shared across cohorts was observed, as indicated by the presence of nodes with unconnected edges, which are represented in both network maps.

These comparisons show that the reactivities that appear during SARS-CoV-2 infection may stem from both the amplification of highly related TCRs, or from the usage of diverse pre-existing T cell populations. This conclusion extended to CDR3 lengths (FIG. 48C), which were tightly distributed for a and/or β-chains in T cells reactive to the top epitopes in A*02, A*24, and A*01, but significantly less so for SPR-B07. To further understand the extent of the public nature of paired a/(3 TCR usage in COVID-19, consensus sequences were generated from select interconnected network clusters (FIG. 48D). This representation provides insight into a/b linkage in the context of public responses, that cannot be afforded by bulk sequencing approaches. Most motifs were represented by multiple sequences and shared by 50% or of the subjects studied, with the exception of KLW-A02 that was shared across only 22%, and SPR-B07 that was shared across only 14%, notably with identical α/β sequences (FIG. 48D). Thus, divergent TCR repertoire utilization, conditioned by HLA and the presence of diverse, pre-existing reactivity resulting from prior viral exposure was observed.

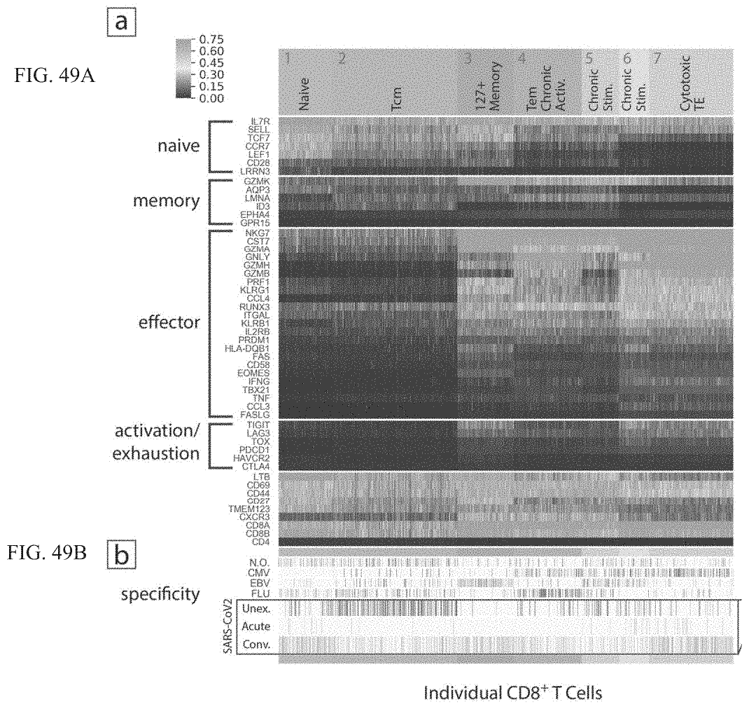

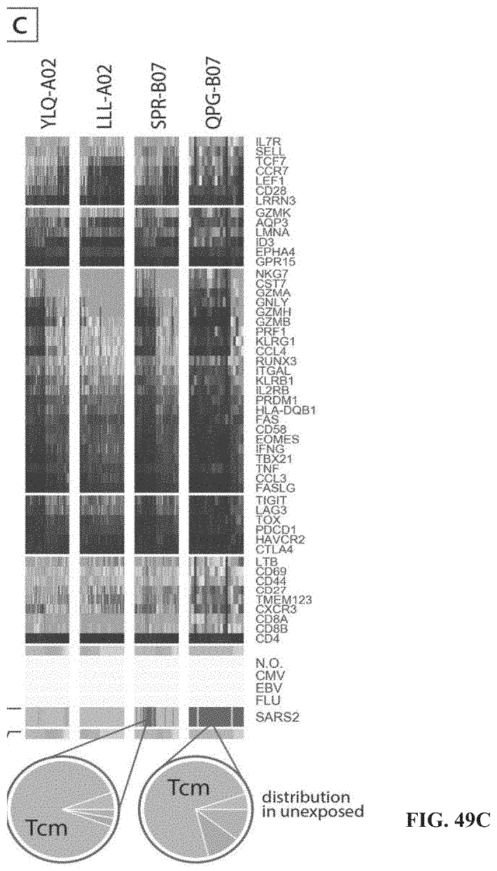

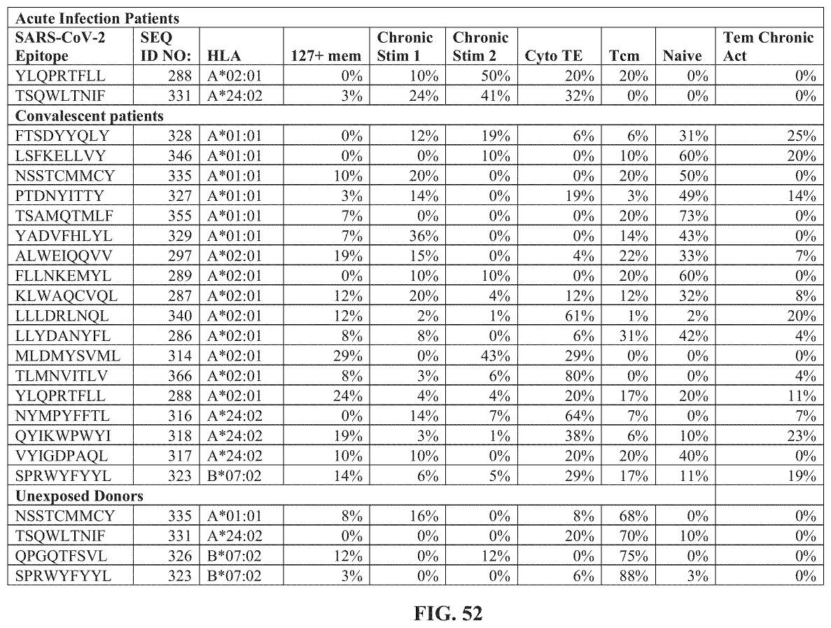

CD8+ Memory T cell Phenotypes vary with recognition of SARS-CoV-2 epitopes in COVID-19: To examine how CD8+ T cell phenotype varied in relation to disease status, HLA epitope specificity, and TCR diversity, a more detailed analysis of the single-cell transcriptomic data was performed. As an internal reference, the transcriptomic phenotype of T cells reactive to common acute and latent infections, including influenza, EBV, and CMV was used. To relate these data to existing knowledge on differentially-expressed genes that delineate CD8⁺ T subsets, supervised partition clustering based on imputed expression of a set of 51 curated transcripts characteristic of naïve, memory, effector, or chronically-activated/exhausted populations was used (FIG. 49A and FIG. 52). This resulted in the identification of seven distinct T cell clusters. Some were easily assigned (naïve cells in C1, central memory in C2, and fully activated cytotoxic effectors in C7). Other memory/effector intermediates were more tentatively labeled, as they did not easily fit into existing categorizations (Szabo et al. (2019) Nat Commun 10:4706; Monaco et al. (2019) Cell Rep 26:1627-1640 e1627). These included a puzzling population (C3, here “CD127+ Memory”), which expresses markers of naïve, memory and effector cells, and 3 other clusters with characteristics of memory or chronically activated cells (C4-6).

SARS-CoV-2 specific T cells were found in all clusters (FIG. 49B, bottom), but at proportions that varied with stage of disease and epitope specificity. Cells from acute patients predominantly showed full effector phenotypes, but also paradoxically naïve types. In convalescent donors, T cells from several epitope specificities were broadly distributed, consistent with the resolution of an infection. Several, epitope-specific T cell pools were predominantly found in central memory (C2), including PTD-A01 (49%) and LLY-A02 (42%), while others predominantly resided in cytotoxic terminal effector (C7) including TLM-A02 (80%), and LLL-A02 (61%). In most other reactivities, including SPR-B07, transcriptional profiles in convalescent patients were fairly broadly distributed across all clusters. In contrast, the reactivity in unexposed subjects was dominated by the central memory pool, confirming that the CD8+ cells likely result from long-term exposure to cross-reactive antigens. This was especially clear in the case of B*07, where epitope-specific T cells for SPR-B07, QPG-B07, and SII-B07 were represented in central memory (C2) at proportions of 88%, 75%, and 67%, respectively. Other notable reactivities associated with central memory include TSQ-A24 (70%) and NSS-A01 (68%), though the source of these memory cells, like QPG-B07 and SII-B07, does not appear to be from HCoV exposure based on lack a of homology. Overall, this analysis provides further evidence that SPR-B07 responses to SARS-CoV-2 are likely drawn from a pre-existing memory pool and that commitment to different T cell fate is dependent on epitope specificity.

Some interesting dynamics were observed between SARS-CoV-2 infection and existing T cell pools specific for common viral infections, with differentiated outcomes likely shaped by exposure history (FIG. 49B). Influenza-specific cells CD8+ T cells, which result from vaccination or past infections, mapped primarily to the central memory (C1) and effector memory (C3) compartments in unexposed individuals. Proportions were stable across epitope specificities in COVID patients with the exception of GIL-A02, where the proportion of effector memory cells decreased from 50% to 0%, and a naïve population representing 30% of the cells paradoxically emerged. CMV- and EBV-specific cells, likely subject to more chronic stimulation from low-level re-activation of these integrated herpesviruses, mapped to more activated pools in unexposed subjects, as has been described by others (van den Berg et al. (2019) Med Microbiol Immunol 208:365-373). After SARS-CoV-2 infection, EBV-specific cells shifted markedly from central memory (C2) and chronically stimulated compartments (C5) into the 127+ memory cluster (C3). These changes may reflect either bystander activation, perhaps as a result of the high cytokine release in COVID-19 patients, or from changes in homing or recirculation patterns that bring into the blood cells normally sequestered in tissues. These observations suggest that, in addition to inducing lymphopenia, COVID-19 strongly reshuffles third-party antiviral T cell pools, the extent of which may be associated with exposure history, and at least to some degree epitope specificity.

### Discussion

This Example demonstrates a unified description of the CD8⁺ T cell response to SARS-CoV-2, highlighting the importance of HLA genetics, TCR repertoire diversity, and epitope-specific navigation through a complex transcriptomic phenotype at various stages of disease. In building a comprehensive map of immunodominant, HLA-restricted epitopes broadly derived from proteins across the entire SARS-CoV-2 proteome, only some HLA haplotypes are associated with the existence of a pre-existing CD8+ T cell memory pool in unexposed individuals. HLA variation plays an important role in shaping the diversity of CD8⁺ T cell repertoires upon exposure to SARS-CoV-2, and that cellular phenotype and commitment to memory can be associated with epitope-specificity in the context of both SARS-CoV-2 and latent EBV infections.

The presence of SARS-CoV-2 reactive CD8⁺ T cells has been linked to milder disease (Rydyznski Moderbacher et al. (2020) Cell 183:996-1012 e1019; Sekine et al. (2020) Cell 183:158-168 e114; Schulien et al. (2021) Nat Med 27:78-85), although, the precise link between cellular immunity and host protection still remains to be further understood (Addetia et al. (2020) J Clin Microbiol 58; Fontanet and Cauchemez (2020) Nat Rev Immunol 20:583-584). Individuals carrying HLA-B*07 were found to show a CD8⁺ T cell response that is dominated by pre-existing memory pools reactive to multiple SARS-CoV-2 epitopes, especially SPR-B07, which is likely induced by previous exposures to benign HCoVs. In contrast, the immunodominant responses in A*02 individuals (e.g., to YLQ-A02, LLY-A02) are driven largely by the expansion of antigen-inexperienced SARS-CoV-2-specific T cells. It is possible that CD8⁺ T cell cross reactivity may be less widespread in unexposed individuals than for CD4⁺ T cross-reactivity, for which ˜50% of unexposed individuals exhibited CD4+ T cell memory (Grifoni et al. (2020) Cell 181:1489-1501 e1415). This data provides a basis for this limited representation of the CD8⁺ T cell repertoire in that only a subpopulation of individuals carrying a specific HLA allele would have these pre-existing memory CD8⁺ T cells.

The interplay between HLA-restricted epitope presentation and available TCR repertoire shapes the cellular response to SARS-CoV-2. There are few limited studies suggesting an influence of HLA genotype on COVID-19 severity (Shkurnikov et al. (2021) Front Immunol 12:641900; Liang et al. (2021) Int J Mol Sci 22:2), an epidemiologic study from Italy reported that HLA-B*44 and HLA-C*01 positive individuals are more susceptible to SARS-CoV-2 when compared with other HLA alleles including HLA-A*25 and HLA-B*08 (Correale et al. (2020) Int J Mol Sci 21:5205). Large-scale, high-resolution HLA mapping, consistent with what was done for select HLAs in this work, may help identify relationships between HLA genotype and protection against severe disease, ideally uncovering mechanism. Here, an interesting connection was observed between TCR repertoire diversity and HLA restriction. Responses seen in A*02, A*24, and A*01 were more often associated with “public” CDR3 motifs and consistent V gene segment usage in α and/or β-chains. In contrast, the dominant immune response in B*07 leveraged a significantly more diverse TCR repertoire.

The results from this study show that in the case of COVID-19, the largest pool of potentially protective, pre-existing cellular immunity is derived from one of the least public epitope-specific repertoires, possibly reflecting the influence of repeated acute infections with HCoVs throughout the life of the individuals.

Beyond the comprehensive deciphering of TCR specificity reported here, a detailed picture of the complex and dynamic transcriptional landscape of the CD8⁺ T response to SARS-CoV-2 has been provided. Importantly, it has been demonstrated that the pre-existing SPR-B07 reactivity, observed in ˜80% of unexposed subjects with HLA-B*07, was predominantly associated with a central memory-like transcriptional profile (88%), confirming that it originates from prior exposures. In convalescent patients, it was observed that a much broader distribution of SPR-B07-reactive T cells spanning every functional state at proportions ranging from 5-29%. This is consistent with late contraction/early memory formation described for SARS-CoV-2 in a recent study (Schulien et al. (2021) Nat Med 27:78-85), where cells spanned naïve, central memory, various classifications of effector memory, and terminally differentiated effector memory expressing RA (TEMRA). There was no evidence for a particularly frequent “exhausted” state among SARS-CoV-2-specific CD8+ T cells, as suggested elsewhere (Zheng et al. (2020) Cell Mol Immunol 17:541-543; Diao et al. (2020) Front Immunol 11:827)(acknowledging that the phenotypic state is a proxy for true reactivity testing, and that blood T cells may not fully reflect what happens in the lung). No evidence of “antigenic sin” resulting from HCoV pre-exposure (Brown and Essigmann (2021) mSphere 6:e00056-21) was found that could stifle an effective response to SARS-CoV-2-unexposed B*07 individuals. It will be interesting to determine whether HLA haplotype plays a role in the durability of the CD8+ T cell responses, especially to SARS-CoV-2 vaccines, which may have profound impact for long term protection across different ethnic groups and geographic regions.

Another interesting observation from this work, is that even at the height of infection or shortly after viral clearance, the cumulative anti-SARS-CoV-2 CD8⁺ T cell response barely reached the frequency of anti-influenza memory responses and was well below the frequencies that could be achieved by CMV-specific cells in the same individuals (FIG. 51). This was particularly evident in the acutely infected individuals, at a time where the contribution of cytotoxic CD8⁺ T cells would have been most important.

In conclusion, this study leveraged a powerful single-cell technology to better elucidate the roles of HLA variation, TCR diversity, and cellular phenotypes in establishing pre-existing immunity to SARS-CoV-2. The presence of a diverse and immune dominant nucleocapsid epitope-specific memory pool was observed in subjects with HLA-B*07 but little evidence was seen of similar reactivity in individuals with other HLA alleles.

### INCORPORATION BY REFERENCE

Each patent, publication, and non-patent literature cited in the application, and Obermair et al. (2021) bioRxiv posted Mar. 4, 2021 under doi.org/10.1101/2021.03.02.433522 and Pregibon et al. (2021) bioRxiv posted Apr. 29, 2021 under doi.org/10.1101/2021.04.29.441258, is hereby incorporated by reference in its entirety as if each was incorporated by reference individually.

1. An isolated peptide comprising an immunodominant SARS-CoV-2 T cell epitope comprising an amino acid sequence selected from the group consisting of SEQ ID NOs: 286, 288, 324, 326, 327, and 328, wherein the peptide is no more than 100 amino acids in length, and an optional pharmaceutically acceptable carrier.

2. The isolated peptide of claim 1, wherein the T cell epitope is a CD8+ epitope.

3-11. (canceled)

12. The isolated peptide of claim 1, wherein the peptide is no more than 20 amino acids in length.

13. The isolated peptide of claim 1, wherein the amino acid sequence of the peptide consists essentially of or consists of an amino acid sequence selected from the group consisting of SEQ ID NOs: 286, 288, 324, 326, 327, and 328.

14. The isolated peptide of claim 1, wherein the peptide is presentable by a major histocompatibility complex (MHC) Class I.

15. (canceled)

16. The isolated peptide of claim 1, where the peptide is synthetic.

17. (canceled)

18. A pharmaceutical composition comprising one or more peptides of claim 1 and a pharmaceutically acceptable carrier or excipient.

19. (canceled)

20. A pharmaceutical composition comprising one or more nucleic acids encoding one or more peptides of claim 1, and a pharmaceutically acceptable carrier or excipient.

21. The pharmaceutical composition of claim 18, further comprising a liposome or a lipid nanoparticle, wherein the one or more peptides are disposed within the liposome or the lipid nanoparticle.

22. (canceled)

23. The pharmaceutical composition of claim 18, further comprising an immunogenicity enhancing adjuvant.

24. The pharmaceutical composition of claim 20, wherein the one or more nucleic acids are synthetic.

25. A vaccine comprising the pharmaceutical composition of claim 18, wherein the vaccine stimulates a T cell mediated immune response when administered to a subject.

26. The vaccine of claim 25, wherein the vaccine is a priming vaccine and/or a booster vaccine.

27. The vaccine of claim 25, wherein the vaccine is a pan-coronavirus vaccine.

28. (canceled)

29. A method of stimulating a T cell immune response to SARS-CoV-2 in a subject, the method comprising administering to the subject an effective amount of the pharmaceutical composition of claim 18.

30. The method of claim 29, wherein the subject expresses an MHC Class I that binds the epitope.

31-42. (canceled)

43. A method of presenting a T cell epitope on the surface of an APC, the method comprising contacting the APC ex vivo with the peptide of claim 14, wherein the APC expresses the MHC Class I.

44. A method of presenting a T cell epitope on the surface of an APC, the method comprising transfecting the APC ex vivo with a nucleic acid encoding the peptide of claim 14, wherein the APC expresses the MHC Class I.

45. The method of claim 44, wherein the nucleic acid comprises an mRNA.

46. A composition comprising an isolated APC that expresses an MHC Class I and presents on an outer cell surface of the APC the peptide of claim 1, and an optional pharmaceutically acceptable carrier.

47. The composition of claim 46, wherein the APC is a dendritic cell, monocyte, macrophage, B cell or an artificial APC.

48. (canceled)

49. A method of producing activated T cells, the method comprising contacting a population of T cells in vitro with the composition of claim 46 to permit activation of one or more T cells in the population for reactivity to a SARS-CoV-2 infected cell, wherein the T cells comprise CD8+ T cells.

50-52. (canceled)

53. A method of stimulating a T cell immune response to SARS-CoV-2 in a subject, the method comprising administering to the subject a composition comprising a population of activated T cells produced by the method of claim 49, wherein the subject expresses the MHC Class I.

54. The composition of claim 46, wherein:
(a) the peptide comprises the amino acid sequence of SEQ ID NO: 328, and the MHC Class I is HLA-A*01:01;
(b) the peptide comprises the amino acid sequence of SEQ ID NO: 286, and the MHC Class I is HLA-A*02:01;
(c) the peptide comprises the amino acid sequence of SEQ ID NO: 327, and the MHC Class I is HLA-A*01:01;
(d) the peptide comprises the amino acid sequence of SEQ ID NO: 326, and the MHC Class I is HLA-B*07:02;
(e) the peptide comprises the amino acid sequence of SEQ ID NO: 324, and the MHC Class I is HLA-B*07:02; and/or
(f) the peptide comprises the amino acid sequence of SEQ ID NO: 288, and the MHC Class I is HLA-A*02:01.

55-56. (canceled)

57. The method of claim 25, wherein the subject is at risk of infection by SARS-CoV-2.

58-73. (canceled)

74. A composition comprising an isolated T cell that binds the peptide of claim 1, and an optional pharmaceutically acceptable carrier.

75-78. (canceled)

79. The composition of claim 74, wherein the T cell is a CD8+ T cell.

80-89. (canceled)

90. An engineered T cell receptor (TCR) having antigenic specificity for a SARS-CoV-2 antigen, the TCR have an alpha chain and a beta chain, wherein:
(a) the SARS-CoV-2 antigen comprises a T cell epitope comprising the amino acid sequence of SEQ ID NO: 286 presented by HLA-A*02:01, and the TCR comprises the CDR3 alpha and CDR3 beta sequences of TCR_22, TCR_27, TCR_47, TCR_65, TCR_69, TCR_77, TCR_84, or TCR_107 set forth in Table 5;
(b) the SARS-CoV-2 antigen comprises a T cell epitope comprising the amino acid sequence of SEQ ID NO: 288 presented by HLA-A*02:01, and the TCR comprises the CDR3 alpha and CDR3 beta sequences of TCR_5, TCR_7, TCR_14, TCR_16, TCR_20, TCR_28, TCR_33, TCR_43, TCR_45, TCR_63, TCR_70, TCR_81, TCR_86, TCR_88, TCR_90, TCR_94, TCR_98, TCR_99, TCR_102, TCR_103, TCR_106, TCR_108, TCR_113, or TCR_123 set forth in Table 5;
(c) the SARS-CoV-2 antigen comprises a T cell epitope comprising the amino acid sequence of SEQ ID NO: 324 presented by HLA-B*07:02, and the TCR comprises the CDR3 alpha and CDR3 beta sequences of TCR_16, TCR_22, TCR_27, TCR_65, TCR_97, or TCR_107 set forth in Table 5;
(d) the SARS-CoV-2 antigen comprises a T cell epitope comprising the amino acid sequence of SEQ ID NO: 326 presented by HLA-B*07:02, and the TCR comprises the CDR3 alpha and CDR3 beta sequences of TCR_11, TCR_112, or TCR_122, set forth in Table 5;
(e) the SARS-CoV-2 antigen comprises a T cell epitope comprising the amino acid sequence of SEQ ID NO: 327 presented by HLA-A*01:01, and the TCR comprises the CDR3 alpha and CDR3 beta sequences of TCR_26, TCR_53, or TCR_54 set forth in Table 5; or
(f) the SARS-CoV-2 antigen comprises a T cell epitope comprising the amino acid sequence of SEQ ID NO: 328 presented by HLA-A*01:01, and the TCR comprises the CDR3 alpha and CDR3 beta sequences of TCR_25 or TCR_41 set forth in Table 5.

91-101. (canceled)

102. A pharmaceutical composition comprising an engineered T cell and a pharmaceutically acceptable carrier, wherein the engineered T cell comprises one or more exogenous nucleic acid sequences that encode a TCR having antigenic specificity for a SARS-CoV-2 antigen, the TCR have an alpha chain and a beta chain, wherein:
(a) the SARS-CoV-2 antigen comprises a T cell epitope comprising the amino acid sequence of SEQ ID NO: 286 presented by HLA-A*02:01, and the TCR comprises the CDR3 alpha and CDR3 beta sequences of TCR_22, TCR_27, TCR_47, TCR_65, TCR_69, TCR_77, TCR_84, or TCR_107 set forth in Table 5;
(b) the SARS-CoV-2 antigen comprises a T cell epitope comprising the amino acid sequence of SEQ ID NO: 288 presented by HLA-A*02:01, and the TCR comprises the CDR3 alpha and CDR3 beta sequences of TCR_5, TCR_7, TCR_14, TCR_16, TCR_20, TCR_28, TCR_33, TCR_43, TCR_45, TCR_63, TCR_70, TCR_81, TCR_86, TCR_88, TCR_90, TCR_94, TCR_98, TCR_99, TCR_102, TCR_103, TCR_106, TCR_108, TCR_113, or TCR_123 set forth in Table 5;
(c) the SARS-CoV-2 antigen comprises a T cell epitope comprising the amino acid sequence of SEQ ID NO: 324 presented by HLA-B*07:02, and the TCR comprises the CDR3 alpha and CDR3 beta sequences of TCR_16, TCR_22, TCR_27, TCR_65, TCR_97, or TCR_107 set forth in Table 5;
(d) the SARS-CoV-2 antigen comprises a T cell epitope comprising the amino acid sequence of SEQ ID NO: 326 presented by HLA-B*07:02, and the TCR comprises the CDR3 alpha and CDR3 beta sequences of TCR_11, TCR_112, or TCR_122, set forth in Table 5;
(e) the SARS-CoV-2 antigen comprises a T cell epitope comprising the amino acid sequence of SEQ ID NO: 327 presented by HLA-A*01:01, and the TCR comprises the CDR3 alpha and CDR3 beta sequences of TCR_26, TCR_53, or TCR_54 set forth in Table 5; or
(f) the SARS-CoV-2 antigen comprises a T cell epitope comprising the amino acid sequence of SEQ ID NO: 328 presented by HLA-A*01:01, and the TCR comprises the CDR3 alpha and CDR3 beta sequences of TCR_25 or TCR_41 set forth in Table 5.

103-107. (canceled)

108. A method of ameliorating a symptom of SARS-CoV-2 infection in a subject in need thereof, the method comprising administering to the subject an effective amount of the pharmaceutical composition of claim 102, thereby to ameliorate the symptom.

109-128. (canceled)
